(19) United States
(12) Patent Application Publication
(10) Pub. No.: US 2015/0031674 A1
(43) Pub. Date: Jan. 29, 2015

(54) SERINE/THREONINE KINASE INHIBITORS

(71) Applicant: GENENTECH, INC., South San Francisco, CA (US)

(72) Inventors: Joachim Rudolph, Burlingame, CA (US); Lewis J. Gazzard, Belmont, CA (US); James J. Crawford, San Francisco, CA (US); Chudi Ndubaku, San Francisco, CA (US); Joy Drobnick, San Francisco, CA (US); Wendy Lee, San Ramon, CA (US)

(73) Assignee: GENENTECH, INC., South San Francisco, CA (US)

(21) Appl. No.: 14/340,792

(22) Filed: Jul. 25, 2014

Related U.S. Application Data
(60) Provisional application No. 61/924,567, filed on Jan. 7, 2014.
(60) Provisional application No. 61/859,050, filed on Jul. 26, 2013.

(51) Int. Cl. C07D 471/04

(52) U.S. Cl. CPC C07D 471/04
USPC 514/210.21; 544/279; 514/255.05; 514/264.11; 514/252.16; 544/117; 514/234.2; 544/58.2; 514/228.5; 544/230; 544/238; 514/252.02

(57) ABSTRACT

Compounds having the formula I wherein R¹, R², R³, R⁴, R⁵, R^{a}, R^{b}, R^{c}, R^{d}, R^{e}, n, r, s and t are as defined herein and which compounds are inhibitors of PAK1. Also disclosed are compositions and methods for treating cancer and hyperproliferative disorders.

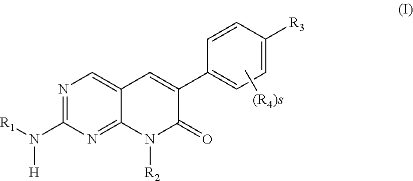

### CROSS REFERENCE TO PRIOR APPLICATIONS

This application claims the benefit of priority to U.S. Ser. No. 61/859,050 filed Jul. 26, 2013 and U.S. Ser. No. 61/924,597 filed Jan. 7, 2014 both of which are incorporated herein by reference in their entirety.

### FIELD OF THE INVENTION

The present invention relates to compounds which inhibit serine/threonine kinases and which are useful for treating hyperproliferative and neoplastic diseases by inhibiting signal transduction pathways which commonly are overactive or over-expressed in cancerous tissue. The present compounds are inhibitors of group 1 p21-activated protein kinases (PAK1, PAK2 and PAK3). The present invention further relates to methods for treating cancer or hyperproliferative diseases with compounds within the scope of the present invention.

### BACKGROUND OF THE INVENTION

Protein kinases are a family of enzymes that catalyze phosphorylation of the hydroxyl groups of specific tyrosine, serine, or threonine residues in proteins. Typically, such phosphorylation can dramatically change the function of the protein and thus protein kinases can be pivotal in the regulation of a wide variety of cellular process, including metabolism, cell proliferation, cell differentiation, and cell survival. The mechanism of these cellular processes provides a basis for targeting protein kinases to treat disease conditions resulting from or involving disorder of these cellular processes. Examples of such diseases include, but are not limited to, cancer and diabetes.

Protein kinases can be broken into two types, protein tyrosine kinases (PTKs) and serine-threonine kinases (STKs). Both PTKs and STKs can be receptor protein kinases or non-receptor protein kinases. PAK is a family of non-receptor STKs. The p21-activated protein kinase (PAK) family of serine/threonine protein kinases plays important roles in cytoskeletal organization, cellular morphogenesis, cellular processes and cell survival (Daniels et al., Trends Biochem. Sci. 1999 24: 350-355; Sells et al., Trends Cell. Biol. 1997 7:162-167). The PAK family consists of six members subdivided into two groups: PAK 1-3 (group I) and PAK 4-6 (group II) which are distinguished based upon sequence homologies and the presence of an autoinhibitory region in group I PAKs. p21-Activated kinases (PAKs) serve as important mediators of Rac and Cdc42 GTPase function as well as pathways required for Ras-driven tumorigenesis. (Manser et al., Nature 1994 367:40-46; B. Dummler et al., Cancer Metathesis Rev. 2009 28:51-63; R. Kumar et al., Nature Rev. Cancer 2006 6:459-473).

Changes in the levels and activities of group 1 PAKs in particular, are frequently associated with human malignancies including, but not limited to bladder carcinoma, breast carcinoma, colorectal carcinoma, gastric carcinoma, glioblastoma, hepatocellular carcinoma, ovarian carcinoma and renal cell carcinoma, primary breast adenocarcinoma, squamous non-small cell lung cancer or a squamous head and necks cancer. (J. V. Kichina et al., Expert. Opin. Ther. Targets 2010 14(7):703) PAK1 genomic amplification at 11q13 was prevalent in luminal breast cancer, and PAK1 protein expression was associated with lymph node metastasis. High expression of PAK2 in mammary invasive ductal carcinomas has been associated with increased survival and resistance of breast tumor cells to chemotherapeutic agents (X. Li et al., J. Biol. Chem. 2011 286(25):2291) Squamous non-small cell lung carcinomas (NSCLCs), and head and neck squamous carcinomas have aberrant cytoplasmic expression of PAK1. (C. C. Ong et al., Proc. Nat. Acad. Sci., USA 2011 108(17):7177) Group I PAKs contribute to squamous NSCLC cell motility, survival and proliferation (C. C. Ong et al., Oncotarget 2011 2(6):491) and PAK2 has been linked to mitosis completion in response to various cell stimuli (M. R. Banko et al., Mol. Cell. 2011, Nov. 30, 2011).

### SUMMARY OF THE INVENTION

There is a continuing need for new and novel therapeutic agents that can be used for cancer and hyperproliferative conditions. The PAK family members are important signaling proteins frequently over-expressed and/or overactive in many cancerous tissues. Design and development of new pharmaceutical compounds that inhibit or modulate their activity is essential. There is a continuing need for improved PAK inhibitors which exhibit selectivity for Group I vs. Group II PAKs and/or which selectively inhibit PAK in the presence of other kinases. In one aspect of the present invention there is provided a compound according to formula I wherein:

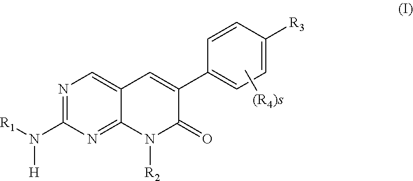

R¹ is H, C₁₋₃ alkyl, C₁₋₃ fluoroalkyl, oxetan-3-yl or 3-methyl-oxetan-3-yl;

R² is (i) C₂₋₆ hydroxyalkyl or C₂₋₆ dihydroxyalkyl, (ii) (alkylene)ₙNR^{a}R^{b} wherein the alkylene chain is optionally substituted by a hydroxyl, (iii) (alkylene)₁₋₃OR⁵ wherein R⁵ is (alkylene)₂₋₄NR^{a}R^{b}, (alkylene)₂₋₄OR^{f} or a heterocycle selected from azetidine, pyrrolidine, piperidine or azepine said heterocycle optionally substituted by C₁₋₆ alkyl, (iv) (alkylene)ᵣC(O)NR^{a}R^{b}, (v) (alkylene)ₙ-CN, (vi) (alkylene)ₜ(C₄₋₆-cycloalkyl NR^{c}R^{d}), (vii) heterocyclyl-(alkylene), wherein heterocyclyl refers to substituted azetidinyl, pyrrolidinyl, 4,4-difluoro-pyrrolidin-3-yl, piperidinyl, 3,3-difluoro-piperidin-3-yl, azepinyl, morpholinyl, 4-methylmorpholin-2-yl, 5-amino-1,3-dioxolan-2-yl, 5-methylamino-1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl, 5,9-dioxa-2-azaspiro[3.5]nonan-7-yl, 1,1-dioxo1-4-thiazinan-2-yl or piperazinyl said heterocyclyl moiety optionally substituted by C(═O)CHR^{f}NH₂, oxo, hydroxyl, amino, C₁₋₃ alkylamino, C₁₋₃ dialkylamino, cyano, oxetan-3-yl or C₁₋₆-hydroxyalkyl, (viii) (alkylene)ᵣS(O)₂R⁵ wherein R⁵ is (alkylene)₀₋₃NR^{a}R^{b} azetidinyl, pyrrolidinyl or piperidinyl, or (ix) 1-imino-1-oxa-thianyl-4-yl or 1,1-dioxidotetrahydro-2H-thiopyran-4-yl;

R^{a} and R^{b} are (a) independently in each occurrence hydrogen, C₁₋₃ alkyl, C₂₋₄-aminoalkyl, C₂₋₄-hydroxyalkyl, pyrrolidinyl, piperidinyl or azetidinyl, or, (b) R^{a} and R^{b} together with the nitrogen to which they are attached form a four to seven-membered mono- or bicyclic-ring optionally containing another heteroatom selected from O, NR^{e} or S(O)₀₋₂ which is optionally substituted by one or two hydroxyl or (CH₂)₀₋₂N(R)₂;

R^{e} and R^{d} are independently in each occurrence hydrogen, C₁₋₃ alkyl or oxetanyl;

R^{e} is hydrogen, C₁₋₃ alkyl or C₁₋₃ alkylsulfonyl;

R^{f} is hydrogen or C₁₋₃ alkyl;

R³ is heteroaryl wherein the heteroaryl ring is pyridine-2-yl, pyrazin-2-yl, thiazol-4-yl, thiazol-2-yl, pyrazol-3-yl, 1,2,4-triazol-2-yl or 1,2,4-oxadiazol-3-yl each optionally substituted by an R⁴ or a C₁₋₃ hydroxyalkyl moiety;

R⁴ is C₁₋₆ alkyl, cyclopropyl or halogen;

n is 2-6;

r is 1-6;

s is 0, 1 or 2;

t is 0-6; or,

a pharmaceutically acceptable salt thereof, with the proviso that the compound of formula I is not:
8-(trans-4-aminocyclohexyl)-6-[2-chloro-4-(6-methyl-2-pyrazinyl)phenyl]-2-(ethylamino)-pyrido[2,3-d]pyrimidin-7(8H)-one,
8-(trans-4-aminocyclohexyl)-6-[2-chloro-4-(6-methyl-2-pyrazinyl)phenyl]-2-[(1-methylethyl)amino]-pyrido[2,3-d]pyrimidin-7(8H)-one,
6-(2-chloro-4-(5-methyl-1,2,4-oxadiazol-3-yl)phenyl)-2-(ethylamino)-8-(piperidin-4-ylmethyl)pyrido[2,3-d]pyrimidin-7(8H)-one, or,
6-(2-chloro-4-(5-methyl-1,2,4-oxadiazol-3-yl)phenyl)-2-(isopropylamino)-8-(piperidin-4-ylmethyl)pyrido[2,3-d]pyrimidin-7(8H)-one.

Another aspect of the present invention relates to a method for treating a hyperproliferative disorder by administering a therapeutically effective quantity of a compound according to formula I to a patient in need thereof. Another aspect of the present invention relates to a method for treating cancer by administering a therapeutically effective quantity of a compound according to formula I to a patient in need thereof. The compound can be administered alone or co-administered with at least one other anti-hyperproliferative or chemotherapeutic compound.

Another aspect of the present invention relates to a method for inhibiting PAK activity in a cell comprising treating a cell with a compound according to formula I in an amount effective to attenuate or eliminate PAK activity.

Another aspect of the present invention relates to pharmaceutical compositions containing a compound of formula I with one or more pharmaceutically acceptable excipients, diluents and/or carriers.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “ [symbol] ” drawn through a bond each refer to the point of attachment of a functional group or other chemical moiety to the rest of the molecule of which it is a part. Thus, for example:

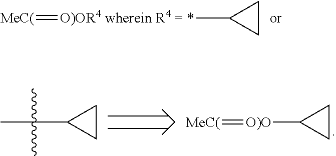

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

It will be appreciated by the skilled artisan that some of the compounds of formula I may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the skilled artisan that substitution of the tropane ring can be in either endo- or exo-configuration, and the present invention covers both configurations. The present invention includes all the individual stereoisomers (e.g. enantiomers), racemic mixtures or partially resolved mixtures of the compounds of formulae I and, where appropriate, the individual tautomeric forms thereof.

Compounds of formula I exhibit tautomerism. Tautomeric compounds can exist as two or more interconvertible species. Prototropic tautomers result from the migration of a covalently bonded hydrogen atom between two atoms. Tautomers generally exist in equilibrium and attempts to isolate an individual tautomer usually produces a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH-D-C(—OH)═CH—), amide/imidic acid (—C(═O)—NH-D-C(—OH)═N—) and amidine (—C(═NR)—NH-D-C(—NHR)═N—) tautomers. The latter two are particularly common in heteroaryl and heterocyclic rings and the present invention encompasses all tautomeric forms of the compounds.

The compounds of formula I may contain an acidic or basic center and suitable salts are formed from acids or bases may form non-toxic salts which have similar antiviral activity. Examples of salts of inorganic acids include the hydrochloride, hydrobromide, hydroiodide, chloride, bromide, iodide, sulfate, bisulfate, nitrate, phosphate, hydrogen phosphate. Examples of salts of organic acids include acetate, fumarate, pamoate, aspartate, besylate, carbonate, bicarbonate, camsylate, D and L-lactate, D and L-tartrate, esylate, mesylate, malonate, orotate, gluceptate, methylsulfate, stearate, glucuronate, 2-napsylate, tosylate, hibenzate, nicotinate, isethionate, malate, maleate, citrate, gluconate, succinate, saccharate, benzoate, esylate, and pamoate salts. For a review on suitable salts see Berge et al, J. Pharm. Sci., 1977 66:1-19 and G. S. Paulekuhn et al. J. Med. Chem. 2007 50:6665.

### DEFINITIONS

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001). The starting materials and reagents used in preparing these compounds generally are either available from commercial suppliers, such as Aldrich Chemical Co., or are prepared by methods known to those skilled in the art following procedures set forth in references. Materials, reagents and the like to which reference are made in the following description and examples are obtainable from commercial sources, unless otherwise noted. General synthetic procedures have been described in treatise such as Fieser and Fieser's Reagents for Organic Synthesis; Wiley & Sons: New York, Volumes 1-21; R. C. Larock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40 and will be familiar to those skilled in the art.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted by at least one substituent selected from the other specifically-named group. Thus, for example, “phenylalkyl” refers to an alkyl group having one to two phenyl substituents, and thus includes benzyl (phenylmethyl) and phenylethyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to a moiety that is either an aryl or a heteroaryl group.

The term “alkyl” as used herein without further limitation, alone or in combination with other groups, denotes an unbranched or branched chain, saturated, monovalent hydrocarbon residue containing 1 to 10 carbon atoms, for example, methyl, ethyl, propyl, isopropyl, n-butyl, iso-butyl, sec-butyl, or tert-butyl. The term “lower alkyl” denotes a straight or branched chain hydrocarbon residue containing 1 to 6 carbon atoms. “C₁₋₆ alkyl” as used herein refers to an alkyl composed of 1 to 6 carbons.

The term “cycloalkyl” denotes a monovalent saturated monocyclic or bicyclic hydrocarbon group of 3 to 10 ring carbon atoms, particularly a monovalent saturated monocyclic hydrocarbon group of 3 to 8 ring carbon atoms. Bicyclic means consisting of two saturated carbocycles having one (e.g., a spirocycle) two or more carbon atoms in common. For example, “C₃₋₇ cycloalkyl” as used herein refers to a cycloalkyl composed of 3 to 7 carbons in the carbocyclic ring. Examples for monocyclic cycloalkyl are cyclopropyl, cyclobutanyl, cyclopentyl, cyclohexyl or cycloheptyl. Examples for bicyclic cycloalkyl are bicyclo[2.2.1]heptanyl, or bicyclo[2.2.2]octanyl.

The term “hydroxyalkyl” denotes an alkyl group wherein at least one of the hydrogen atoms of the alkyl group has been replaced by a hydroxy group. Examples of hydroxyalky include hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl or 2-(hydroxymethyl)-3-hydroxypropyl.

The term “alkylene” as used herein denotes a divalent saturated linear hydrocarbon radical of 1 to 10 carbon atoms (e.g., (CH₂)ₙ) or a branched saturated divalent hydrocarbon radical of greater than 2 to 6 carbon atoms (e.g., —CHMe- or —CH₂CH(i-Pr)CH₂—), unless otherwise indicated. “C₀₋₄ alkylene” or “(CH₂)₄” refers to a linear or branched saturated divalent hydrocarbon radical comprising 1-4 carbon atoms or, in the case of C₀, the alkylene radical is omitted. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “(alkylene)ₜ (C₄₋₆-cycloalkyl NR^{c}R^{d})” as used herein refers to the radical R′R″—, wherein R′ is a cycloalkyl as defined herein substituted by NR^{c}R^{d}, and R″ is an alkylene radical as defined herein with the understanding that the attachment point of the aminocycloalkylalkyl moiety will be on the alkylene radical. Amino-C₃₋₇ cycloalkyl-C₁₋₃ alkyl refers to the radical R′R″ where R′ is amino substituted C₃₋₇ cycloalkyl and R″ is C₁₋₃ alkylene as defined herein. The moiety (alkylene), (C₄₋₆-cycloalkyl NR^{c}R^{d}) refers to (i).

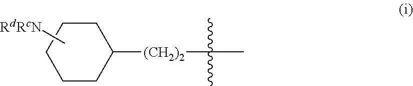

The term “(alkylene)ₙ-heterocyclyl” denotes the radical of the formula R′R″, wherein R′ is a heterocyclic radical as defined herein, and R″ is an alkylene radical as defined herein and the attachment point of the heterocycloalkyl radical will be on the alkylene radical. Optionally substituted heterocyclyl as used herein refers at least to the following moieties wherein 2 hydrogen atoms on a carbon are optionally replaced by an oxo moiety and R^{e} is hydrogen, C₁₋₃ alkyl or C₁₋₃ alkylsulfonyl:

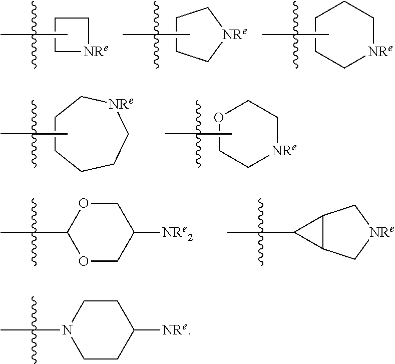

The terms “treat” and “treatment” refer to therapeutic treatment wherein the object is to slow down (lessen) an undesired physiological change or disorder, such as the spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include erlotinib (TARCEVA®, Genentech/OSI Pharm.), bortezomib (VELCADE®, Millennium Pharm.), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG 1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; (ix) anti-angiogenic agents such as bevacizumab (AVASTIN®), Genentech); and (x) pharmaceutically acceptable salts, acids and derivatives of any of the above.

### EMBODIMENTS

In one embodiment of the present invention there is provided a compound according to formula I wherein R¹, R², R³, R⁴, R⁵, R^{a}, R^{b}, R^{c}, R^{d}, R^{e}, n, r, s and t are as defined hereinabove.

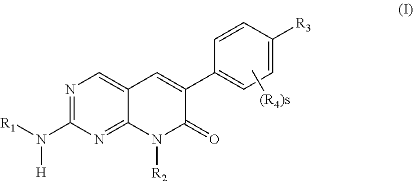

In one embodiment of the present invention there is provided a compound according to formula I wherein R¹ is optionally substituted C₁₋₃ alkyl, R² is (CH₂)ₙNR^{a}R^{b}; R³ is pyridinyl or pyrazinyl optionally substituted by methyl; R^{a} and R^{b} are (a) independently hydrogen or C₁₋₃ alkyl, or, (b) R^{a} and R^{b} together with the nitrogen to which they are attached form a four to seven-membered ring optionally containing another heteroatom selected from O, NR^{e} or S(O)₀₋₂; n is 2-6. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In another subembodiment of the present invention there is provided a compound according to formula I wherein R¹ is optionally substituted C₁₋₃ alkyl, R² is (CH₂)ₙNR^{a}R^{b}; n is 2-4 and R^{a} and R^{b} are independently hydrogen or C₁₋₃ alkyl; R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In another subembodiment of the present invention there is provided a compound according to formula I wherein R¹ is optionally substituted C₁₋₃ alkyl, R² is (CH₂)ₙNR^{a}R^{b}; n is 2-4 and R^{a} and R^{b} together with the nitrogen to which they are attached form a four to seven-membered ring optionally containing another heteroatom selected from O, NR^{e} or S(O)₀₋₂; R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is (alkylene)₁₋₃OR⁵ wherein R⁵ is (alkylene)₂₋₄NR^{a}R^{b} or a heterocycle selected from azetidine, pyrrolidine, piperidine or azepine; R³ is pyridinyl or pyrazinyl optionally substituted by methyl; In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is (alkylene)₁₋₃OR⁵ wherein R⁵ is (alkylene)²⁴NR^{a}R^{b}; R³ is pyridinyl or pyrazinyl optionally substituted by methyl; R^{a} and R^{b} are (a) independently hydrogen or C₁₋₃ alkyl, or, (b) R^{a} and R^{b} together with the nitrogen to which they are attached form a four to seven-membered ring optionally containing another heteroatom selected from O, NR^{e} or S(O)₀₋₂. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is (alkylene)₁₋₃OR⁵ wherein R⁵ is a heterocycle selected from azetidine, pyrrolidine, piperidine or azepine; R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In another subembodiment of the present invention there is provided a compound according to formula I wherein R¹ is optionally substituted C₁₋₃ alkyl, R² is (CH₂)₂O(CH₂)₂NR^{a}R^{b}; R^{a} and R^{b} are (a) independently hydrogen or C₁₋₃ alkyl, or, (b) R^{a} and R^{b} together with the nitrogen to which they are attached form a four to seven-membered ring optionally containing another heteroatom selected from O, NR^{e} or S(O)₀₋₂; R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In another embodiment of the present invention there is provided a compound according to formula I wherein R¹ is optionally substituted C₁₋₃ alkyl, R² is (CH₂)ᵣC(O)NR^{a}R^{b} and R^{a} and R^{b} are (a) independently hydrogen or C₁₋₃ alkyl, or, (b) R^{a} and R^{b} together with the nitrogen to which they are attached form a four to seven-membered ring optionally containing another heteroatom selected from O, NR^{e} or S(O)₀₋₂; R^{e} is hydrogen, C₁₋₃ alkyl or C₁₋₃ alkylsulfonyl; and r is 1 to 6; R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In another embodiment of the present invention there is provided a compound according to formula I wherein R¹ is optionally substituted C₁₋₃ alkyl, R² is (CH₂)ᵣC(O)NR^{a}R^{b} and R^{a} and R^{b} are independently hydrogen or C₁₋₃ alkyl and r is 1 to 6; R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In another embodiment of the present invention there is provided a compound according to formula I wherein R¹ is optionally substituted C₁₋₃ alkyl, R² is (CH₂)ᵣC(O)NR^{a}R^{b} and R^{a} and R^{b} together with the nitrogen to which they are attached form a four to seven-membered ring optionally containing another heteroatom selected from O, NR^{e} or S(O)₀₋₂; R^{e} is hydrogen, C₁₋₃ alkyl or C₁₋₃ alkylsulfonyl and r is 1 to 6. R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is a moiety of formula (I), R³ is pyridinyl or pyrazinyl

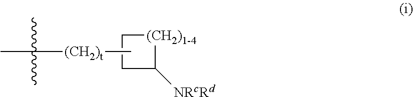

optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is 3-amino-cyclopentylmethyl, R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R¹ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is 3-amino-cyclopentylethyl, R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is 3-amino-cyclohexylmethyl, R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is 3-amino-cyclohexylethyl, R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is 4-amino-cyclohexylmethyl, R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is 4-amino-cyclohexylethyl, R³ is pyridinyl or pyrazinyl optionally substituted by methyl. In one subembodiment R³ is 2-methyl-pyrazin-6-yl. In another subembodiment R³ is 2-methyl-pyridin-6-yl. In another subembodiment R³ is 3-methyl-pyridin-2-yl. In another subembodiment R³ is 2,4-dimethyl-pyridin-6-yl. In another subembodiment R¹ is ethyl or methyl.

In one embodiment of the present invention there is provided a compound according to formula I wherein R¹ is optionally substituted C₁₋₃ alkyl, R² is (i), (ii), (iii), (iv), (v), (vi), (vii) or (viii), n is 2-6, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

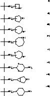

In one embodiment of the present invention there is provided a compound of formula I wherein R² is 2-morpholin-2-ylethyl or 2-morpholin-2-ylmethyl.

In one embodiment of the present invention there is provided a compound of formula I wherein R² is 2-(5-amino-1,3-dioxan-2-yl)ethyl or 2-(5-amino-1,3-dioxan-2-yl)methyl.

In one embodiment of the present invention there is provided a compound of formula I wherein R² is (3 3-difluoro-4-piperidyl)methyl or (3 3-difluoro-4-piperidyl)ethyl.

In one embodiment of the present invention there is provided a compound according to formula I wherein R¹ is optionally substituted C₁₋₃ alkyl, R² is azetidin-3-ylethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R¹ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is azetidin-3-ylpropyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is pyrrolidin-3-ylethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is pyrrolidin-3-ylpropyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is piperidin-4-ylpropyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is piperidin-4-ylethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is (vii), R³ is pyridinyl or pyrazinyl optionally substituted by methyl. In one subembodiment R³ is 2-methyl-pyrazin-6-yl In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is 5-amino-[1,3]dioxan-2-ylmethyl, R¹ is pyridinyl or pyrazinyl optionally substituted by methyl. In one subembodiment R¹ is 2-methyl-pyrazin-6-yl In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is 5-amino-[1,3]dioxan-2-ylethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is piperazin-1-ylmethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is piperazin-1-ylethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is 4-methyl-piperazin-1-ylethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In one embodiment of the present invention there is provided a compound according to formula I wherein R¹ is optionally substituted C₁₋₃ alkyl, R² is 3-aza-bicyclo[3.1.0]hex-6-ylamino)-ethyl]-2-ethyl, R³ is pyridinyl or pyrazinyl optionally substituted by methyl. In one subembodiment R³ is 2-methyl-pyrazin-6-yl. In another subembodiment R³ is 2-methyl-pyridinyl-6-yl. In another subembodiment R³ is 3-methyl-pyridin-2-yl. In another subembodiment R³ is 2,4-dimethyl-pyridinyl-6-yl. In another subembodiment R¹ is ethyl or methyl.

In another embodiment of the present invention there is provided a compound according to formula I wherein R³ is optionally substituted pyrazinyl.

In another embodiment of the present invention there is provided a compound according to formula I wherein R³ is optionally substituted pyrazinyl and R⁴ is 2-chloro or 2-methyl. In a subembodiment R³ is 6-methylpyrazin-2-yl.

In another embodiment of the present invention there is provided a compound according to formula I wherein R³ is optionally substituted pyridinyl.

In another embodiment of the present invention there is provided a compound according to formula I wherein R³ is optionally substituted pyridinyl and R⁴ is 2-chloro or 2-methyl. In another embodiment R³ is 6-methylpyridin-2-yl. In another subembodiment R³ is 3-methylpyridin-2-yl. In yet another subembodiment R³ is 4,6-dimethylpyridin-2-yl.

In another embodiment of the present invention there is provided any one compound selected from compounds I-1 to I-202 in Table I.

In another embodiment of the present invention there is provided a compound selected from the group consisting of:
6-[2-chloro-4-(6-methylpyrazin-2-yl)phenyl]-2-(methylamino)-8-[[(2R)-morpholin-2-yl]methyl]pyrido[2,3-d]pyrimidin-7-one;
2-[(2R)-2-[[6-[2-chloro-4-(6-methylpyrazin-2-yl)phenyl]-2-(methylamino)-7-oxo-pyrido[2,3-d]pyrimidin-8-yl]methyl]morpholin-4-yl]acetamide;
6-[2-chloro-4-(6-methylpyrazin-2-yl)phenyl]-8-[[(2R)-morpholin-2-yl]methyl]-2-(oxetan-3-ylamino)pyrido[2,3-d]pyrimidin-7-one;
6-[2-methyl-4-(6-methyl-2-pyridyl)phenyl]-8-[[(2R)-morpholin-2-yl]methyl]-2-(oxetan-3-ylamino)pyrido[2,3-d]pyrimidin-7-one;
2-(methylamino)-6-[2-methyl-4-(6-methyl-2-pyridyl)phenyl]-8-[[(2R)-morpholin-2-yl]methyl]pyrido[2,3-d]pyrimidin-7-one;
6-[2-chloro-4-(6-methylpyrazin-2-yl)phenyl]-8-[[(2S)-morpholin-2-yl]methyl]-2-(oxetan-3-ylamino)pyrido[2,3-d]pyrimidin-7-one;
6-[2-chloro-4-(6-methylpyrazin-2-yl)phenyl]-2-(methylamino)-8-[[(2S)-morpholin-2-yl]methyl]pyrido[2,3-d]pyrimidin-7-one;
6-[2-chloro-4-(6-methyl-2-pyridyl)phenyl]-8-[[(2R)-morpholin-2-yl]methyl]-2-(oxetan-3-ylamino)pyrido[2,3-d]pyrimidin-7-one;
6-[2-chloro-4-(6-methyl-2-pyridyl)phenyl]-2-(methylamino)-8-[[(2R)-morpholin-2-yl]methyl]pyrido[2,3-d]pyrimidin-7-one;
6-[2-chloro-4-(6-methyl-2-pyridyl)phenyl]-2-(methylamino)-8-[[(2R)-4-methylmorpholin-2-yl]methyl]pyrido[2,3-d]pyrimidin-7-one;
2-(methylamino)-6-[2-methyl-4-(6-methyl-2-pyridyl)phenyl]-8-[[(2S)-morpholin-2-yl]methyl]pyrido[2,3-d]pyrimidin-7-one;
6-[2-chloro-4-(6-methyl-2-pyridyl)phenyl]-2-(methylamino)-8-[[(2S)-morpholin-2-yl]methyl]pyrido[2,3-d]pyrimidin-7-one;
6-[2-chloro-4-(2-methylthiazol-4-yl)phenyl]-2-(methylamino)-8-[[(2S)-morpholin-2-yl]methyl]pyrido[2,3-d]pyrimidin-7-one;
6-[2-chloro-4-(2-methylthiazol-4-yl)phenyl]-2-(methylamino)-8-[[(2R)-morpholin-2-yl]methyl]pyrido[2,3-d]pyrimidin-7-one;
6-[2-chloro-4-(6-methyl-2-pyridyl)phenyl]-2-(methylamino)-8-[[(2S)-4-methylmorpholin-2-yl]methyl]pyrido[2,3-d]pyrimidin-7-one;
6-[2-chloro-4-(2-cyclopropylthiazol-4-yl)phenyl]-2-(methylamino)-8-[[(2S)-morpholin-2-yl]methyl]pyrido[2,3-d]pyrimidin-7-one;
6-[2-chloro-4-(2-cyclopropylthiazol-4-yl)phenyl]-2-(methylamino)-8-[[(2R)-morpholin-2-yl]methyl]pyrido[2,3-d]pyrimidin-7-one;
6-[2-methyl-4-(6-methyl-2-pyridyl)phenyl]-8-[[(2S)-morpholin-2-yl]methyl]-2-(oxetan-3-ylamino)pyrido[2,3-d]pyrimidin-7-one;
6-[2-chloro-4-(6-methyl-2-pyridyl)phenyl]-8-[[(2S)-morpholin-2-yl]methyl]-2-(oxetan-3-ylamino)pyrido[2,3-d]pyrimidin-7-one;
6-[2-chloro-4-(4-methylthiazol-2-yl)phenyl]-2-(methylamino)-8-[[(2R)-morpholin-2-yl]methyl]pyrido[2,3-d]pyrimidin-7-one;
6-[2-chloro-4-(4 5-dimethylthiazol-2-yl)phenyl]-2-(methylamino)-8-[[(2R)-morpholin-2-yl]methyl]pyrido[2,3-d]pyrimidin-7-one;
6-[2-chloro-4-(1-methylpyrazol-3-yl)phenyl]-2-(methylamino)-8-[[(2R)-morpholin-2-yl]methyl]pyrido[2,3-d]pyrimidin-7-one;
6-[2-ethyl-4-(6-methyl-2-pyridyl)phenyl]-8-[[(2R)-morpholin-2-yl]methyl]-2-(oxetan-3-ylamino)pyrido[2,3-d]pyrimidin-7-one;
6-[2-ethyl-4-(6-methyl-2-pyridyl)phenyl]-8-[[(2S)-morpholin-2-yl]methyl]-2-(oxetan-3-ylamino)pyrido[2,3-d]pyrimidin-7-one;
6-[2-chloro-4-(1 4-dimethylimidazol-2-yl)phenyl]-2-(methylamino)-8-[[(2R)-morpholin-2-yl]methyl]pyrido[2,3-d]pyrimidin-7-one;
6-[2-chloro-4-[6-(hydroxymethyl)-2-pyridyl]phenyl]-2-(methylamino)-8-[[(2S)-morpholin-2-yl]methyl]pyrido[2,3-d]pyrimidin-7-one;
6-(2-chloro-4-cyclopropyl-phenyl)-2-(methylamino)-8-[[(2R)-morpholin-2-yl]methyl]pyrido[2,3-d]pyrimidin-7-one;
6-[2-chloro-4-(6-methylpyrazin-2-yl)phenyl]-2-(ethylamino)-8-(2-morpholin-2-ylethyl)pyrido[2,3-d]pyrimidin-7-one;
6-[2-chloro-4-(6-methylpyrazin-2-yl)phenyl]-2-(ethylamino)-8-(2-morpholin-2-ylethyl)pyrido[2,3-d]pyrimidin-7-one;
2-(methylamino)-6-[2-methyl-4-(6-methyl-2-pyridyl)phenyl]-8-(2-morpholin-2-ylethyl)pyrido[2,3-d]pyrimidin-7-one;
2-(methylamino)-6-[2-methyl-4-(6-methyl-2-pyridyl)phenyl]-8-(2-morpholin-2-ylethyl)pyrido[2,3-d]pyrimidin-7-one;
2-(ethylamino)-6-[2-methyl-4-(6-methyl-2-pyridyl)phenyl]-8-(2-morpholin-2-ylethyl)pyrido[2,3-d]pyrimidin-7-one;
6-[2-methyl-4-(6-methyl-2-pyridyl)phenyl]-8-(2-morpholin-2-ylethyl)-2-(oxetan-3-ylamino)pyrido[2,3-d]pyrimidin-7-one;
6-[2-methyl-4-(6-methyl-2-pyridyl)phenyl]-8-(2-morpholin-2-ylethyl)-2-(oxetan-3-ylamino)pyrido[2,3-d]pyrimidin-7-one;
2-(ethylamino)-6-[2-methyl-4-(6-methyl-2-pyridyl)phenyl]-8-(2-morpholin-2-ylethyl)pyrido[2,3-d]pyrimidin-7-one;
2-(2 2-difluoroethylamino)-6-[2-methyl-4-(6-methyl-2-pyridyl)phenyl]-8-(2-morpholin-2-ylethyl)pyrido[2,3-d]pyrimidin-7-one;
6-[2-chloro-(6-methylpyrazin-2-yl)phenyl]-2-(methylamino)-8-(2-morpholin-2-ylethyl)pyrido[2,3-d]pyrimidin-7-one;
6-[2-chloro-4-(6-methyl-2-pyridyl)phenyl]-2-(methylamino)-8-(2-morpholin-2-ylethyl)pyrido[2,3-d]pyrimidin-7-one;
6-[2-chloro-4-(6-methylpyrazin-2-yl)phenyl]-2-(methylamino)-8-(2-morpholin-2-ylethyl)pyrido[2,3-d]pyrimidin-7-one;
2-amino-6-[2-chloro-4-(6-methyl-2-pyridyl)phenyl]-8-(2-morpholin-2-ylethyl)pyrido[2,3-d]pyrimidin-7-one;
6-[2-chloro-4-(6-methyl-2-pyridyl)phenyl]-2-(methylamino)-8-(2-morpholin-2-ylethyl)pyrido[2,3-d]pyrimidin-7-one;
6-[2-chloro-4-(2-methylthiazol-4-yl)phenyl]-2-(methylamino)-8-(2-morpholin-2-ylethyl)pyrido[2,3-d]pyrimidin-7-one;
6-[2-chloro-4-(2-methylthiazol-4-yl)phenyl]-2-(methylamino)-8-(2-morpholin-2-ylethyl)pyrido[2,3-d]pyrimidin-7-one;
6-[2-chloro-4-(6-methyl-2-pyridyl)phenyl]-2-(methylamino)-8-(2-morpholin-2-ylethyl)pyrido[2,3-d]pyrimidin-7-one;
6-[2-chloro-4-(6-methyl-2-pyridyl)phenyl]-2-(methylamino)-8-(2-morpholin-2-ylethyl)pyrido[2,3-d]pyrimidin-7-one;
6-[2-chloro-4-(6-methyl-2-pyridyl)phenyl]-2-(methylamino)-8-[2-(4-methylmorpholin-2-yl)ethyl]pyrido[2,3-d]pyrimidin-7-one; and,
6-[2-chloro-4-(6-methyl-2-pyridyl)phenyl]-2-(methylamino)-8-[2-(4-methylmorpholin-2-yl)ethyl]pyrido[2,3-d]pyrimidin-7-one; or, a stereoisomer or pharmaceutically acceptable salt thereof.

In another embodiment of the present invention there is provided a compound selected from the group consisting of:
8-[(5-amino-1,3-dioxan-2-yl)methyl]-6-[2-chloro-4-(6-methylpyrazin-2-yl)phenyl]-2-(ethylamino)pyrido[2,3-d]pyrimidin-7-one;
tert-butyl N-[2-[[6-[2-chloro-4-(6-methylpyrazin-2-yl)phenyl]-2-(oxetan-3-ylamino)-7-oxo-pyrido[2,3-d]pyrimidin-8-yl]methyl]-1,3-dioxan-5-yl]carbamate;
2-amino-8-[(5-amino-1,3-dioxan-2-yl)methyl]-6-[2-chloro-4-(6-methylpyrazin-2-yl)phenyl]pyrido[2,3-d]pyrimidin-7-one;
8-[(5-amino-1,3-dioxan-2-yl)methyl]-6-[2-chloro-4-(6-methylpyrazin-2-yl)phenyl]-2-(ethylamino)pyrido[2,3-d]pyrimidin-7-one;
8-[(5-amino-1,3-dioxan-2-yl)methyl]-6-[2-chloro-4-(6-methylpyrazin-2-yl)phenyl]-2-(methylamino)pyrido[2,3-d]pyrimidin-7-one;
2-amino-8-[(5-amino-1,3-dioxan-2-yl)methyl]-6-[2-chloro-4-(6-methylpyrazin-2-yl)phenyl]pyrido[2,3-d]pyrimidin-7-one;
8-[(5-amino-1,3-dioxan-2-yl)methyl]-6-[2-chloro-4-(6-methylpyrazin-2-yl)phenyl]-2-(oxetan-3-ylamino)pyrido[2,3-d]pyrimidin-7-one;
8-[(5-amino-1,3-dioxan-2-yl)methyl]-6-[2-chloro-4-(6-methyl-2-pyridyl)phenyl]-2-(methylamino)pyrido[2,3-d]pyrimidin-7-one;
8-[(5-amino-1,3-(dioxan-2-yl)methyl]-2-(methylamino)-6-[2-dimethyl-4-(6-methyl-2-pyridyl)phenyl]pyrido[2,3-d]pyrimidin-7 one;
6-[2-chloro-4-(6-methylpyrazin-2-yl)phenyl]-2-(methylamino)-8-[[5-(methylamino)-1,3-dioxan-2-yl]methyl]pyrido[2,3-d]pyrimidin-7-one;
8-[(5-amino-1,3-dioxan-2-yl)methyl]-6-[2-chloro-4-(2-methylthiazol-4-yl)phenyl]-2-(methylamino)pyrido[2,3-d]pyrimidin-7-one;
8-[(5-amino-1,3-dioxan-2-yl)methyl]-6-[2-chloro-4-(6-methyl-2-pyridyl)phenyl]-2-(dimethylamino)pyrido[2,3-d]pyrimidin-7-one;
8-[(5-amino-1,3-dioxan-2-yl)methyl]-6-[2-chloro-4-(3-methyl-2-pyridyl)phenyl]-2-(methylamino)pyrido[2,3-d]pyrimidin-7-one;
8-[(5-amino-1,3-dioxan-2-yl)methyl]-6-[2-ethyl-4-(6-methyl-2-pyridyl)phenyl]-2-(methylamino)pyrido[2,3-d]pyrimidin-7-one;
6-[2-chloro-4-(6-methyl-2-pyridyl)phenyl]-2-(methylamino)-8-[[5-(methylamino)-1,3-dioxan-2-yl]methyl]pyrido[2,3-d]pyrimidin-7-one;
8-[(5-amino-1,3-dioxan-2-yl)methyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(ethylamino)pyrido[2,3-d]pyrimidin-7-one;
8-[2-(5-amino-1,3-dioxan-2-yl)ethyl]-2-(3-azabicyclo[3.1.0]hexan-6-ylamino)-6-[2-chloro-4-(6-methylpyrazin-2-yl)phenyl]pyrido[2,3-d]pyrimidin-7-one;
8-[2-(5-amino-1,3-dioxan-2-yl)ethyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one;
8-[2-(5-amino-1,3-dioxan-2-yl)ethyl]-6-[2-chloro-4-(2-methylthiazol-4-yl)phenyl]-2-(methylamino)pyrido[2,3-d]pyrimidin-7-one;
8-[2-(5-amino-1,3-dioxan-2-yl)ethyl]-6-[2-ethyl-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(ethylamino)pyrido[2,3-d]pyrimidin-7-one;
8-[2-(5-amino-1,3-dioxan-2-yl)ethyl]-2-(3-azabicyclo[3.1.0]hexan-6-ylamino)-6-[2-chloro-4-(6-methylpyrazin-2-yl)phenyl]pyrido[2,3-d]pyrimidin-7-one;
8-[2-(5-amino-1 3-dioxan-2-yl)ethyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one;
8-[2-(5-amino-1,3-dioxan-2-yl)ethyl]-6-[2-chloro-4-(2-methylthiazol-4-yl)phenyl]-2-(methylamino)pyrido[2,3-d]pyrimidin-7-one;
8-[2-(5-amino-1,3-dioxan-2-yl)ethyl]-6-[2-ethyl-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(ethylamino)pyrido[2,3-d]pyrimidin-7-one;
8-[2-(5-amino-1,3-dioxan-2-yl)ethyl]-2-(3-azabicyclo[3.1.0]hexan-6-ylamino)-6-[2-chloro-4-(6-methylpyrazin-2-yl)phenyl]pyrido[2,3-d]pyrimidin-7-one;
8-[2-(5-amino-1,3-dioxan-2-yl)ethyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(oxetan-3-ylamino)pyrido[2,3-d]pyrimidin-7-one;
8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one;
8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one;
8-[2-(5-amino-1,3-dioxan-2-yl)ethyl]-6-[2-chloro-4-(2-methylthiazol-4-yl)phenyl]-2-(methylamino)pyrido[2,3-d]pyrimidin-7-one; and,
8-[2-(5-amino-1,3-dioxan-2-yl)ethyl]-6-[2-ethyl-4-(6-methyl-2-pyridyl)phenyl]-2-(oxetan-3-ylamino)pyrido[2,3-d]pyrimidin-7-one; or a or, a stereoisomer or pharmaceutically acceptable salt thereof.

In another embodiment of the present invention there is provided a method of inhibiting PAK activity in a patient in need thereof comprising the step of administering to said patient a compound according to formula I wherein R¹, R², R³, R⁴, R⁵, R^{a}, R^{b}, R^{c}, R^{d}, R^{e}, n, r, s and t are as defined hereinabove.

In another embodiment of the present invention there is provided a method of treating or ameliorating the severity of cancer or a hyperproliferative disorder in a patient in need thereof comprising administering to said patient a compound according to formula I wherein R¹, R², R³, R⁴, R⁵, R^{a}, R^{b}, R^{c}, R^{d}, R^{e}, n, r, s and t are as defined hereinabove.

In another embodiment of the present invention there is provided a method of treating a cancer or hyperproliferative disorder selected from the group consisting of adenoma, bladder cancer, brain cancer, breast cancer, colon cancer, epidermal carcinoma, follicular carcinoma, cancer of the genitourinary tract, glioblastoma, Hodgkin's disease, head and neck cancers, heptoma, keratoacanthoma, kidney cancer, large cell carcinoma, leukemias, lung adenocarcinoma, lung cancer, lymphoid disorders, melanoma and non-melanoma skin cancer, myelodysplastic syndrome, neuroblastoma, non-Hodgkins lymphoma, ovarian cancer, papillary carcinoma, pancreatic cancer, prostate cancer, rectal cancer, sarcoma, small cell carcinoma, testicular cancer, tetracarcinomas, thyroid cancer, and undifferentiated carcinoma comprising administering to a patient in need thereof a compound according to formula I wherein R¹, R², R³, R⁴, R⁵, R^{a}, R^{b}, R^{c}, R^{d}, R^{e}, n, r, s and t are as defined hereinabove.

In another embodiment of the present invention there is provided a method of treating a cancer selected from the group consisting of lung cancer, breast cancer, ovarian cancer, bladder cancer and head and neck cancer comprising administering to a patient in need thereof a compound according to formula I wherein R¹, R², R³, R⁴, R⁵, R^{a}, R^{b}, R^{c}, R^{d}, R^{e}, n, r, s and t are as defined hereinabove.

In another embodiment of the present invention there is provided a method of treating a cancer selected from the group consisting of primary breast adenocarcinoma, squamous non-small cell lung cancer or a squamous head and neck cancer comprising administering to a patient in need thereof a compound according to formula I wherein R¹, R², R³, R⁴, R⁵, R^{a}, R^{b}, R^{c}, R^{d}, R^{e}, n, r, s and t are as defined hereinabove.

In another embodiment of the present invention there is provided a method of treating cancer or a hyperproliferative disease comprising co-administering to a patient in need thereof a compound according to formula I wherein R¹, R², R³, R⁴, R⁵, R^{a}, R^{b}, R^{c}, R^{d}, R^{e}, n, r, s and t are as defined hereinabove and at least one other chemotherapeutic agent used to treat or ameliorate cancer or a hyperproliferative disorder.

In another embodiment of the present invention there is provided a method of treating cancer or a hyperproliferative disease comprising co-administering to a patient in need thereof a compound according to formula I wherein R¹, R², R³, R⁴, R⁵, R^{a}, R^{b}, R^{c}, R^{d}, R^{e}, n, r, s and t are as defined hereinabove and at least one other chemotherapeutic agent selected from the group consisting of inhibitor of apoptosis proteins (IAP), an EGFR inhibitor or antagonist, an inhibitor of Ras/Raf/Mek/Erk signaling cascade, an inhibitor of Akt kinase and a Src kinase inhibitor.

In another embodiment of the present invention there is provided a pharmaceutical composition comprising a compound according to formula I wherein R¹, R², R³, R⁴, R⁵, R^{a}, R^{b}, R^{c}, R^{d}, R^{e}, n, r, s and t are as defined hereinabove and at least one pharmaceutically acceptable carrier, excipient or diluent.

In another embodiment of the present invention there is provided the use of a composition containing a compound of according to formula I wherein R¹, R², R³, R⁴, R⁵, R^{a}, R^{b}, R^{c}, R^{d}, R^{e}, n, r, s and t are as defined hereinabove for use in the manufacture of a medicament for the inhibition of abnormal cell growth or for treatment of a hyperproliferative disorder in a mammal.

In another embodiment of the present invention there is provided a p21-activated kinase (PAK) inhibitor for use in treating cancer or a hyperproliferative disorder.

In another embodiment of the present invention there is provided the use of a composition containing a compound of selected from I-1 to I-202 in TABLE 1 for use in the manufacture of a medicament for the inhibition of abnormal cell growth or for treatment of a hyperproliferative disorder in a mammal.

In another embodiment of the present invention there is provided a compound according to formula I wherein R¹ is H or optionally substituted C₁₋₃ alkyl; R² is (i) C₂₋₆ hydroxyalkyl, (ii) (alkylene)ₙNR^{a}R^{b} wherein the alkylene chain is optionally substituted by a hydroxyl, (iii) (alkylene)₁₋₃OR⁵ wherein R⁵ is (alkylene)₂₋₄NR^{a}R^{b} or heterocycle selected from azetidine, pyrrolidine, piperidine or azepine, (iv) (alkylene)ᵣC(O)NR^{a}R^{b}, (v) (alkylene)ₜ-CN, (vi) (alkylene)ₜ-(C₄₋₆-cycloalkyl NR^{c}R^{d}), (vii) heterocyclyl-(alkylene) wherein heterocyclyl refers to optionally substituted azetidinyl, pyrrolidinyl, piperidinyl, azepinyl, morpholinyl, 4-amino-1,3-dioxolan-2-yl, 3-aza-bicyclo[3.1.0]hexan-6-yl or piperazinyl; R^{a} and R^{b} are (a) independently in each occurrence hydrogen or C₁₋₃ alkyl, or, (b) R^{a} and R^{b} together with the nitrogen to which they are attached form a four to seven-membered ring optionally containing another heteroatom selected from O, NR^{e} or S(O)₀₋₂; R^{c} and R^{d} are independently in each occurrence hydrogen or C₁₋₃ alkyl; R^{e} is hydrogen, C₁₋₃ alkyl or C₁₋₃ alkylsulfonyl; R³ is heteroaryl wherein the heteroaryl ring is pyridinyl, pyrazinyl or 1,2,4-oxadiazol-3-yl each optionally substituted by an R⁴ moiety; R⁴ is C₁₋₆ alkyl or halogen; n is 2-6; r is 1-6; or; t is 0-6; or, a pharmaceutically acceptable salt thereof.

In yet another embodiment of the present invention there is provided a compound according to formula I wherein R¹ is H, optionally substituted C₁₋₃ alkyl, oxetan-3-yl or 1-hydroxy-oxetan-3-yl; R² is (i) C₂₋₄ hydroxyalkyl or C₂₋₆ dihydroxyalkyl, (ii) (alkylene)ₙNR^{a}R^{b} wherein the alkylene chain is optionally substituted by a hydroxyl, (iii) (alkylene)₁₋₃OR⁵ wherein R⁵ is (alkylene)₂₋₄NR^{a}R^{b} or a heterocycle selected from azetidine, pyrrolidine, piperidine or azepine; (iv) (alkylene)ᵣC(O)NR^{a}R^{b}, (v) (alkylene)ₙ-CN, (vi) (alkylene)ₜ-(C₄₋₄-cycloalkyl NR^{c}R^{d}), (vii) heterocyclyl-(alkylene) wherein heterocyclyl refers to stituted azetidinyl, pyrrolidinyl, piperidinyl, azepinyl, morpholinyl, 5-amino-1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl or piperazinyl optionally substituted by C(═O)CHR^{f}NH₂, oxo, hydroxyl, amino, C₁₋₃ alkylamino, C₁₋₃ dialkylamino or C₁₋₆-hydroxyalkyl; R^{a} and R^{b} are (a) independently in each occurrence hydrogen, C₁₋₃ alkyl or C₂₋₄-hydroxyalkyl, or, (b) R^{a} and R^{b} together with the nitrogen to which they are attached form a four to seven-membered ring optionally containing another heteroatom selected from O, NR^{e} or S(O)₀₋₂; R^{c} and R^{d} are independently in each occurrence hydrogen, C₁₋₃ alkyl or oxetanyl; R^{e} is hydrogen, C₁₋₃ alkyl or C₁₋₃ alkylsulfonyl; R^{f} is hydrogen or C₁₋₃ alkyl; R³ is heteroaryl wherein the heteroaryl ring is pyridinyl, pyrazinyl or 1,2,4-oxadiazol-3-yl each optionally substituted by an R⁴ moiety; R⁴ is C₁₋₆ alkyl or halogen; n is 2-6; r is 1-6; t is 0-6; or, a pharmaceutically acceptable salt thereof, with the proviso that the compound of formula I is not: 8-(trans-4-aminocyclohexyl)-6-[2-chloro-4-(6-methyl-2-pyrazinyl)phenyl]-2-(ethylamino)-pyrido[2,3-d]pyrimidin-7(8H)-one, 8-(trans-4-aminocyclohexyl)-6-[2-chloro-4-(6-methyl-2-pyrazinyl)phenyl]-2-[(1-methylethyl)amino]-pyrido[2,3-d]pyrimidin-7(8H)-one, 6-(2-chloro-4-(5-methyl-1,2,4-oxadiazol-3-yl)phenyl)-2-(ethylamino)-8-(piperidin-4-ylmethyl)pyrido[2,3-d]pyrimidin-7(8H)-one, or, 6-(2-chloro-4-(5-methyl-1,2,4-oxadiazol-3-yl)phenyl)-2-(isopropylamino)-8-(piperidin-4-ylmethyl)pyrido[2,3-d]pyrimidin-7(8H)-one.

Commonly used abbreviations include: acetyl (Ac), aqueous (aq.), atmospheres (Atm), tert-butoxycarbonyl (Boc), di-tert-butyl pyrocarbonate or boc anhydride (BOC₂O), benzyl (Bn), benzotriazol-1-yloxy-tris-(dimethylamino)phosphonium hexafluorophosphate (BOP), butyl (Bu), benzoyl (Bz), Chemical Abstracts Registration Number (CASRN), benzyloxycarbonyl (CBZ or Z), carbonyl diimidazole (CDI), dibenzylideneacetone (DBA), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), methanol (MeOH), melting point (mp), MeSO₂— (mesyl or Ms), methyl (Me), acetonitrile (MeCN), m-chloroperbenzoic acid (MCPBA), mass spectrum (ms), methyl tert-butyl ether (MTBE), N-methylmorpholine (NMM), N-methylpyrrolidone (NMP), petroleum ether (pet ether, i.e. hydrocarbons),) phenyl (Ph), bis(pinacolato)dibo [(PinB)₂], propyl (Pr), iso-propyl (i-Pr), pounds per square inch (psi), bromo-tris-pyrrolidinophosphonium hexafluorophosphate (PyBrOP), pyridine (pyr), room temperature (rt or RT), satd. (saturated), tert-butyldimethylsilyl or t-BuMe2Si (TBDMS), triethylamine (TEA or Et₃N), triflate or CF₃SO₂— (Tf), trifluoroacetic acid (TFA), O-benzotriazol-1-yl-N,N,N′,N′-tetramethyluronium tetrafluoroborate (TBTU), thin layer chromatography (TLC), tetrahydrofuran (THF), tetramethylethylenediamine (TMEDA), trimethylsilyl or Me₃Si (TMS), 2-(trimethylsilyl)ethoxymethyl (SEM); p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert- or -t) and neo- have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it. The following numbering system is used herein.

TABLE I

| Compound | | Kᵢ | |
|---|---|---|---|
| No. | Structure | (μM)¹ | Name |
| I-1 | 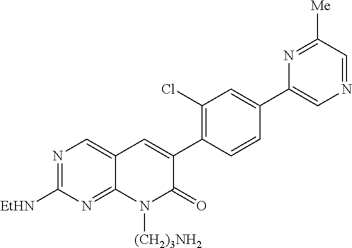 | 0.00823 | 8-(3-aminopropyl)-6-(2-chloro-4- (6-methylpyrazin-2-yl)phenyl)-2- (ethylamino)pyrido[2,3- d]pyrimidin-7(8H)-one |
| I-2 | 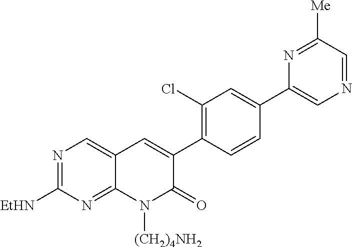 | 0.00609 | 8-(4-aminobutyl)-6-(2-chloro-4-(6- methylpyrazin-2-yl)phenyl)-2- (ethylamino)pyrido[2,3- d]pyrimidin-7(8H)-one |
| I-3 | 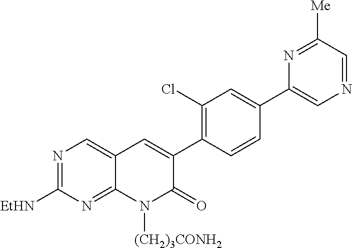 | 0.0474 | 4-(6-(2-chloro-4-(6-methylpyrazin- 2-yl)phenyl)-2-(ethylamino)-7- oxopyrido[2,3-d]pyrimidin-8(7H)- yl)butanamide |
| I-4 | 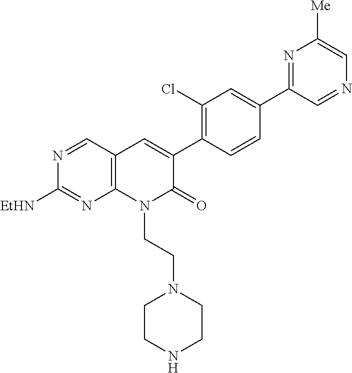 | 0.0064 | 6-(2-chloro-4-(6-methylpyrazin-2- yl)phenyl)-2-(ethylamino)-8-(2- (piperazin-1-yl)ethyl)pyrido[2,3- d]pyrimidin-7(8H)-one |
| I-5 | 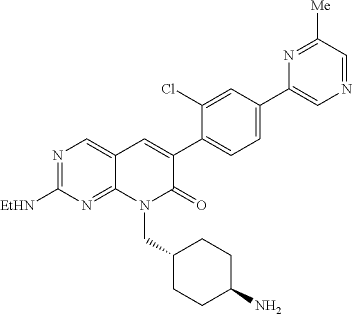 | 0.00199 | 8-(((1r,4r)-4- aminocyclohexyl)methyl)-6-(2- chloro-4-(6-methylpyrazin-2- yl)phenyl)-2- (ethylamino)pyrido[2,3- d]pyrimidin-7(8H)-one |
| I-6 | 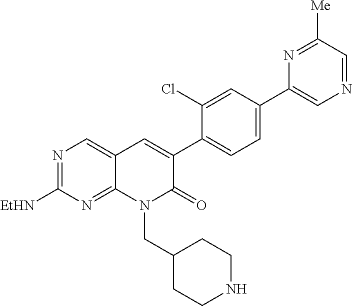 | 0.0065 | 6-(2-chloro-4-(6-methylpyrazin-2- yl)phenyl)-2-(ethylamino)-8- (piperidin-4-ylmethyl)pyrido[2,3- d]pyrimidin-7(8H)-one |
| I-7 | 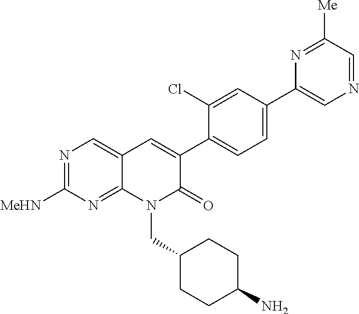 [SMILES: CNc1ncc2cc(-c3ccc(-c4cncc(C)n4)cc3Cl)c(=O)n(C[C@H]3CC[C@H](N)CC3)c2n1] | 0.0078 | 1-(((1r,4r)-4- aminocyclohexyl)methyl)-3-(2- chloro-4-(6-methylpyrazin-2- yl)phenyl)-7- (methylamino)pyrido[2,3- d]pyrimidin-2(1H)-one |
| I-8 | 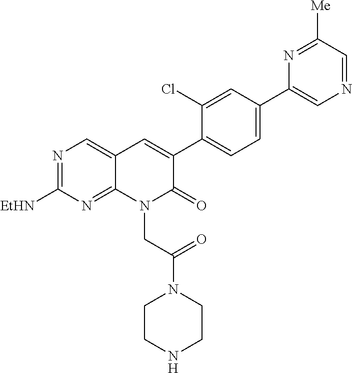 | 0.0309 | 6-(2-chloro-4-(6-methylpyrazin-2- yl)phenyl)-2-(ethylamino)-8-(2- oxo-2-(piperazin-1- yl)ethyl)pyrido[2,3-d]pyrimidin- 7(8H)-one |
| I-9 | 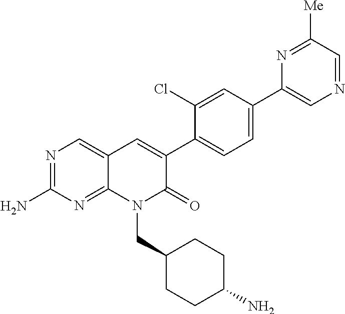 [SMILES: Cc1cncc(-c2ccc(-c3cc4cnc(N)nc4n(C[C@H]4CC[C@H](N)CC4)c3=O)c(Cl)c2)n1] | 0.014 | 2-amino-8-(((1r,4r)-4- aminocyclohexyl)methyl)-6-(2- chloro-4-(6-methylpyrazin-2- yl)phenyl)pyrido[2,3-d]pyrimidin- 7(8H)-one |
| I-10 | 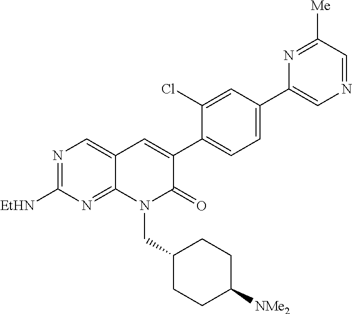 | 0.0213 | 6-(2-chloro-4-(6-methylpyrazin-2- yl)phenyl)-8-(((1r,4r)-4- (dimethylamino)cyclohexyl)methyl)- 2-(ethylamino)pyrido[2,3- d]pyrimidin-7(8H)-one |
| I-11 | 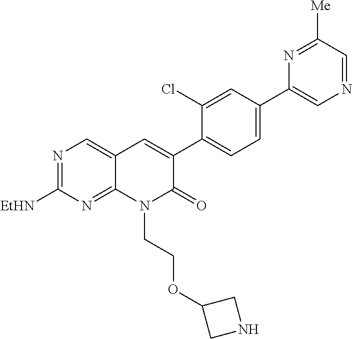 | 0.00694 | 8-(2-(azetidin-3-yloxy)ethyl)-6-(2- chloro-4-(6-methylpyrazin-2- yl)phenyl)-2- (ethylamino)pyrido[2,3- d]pyrimidin-7(8H)-one |
| I-12 | 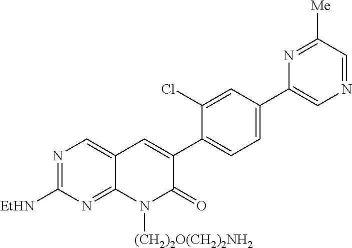 | 0.0043 | 8-(2-(2-aminoethoxy)ethyl)-6-(2- chloro-4-(6-methylpyrazin-2- yl)phenyl)-2- (ethylamino)pyrido[2,3- d]pyrimidin-7(8H)-one |
| I-13 | 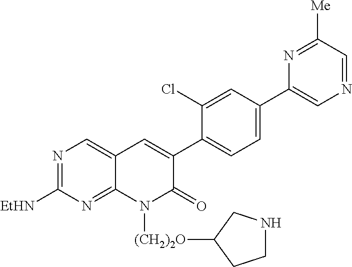 | 0.0058 | 6-(2-chloro-4-(6-methylpyrazin-2- yl)phenyl)-2-(ethylamino)-8-(2- (pyrrolidin-3- yloxy)ethyl)pyrido[2,3 d]pyrimidin- 7(8H)-one |
| I-14 | 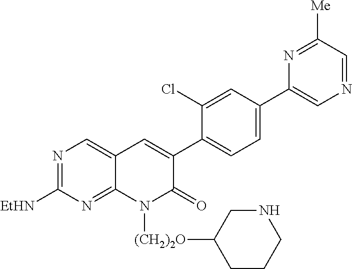 | 0.0148 | 6-(2-chloro-4-(6-methylpyrazin-2- yl)phenyl)-2-(ethylamino)-8-(2- (piperidin-3-yloxy)ethyl)pyrido[2,3- d]pyrimidin-7(8H)-one |
| I-15 | 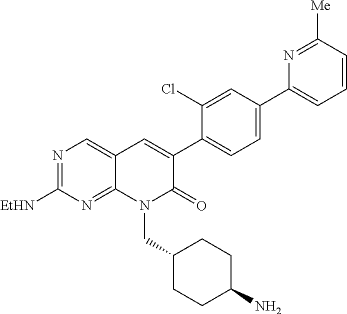 | 0.0017 | 8-(((1r,4r)-4- aminocyclohexyl)methyl)-6-(2- chloro-4-(6-methylpyridin-2- yl)phenyl)-2- (ethylamino)pyrido[2,3- d]pyrimidin-7(8H)-one |
| I-16 | 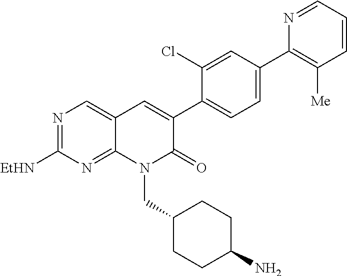 | 0.0072 | 8-(((1r,4r)-4- aminocyclohexyl)methyl)-6-(2- chloro-4-(3-methylpyridin-2- yl)phenyl)-2- (ethylamino)pyrido[2,3- d]pyrimidin-7(8H)-one |
| I-17 | 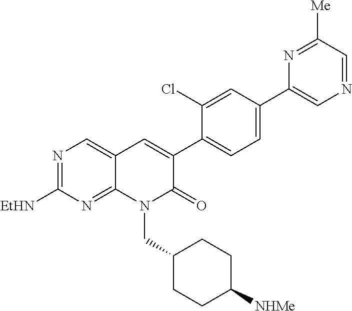 | 0.0048 | 6-(2-Chloro-4-(6-methylpyrazin-2- yl)phenyl)-2-(ethylamino)-8- (((1r,4r)-4- (methylamino)cyclohexyl)methyl) pyrido[2,3-d]pyrimidin-7(8H)-one |
| I-18 | 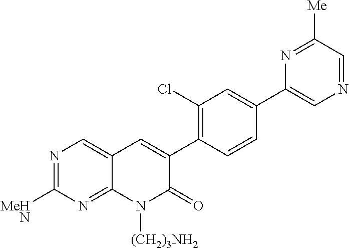 | 0.0137 | 8-(3-Aminopropyl)-6-[2-chloro-4- (6-methylpyrazin-2-yl)phenyl]-2- (methylamino)pyrido[2,3- d]pyrimidin-7-one |
| I-19 Isomer A I-20 Isomer B | 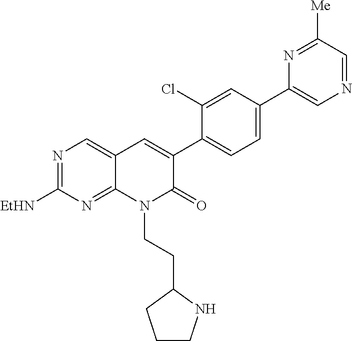 | 0.0268 0.0204 | 6-(2-Chloro-4-(6-methylpyrazin-2- yl)phenyl)-2-(ethylamino)-8-(2- (pyrrolidin-2-yl)ethyl)pyrido[2,3- d]pyrimidin-7(8H)-one |
| I-21 Isomer A I-22 Isomer B | 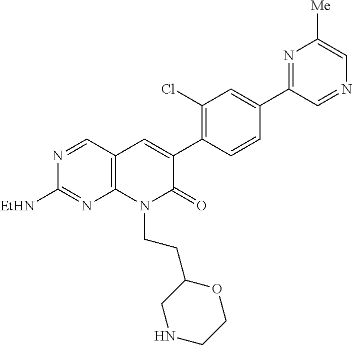 | 0.0098 0.0089 | 6-(2-chloro-4-(6-methylpyrazin-2- yl)phenyl)-2-(ethylamino)-8-(2- (morpholin-2-yl)ethyl)pyrido[2,3- d]pyrimidin-7(8H)-one |
| I-23 | 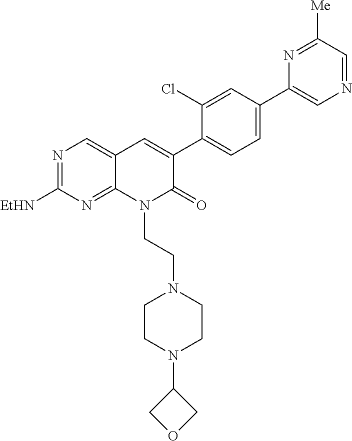 | 0.136 | 6-(2-chloro-4(6-methylpyrazin-2- yl)phenyl)-2-(ethylamino)-8-(2-(4- (oxetan-3-yl)piperazin-1- yl)ethyl)pyrido[2,3-d]pyrimidin- 7(8H)-one |
| I-24 | 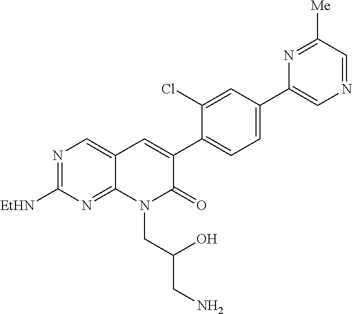 | 0.0273 | 8-(3-amino-2-hydroxy-propyl)-6-[2- chloro-4-(6-methylpyrazin-2- yl)phenyl]-2- (ethylamino)pyrido[2,3- d]pyrimidin-7-one |
| I-25 | 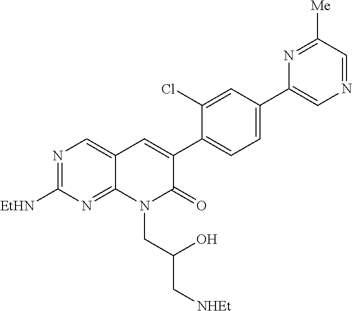 | 0.0671 | 6-[2-chloro-4-(6-methylpyrazin-2- yl)phenyl]-2-(ethylamino)-8-[3- (ethylamino)-2-hydroxy- propyl]pyrido[2,3-d]pyrimidin-7- one |
| I-26 | 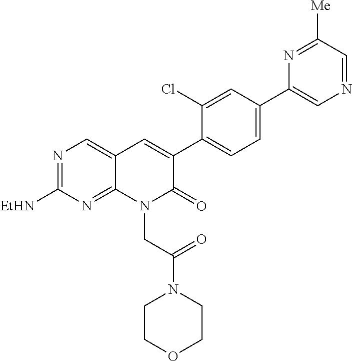 | 0.137 | 6-[2-chloro-4-(6-methylpyrazin-2- yl)phenyl]-2-(ethylamino)-8-(2- morpholino-2-oxo-ethyl)pyrido[2,3- d]pyrimidin-7-one |
| I-27 | 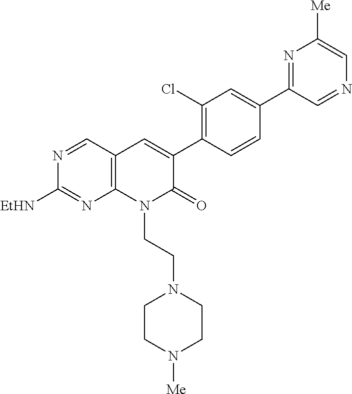 | 0.0476 | 6-(2-chloro-4-(6-methylpyrazin-2- yl)phenyl)-2-(ethylamino)-8-(2-(4- methylpiperazin-1- yl)ethyl)pyrido[2,3-d]pyrimidin- 7(8H)-one |
| I-28 | 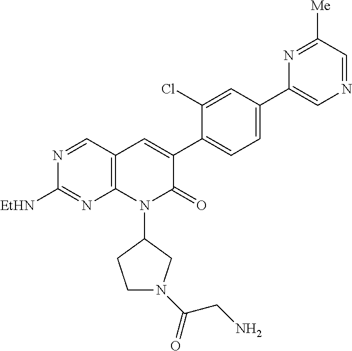 | 0.0257 | 8-[1-(2-aminoacetyl)pyrrolidin-3- yl]-6-[2-chloro-4-(6-methylpyrazin- 2-yl)phenyl]-2- (ethylamino)pyrido[2,3- d]pyrimidin-7-one |
| I-29 | 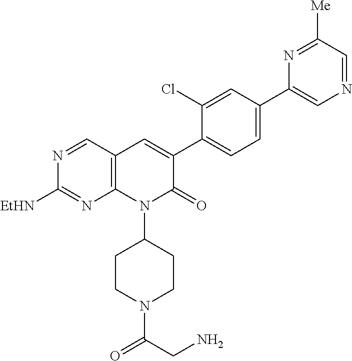 | 0.0171 | 8-[1-(2-aminoacetyl)-4-piperidyl]- 6-[2-chloro-4-(6-methylpyrazin-2- yl)phenyl]-2- (ethylamino)pyrido[2,3- d]pyrimidin-7-one |
| I-30 | 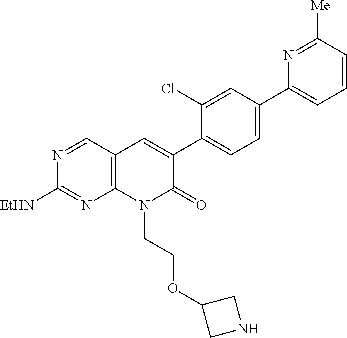 | 0.0099 | 8-[2-(azetidin-3-yloxy)ethyl]-6-[2- chloro-4-(6-methyl-2- pyridyl)phenyl]-2- (ethylamino)pyrido[2,3- d]pyrimidin-7-one |
| I-31 | 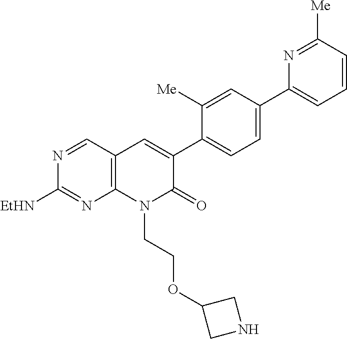 | 0.0046 | 8-[2-(azetidin-3-yloxy)ethyl]-2- (ethylamino)-6-[2-methyl-4-(6- methyl-2- pyridyl)phenyl]pyrido[2,3- d]pyrimidin-7-one |
| I-32 | 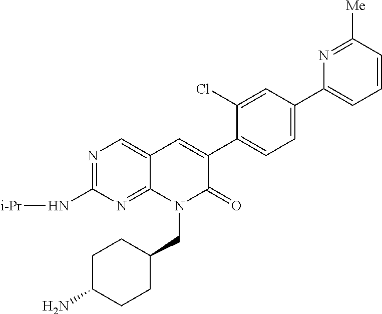 [SMILES: Cc1cccc(-c2ccc(-c3cc4cnc(NC(C)C)nc4n(C[C@H]4CC[C@H](N)CC4)c3=O)c(Cl)c2)n1] | 0.0005 | 8-[(4-aminocyclohexyl)methyl]-6- [2-chloro-4-(6-methyl-2- pyridyl)phenyl]-2- (isopropylamino)pyrido[2,3- d]pyrimidin-7-one |
| I-33 | 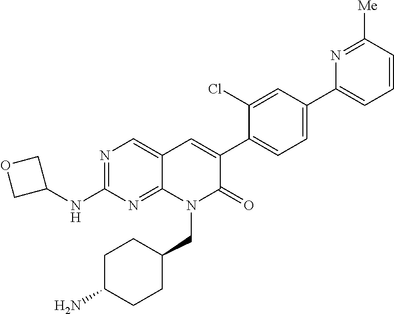 [SMILES: Cc1cccc(-c2ccc(-c3cc4cnc(NC5COC5)nc4n(C[C@H]4CC[C@H](N)CC4)c3=O)c(Cl)c2)n1] | 0.0005 | 8-[(4-aminocyclohexyl)methyl]-6- [2-chloro-4-(6-methyl-2- pyridyl)phenyl]-2-(oxetan-3- ylamino)pyrido[2,3-d]pyrimidin-7- one |
| I-34 | 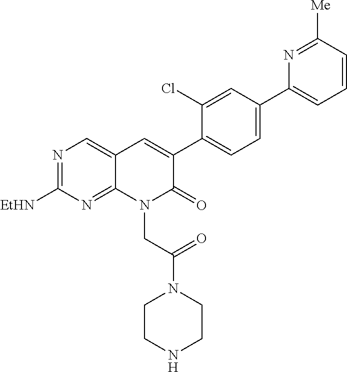 | 0.004 | 6-[2-chloro-4-(6-methyl-2- pyridyl)phenyl]-2-(ethylamino)-8- (2-oxo-2-piperazin-1-yl- ethyl)pyrido[2,3-d]pyrimidin-7-one |
| I-35 | 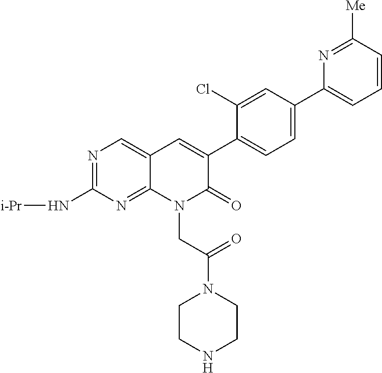 [SMILES: Cc1cccc(-c2ccc(-c3cc4cnc(NC(C)C)nc4n(CC(=O)N4CCNCC4)c3=O)c(Cl)c2)n1] | 0.0042 | 6-[2-chloro-4-(6-methyl-2- pyridyl)phenyl]-2- (isopropylamino)-8-(2-oxo-2- piperazin-1-yl-ethyl)pyrido[2,3- d]pyrimidin-7-one |
| I-36 | 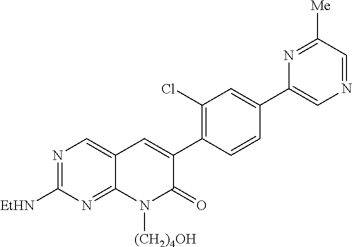 | 0.0718 | 6-[2-chloro-4-(6-methylpyrazin-2- yl)phenyl]-2-(ethylamino)-8-(4- hydroxybutyl)pyrido[2,3- d]pyrimidin-7-one |
| I-37 | 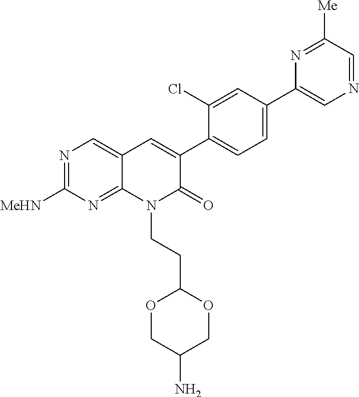 [SMILES: CNc1ncc2cc(-c3ccc(-c4cncc(C)n4)cc3Cl)c(=O)n(CCC3OCC(N)CO3)c2n1] | 0.0075 | 8-[2-(5-amino-1,3-dioxan-2- yl)ethyl]-6-[2-chloro-4-(6- methylpyrazin-2-yl)phenyl]-2- (methylamino)pyrido[2,3- d]pyrimidin-7-one |
| I-38 | 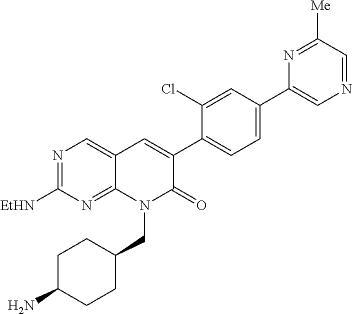 | 0.0035 | 8-[(4-aminocyclohexyl)methyl]-6- [2-chloro-4-(6-methylpyrazin-2- yl)phenyl]-2- (ethylamino)pyrido[2,3- d]pyrimidin-7-one |
| I-39 | 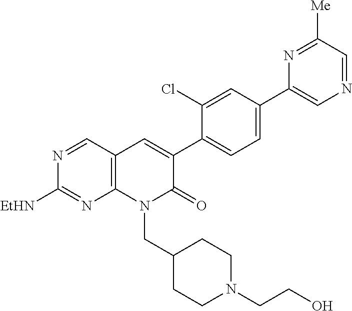 | 0.040 | 6-[2-chloro-4-(6-methylpyrazin-2- yl)phenyl]-2-(ethylamino)-8-[[1-(2- hydroxyethyl)-4- piperidyl]methyl]pyrido[2,3- d]pyrimidin-7-one |
| I-40 | 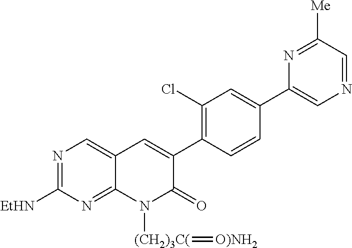 | 0.0417 | 4-[6-[2-chloro-4-(6-methylpyrazin- 2-yl)phenyl]-2-(methylamino)-7- oxo-pyrido[2,3-d]pyrimidin-8- yl]butanamide |
| I-41 | 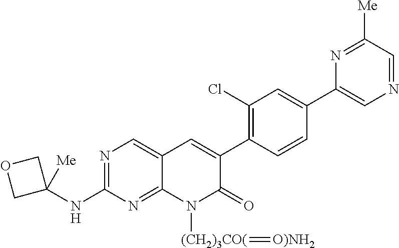 | 0.031 | 4-[6-[2-chloro-4-(6-methylpyrazin- 2-yl)phenyl]-2-[(3-methyloxetan-3- yl)amino]-7-oxo-pyrido[2,3- d]pyrimidin-8-yl]butanamide |
| I-42 | 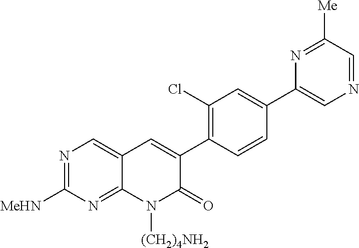 | 0.0232 | 8-(4-aminobutyl)-6-[2-chloro-4-(6- methylpyrazin-2-yl)phenyl]-2- (methylamino)pyrido[2,3- d]pyrimidin-7-one |
| I-43 | 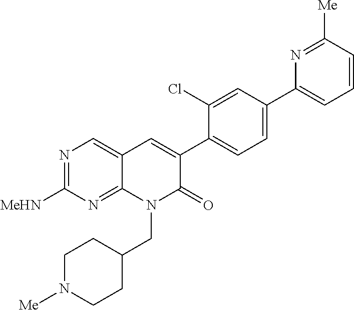 [SMILES: CNc1ncc2cc(-c3ccc(-c4cccc(C)n4)cc3Cl)c(=O)n(CC3CCN(C)CC3)c2n1] | 0.0198 | 6-[2-chloro-4-(6-methyl-2- pyridyl)phenyl]-2-(methylamino)-8- [(1-methyl-4- piperidyl)methyl]pyrido[2,3- d]pyrimidin-7-one |
| I-44 | 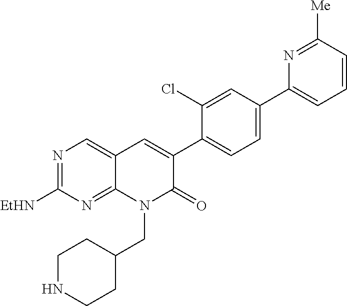 | 0.0046 | 6-[2-chloro-4-(6-methyl-2- pyridyl)phenyl]-2-(ethylamino)-8- (4-piperidylmethyl)pyrido[2,3- d]pyrimidin-7-one |
| I-45 | 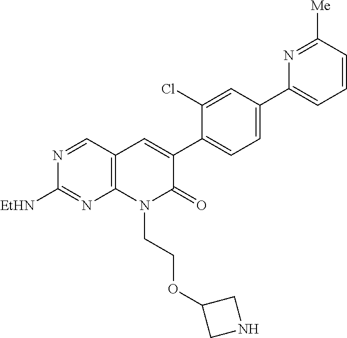 | 0.0099 | 8-[2-(azetidin-3-yloxy)ethyl]-6-[2- chloro-4-(6-methyl-2- pyridyl)phenyl]-2- (ethylamino)pyrido[2,3- d]pyrimidin-7-one |
| I-46 | 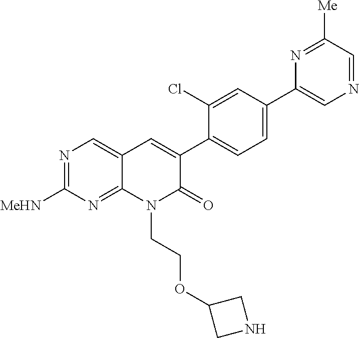 [SMILES: CNc1ncc2cc(-c3ccc(-c4cncc(C)n4)cc3Cl)c(=O)n(CCOC3CNC3)c2n1] | 0.0139 | 8-[2-(azetidin-3-yloxy)ethyl]-6-[2- chloro-4-(6-methylpyrazin-2- yl)phenyl]-2- (methylamino)pyrido[2,3- d]pyrimidin-7-one |
| I-47 | 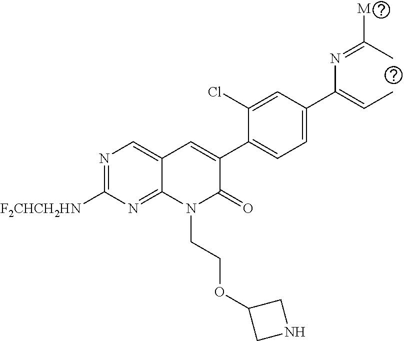 | 0.0252 | 8-[2-(azetidin-3-yloxy)ethyl]-6-[2- chloro-4-(6-methylpyrazin-2- yl)phenyl]-2-(2,2- difluoroethylamino)pyrido[2,3- d]pyrimidin-7-one |
| I-48 | 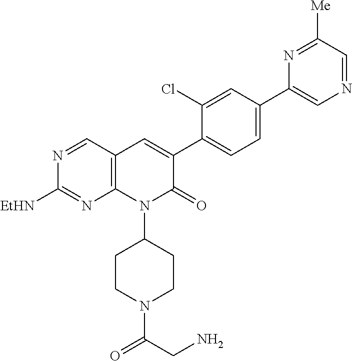 | 0.0171 | 8-[1-(2-aminoacetyl)-4-piperidyl]- 6-[2-chloro-4-(6-methylpyrazin-2- yl)phenyl]-2- (ethylamino)pyrido[2,3- d]pyrimidin-7-one |
| I-49 | 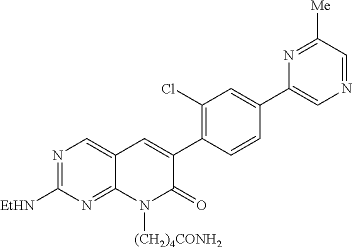 | 0.0753 | 5-[6-[2-chloro-4-(6-methylpyrazin- 2-yl)phenyl]-2-(ethylamino)-7-oxo- pyrido[2,3-d]pyrimidin-8- yl]pentanamide |
| I-50 | 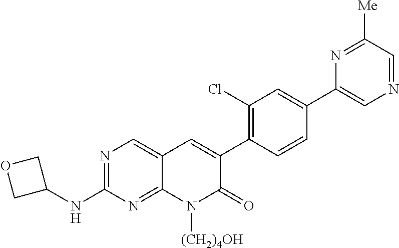 | 0.0456 | 6-[2-chloro-4-(6-methylpyrazin-2- yl)phenyl]-8-(4-hydroxybutyl)-2- (oxetan-3-ylamino)pyrido[2,3- d]pyrimidin-7-one |
| I-51 Isomer A I-52 Isomer B | 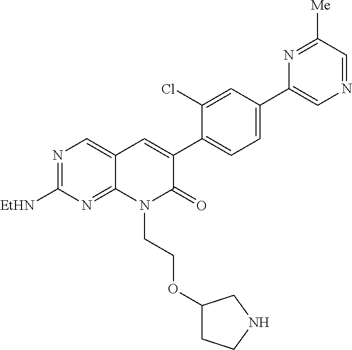 | 0.0132 0.0056 | 6-[2-chloro-4-(6-methylpyrazin-2- yl)phenyl]-2-(ethylamino)-8-(2- pyrrolidin-3-yloxyethyl)pyrido[2,3- d]pyrimidin-7-one |
| I-53 | 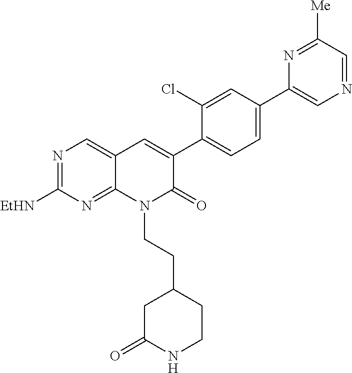 | 0.0962 | 6-[2-chloro-4-(6-methylpyrazin-2- yl)phenyl]-2-(ethylamino)-8-[2-(2- oxo-4-piperidyl)ethyl]pyrido[2,3- d]pyrimidin-7-one |
| I-54 | 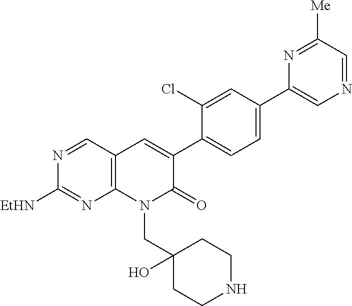 | 0.107 | 6-[2-chloro-4-(6-methylpyrazin-2- yl)phenyl]-2-(ethylamino)-8-[(4- hydroxy-4- piperidyl)methyl]pyrido[2,3- d]pyrimidin-7-one |
| I-55 | 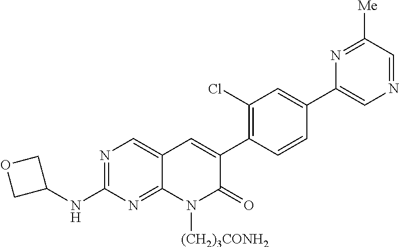 | 0.0298 | 4-[6-[2-chloro-4-(6-methylpyrazin- 2-yl)phenyl]-2-(oxetan-3-ylamino)- 7-oxo-pyrido[2,3-d]pyrimidin-8- yl]butanamide |
| I-56 | 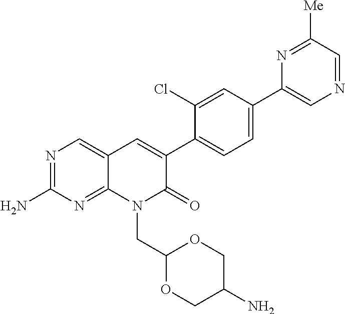 [SMILES: Cc1cncc(-c2ccc(-c3cc4cnc(N)nc4n(CC4OCC(N)CO4)c3=O)c(Cl)c2)n1] | 0.0186 | 2-amino-8-[(5-amino-1,3-dioxan-2- yl)methyl]-6-[2-chloro-4-(6- methylpyrazin-2- yl)phenyl]pyrido[2,3-d]pyrimidin-7- one |
| I-57 | 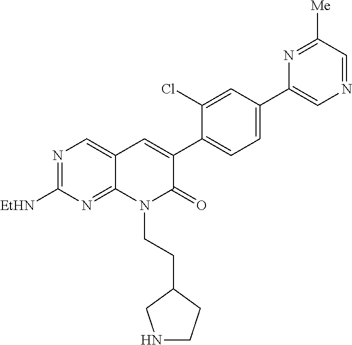 | 0.0041 | 6-[2-chloro-4-(6-methylpyrazin-2- yl)phenyl]-2-(ethylamino)-8-(2- pyrrolidin-3-ylethyl)pyrido[2,3- d]pyrimidin-7-one |
| I-58 | 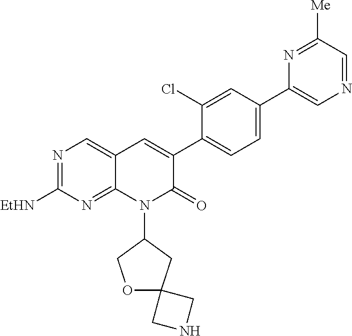 | 0.0711 | 6-[2-chloro-4-(6-methylpyrazin-2- yl)phenyl]-2-(ethylamino)-8-(5-oxa- 2-azaspiro[3.4]octan-7- yl)pyrido[2,3-d]pyrimidin-7-one |
| I-59 | 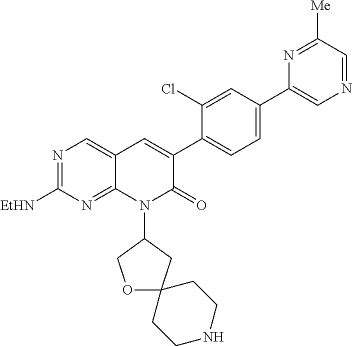 | 0.0244 | 6-[2-chloro-4-(6-methylpyrazin-2- yl)phenyl]-2-(ethylamino)-8-(1-oxa- 8-azaspiro[4.5]decan-3- yl)pyrido[2,3-d]pyrimidin-7-one |
| I-60 | 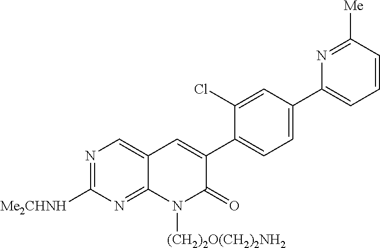 | 0.0011 | 8-[2-(2-aminoethoxy)ethyl]-6-[2- chloro-4-(6-methyl-2- pyridyl)phenyl]-2- osopropylamino)pyrido[2,3- d]pyrimidin-7-one |
| I-61 | 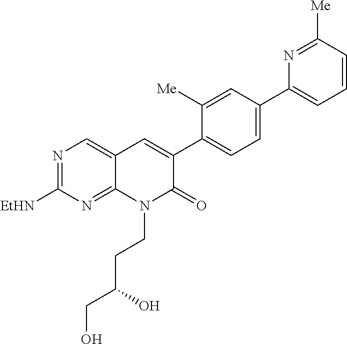 | 0.0139 | 8-[(3R)-3,4-dihydroxybutyl]-2- (ethylamino)-6-[2-methyl-4-(6- methyl-2- pyridyl)phenyl]pyrido[2,3- d]pyrimidin-7-one |
| I-62 | 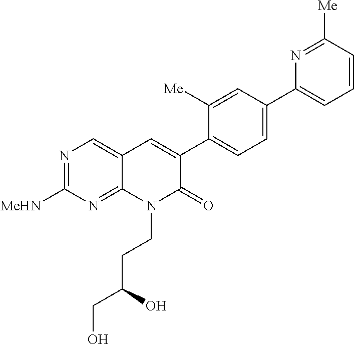 [SMILES: CNc1ncc2cc(-c3ccc(-c4cccc(C)n4)cc3C)c(=O)n(CC[C@@H](O)CO)c2n1] | 0.0127 | 8-[(3R)-3,4-dihydroxybutyl]-2- (ethylamino)-6-[2-methyl-4-(6- methyl-2- pyridyl)phenyl]pyrido[2,3- d]pyrimidin-7-one |
| I-63 | 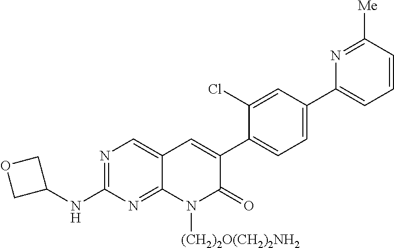 | 0.00030 | 8-[2-(2-aminoethoxy)ethyl]-6-[2- chloro-4-(6-methyl-2- pyridyl)phenyl]-2-(oxetan-3- ylmino)pyrido[2,3-d]pyrimidin-7- one |
| I-64 | 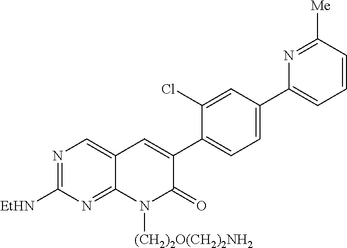 | 0.0028 | 8-[2-(2-aminoethoxy)ethyl]-6-[2- chloro-4-(6-methyl-2- pyridyl)phenyl]-2- (ethylamino)pyrido[2,3- d]pyrimidin-7-one |
| I-65 | 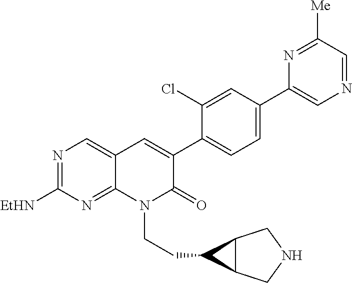 | 0.0145 | 8-[2-[(1R,5S)-3- azabicyclo[3.1.0]hexan-6-yl]ethyl]- 6-[2-chloro-4-(6-methylpyrazin-2- yl)phenyl]-2- (ethylamino)pyrido[2,3- d]pyrimidin-7-one |
| I-66 | 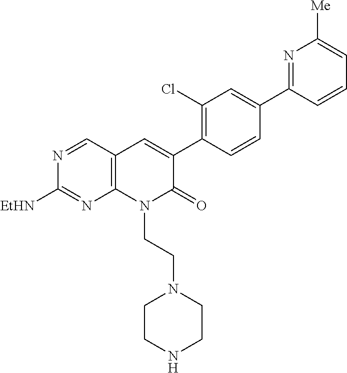 | 0.00090 | 6-[2-chloro-4-(6-methyl-2- pyridyl)phenyl]-2-(ethylamino)-8- (2-piperazin-1-ylethyl)pyrido[2,3- d]pyrimidin-7-one |
| I-67 | 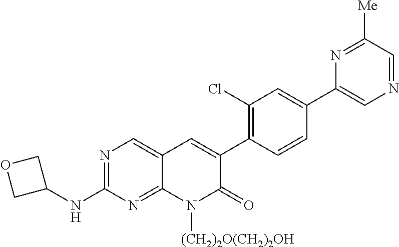 | 0.0778 | 6-[2-chloro-4-(6-methylpyrazin-2- yl)phenyl]-8-[2-(2- hydroxyethoxy)ethyl]-2-(oxetan-3- ylamino)pyrido[2,3-d]pyrimidin-7- one |
| I-68 | 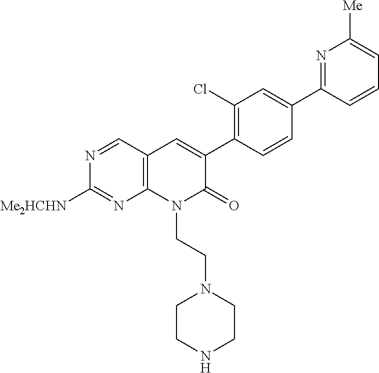 | 0.0016 | 6-[2-chloro-4-(6-methyl-2- pyridyl)phenyl]-2- (isopropylamino)-8-(2-piperazin-1- ylethyl)pyrido[2,3-d]pyrimidin-7- one |
| I-69 | 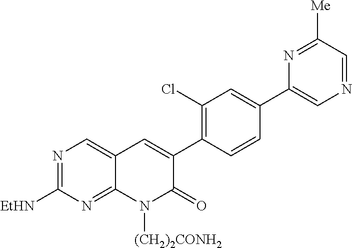 | 0.103 | 3-[6-[2-chloro-4-(6-methylpyrazin- 2-yl)phenyl]-2-(ethylamino)-7-oxo- pyrido[2,3-d]pyrimidin-8- yl]propanamide |
| I-70 | 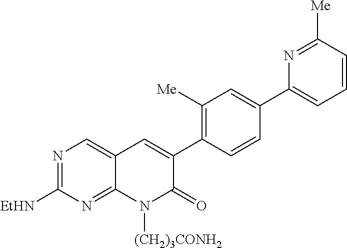 | 0.0174 | 4-[2-(ethylamino)-6-[2-methyl-4-(6- methyl-2-pyridyl)phenyl]-7-oxo- pyrido[2,3-d]pyrimidin-8- yl]butanamide |
| I-71 | 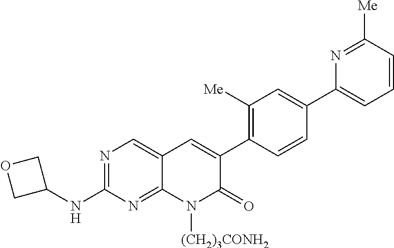 | 0.0113 | 4-[6-[2-methyl-4-(6-methyl-2- pyridyl)phenyl]-2-(oxetan-3- ylamino)-7-oxo-pyrido[2,3- d]pyrimidin-8-yl]butanamide |
| I-72 | 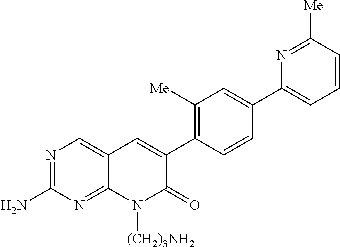 | 0.0211 | 2-amino-8-(3-aminopropyl)-6-[2- methyl-4-(6-methyl-2- pyridyl)phenyl]pyrido[2,3- d]pyrimidin-7-one |
| I-73 | 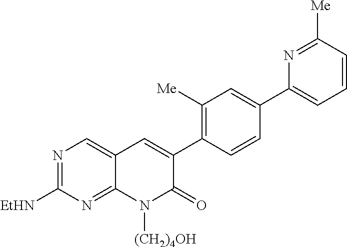 | 0.0189 | 2-(ethylamino)-8-(4-hydroxybutyl)- 6-[2-methyl-4-(6-methyl-2- pyridyl)phenyl]pyrido[2,3- d]pyrimidin-7-one |
| I-74 | 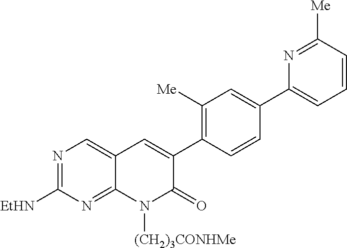 | 0.0155 | 4-[2-(ethylamino)-6-[2-methyl-4-(6- methyl-2-pyridyl)phenyl]-7-oxo- pyrido[2,3-d]pyrimidin-8-yl]-N- methyl-butanamide |
| I-75 | 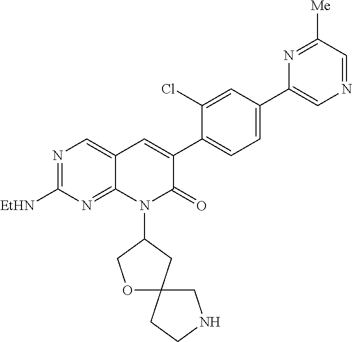 | 0.0203 | 6-[2-chloro-4-(6-methylpyrazin-2- yl)phenyl]-2-(ethylamino)-8-(1-oxa- 7-azaspiro[4,4]nonan-3- yl)pyrido[2,3-d]pyrimidin-7-one |
| I-76 | 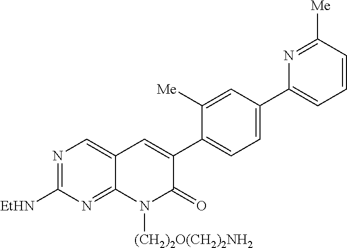 | 0.0033 | 8-[2-(2-aminoethoxy)ethyl]-2- (ethylamino)-6-[2-methyl-4-(6- methyl-2- pyridyl)phenyl]pyrido[2,3- d]pyrimidin-7-one |
| I-77 | 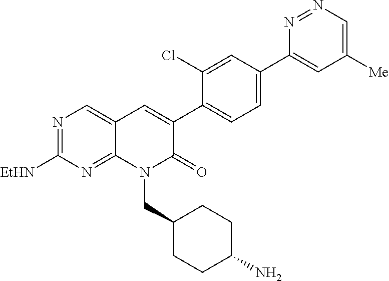 | 0.0307 | 8-[(4-aminocyclohexyl)methyl]-6- [2-chloro-4-(5-methylpyridazin-3- yl)phenyl]-2- (ethylamino)pyrido[2,3- d]pyrimidin-7-one |
| I-78 | 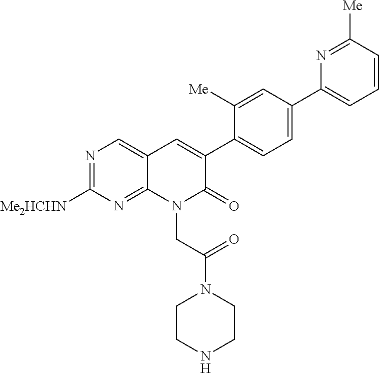 | 0.016 | 2-(isopropylamino)-6-[2-methyl-4- (6-methyl-2-pyridyl)phenyl]-8-(2- oxo-2-piperazin-1-yl- ethyl)pyrido[2,3-d]pyrimidin-7-one |
| I-79 | 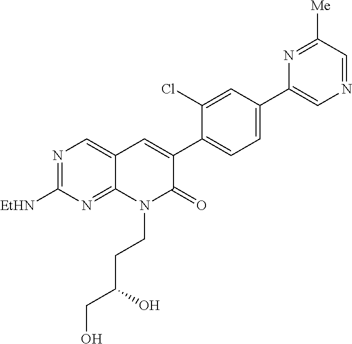 | 0.0435 | 6-[2-chloro-4-(6-methylpyrazin-2- yl)phenyl]-8-[(3R)-3,4- dihydroxybutyl]-2- (ethylamino)pyrido[2,3- d]pyrimidin-7-one |
| I-80 | 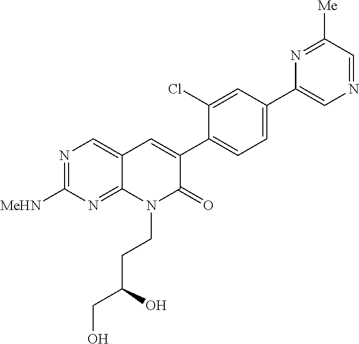 [SMILES: CNc1ncc2cc(-c3ccc(-c4cncc(C)n4)cc3Cl)c(=O)n(CC[C@@H](O)CO)c2n1] | 0.104 | 6-[2-chloro-4-(6-methylpyrazin-2- yl)phenyl]-8-(3,4-dihydroxybutyl)- 2-(methylamino)pyrido[2,3- d]pyrimidin-7-one |
| I-81 | 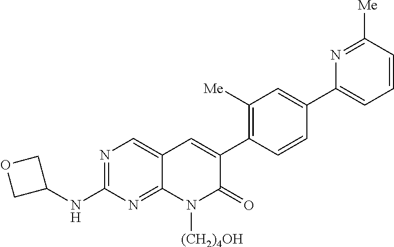 | 0.0065 | 8-(4-hydroxybutyl)-6-[2-methyl-4- (6-methyl-2-pyridyl)phenyl]-2- (oxetan-3-ylamino)pyrido[2,3- d]pyrimidin-7-one |
| I-82 | 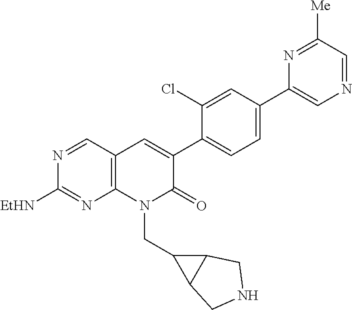 | 0.0096 | 8-(3-azabicyclo[3.1.0]hexan-6- ylmethyl)-6-[2-chloro-4-(6- methylpyrazin-2-yl)phenyl]-2- (ethylamino)pyrido[2,3- d]pyrimidin-7-one |
| I-83 | 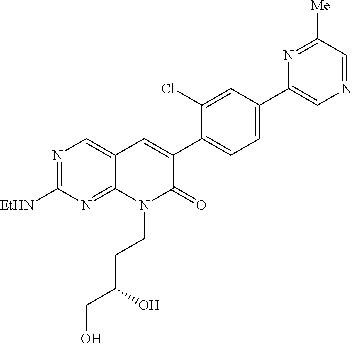 | 0.107 | 6-[2-chloro-4-(6-methylpyrazin-2- yl)phenyl]-8-[(3S)-3,4- dihydroxybutyl]-2- (ethylamino)pyrido[2,3- d]pyrimidin-7-one |
| I-84 | 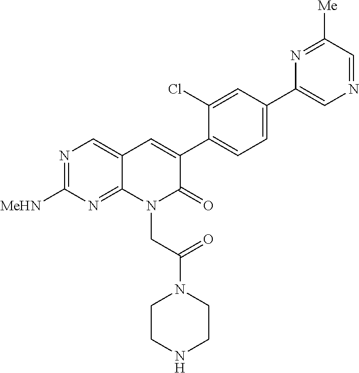 [SMILES: CNc1ncc2cc(-c3ccc(-c4cncc(C)n4)cc3Cl)c(=O)n(CC(=O)N3CCNCC3)c2n1] | 0.0592 | 6-[2-chloro-4-(6-methylpyrazin-2- yl)phenyl]-2-(methylamino)-8-(2- oxo-2-piperazin-1-yl- ethyl)pyrido[2,3-d]pyrimidin-7-one |
| I-85 | 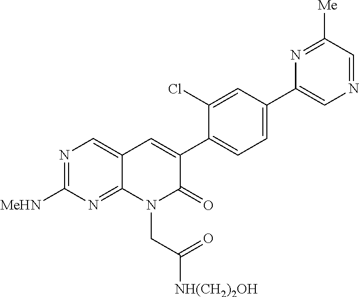 | 0.0929 | 2-[6-[2-chloro-4-(6-methylpyrazin- 2-yl)phenyl]-2-(methylamino)-7- oxo-pyrido[2,3-d]pyrimidin-8-yl]- N-(2-hydroxyethyl)acetamide |
| I-86 | 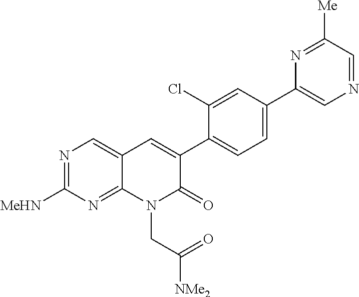 [SMILES: CNc1ncc2cc(-c3ccc(-c4cncc(C)n4)cc3Cl)c(=O)n(CC(=O)N(C)C)c2n1] | 0.123 | 2-[6-[2-chloro-4-(6-methylpyrazin- 2-yl)phenyl]-2-(methylamino)-7- oxo-pyrido[2,3-d]pyrimidin-8-yl]- N,N-dimethyl-acetamide |
| I-87 | 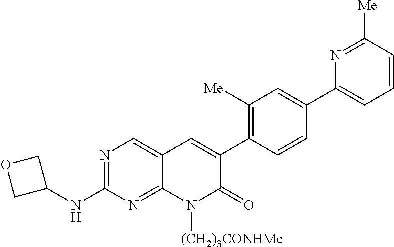 | 0.0305 | N-methyl-4-[6-[2-methyl-4-(6- methyl-2-pyridyl)phenyl]-2- (oxetan-3-ylamino)-7-oxo- pyrido[2,3-d]pyrimidin-8- yl]butanamide |
| I-88 | 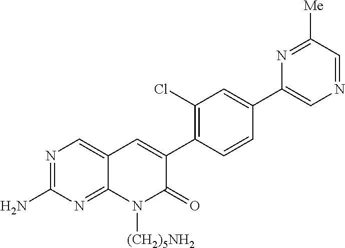 | 0.313 | 2-amino-8-(5-aminopentyl)-6-[2- chloro-4-(6-methylpyrazin-2- yl)phenyl]pyrido[2,3-d]pyrimidin-7- one |
| I-89 | 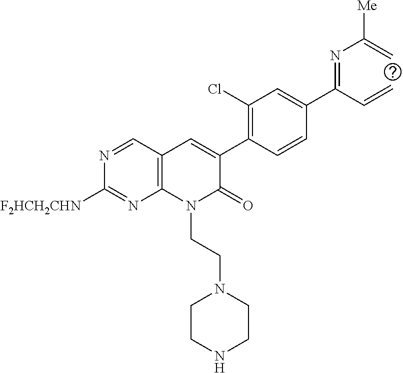 | 0.0078 | 6-[2-chloro-4-(6-methyl-2- pyridyl)phenyl]-2-(2,2- difluoroethylamino)-8-(2-piperazin- 1-ylethyl)pyrido[2,3-d]pyrimidin-7- one |
| I-90 | 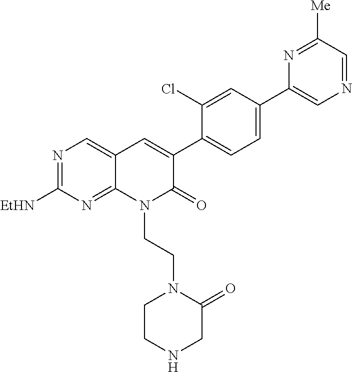 | 0.322 | 6-[2-chloro-4-(6-methylpyrazin-2- yl)phenyl]-2-(ethylamino)-8-[2-(2- oxopiperazin-1-yl)ethyl]pyrido[2,3- d]pyrimidin-7-one |
| I-91 Isomer A I-92 Isomer B | 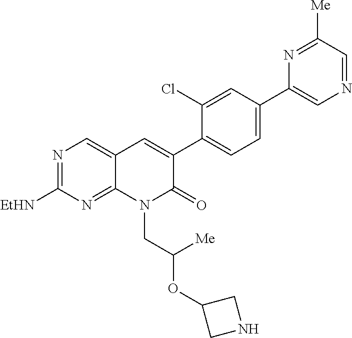 | 0.0071 0.0476 | 8-[2-(azetidin-3-yloxy)propyl]-6-[2- chloro-4-(6-methylpyrazin-2- yl)phenyl]-2- (ethylamino)pyrido[2,3- d]pyrimidin-7-one |
| I-93 | 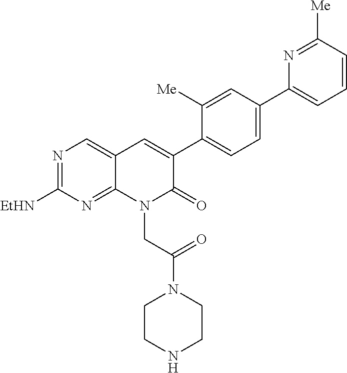 | 0.0291 | 2-(ethylamino)-6-[2-methyl-4-(6- methyl-2-pyridyl)phenyl]-8-(2-oxo- 2-piperazin-1-yl-ethyl)pyrido[2,3- d]pyrimidin-7-one |
| I-94 | 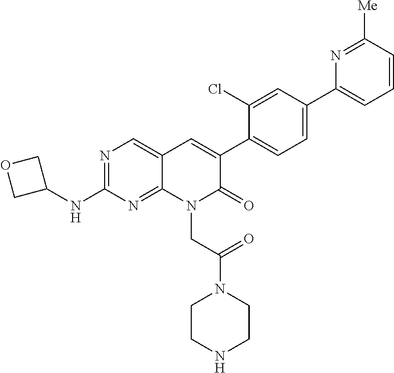 [SMILES: Cc1cccc(-c2ccc(-c3cc4cnc(NC5COC5)nc4n(CC(=O)N4CCNCC4)c3=O)c(Cl)c2)n1] | 0.0159 | 6-[2-chloro-4-(6-methyl-2- pyridyl)phenyl]-2-(oxetan-3- ylamino)-8-(2-oxo-2-piperazin-1-yl- ethyl)pyrido[2,3-d]pyrimidin-7-one |
| I-95 | 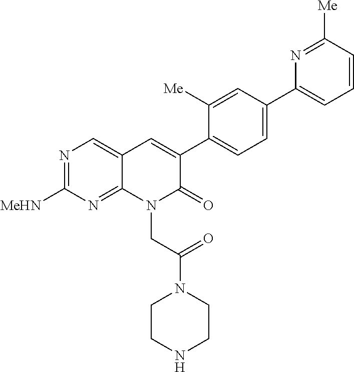 [SMILES: CNc1ncc2cc(-c3ccc(-c4cccc(C)n4)cc3C)c(=O)n(CC(=O)N3CCNCC3)c2n1] | 0.0273 | 2-(methylamino)-6-[2-methyl-4-(6- methyl-2-pyridyl)phenyl]-8-(2-oxo- 2-piperazin-1-yl-ethyl)pyrido[2 3- d]pyrimidin-7-one |
| I-96 | 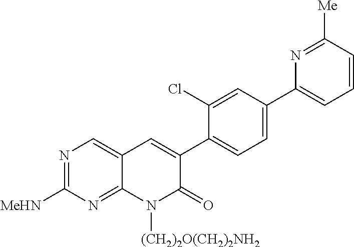 | 0.0014 | 8-[2-(2-aminoethoxy)ethyl]-6-[2- chloro-4-(6-methyl-2- pyridyl)phenyl]-2- (methylamino)pyrido[2,3- d]pyrimidin-7-one |
| I-97 | 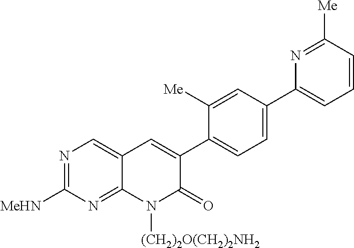 | 0.0036 | 8-[2-(2-aminoethoxy)ethyl]-2- (methylamino)-6-[2-methyl-4-(6- methyl-2- pyridyl)phenyl]pyrido[2,3- d]pyrimidin-7-one |
| I-98 Isomer A I-99 Isomer B | 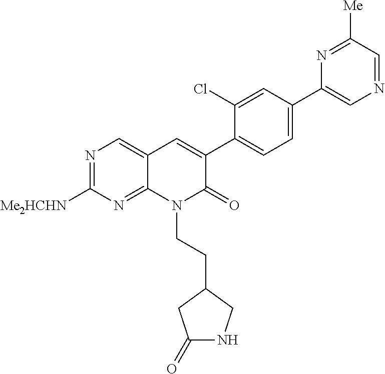 | 0.0218 0.0191 | 6-[2-chloro-4-(6-methylpyrazin-2- yl)phenyl]-2-(isopropylamino)-8-[2- (5-oxopyrrolidin-3- yl)ethyl]pyrido[2,3-d]pyrimidin-7- one |
| I-100 I-101 | 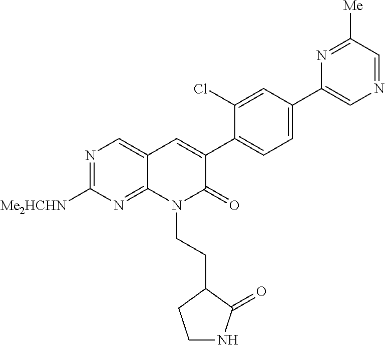 | 0.0185 N/A | 6-[2-chloro-4-(6-methylpyrazin-2- yl)phenyl]-2-(isopropylamino)-8-[2- (2-oxopyrrolidin-3- yl)ethyl]pyrido[2,3-d]pyrimidin-7- one |
| I-102 Isomer A I-103 Isomer B | 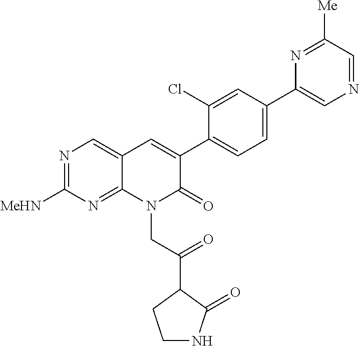 [SMILES: CNc1ncc2cc(-c3ccc(-c4cncc(C)n4)cc3Cl)c(=O)n(CC(=O)C3CCNC3=O)c2n1] | 0.0381 0.019 | 6-[2-chloro-4-(6-methylpyrazin-2- yl)phenyl]-2-(isopropylamino)-8-[2- (2-oxopyrrolidin-3- yl)ethyl]pyrido[2,3-d]pyrimidin-7- one |
| I-104 Isomer A I-110 Isomer B | 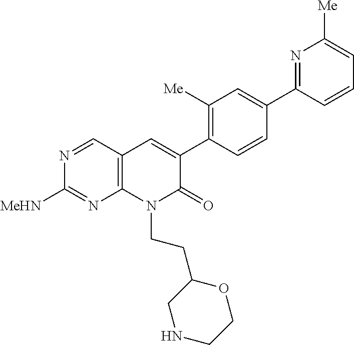 [SMILES: CNc1ncc2cc(-c3ccc(-c4cccc(C)n4)cc3C)c(=O)n(CCC3CNCCO3)c2n1] | 0.0023 0.003 | 2-(methylamino)-6-[2-methyl-4-(6- methyl-2-pyridyl)phenyl]-8-(2- morpholin-2-ylethyl)pyrido[2,3- d]pyrimidin-7-one |
| I-105 | 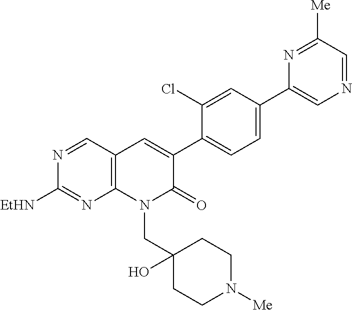 | 0.114 | 6-[2-chloro-4-(6-methylpyrazin-2- yl)phenyl]-2-(ethylamino)-8-[(4- hydroxy-1-methyl-4- piperidyl)methyl]pyrido[2,3- d]pyrimidin-7-one |
| I-106 | 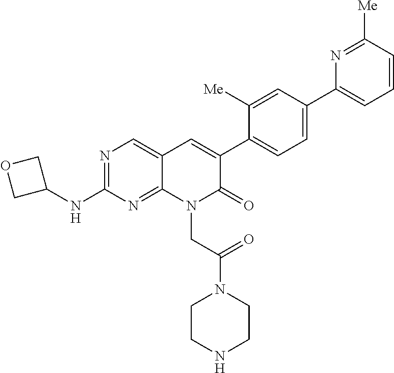 [SMILES: Cc1cccc(-c2ccc(-c3cc4cnc(NC5COC5)nc4n(CC(=O)N4CCNCC4)c3=O)c(C)c2)n1] | 0.0545 | 6-[2-methyl-4-(6-methyl-2- pyridyl)phenyl]-2-(oxetan-3- ylamino)-8-(2-oxo-2-piperazin-1-yl- ethyl)pyrido[2,3-d]pyrimidin-7-one |
| I-107 | 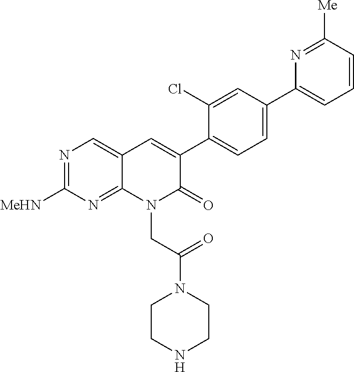 [SMILES: CNc1ncc2cc(-c3ccc(-c4cccc(C)n4)cc3Cl)c(=O)n(CC(=O)N3CCNCC3)c2n1] | 0.0143 | 6-[2-chloro-4-(6-methyl-2- pyridyl)phenyl]-2-(methylamino)-8- (2-oxo-2-piperazin-1-yl- ethyl)pyrido[2,3-d]pyrimidin-7-one |
| I-108 | 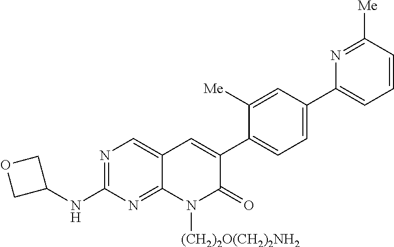 | 0.0025 | 8-[2-(2-aminoethoxy)ethyl]-6-[2- methyl-4-(6-methyl-2- pyridyl)phenyl]-2-(oxetan-3- ylamino)pyrido[2,3-d]pyrimidin-7- one |
| I-109 | 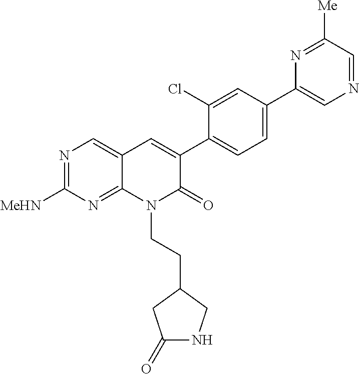 [SMILES: CNc1ncc2cc(-c3ccc(-c4cncc(C)n4)cc3Cl)c(=O)n(CCC3CNC(=O)C3)c2n1] | 0.0433 | 6-[2-chloro-4-(6-methylpyrazin-2- yl)phenyl]-2-(methylamino)-8-[2- (5-oxopyrrolidin-3- yl)ethyl]pyrido[2,3-d]pyrimidin-7- one |
| I-111 Isomer A I-128 Isomer B | 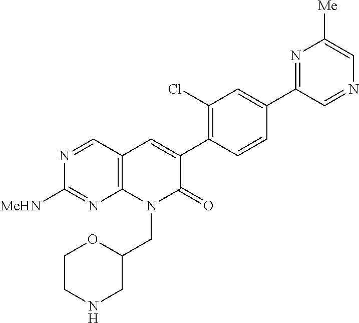 [SMILES: CNc1ncc2cc(-c3ccc(-c4cncc(C)n4)cc3Cl)c(=O)n(CC3CNCCO3)c2n1] | 0.0194 0.0113 | 6-[2-chloro-4-(6-methylpyrazin-2- yl)phenyl]-2-(methylamino)-8- [[(2R)-morpholin-2- yl]methyl]pyrido[2,3-d]pyrimidin- 7-one |
| I-112 Isomer A I-115 Isomer B | 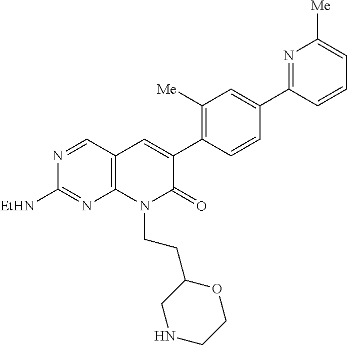 | 0.0010 0.0014 | 2-(ethylamino)-6-[2-methyl-4-(6- methyl-2-pyridyl)phenyl]-8-(2- morpholin-2-ylethyl)pyrido[2,3- d]pyrimidin-7-one |
| I-113 Isomer A I-114 Isomer B | 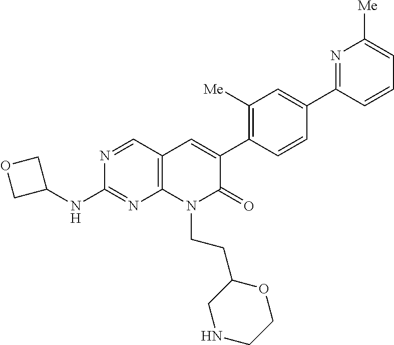 [SMILES: Cc1cccc(-c2ccc(-c3cc4cnc(NC5COC5)nc4n(CCC4CNCCO4)c3=O)c(C)c2)n1] | 0.0017 0.00070 | 6-[2-methyl-4-(6-methyl-2- pyridyl)phenyl]-8-(2-morpholin-2- ylethyl)-2-(oxetan-3- ylamino)pyrido[2,3-d]pyrimidin-7- one |
| I-116 | 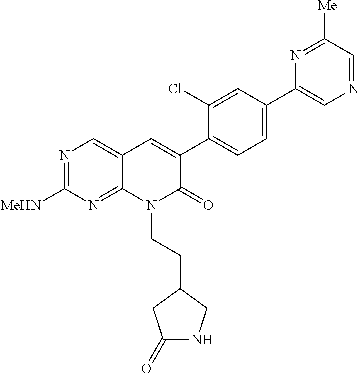 [SMILES: CNc1ncc2cc(-c3ccc(-c4cncc(C)n4)cc3Cl)c(=O)n(CCC3CNC(=O)C3)c2n1] | N/A | 6-[2-chloro-4-(6-methylpyrazin-2- yl)phenyl]-2-(methylamino)-8-[2- (5-oxopyrrolidin-3- yl)ethyl]pyrido[2,3-d]pyrimidin-7- one |
| I-117 | 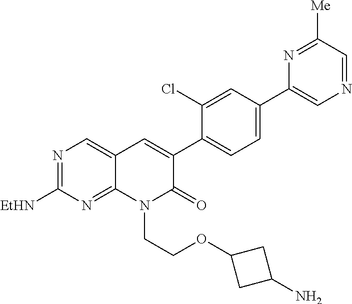 | 0.0037 | 8-[2-(3-aminocyclobutoxy)ethyl]-6- [2-chloro-4-(6-methylpyrazin-2- yl)phenyl]-2- (ethylamino)pyrido[2,3- d]pyrimidin-7-one |
| I-118 Isomer A I-119 Isomer B | 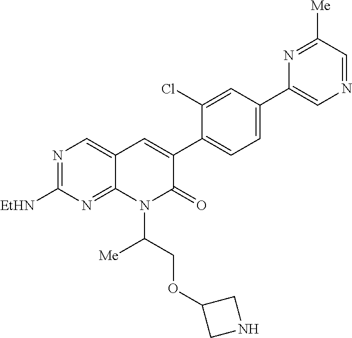 | 0.0183 0.077 | 8-[2-(azetidin-3-yloxy)-1-methyl- ethyl]-6-[2-chloro-4-(6- methylpyrazin-2-yl)phenyl]-2- (ethylamino)pyrido[2,3- d]pyrimidin-7-one |
| I-120 | 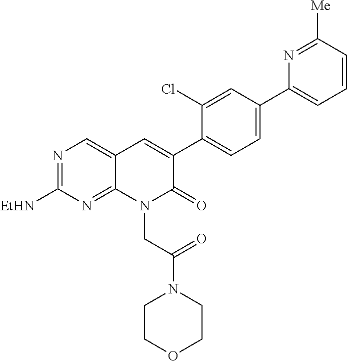 | 0.0208 | 6-[2-chloro-4-(6-methyl-2- pyridyl)phenyl]-2-(ethylamino)-8- (2-morpholino-2-oxo- ethyl)pyrido[2,3-d]pyrimidin-7-one |
| I-121 | 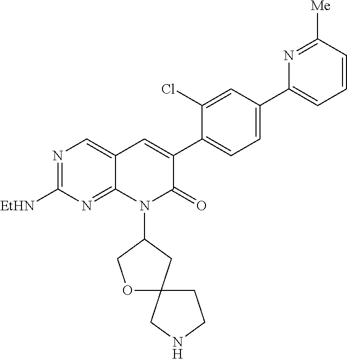 | 0.0037 | 6-[2-chloro-4-(6-methyl-2- pyridyl)phenyl]-2-(ethylamino)-8- (1-oxa-7-azaspiro[4.4]nonan-3- yl)pyrido[2,3-d]pyrimidin-7-one |
| I-122 | 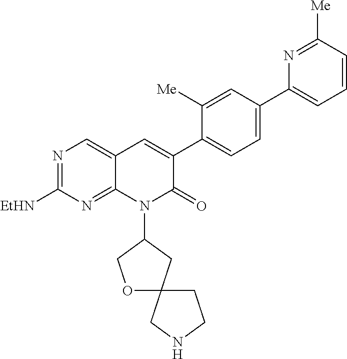 | 0.0045 | 2-(ethylamino)-6-[2-methyl-4-(6- methyl-2-pyridyl)phenyl]-8-(1-oxa- 7-azaspiro[4.4]nonan-3- yl)pyrido[2,3-d]pyrimidin-7-one |
| I-123 | 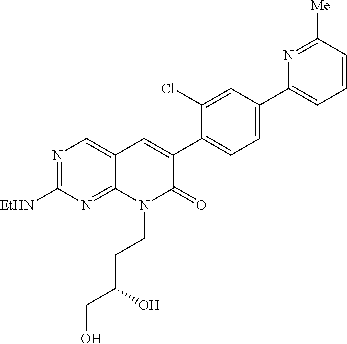 | 0.00708 | 6-[2-chloro-4-(6-methyl-2- pyridyl)phenyl]-8-[(3S)-3,4- dihydroxybutyl]-2- (ethylamino)pyrido[2,3- d]pyrimidin-7-one |
| I-124 Isomer A I-125 Isomer B | 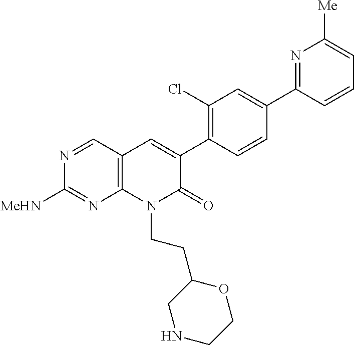 [SMILES: CNc1ncc2cc(-c3ccc(-c4cccc(C)n4)cc3Cl)c(=O)n(CCC3CNCCO3)c2n1] | 0.0015 0.0047 | 6-[2-chloro-4-(6-methyl-2- pyridyl)phenyl]-2-(methylamino)-8- (2-morpholin-2-ylethyl)pyrido[2,3- d]pyrimidin-7-one |
| I-126 | 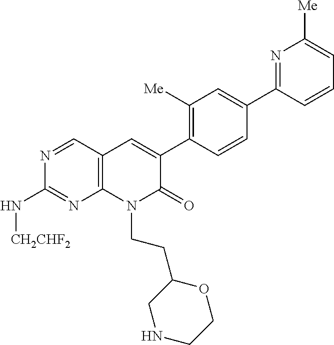 | 0.0025 | 2-(2 2-difluoroethylamino)-6-[2- methyl-4-(6-methyl-2- pyridyl)phenyl]-8-(2-morpholin-2- ylethyl)pyrido[2,3-d]pyrimidin-7- one |
| I-127 | 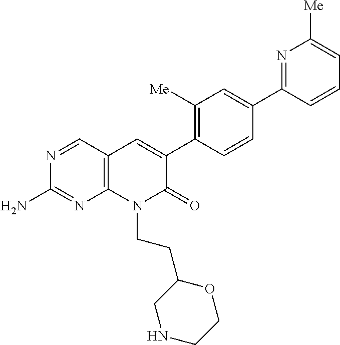 [SMILES: Cc1cccc(-c2ccc(-c3cc4cnc(N)nc4n(CCC4CNCCO4)c3=O)c(C)c2)n1] | 0.0038 | 2-amino-6-[2-chloro-4-(6-methyl-2- pyridyl)phenyl]-8-(2-morpholin-2- ylethyl)pyrido[2,3-d]pyrimidin-7- one |
| I-128 | 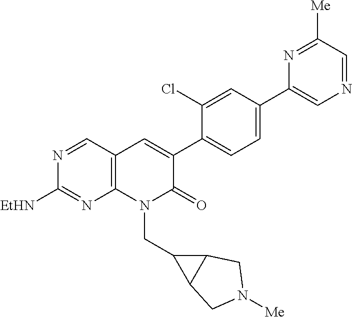 | 0.0118 | 6-[2-chloro-4-(6-methylpyrazin-2- yl)phenyl]-2-(ethylamino)-8-[(3- methyl-3-azabicyclo[3.1.0]hexan-6- yl)methyl]pyrido[2,3-d]pyrimidin- 7-one |
| I-129 | 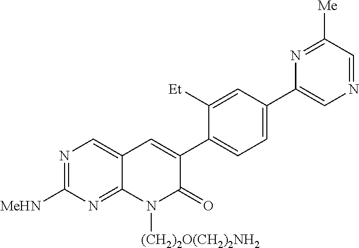 | 0.0275 | 8-[2-(2-aminoethoxy)ethyl]-6-[2- ethyl-4-(6-methylpyrazin-2- yl)phenyl]-2- (methylamino)pyrido[2,3- d]pyrimidin-7-one |
| I-130 | 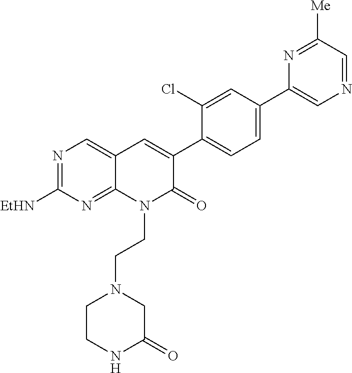 | 0.0522 | 6-[2-chloro-4-(6-methylpyrazin-2- yl)phenyl]-2-(ethylamino)-8-[2-(3- oxopiperazin-1-yl)ethyl]pyrido[2,3- d]pyrimidin-7-one |
| I-131 | 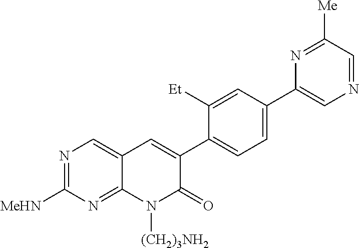 | 0.0325 | 8-(3-aminopropyl)-6-[2-ethyl-4-(6- methylpyrazin-2-yl)phenyl]-2- (methylamino)pyrido[2,3- d]pyrimidin-7-one |
| I-132 | 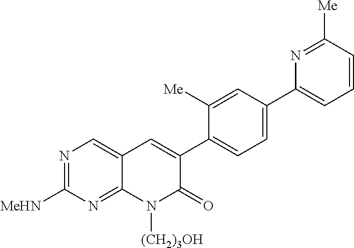 | 0.0145 | 8-(3-hydroxypropyl)-2- (methylamino)-6-[2-methyl-4-(6- methyl-2- pyridyl)phenyl]pyrido[2,3- d]pyrimidin-7-one |
| I-133 | 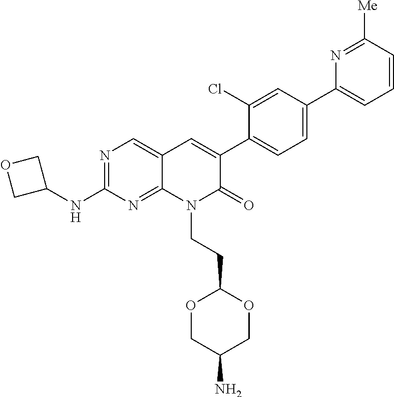 [SMILES: Cc1cccc(-c2ccc(-c3cc4cnc(NC5COC5)nc4n(CC[C@H]4OC[C@@H](N)CO4)c3=O)c(Cl)c2)n1] | 0.0009 | 8-[2-(5-amino-1,3-dioxan-2- yl)ethyl]-6-[2-chloro-4-(6-methyl- 2-pyridyl)phenyl]-2-(oxetan-3- ylamino)pyrido[2,3-d]pyrimidin-7- one |
| I-134 | 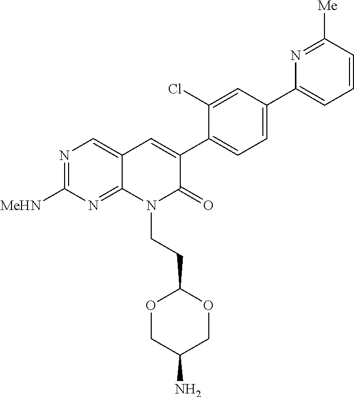 [SMILES: CNc1ncc2cc(-c3ccc(-c4cccc(C)n4)cc3Cl)c(=O)n(CC[C@H]3OC[C@@H](N)CO3)c2n1] | 0.00345 | 8-[2-(5-amino-1,3-dioxan-2- yl)ethyl]-6-[2-chloro-4-(6-methyl- 2-pyridyl)phenyl]-2- (methylamino)pyrido[2,3- d]pyrimidin-7-one |
| I-135 | 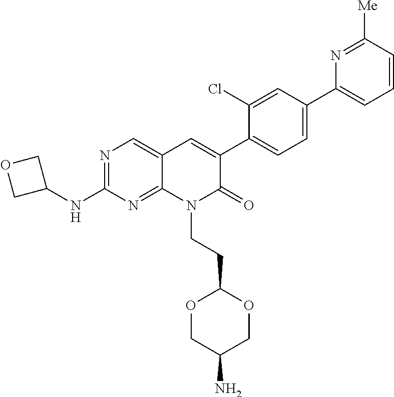 [SMILES: Cc1cccc(-c2ccc(-c3cc4cnc(NC5COC5)nc4n(CC[C@H]4OC[C@@H](N)CO4)c3=O)c(Cl)c2)n1] | 0.000654 | 8-[2-(5-amino-1,3-dioxan-2- yl)ethyl]-6-[2-chloro-4-(6-methyl- 2-pyridyl)phenyl]-2-(oxetan-3- ylamino)pyrido[2,3-d]pyrimidin-7- one |
| I-136 | 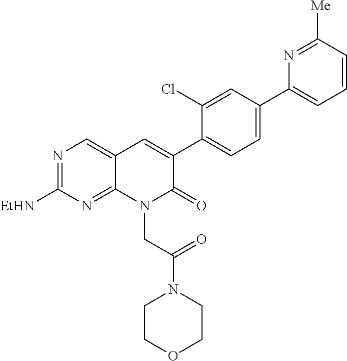 | 0.0208 | 6-[2-chloro-4-(6-methyl-2- pyridyl)phenyl]-2-(ethylamino)-8- (2-morpholino-2-oxo- ethyl)pyrido[2,3-d]pyrimidin-7-one |
| I-137 | 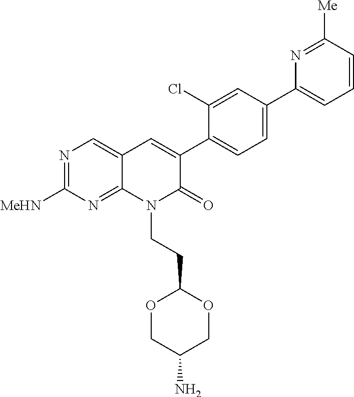 [SMILES: CNc1ncc2cc(-c3ccc(-c4cccc(C)n4)cc3Cl)c(=O)n(CC[C@H]3OC[C@H](N)CO3)c2n1] | 0.00092 | 8-[2-(5-amino-1,3-dioxan-2- yl)ethyl]-6-[2-chloro-4-(6-methyl- 2-pyridyl)phenyl]-2 (methylamino)pyrido[2,3- d]pyrimidin-7-one |
| I-138 | 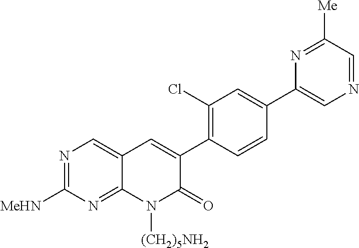 | 0.0096 | 8-(5-aminopentyl)-6-[2-chloro-4-(6- methylpyrazin-2-yl)phenyl]-2- (methylamino)pyrido[2,3- d]pyrimidin-7-one |
| I-139 | 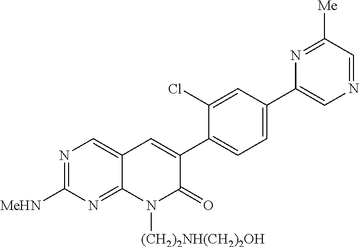 | 0.056 | 6-[2-chloro-4-(6-methylpyrazin-2- yl)phenyl]-8-[2-(2- hydroxyethylamino)ethyl]-2- (methylamino)pyrido[2,3- d]pyrimidin-7-one |
| I-140 | 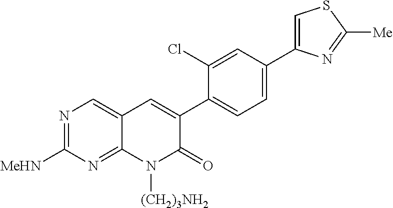 | 0.00806 | 8-(3-aminopropyl)-6-[2-chloro-4- (2-methylthiazol-4-yl)phenyl]-2- (methylamino)pyrido[2,3- d]pyrimidin-7-one |
| I-141 | 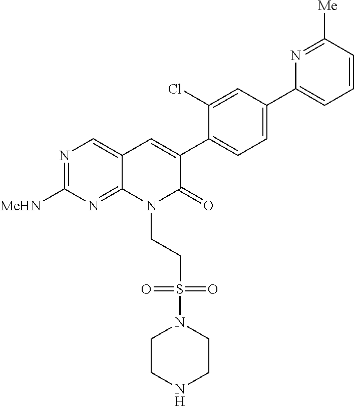 [SMILES: CNc1ncc2cc(-c3ccc(-c4cccc(C)n4)cc3Cl)c(=O)n(CCS(=O)(=O)N3CCNCC3)c2n1] | 0.004 | 6-[2-chloro-4-(6-methyl-2- pyridyl)phenyl]-2-(methylamino)-8- (2-piperazin-1- ylsulfonylethyl)pyrido[2,3- d]pyrimidin-7-one |
| I-142 | 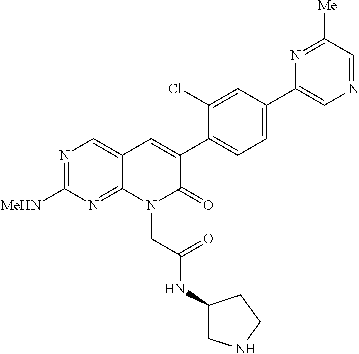 [SMILES: CNc1ncc2cc(-c3ccc(-c4cncc(C)n4)cc3Cl)c(=O)n(CC(=O)N[C@H]3CCNC3)c2n1] | 0.014 | 2-[6-[2-chloro-4-(6-methylpyrazin- 2-yl)phenyl]-2-(methylamino)-7- oxo-pyrido[2,3-d]pyrimidin-8-yl]- N-[(3S)-pyrrolidin-3-yl]acetamide |
| I-143 | 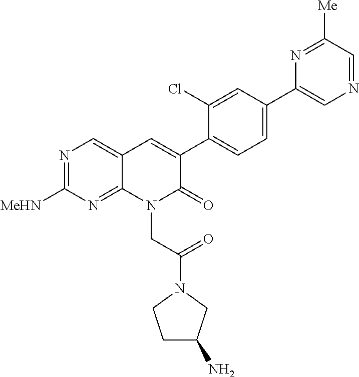 [SMILES: CNc1ncc2cc(-c3ccc(-c4cncc(C)n4)cc3Cl)c(=O)n(CC(=O)N3CC[C@H](N)C3)c2n1] | 0.0125 | 8-[2-[(3S)-3-aminopyrrolidin-1-yl]- 2-oxo-ethyl]-6-[2-chloro-4-(6- methylpyrazin-2-yl)phenyl]-2- (methylamino)pyrido[2,3- d]pyrimidin-7-one |
| I-144 | 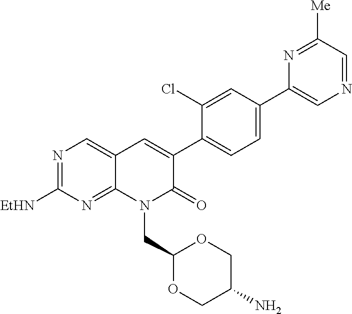 | 0.0097 | 8-[(5-amino-1,3-dioxan-2- yl)methyl]-6-[2-chloro-4-(6- methylpyrazin-2-yl)phenyl]-2- (ethylamino)pyrido[2,3- d]pyrimidin-7-one |
| I-145 | 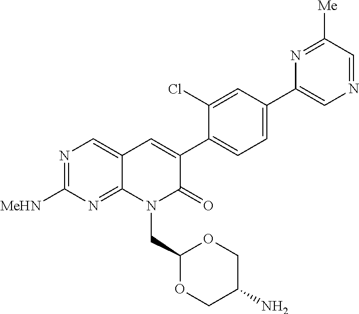 [SMILES: CNc1ncc2cc(-c3ccc(-c4cncc(C)n4)cc3Cl)c(=O)n(C[C@H]3OC[C@H](N)CO3)c2n1] | 0.00854 | 8-[(5-amino-1,3-dioxan-2- yl)methyl]-6-[2-chloro-4-(6- methylpyrazin-2-yl)phenyl]-2- (methylamino)pyrido[2,3- d]pyrimidin-7-one |
| I-146 | 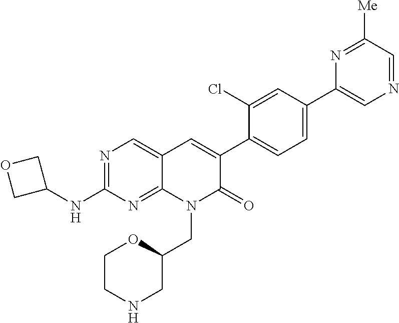 [SMILES: Cc1cncc(-c2ccc(-c3cc4cnc(NC5COC5)nc4n(C[C@H]4CNCCO4)c3=O)c(Cl)c2)n1] | 0.0107 | 6-[2-chloro-4-(6-methylpyrazin-2- yl)phenyl]-8-[[(2R)-morpholin-2- yl]methyl]-2-(oxetan-3- ylamino)pyrido[2,3-d]pyrimidin-7- one |
| I-147 | 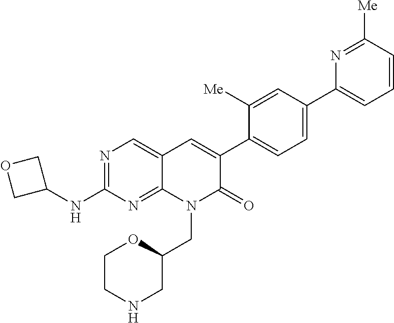 [SMILES: Cc1cccc(-c2ccc(-c3cc4cnc(NC5COC5)nc4n(C[C@H]4CNCCO4)c3=O)c(C)c2)n1] | 0.00612 | 6-[2-methyl-4-(6-methyl-2- pyridyl)phenyl]-8-[[(2R)- morpholin-2-yl]methyl]-2-(oxetan- 3-ylamino)pyrido[2,3-d]pyrimidin- 7-one |
| I-148 | 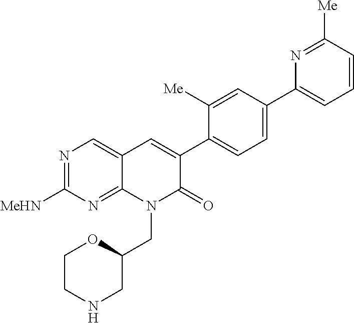 [SMILES: CNc1ncc2cc(-c3ccc(-c4cccc(C)n4)cc3C)c(=O)n(C[C@H]3CNCCO3)c2n1] | 0.0073 | 2-(methylamino)-6-[2-methyl-4-(6- methyl-2-pyridyl)phenyl]-8-[[(2R)- morpholin-2-yl]methyl]pyrido[2,3- d]pyrimidin-7-one |
| I-149 | 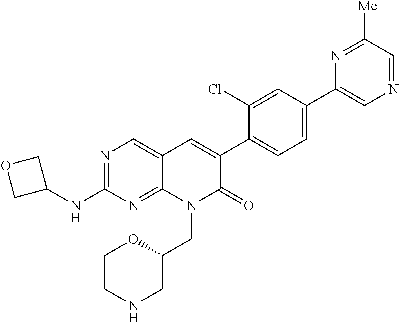 [SMILES: Cc1cncc(-c2ccc(-c3cc4cnc(NC5COC5)nc4n(C[C@@H]4CNCCO4)c3=O)c(Cl)c2)n1] | 0.009 | 6-[2-chloro-4-(6-methylpyrazin-2- yl)phenyl]-8-[[(2S)-morpholin-2- yl]methyl]-2-(oxetan-3- ylamino)pyrido[2,3-d]pyrimidin-7- one |
| I-150 | 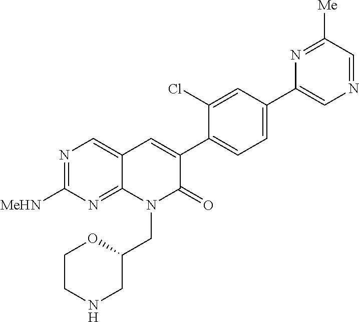 [SMILES: CNc1ncc2cc(-c3ccc(-c4cncc(C)n4)cc3Cl)c(=O)n(C[C@@H]3CNCCO3)c2n1] | 0.0113 | 6-[2-chloro-4-(6-methylpyrazin-2- yl)phenyl]-2-(methylamino)-8- [[(2S)-morpholin-2- yl]methyl]pyrido[2,3-d]pyrimidin- 7-one |
| I-151 | 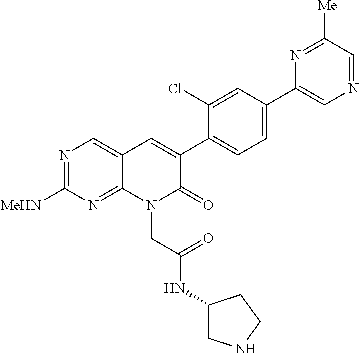 [SMILES: CNc1ncc2cc(-c3ccc(-c4cncc(C)n4)cc3Cl)c(=O)n(CC(=O)N[C@@H]3CCNC3)c2n1] | 0.0124 | 2-[6-[2-chloro-4-(6-methylpyrazin- 2-yl)phenyl]-2-(methylamino)-7- oxo-pyrido[2,3-d]pyrimidin-8-yl]- N-[(3R)-pyrrolidin-3-yl]acetamide |
| I-152 | 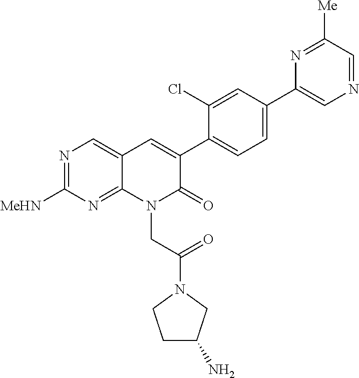 [SMILES: CNc1ncc2cc(-c3ccc(-c4cncc(C)n4)cc3Cl)c(=O)n(CC(=O)N3CC[C@@H](N)C3)c2n1] | 0.0211 | 8-[2-[(3R)-3-aminopyrrolidin-1-yl]- 2-oxo-ethyl]-6-[2-chloro-4-(6- methylpyrazin-2-yl)phenyl]-2- (methylamino)pyrido[2,3- d]pyrimidin-7-one |
| I-153 | 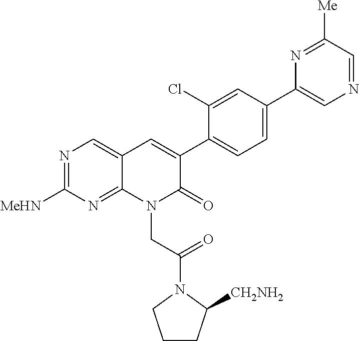 | 0.0085 | 8-[2-[(2R)-2- (aminomethyl)pyrrolidin-1-yl]-2- oxo-ethyl]-6-[2-chloro-4-(6- methylpyrazin-2-yl)phenyl]-2- (methylamino)pyrido[2,3- d]pyrimidin-7-one |
| I-154 | 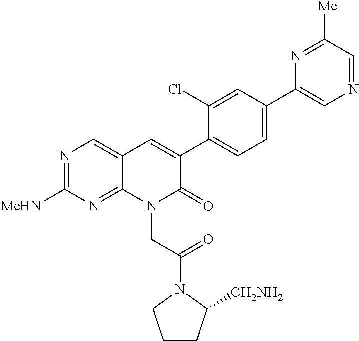 | 0.0394 | 8-[2-[(2S)-2- (aminomethyl)pyrrolidin-1-yl]-2- oxo-ethyl]-6-[2-chloro-4-(6- methylpyrazin-2-yl)phenyl]-2- (methylamino)pyrido[2,3- d]pyrimidin-7-one |
| I-155 | 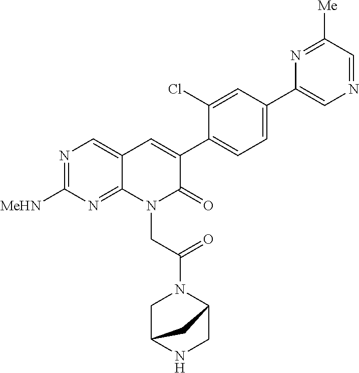 [SMILES: CNc1ncc2cc(-c3ccc(-c4cncc(C)n4)cc3Cl)c(=O)n(CC(=O)N3C[C@@H]4C[C@H]3CN4)c2n1] | 0.0392 | 6-[2-chloro-4-(6-methylpyrazin-2- yl)phenyl]-8-[2-[(1S,4S)-2 5- diazabicyclo[2.2.1]heptan-2-yl]-2- oxo-ethyl]-2- (methylamino)pyrido[2,3- d]pyrimidin-7-one |
| I-156 | 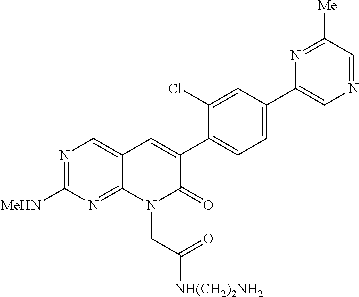 | 0.0144 | N-(2-aminoethyl)-2-[6-[2-chloro-4- (6-methylpyrazin-2-yl)phenyl]-2- (methylamino)-7-oxo-pyrido[2,3- d]pyrimidin-8-yl]acetamide |
| I-157 | 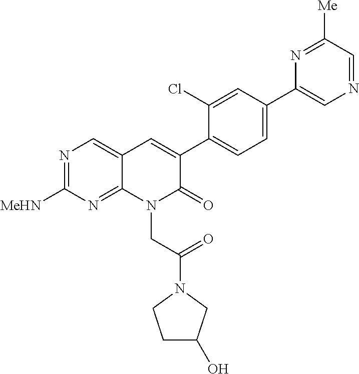 [SMILES: CNc1ncc2cc(-c3ccc(-c4cncc(C)n4)cc3Cl)c(=O)n(CC(=O)N3CCC(O)C3)c2n1] | 0.0449 | 6-[2-chloro-4-(6-methylpyrazin-2- yl)phenyl]-8-[2-(3- hydroxypyrrolidin-1-yl)-2-oxo- ethyl]-2-(methylamino)pyrido[2,3- d]pyrimidin-7-one |
| I-158 | 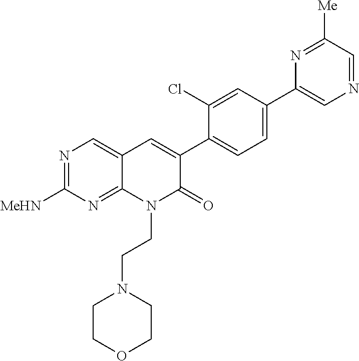 [SMILES: CNc1ncc2cc(-c3ccc(-c4cncc(C)n4)cc3Cl)c(=O)n(CCN3CCOCC3)c2n1] | 0.0782 | 6-[2-chloro-4-(6-methylpyrazin-2- yl)phenyl]-2-(methylamino)-8-(2- morpholinoethyl)pyrido[2,3- d]pyrimidin-7-one |
| I-159 | 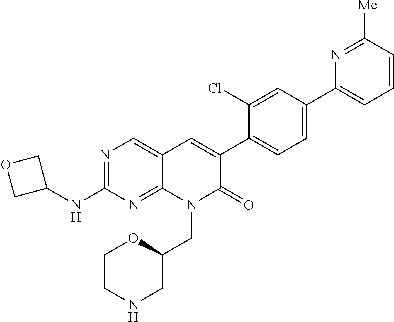 [SMILES: Cc1cccc(-c2ccc(-c3cc4cnc(NC5COC5)nc4n(C[C@H]4CNCCO4)c3=O)c(Cl)c2)n1] | 0.0108 | 6-[2-chloro-4-(6-methyl-2- pyridyl)phenyl]-8-[[(2R)- morpholin-2-yl]methyl]-2-(oxetan- 3-ylamino)pyrido[2,3-d]pyrimidin- 7-one |
| I-160 | 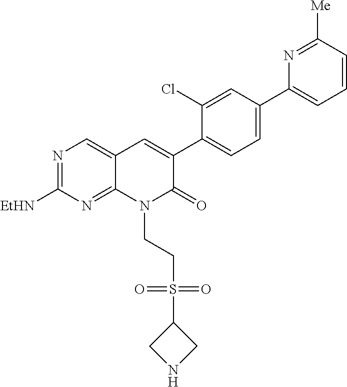 | 0.0103 | 8-[2-(azetidin-3-ylsulfonyl)ethyl]-6- [2-chloro-4-(6-methyl-2- pyridyl)phenyl]-2- (ethylamino)pyrido[2,3- d]pyrimidin-7-one |
| I-161 | 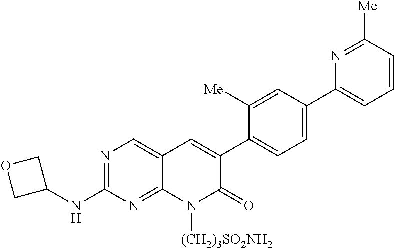 | 0.022 | 3-[6-[2-methyl-4-(6-methyl-2- pyridyl)phenyl]-2-(oxetan-3- ylamino)-7-oxo-pyrido[2,3- d]pyrimidin-8-yl]propane-1- sulfonamide |
| I-162 | 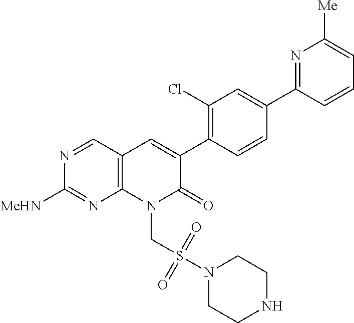 [SMILES: CNc1ncc2cc(-c3ccc(-c4cccc(C)n4)cc3Cl)c(=O)n(CS(=O)(=O)N3CCNCC3)c2n1] | 0.0147 | 6-[2-chloro-4-(6-methyl-2- pyridyl)phenyl]-2-(methylamino)-8- (piperazin-1- ylsulfonylmethyl)pyrido[2,3- d]pyrimidin-7-one |
| I-163 | 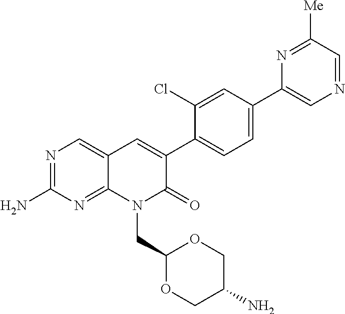 [SMILES: Cc1cncc(-c2ccc(-c3cc4cnc(N)nc4n(C[C@H]4OC[C@H](N)CO4)c3=O)c(Cl)c2)n1] | 0.0269 | 2-amino-8-[(5-amino-1,3-dioxan-2- yl)methyl]-6-[2-chloro-4-(6- methylpyrazin-2- yl)phenyl]pyrido[2,3-d]pyrimidin- 7-one |
| I-164 | 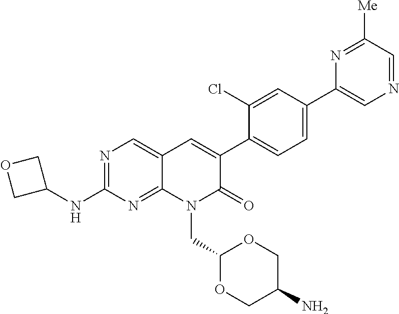 [SMILES: Cc1cncc(-c2ccc(-c3cc4cnc(NC5COC5)nc4n(C[C@H]4OC[C@H](N)CO4)c3=O)c(Cl)c2)n1] | 0.0099 | 8-[(5-amino-1,3-dioxan-2- yl)methyl]-6-[2-chloro-4-(6- methylpyrazin-2-yl)phenyl]-2- (oxetan-3-ylamino)pyrido[2,3- d]pyrimidin-7-one |
| I-165 | 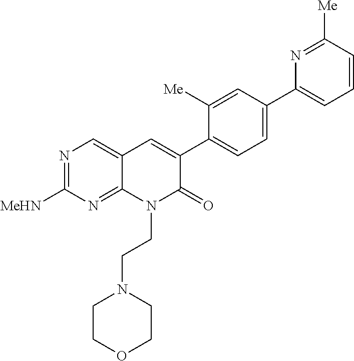 [SMILES: CNc1ncc2cc(-c3ccc(-c4cccc(C)n4)cc3C)c(=O)n(CCN3CCOCC3)c2n1] | 0.0167 | 2-(methylamino)-6-[2-methyl-4-(6- methyl-2-pyridyl)phenyl]-8-(2- morpholinoethyl)pyrido[2,3- d]pyrimidin-7-one |
| I-166 | 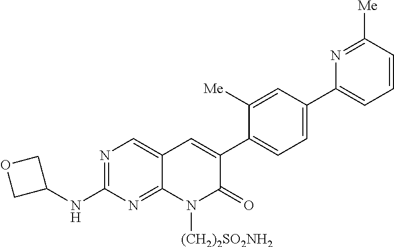 | 0.017 | 2-[6-[2-methyl-4-(6-methyl-2- pyridyl)phenyl]-2-(oxetan-3- ylamino)-7-oxo-pyrido[2,3- d]pyrimidin-8-yl]ethanesulfonate |
| I-167 | 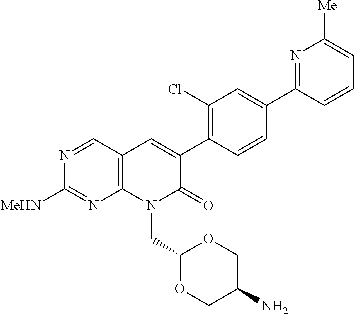 [SMILES: CNc1ncc2cc(-c3ccc(-c4cccc(C)n4)cc3Cl)c(=O)n(C[C@H]3OC[C@H](N)CO3)c2n1] | 0.00369 | 8-[(5-amino-1,3-dioxan-2- yl)methyl]-6-[2-chloro-4-(6-methyl- 2-pyridyl)phenyl]-2- (methylamino)pyrido[2,3- d]pyrimidin-7-one |
| I-168 | 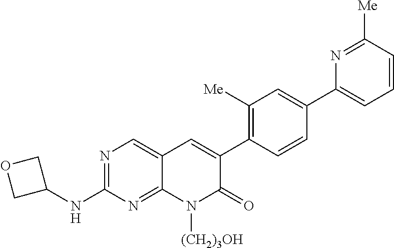 | 0.0157 | 8-(3-hydroxypropyl)-6-[2-methyl-4- (6-methyl-2-pyridyl)phenyl]-2- (oxetan-3-ylamino)pyrido[2,3- d]pyrimidin-7-one |
| I-169 | 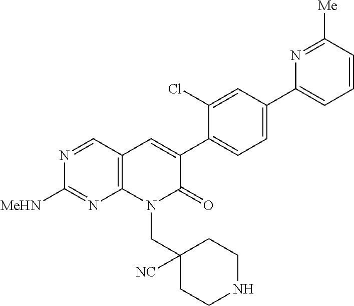 [SMILES: CNc1ncc2cc(-c3ccc(-c4cccc(C)n4)cc3Cl)c(=O)n(CC3(C#N)CCNCC3)c2n1] | 0.012 | 4-[[6-[2-chloro-4-(6-methyl-2- pyridyl)phenyl]-2-(methylamino)-7- oxo-pyrido[2,3-d]pyrimidin-8- yl]methyl]piperidine-4-carbonitrile |
| I-170 | 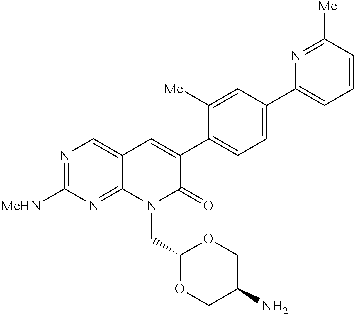 [SMILES: CNc1ncc2cc(-c3ccc(-c4cccc(C)n4)cc3C)c(=O)n(C[C@H]3OC[C@H](N)CO3)c2n1] | 0.0053 | 8-[(5-amino-1,3-dioxan-2- yl)methyl]-2-(methylamino)-6-[2- methyl-4-(6-methyl-2- pyridyl)phenyl]pyrido[2,3- d]pyrimidin-7-one |
| I-171 | 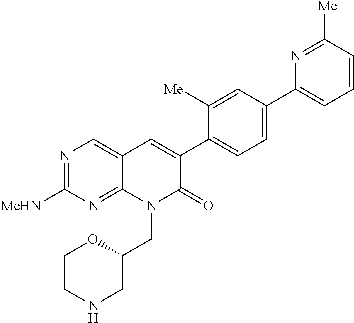 [SMILES: CNc1ncc2cc(-c3ccc(-c4cccc(C)n4)cc3C)c(=O)n(C[C@@H]3CNCCO3)c2n1] | 0.005 | 8-[2-(5-amino-1,3-dioxan-2- yl)ethyl]-6-[2-chloro-4-(6- methylpyrazin-2-yl)phenyl]-2- (methylamino)pyrido[2,3- d]pyrimidin-7-one |
| I-172 | 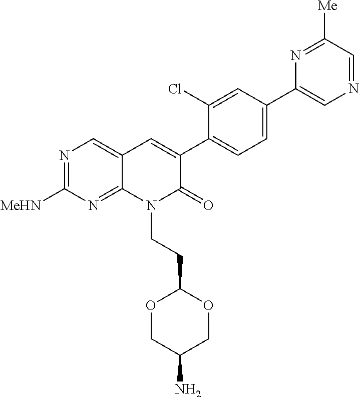 [SMILES: CNc1ncc2cc(-c3ccc(-c4cncc(C)n4)cc3Cl)c(=O)n(CC[C@H]3OC[C@@H](N)CO3)c2n1] | 0.0119 | 8-[2-(5-amino-1,3-dioxan-2- yl)ethyl]-6-[2-chloro-4-(6- methylpyrazin-2-yl)phenyl]-2- (methylamino)pyrido[2,3- d]pyrimidin-7-one |
| I-173 | 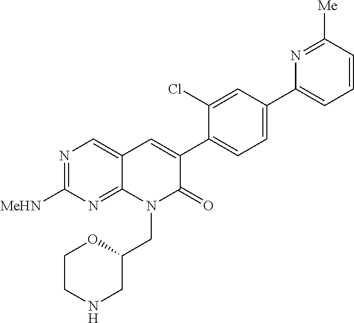 [SMILES: CNc1ncc2cc(-c3ccc(-c4cccc(C)n4)cc3Cl)c(=O)n(C[C@@H]3CNCCO3)c2n1] | 0.0129 | 6-[2-chloro-4-(6-methyl-2- pyridyl)phenyl]-2-(methylamino)-8- [[(2S)-morpholin-2- yl]methyl]pyrido[2,3-d]pyrimidin- 7-one |
| I-174 | 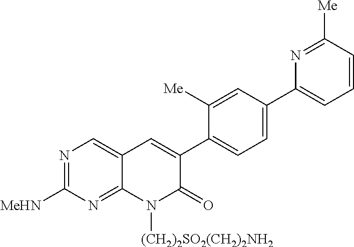 | 0.0072 | 8-[2-(2-aminoethylsulfonyl)ethyl]- 2-(methylamino)-6-[2-methyl-4-(6- methyl-2- pyridyl)phenyl]pyrido[2,3- d]pyrimidin-7-one |
| I-175 | 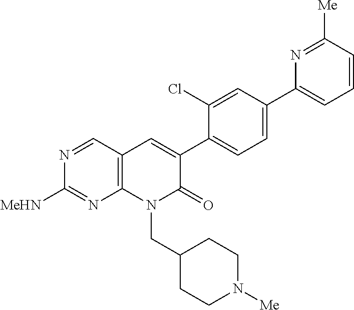 [SMILES: CNc1ncc2cc(-c3ccc(-c4cccc(C)n4)cc3Cl)c(=O)n(CC3CCN(C)CC3)c2n1] | 0.0198 | 6-[2-chloro-4-(6-methyl-2- pyridyl)phenyl]-2-(methylamino)-8- [(1-methyl-4- piperidyl)methyl]pyrido[2,3- d]pyrimidin-7-one |
| I-176 | 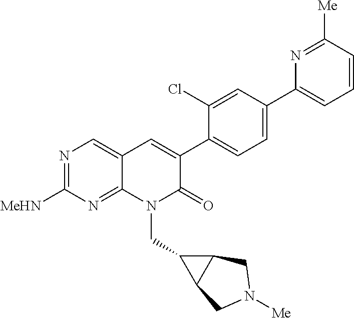 [SMILES: CNc1ncc2cc(-c3ccc(-c4cccc(C)n4)cc3Cl)c(=O)n(C[C@H]3[C@@H]4CN(C)C[C@@H]43)c2n1] | 0.011 | 6-[2-chloro-4-(6-methyl-2- pyridyl)phenyl]-2-(methylamino)-8- [[(1R,5S)-3-methyl-3- azabicyclo[3.1.0]hexan-6- yl]methyl]pyrido[2,3-d]pyrimidin- 7-one |
| I-177 | 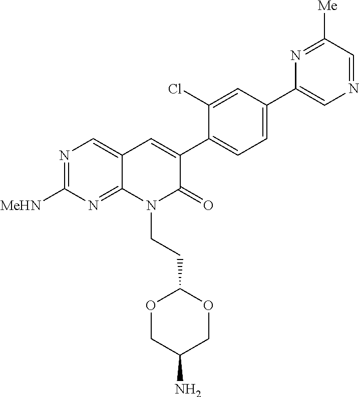 [SMILES: CNc1ncc2cc(-c3ccc(-c4cncc(C)n4)cc3Cl)c(=O)n(CC[C@H]3OC[C@H](N)CO3)c2n1] | 0.00682 | 8-[2-(5-amino-1,3-dioxan-2- yl)ethyl]-6-[2-chloro-4-(6- methylpyrazin-2-yl)phenyl]-2- (methylamino)pyrido[2,3- d]pyrimidin-7-one |
| I-178 | 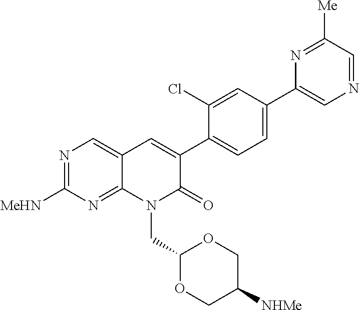 [SMILES: CNc1ncc2cc(-c3ccc(-c4cncc(C)n4)cc3Cl)c(=O)n(C[C@H]3OC[C@H](NC)CO3)c2n1] | 0.0135 | 6-[2-chloro-4-(6-methylpyrazin-2- yl)phenyl]-2-(methylamino)-8-[[5- (methylamino)-1,3-dioxan-2- yl]methyl]pyrido[2,3-d]pyrimidin- 7-one |
| I-179 | 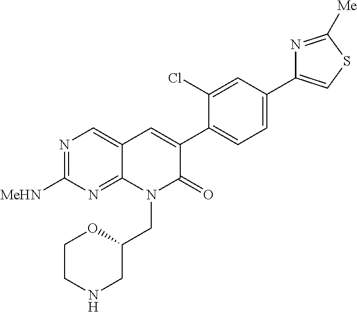 [SMILES: CNc1ncc2cc(-c3ccc(-c4csc(C)n4)cc3Cl)c(=O)n(C[C@@H]3CNCCO3)c2n1] | 0.0666 | 6-[2-chloro-4-(2-methylthiazol-4- yl)phenyl]-2-(methylamino)-8- [[(2S)-morpholin-2- yl]methyl]pyrido[2,3-d]pyrimidin- 7-one |
| I-180 | 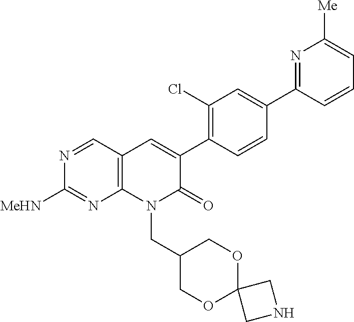 [SMILES: CNc1ncc2cc(-c3ccc(-c4cccc(C)n4)cc3Cl)c(=O)n(CC3COC4(CNC4)OC3)c2n1] | 0.00415 | 6-[2-chloro-4-(6-methyl-2- pyridyl)phenyl]-8-(5,9-dioxa-2- azaspiro[3.5]nonan-7-ylmethyl)-2- (methylamino)pyrido[2,3- d]pyrimidin-7-one |
| I-181 | 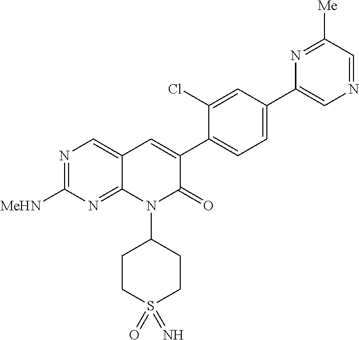 [SMILES: CNc1ncc2cc(-c3ccc(-c4cncc(C)n4)cc3Cl)c(=O)n(C3CCS(=N)(=O)CC3)c2n1] | 0.11 | 6-[2-chloro-4-(6-methylpyrazin-2- yl)phenyl]-8-(1-imino-1-oxo-thian- 4-yl)-2-(methylamino)pyrido[2,3- d]pyrimidin-7-one |
| I-182 | 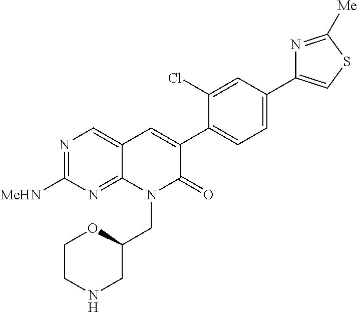 [SMILES: CNc1ncc2cc(-c3ccc(-c4csc(C)n4)cc3Cl)c(=O)n(C[C@H]3CNCCO3)c2n1] | 0.0447 | 6-[2-chloro-4-(2-methylthiazol-4- yl)phenyl]-2-(methylamino)-8- [[(2R)-morpholin-2- yl]methyl]pyrido[2,3-d]pyrimidin- 7-one |
| I-183 | 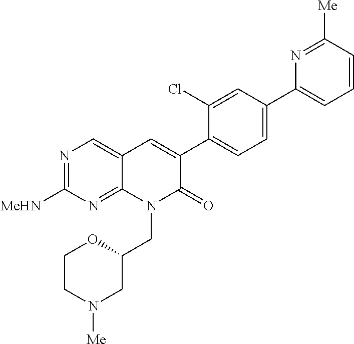 [SMILES: CNc1ncc2cc(-c3ccc(-c4cccc(C)n4)cc3Cl)c(=O)n(C[C@@H]3CN(C)CCO3)c2n1] | 0.0268 | 6-[2-chloro-4-(6-methyl-2- pyridyl)phenyl]-2-(methylamino)-8- [[(2S)-4-methylmorpholin-2- yl]methyl]pyrido[2,3-d]pyrimidin- 7-one |
| I-184 | 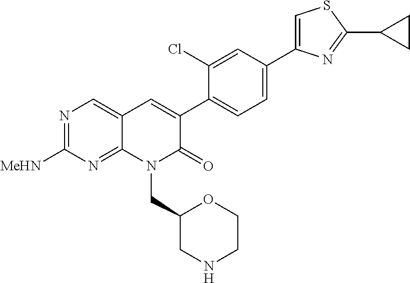 [SMILES: CNc1ncc2cc(-c3ccc(-c4csc(C5CC5)n4)cc3Cl)c(=O)n(C[C@@H]3CNCCO3)c2n1] | 0.0431 | 6-[2-chloro-4-(2- cyclopropylthiazol-4-yl)phenyl]-2- (methylamino)-8-[[(2S)-morpholin- 2-yl]methyl]pyrido[2,3- d]pyrimidin-7-one |
| I-185 | 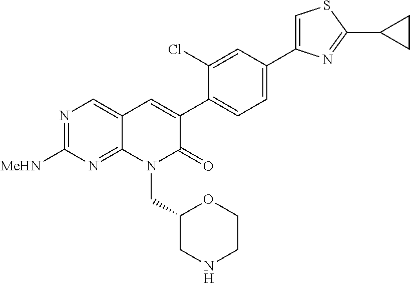 [SMILES: CNc1ncc2cc(-c3ccc(-c4csc(C5CC5)n4)cc3Cl)c(=O)n(C[C@H]3CNCCO3)c2n1] | 0.0473 | 6-[2-chloro-4-(2- cyclopropylthiazol-4-yl)phenyl]-2- (methylamino)-8-[[(2R)-morpholin- 2-yl]methyl]pyrido[2,3- d]pyrimidin-7-one |
| I-186 | 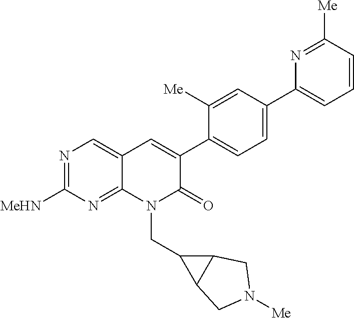 [SMILES: CNc1ncc2cc(-c3ccc(-c4cccc(C)n4)cc3C)c(=O)n(CC3C4CN(C)CC43)c2n1] | 0.0119 | 2-(methylamino)-8-[(3-methyl-3- azabicyclo[3.1.0]hexan-6- yl)methyl]-6-[2-methyl-4-(6- methyl-2- pyridyl)phenyl]pyrido[2,3- d]pyrimidin-7-one |
| I-187 | 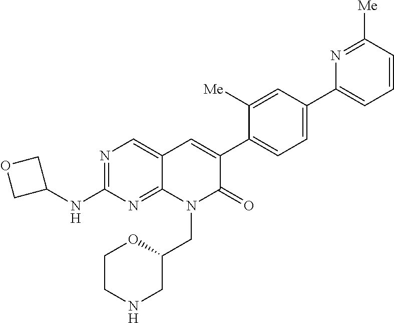 [SMILES: Cc1cccc(-c2ccc(-c3cc4cnc(NC5COC5)nc4n(C[C@@H]4CNCCO4)c3=O)c(C)c2)n1] | 0.0059 | 6-[2-methyl-4-(6-methyl-2- pyridyl)phenyl]-8-[[(2S)- morpholin-2-yl]methyl]-2-(oxetan- 3-ylamino)pyrido[2,3-d]pyrimidin- 7-one |
| I-188 | 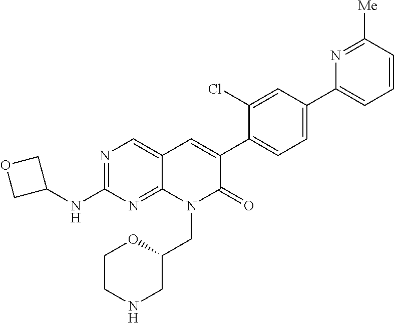 [SMILES: Cc1cccc(-c2ccc(-c3cc4cnc(NC5COC5)nc4n(C[C@@H]4CNCCO4)c3=O)c(Cl)c2)n1] | 0.0081 | 6-[2-chloro-4-(6-methyl-2- pyridyl)phenyl]-8-[[(2S)- morpholin-2-yl]methyl]-2-(oxetan- 3-ylamino)pyrido[2,3-d]pyrimidin- 7-one |
| I-189 | 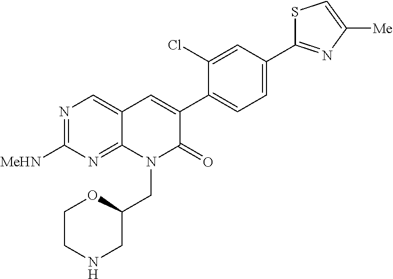 [SMILES: CNc1ncc2cc(-c3ccc(-c4nc(C)cs4)cc3Cl)c(=O)n(C[C@H]3CNCCO3)c2n1] | 0.0258 | 6-[2-chloro-4-(4-methylthiazol-2- yl)phenyl]-2-(methylamino)-8- [[(2R)-morpholin-2- yl]methyl]pyrido[2,3-d]pyrimidin- 7-one |
| I-190 | 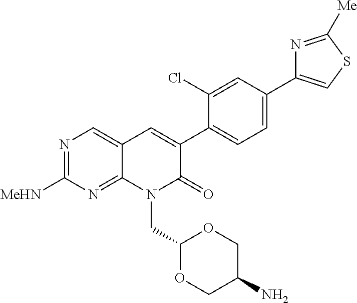 [SMILES: CNc1ncc2cc(-c3ccc(-c4csc(C)n4)cc3Cl)c(=O)n(C[C@H]3OC[C@H](N)CO3)c2n1] | 0.0152 | 8-[(5-amino-1,3-dioxan-2- yl)methyl]-6-[2-chloro-4-(2- methylthiazol-4-yl)phenyl]-2- (methylamino)pyrido[2,3- d]pyrimidin-7-one |
| I-191 | 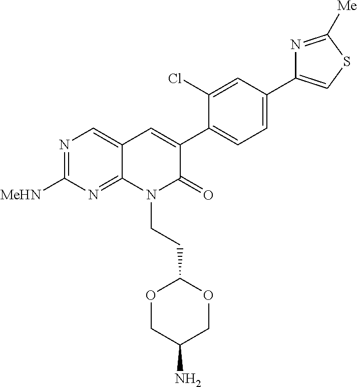 [SMILES: CNc1ncc2cc(-c3ccc(-c4csc(C)n4)cc3Cl)c(=O)n(CC[C@H]3OC[C@H](N)CO3)c2n1] | 0.0126 | 8-[2-(5-amino-1,3-dioxan-2- yl)ethyl]-6-[2-chloro-4-(2- methylthiazol-4-yl)phenyl]-2- (methylamino)pyrido[2,3- d]pyrimidin-7-one |
| I-191 | 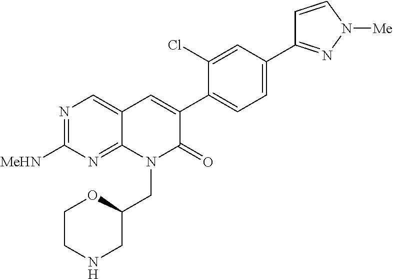 [SMILES: CNc1ncc2cc(-c3ccc(-c4ccn(C)n4)cc3Cl)c(=O)n(C[C@H]3CNCCO3)c2n1] | 0.107 | 6-[2-chloro-4-(1-methylpyrazol-3- yl)phenyl]-2-(methylamino)-8- [[(2R)-morpholin-2- yl]methyl]pyrido[2,3-d]pyrimidin- 7-one |
| I-192 | 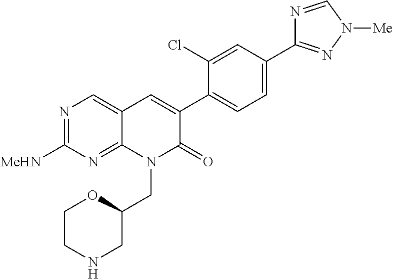 [SMILES: CNc1ncc2cc(-c3ccc(-c4ncn(C)n4)cc3Cl)c(=O)n(C[C@H]3CNCCO3)c2n1] | 4.4 | 6-[2-chloro-4-(1-methyl-1 2 4- triazol-3-yl)phenyl]-2- (methylamino)-8-[[(2R)-morpholin- 2-yl]methyl]pyrido[2,3- d]pyrimidin-7-one |
| I-193 | 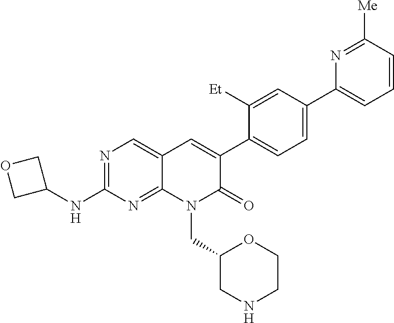 [SMILES: CCc1cc(-c2cccc(C)n2)ccc1-c1cc2cnc(NC3COC3)nc2n(C[C@H]2CNCCO2)c1=O] | 0.0208 | 6-[2-ethyl-4-(6-methyl-2- pyridyl)phenyl]-8-[[(2R)- morpholin-2-yl]methyl]-2-(oxetan- 3-ylamino)pyrido[2,3-d]pyrimidin- 7-one |
| I-194 | 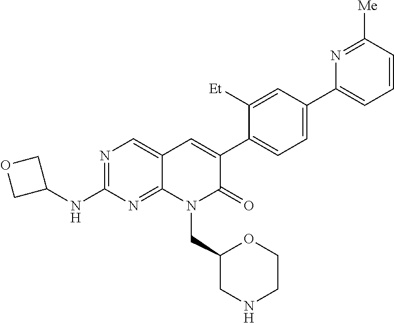 [SMILES: CCc1cc(-c2cccc(C)n2)ccc1-c1cc2cnc(NC3COC3)nc2n(C[C@@H]2CNCCO2)c1=O] | 0.0265 | 6-[2-ethyl-4-(6-methyl-2- pyridyl)phenyl]-8-[[(2S)- morpholin-2-yl]methyl]-2-(oxetan- 3-ylamino)pyrido[2,3-d]pyrimidin- 7-one |
| I-195 | 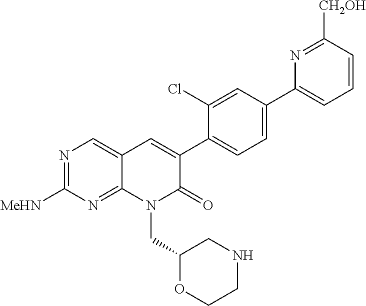 [SMILES: CNc1ncc2cc(-c3ccc(-c4cccc(CO)n4)cc3Cl)c(=O)n(C[C@@H]3CNCCO3)c2n1] | 0.0354 | 6-[2-chloro-4-[6-(hydroxymethyl)- 2-pyridyl]phenyl]-2-(methylamino)- 8-[[(2S)-morpholin-2- yl]methyl]pyrido[2,3-d]pyrimidin- 7-one |
| I-196 | 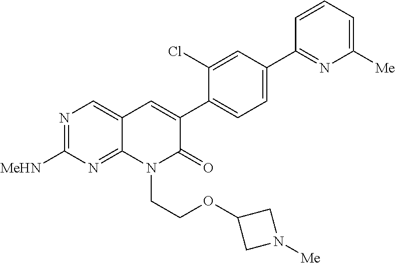 [SMILES: CNc1ncc2cc(-c3ccc(-c4cccc(C)n4)cc3Cl)c(=O)n(CCOC3CN(C)C3)c2n1] | 0.0185 | 6-[2-chloro-4-(6-methyl-2- pyridyl)phenyl]-2-(methylamino)-8- [2-(1-methylazetidin-3- yl)oxyethyl]pyrido[2,3- d]pyrimidin-7-one |
| I-197 Isomer A I-197 Isomer B | 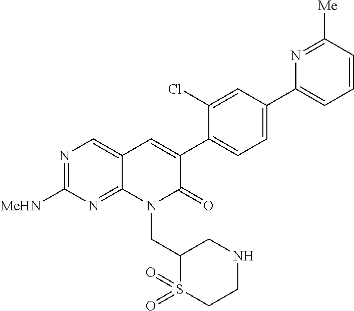 [SMILES: CNc1ncc2cc(-c3ccc(-c4cccc(C)n4)cc3Cl)c(=O)n(CC3CNCCS3(=O)=O)c2n1] | 0.0161 0.0316 | 6-[2-chloro-4-(6-methyl-2- pyridyl)phenyl]-8-[(1,1-dioxo-1 4- thiazinan-2-yl)methyl]-2- (methylamino)pyrido[2,3- d]pyrimidin-7-one |
| I-198 | 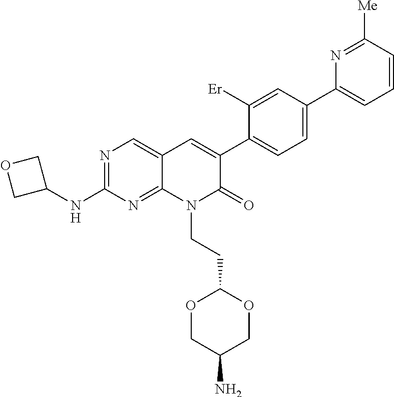 | 0.0012 | 8-[2-(5-amino-1,3-dioxan-2- yl)ethyl]-6-[2-ethyl-4-(6-methyl-2- pyridyl)phenyl]-2-(oxetan-3- ylamino)pyrido[2,3-d]pyrimidin-7- one |
| I-199 | 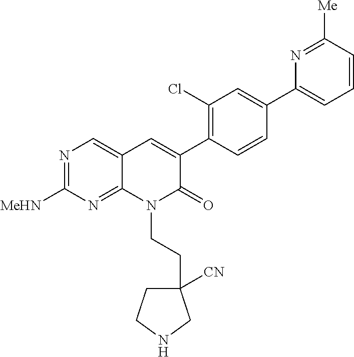 [SMILES: CNc1ncc2cc(-c3ccc(-c4cccc(C)n4)cc3Cl)c(=O)n(CCC3(C#N)CCNC3)c2n1] | 0.0028 | 3-[2-[6-[2-chloro-4-(6-methyl-2- pyridyl)phenyl]-2-(methylamino)-7- oxo-pyrido[2,3-d]pyrimidin-8- yl]ethyl]pyrrolidine-3-carbonitrile |
| I-200 | 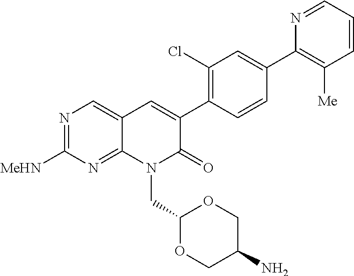 [SMILES: CNc1ncc2cc(-c3ccc(-c4ncccc4C)cc3Cl)c(=O)n(C[C@H]3OC[C@H](N)CO3)c2n1] | 0.0207 | 8-[(5-amino-1,3-dioxan-2- yl)methyl]-6-[2-chloro-4-(3-methyl- 2-pyridyl)phenyl]-2- (methylamino)pyrido[2,3- d]pyrimidin-7-one |
| I-201 | 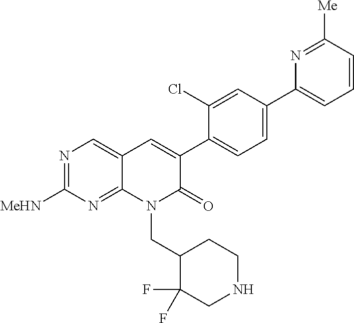 [SMILES: CNc1ncc2cc(-c3ccc(-c4cccc(C)n4)cc3Cl)c(=O)n(CC3CCNCC3(F)F)c2n1] | 0.005 | 6-[2-chloro-4-(6-methyl-2- pyridyl)phenyl]-8-[(3,3-difluoro-4- piperidyl)methyl]-2- (methylamino)pyrido[2,3- d]pyrimidin-7-one |
| I-202 | 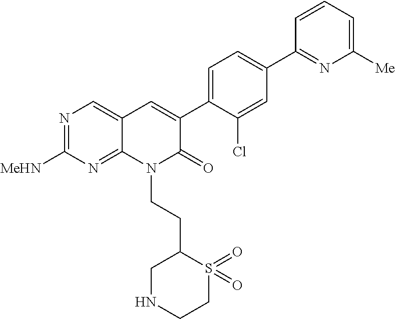 [SMILES: CNc1ncc2cc(-c3ccc(-c4cccc(C)n4)cc3Cl)c(=O)n(CCC3CNCCS3(=O)=O)c2n1] | 0.0071 | 6-[2-chloro-4-(6-methyl-2- pyridyl)phenyl]-8-[2-(1,1-dioxo- 14-thiazinan-2-yl)ethyl]-2- (methylamino)pyrido[2,3- d]pyrimidin-7-one |

¹Biological Example 1

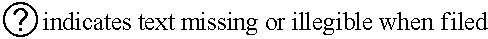

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, or, about 20° C.

Some compounds in following schemes are depicted with generalized substituents; however, one skilled in the art will immediately appreciate that the nature of the R groups can varied to afford the various compounds contemplated in this invention. Moreover, the reaction conditions are exemplary and alternative conditions are well known. The reaction sequences in the following examples are not meant to limit the scope of the invention as set forth in the claims.

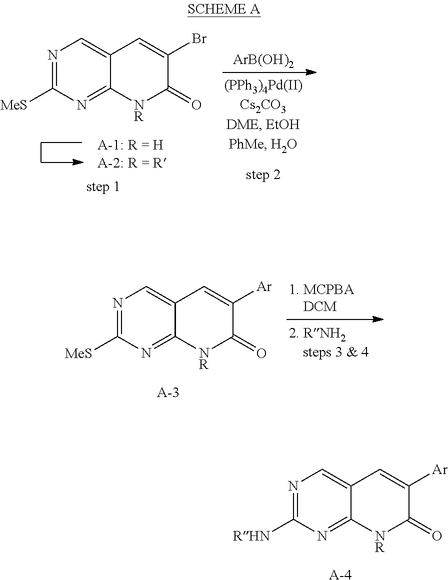

Compounds of the present invention can be prepared starting from 6-bromo-2-(methylthio)-pyrido[2,3-d]pyrimidin-7(8H)-one (CASRN 352328-87-1) by the process depicted in SCHEME A. The first step can be accomplished by alkylation of the lactam nitrogen with an alkyl halide or sulfonate in the presence of base such as Cs₂CO₃ in a polar aprotic solvent or by coupling utilizing a Mitsunobu coupling protocol. Mitsunobu conditions (D. L. Hughes, The Mitsunobu Reaction, in Organic Reactions, Volume 42, 1992, John Wiley & Sons, New York; pp. 335-656) comprise activating alcohols with a mixture of a phosphine such as a trialkylphosphine like tributylphosphine ((n-Bu)₃P), a triphenylphosphine (Ph₃P) and the like and a diethyl-azodicarboxylate (DEAD), diisopropyl-azodicarboxylate (DIAD) or di-tert-butyl-azodicarboxylate in an inert solvent such as THF, toluene, DCM. The reaction can take place over a wide range of temperatures ranging from ambient temperatures to the reflux temperature of the solvent employed

In the second step the aryl substituent is introduced utilizing a Suzuki protocol. The Suzuki reaction is a palladium-catalyzed coupling of a boronic acid (R—B(OH)₂) wherein R is aryl or vinyl) with an aryl or vinyl halide or triflate (R′Y wherein R′=aryl or vinyl; Y=halide or OSO₂CF₃) to afford a compound R—R′. Typical catalysts include Pd(PPh₃)₃, Pd(OAc)₂ and PdCl₂(dppf). With PdCl₂(dppf), primary alkyl borane compounds can be coupled to aryl or vinyl halide or triflate without β-elimination. Highly active catalysts have been identified (see, e.g. J. P. Wolfe et al., J. Am. Chem. Soc. 1999 121(41):9550-956 and A. F. Littke et al., J. Am. Chem. Soc. 2000 122(17):4020-4028). The reaction can be carried out in a variety of organic solvents including toluene, THF, dioxane, 1,2-dichloroethane, DMF, PhMe, MeOH, DMSO and acetonitrile, aqueous solvents and under biphasic conditions. Reactions are typically run from about room temperature to about 150° C. Additives (e.g. CsF, KF, TlOH, NaOEt and KOH) frequently accelerate the coupling reaction. There are a large number of parameters in the Suzuki reaction including the palladium source, ligand, additives and temperature and optimum conditions sometimes require optimization of the parameters for a given pair of reactants. One skilled in the art can determine optimal conditions without undue experimentation.

Finally the alkyl amine substituent on the pyrimidine ring is introduced in steps 3 and 4 by oxidation of the thiol to the corresponding sulfone and displacement of the sulfone with the desired amine. Oxidation of the sulfide can be conveniently carried out with MCPBA or with potassium hydrogen persulfate. (B. M. Trost and D. P. Curran, Tetrahedron Lett. 1981 22(14):1287) The sulfone can then be directly displaced by the requisite amine.

One skilled in the art will appreciate the sequence of steps in Schemes A and B can be varied without limiting the general utility of the procedure.

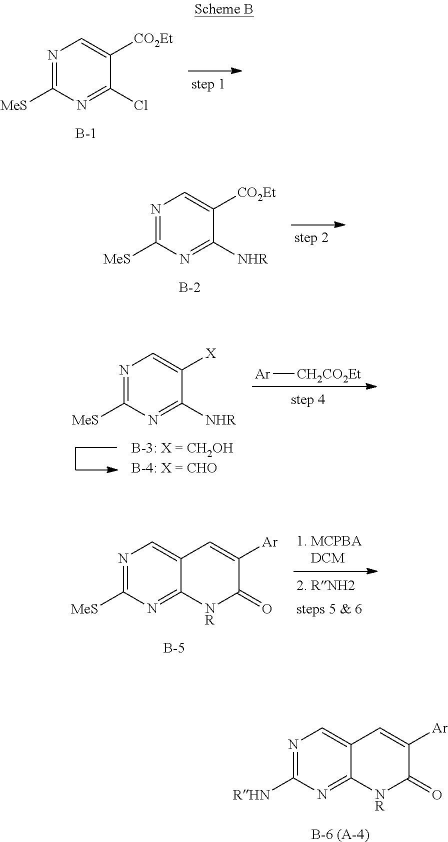

Alternatively the compounds of the invention can be prepared from ethyl 4-chloro-2-(methylthio)-5-pyrimidinecarboxylate (B-1, CASRN 5909-24-0). Displacement of the chloride introduces the lactam nitrogen with requisite amine side chain in step 1. Reduction of the ester and reoxidation of the resulting alcohol to an aldehyde B-4 is carried out by standard methodology. Condensation of the resulting aldehyde B-4 with an appropriate arylacetic acid and formation of the lactam affords the requisite 6-phenylpyrido[2,3-d]pyrimidin-7(8H)-one B-5. Introduction of the amine substituent on the pyrimidine ring is again accomplished by oxidation of the thiol and displacement of the resulting sulfone as describe previously. If R is hydrogen in B-5 or B-6, substitution of the nitrogen can accomplished by base-catalysed alkylation or by a Mitsunobu coupling as described previously.

Biological Activity

Determination of the PAK inhibitory activity of a compound of formula I was accomplished using the PAK1 inhibition assay in Biological Example 1. Potency values of exemplary compounds in PAK1 assays are reported TABLE 1. A cell-based mechanistic assay (Biological Example 2) was used to determine the effect of PAK inhibitors on down-stream signaling. Representative values for these assays can be found in TABLE 1.

Dosage & Administration

The present invention provides pharmaceutical compositions or medicaments containing the compounds of the invention and at least one therapeutically inert carrier, diluent or excipient, as well as methods of using the compounds of the invention to prepare such compositions and medicaments. In one example, compounds of formula I with the desired degree of purity may be formulated by mixing with physiologically acceptable carriers, i.e., carriers that are non-toxic to recipients at the dosages and concentrations employed into a dosage form at ambient temperature and at the appropriate pH. The pH of the formulation depends mainly on the particular use and the concentration of compound, but typically ranges anywhere from about 3 to about 8. In one example, a compound of formula I is formulated in an acetate buffer, at pH 5. In another embodiment, the compounds of formula I are sterile. The compound may be stored, for example, as a solid or amorphous composition, as a lyophilized formulation or as an aqueous solution.

Compositions are formulated, dosed, and administered in a fashion consistent with good medical practice. The term “therapeutically effective amount” denotes an amount of a compound of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the particular disorder being treated, the severity of the disorder, the particular patient being treated, the clinical condition of the individual patient, the cause of the disorder, the site of delivery of the agent, the method of administration, the scheduling of administration, and other factors known to medical practitioners

The term “treating” or “treatment” of a disease state includes (1) inhibiting the disease state, i.e., arresting the development of the disease state or its clinical symptoms, or (2) relieving the disease state, i.e., causing temporary or permanent regression of the disease state or its clinical symptoms.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the alt and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Sustained-release preparations may be prepared. Suitable examples of sustained-release preparations include semipermeable matrices of solid hydrophobic polymers containing a compound of formula I, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A dose to treat human patients may range from about 0.1 mg to about 1000 mg of a compound of formula I. A typical dose may be about 1 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

For oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

An example of a suitable oral dosage form is a tablet containing about 25 mg, 50 mg, 100 mg, 250 mg or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

In one embodiment, the pharmaceutical composition also includes at least one additional anti-proliferative agent.

An embodiment, therefore, includes a pharmaceutical composition comprising a compound of formula I, or a stereoisomer or pharmaceutically acceptable salt thereof. In a further embodiment includes a pharmaceutical composition comprising a compound of formula I, or a stereoisomer or pharmaceutically acceptable salt thereof, together with a pharmaceutically acceptable carrier or excipient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

Combination Therapy

The compounds of formula I may be employed alone or in combination with other therapeutic agents for the treatment of a disease or disorder described herein, such as a hyperproliferative disorder (e.g., cancer). In certain embodiments, a compound of formula I is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of formula I such that they do not adversely affect each other. The combination therapy may provide “synergy” and prove “synergistic”, i.e., the effect achieved when the active ingredients used together is greater than the sum of the effects that results from using the compounds separately.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Suitable dosages for any of the above co-administered agents are those presently used and may be lowered due to the combined action (synergy) of the newly identified agent and other chemotherapeutic agents or treatments.

Combination therapies according to the present invention thus comprise the administration of at least one compound of formula I, or a stereoisomer, geometric isomer, tautomer, metabolite, or pharmaceutically acceptable salt and the use of at least one other cancer treatment method. The amounts of the compound(s) of formula I and the other pharmaceutically active chemotherapeutic agent(s) and the relative timings of administration will be selected in order to achieve the desired combined therapeutic effect.

Articles of Manufacture

In another embodiment of the invention, an article of manufacture, or “kit”, containing materials useful for the treatment of the diseases and disorders described above is provided. In one embodiment, the kit comprises a container comprising a compound of formula I, or a stereoisomer, tautomer, or pharmaceutically acceptable salt thereof. The kit may further comprise a label or a package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of formula I or a formulation thereof which is effective for treating the condition and may have a sterile access port (for example, the container may be an intravenous solution bag or a vial having a stopper pierceable by a hypodermic injection needle). At least one active agent in the composition is a compound of formula I. Alternatively, or additionally, the article of manufacture may further comprise a second container comprising a pharmaceutically diluent, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In another embodiment, the kits are suitable for the delivery of solid oral forms of a compound of formula I, such as tablets or capsules. Such a kit can include a number of unit dosages. An example of such a kit is a “blister pack”. Blister packs are well known in the packaging industry and are widely used for packaging pharmaceutical unit dosage forms.

According to one embodiment, a kit may comprise (a) a first container with a compound of formula I contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### Biological Example 1

### PAK1-KD (Kinase Domain) IC₅₀ Zlyte Assay Protocol

Activity of human recombinant PAK1-KD protein may be assessed in vitro by assay of the phosphorylation of a FRET peptide substrate. Catalytically active human recombinant PAK1-KD protein is obtained by purification from Tini cells infected with a human PAK1-KD recombinant baculovirus expression vector.

The activity/inhibition of PAK1-KD was estimated by measuring the phosphorylation of a FRET peptide substrate (Ser/Thr19) labeled with Coumarin and Fluorescein using Z′-LYTE™ assay (Invitrogen). The peptide substrate is a consensus sequence (KKRNRRLSVA) based on various PAK substrates reported in the scientific literature. The 10 μL assay mixtures contained 50 mM HEPES (pH 7.5), 0.01% Brij-35, 10 mM MgCl₂, 1 mM EGTA, 2 pM FRET peptide substrate, and 20 pM PAK1-KD. Incubations were carried out at 22° C. in black polypropylene 384-well plates (Corning Costar). Prior to the assay, PAK1-KD, FRET peptide substrate and serially diluted test compounds were preincubated together in assay buffer (7.5 μL) for 10 minutes, and the assay was initiated by the addition of 2.5 μL assay buffer containing 160 μM ATP (4×). Following the 60-minute incubation, the assay mixtures were quenched by the addition of 5 μL of Z′-LYTE™ development reagent, and 1 hour later the emissions of Coumarin (445 nm) and Fluorescein (520 nm) were determined after excitation at 400 nm using an Envision plate reader (Perkin Elmer). An emission ratio (445 nm/520 nm) was determined to quantify the degree of substrate phosphorylation. Data incorporated into TABLE 1.

### Biological Example 2

### Cellular PAK IC₅₀ Assay Protocol

Group I PAKs (PAK1-3) are activated upon binding to the Rho GTPases, Rac1 and Cdc42. Activated group I PAKs phosphorylate MEK1 at Serine 298 (S298), one of the two sites in the catalytic domain that is important for stable association between Raf and MEK1 and subsequent MAPK activation. The inhibition of group I PAKs in EBC1 cells is assessed by detecting changes in the level of MEK1 phosphorylation at S298 using homogenous time-resolved fluorescence (HTRF). Inhibitory activity was estimated by treating 2×10⁴ EBC1 cells for 2 h with PAK inhibitors in media containing 0.1% FBS. Following inhibitor treatment, cells were lysed with 25 μL of 1× cellular kinase lysis buffer (Cisbio) containing 1× cellular kinase blocking reagent (Cisbio). Cellular lysis was carried out at 4° C. for 2 h with constant shaking before lysate (16 μL) was transferred to white 384-well ProxiPlates™ (Perkin Elmer). Anti-total MEK1 antibody labeled with Europium cryptate donor (1 ng/well) (Cell Signaling Technologies catalog number 2352) and anti-phospho MEK1 (S298) antibody labeled with d2 acceptor (Cell Signaling Technologies catalog number 9128) (10 ng/well) were prepared in 1× detection buffer (CisBio) and added to each well of the assay plate and allowed to incubate at RT overnight. The following day the fluorescence emission from each well was measured in EnVision® (Perkin Elmer) at an excitation of 330 nm and dual emission wavelengths of 615 nm and 665 nm. The signal in each well at 665 nm was multiplied by 10,000 and divided by the signal in the same well at 615 nm to obtain a ratio. Ratio values ([665·10,000]÷615) were plotted as a function of the concentration of compound to determine IC₅₀ values. Representative data tabulated in TABLE II.

TABLE II

| | IC₅₀ |
|---|---|
| compound | (μM) |
| I-1 | 0.249 |
| I-2 | 0.146 |
| I-3 | 0.506 |
| I-4 | 0.096 |
| I-5 | 0.147 |
| I-6 | 0.17 |
| I-7 | 0.34 |
| I-8 | 1.5 |
| I-9 | 0.358 |
| I-10 | 0.274 |
| I-11 | 0.189 |
| I-12 | 0.131 |
| I-13 | 0.18 |
| I-14 | 0.246 |
| I-15 | 0.0468 |
| I-16 | 0.0756 |
| I-17 | 0.096 |
| I-18 | 0.508 |
| I-19 | 0.286 |
| I-22 | 0.0635 |
| I-31 | 0.127 |
| I-44 | 0.0723 |
| I-52 | 0.156 |
| I-56 | 0.426 |
| I-61 | 0.2779 |
| I-64 | 0.0357 |
| I-70 | 0.289 |
| I-75 | 0.197 |
| I-89 | 0.0577 |
| I-91 | 0.108 |
| I-108 | 0.0835 |
| I-127 | 0.0382 |

### Referential Example 1

### tert-Butyl 4-(2-methylsulfonyloxyethyl)piperazine-1-carboxylate

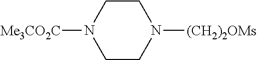

To a solution of tert-butyl 4-(2-hydroxyethyl)piperazine-1-carboxylate (887 mg, 3.85 mmol) in anhydrous DCM (10.2 mL) at 0° C. was added TEA (591 mg, 5.78 mmol). After 5 min methanesulfonyl chloride (529 mg, 4.62 mmol) was added, and the reaction mixture was stirred at RT under N₂ for 3 h. The reaction mixture was diluted with EtOAc. The organic layer was washed with brine, dried (Na₂SO₄), filtered, and concentrated to give tert-butyl 4-(2-methylsulfonyloxyethyl)piperazine-1-carboxylate (1.18 g, 99.9%) as an oil. ¹H NMR (400 MHz, CDCl₃) δ 4.37 (2H), 3.46 (4H), 3.07 (s, 3H), 2.80 (2H), 2.62-2.42 (m, 4H), 1.46 (s, 9H).

### Referential Example 2

### Benzyl 2-(2-methylsulfonyloxyethyl)pyrrolidine-1-carboxylate

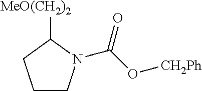

step 1: To a solution of 2-pyrrolidin-2-yl EtOH (1.0 g) and TEA (1.76 g, 17.4 mmol) in anhydrous DCM (17.4 mL) at 0° C. was added dropwise benzyl chloroformate (1.48 g, 8.68 mmol), and the reaction mixture was stirred at RT under N₂ for 20 h. The reaction mixture was diluted with EtOAc. The organic layer was washed with 10% aq. citric acid, water and brine, dried (Na₂SO₄), filtered, and concentrated in vacuo. The crude residue was purified by SiO₂ chromatography eluting with heptane:/EtOAc to afford benzyl 2-(2-hydroxyethyl)pyrrolidine-1-carboxylate (1.39 g, 64.3%) as a clear oil. ¹H NMR (400 MHz, CDCl₃) δ 7.43-7.28 (m, 5H), 5.15 (d, J=2.1 Hz, 2H), 4.21 (br s, 1H), 3.60 (dt, J=9.9, 3.6 Hz, 2H), 3.52-3.24 (m, 2H), 3.22 (br s, 1H), 2.01 (dtd, J=11.2, 9.2, 7.6 Hz, 1H), 1.95-1.85 (m, 2H), 1.80-1.60 (m, 2H), 1.79-1.60 (m, 1H).

step 2: Benzyl 2-(2-methylsulfonyloxyethyl)pyrrolidine-1-carboxylate was prepared using a procedure analogous to referential example 1 except benzyl 2-(2-hydroxyethyl)pyrrolidine-1-carboxylate was the starting material. ¹H NMR (400 MHz, CDCl₃) δ 7.40-7.27 (m, 5H), 5.12 (s, 2H), 4.40-4.14 (m, 2H), 4.02 (br s, 1H), 2.84 (br s, 1H), 3.59-3.39 (m, 2H), 3.07-2.95 (m, 2H), 2.26-2.10 (m, 1H), 2.03 (dq, J=12.0, 8.1 Hz, 1H), 1.93-1.77 (m, 3H), 1.72 (ddt, J=13.9, 5.8, 3.1 Hz, 1H).

### Referential Example 3

### tert-Butyl 4-(2-chloroacetyl)piperazine-1-carboxylate

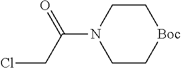

To a mixture tert-butyl piperazine-1-carboxylate (2 g, 10.1 mmol) in DCM (20 mL) at 0° C. was added 2-chloroacetyl chloride (570 mg, 5.05 mmol). After stirring for 5 h at RT, the mixture was diluted with DCM (25 mL), washed with dilute citric acid, water and brine. The organic layer was dried (MgSO₄), filtered and concentrated in vacuo to afford tert-butyl 4-(2-chloroacetyl)piperazine-1-carboxylate as a pale yellow solid (1.4 g, 50%). MS (ESI): m/z=263.2 [M+1]⁺.

### Referential Example 4

### tert-Butyl 3-(2-hydroxyethoxy)azetidine-1-carboxylate

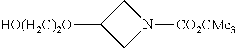

tert-Butyl 3-hydroxyazetidine-1-carboxylate (200 mg, 1.16 mmol), TEA (120 mg, 1.16 mmol), tetrabutylammonium bromide (30 mg, 0.093 mmol), and ethylene carbonate (155 mg, 1.74 mmol) were heated at 100° C. (internal temperature) for 36 h. The reaction mixture was concentrated in vacuo at RT, and the residue was purified by SiO₂ chromatography eluting with MeOH/DCM (100:6) to afford tert-butyl 3-(2-hydroxyethoxy)azetidine-1-carboxylate (100 mg, 39%) as yellow oil. LCMS (ESI): m/z=162.3 [M−55]⁺.

### Referential Example 5

### tert-Butyl 3-(2-hydroxyethoxy)piperidine-1-carboxylate

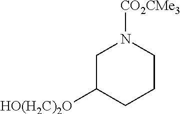

A mixture of tert-butyl 3-hydroxypiperidine-1-carboxylate (4.0 g, 0.02 mol), 1,3-dioxolan-2-one (1.926 g, 0.022 mol) and tetrabutylammonium bromide (128 mg, 0.4 mmol) in TEA (2.22 g, 0.022 mol) was stirred at 120° C. under N₂ for 18 h followed by cooling to RT. The reaction mixture was poured into water (150 mL), extracted with DCM (3×50 mL), then concentrated under reduced pressure and then purified by SiO₂ chromatography eluting with PE/EtOAc (1:1) to afford the target compound as yellow oil (981 mg, 20.1%). MS (ESI): m/z=190.1 [M−55].

### Referential Example 6

### tert-Butyl 3-(2-(tosyloxy)ethoxy)pyrrolidine-1-carboxylate

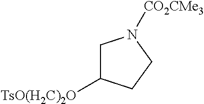

step 1: To a solution of sodium hydride (0.94 g, 39 mmol) in anhydrous THF (200 mL) at 0° C. was added tert-butyl 3-hydroxypyrrolidine-1-carboxylate (5.61 g, 30 mmol). After stirring for 40 min, ethyl chloroacetate (4.14 mL, 30 mmol) was added dropwise, and the resulting dark purple solution was stirred overnight. The reaction mixture was quenched with 5% HCl solution and extracted with EtOAc (150 mL). The extracts were combined, washed with brine, dried (Na₂SO₄), filtered and concentrated under reduced pressure. The residue was purified by SiO₂ chromatography eluting with a EtOAc/PE gradient (1:5 to 1:3) to afford tert-butyl 3-(2-ethoxy-2-oxoethoxy)pyrrolidine-1-carboxylate as a yellow oil (3.4 g, 42%). LCMS (ESI): m/z=218 [M−55]⁺.

step 2: Under a nitrogen atmosphere, a solution of tert-butyl 3-(2-ethoxy-2-oxoethoxy)pyrrolidine-1-carboxylate (884 mg, 3.24 mmol) in anhydrous THF (20.0 mL) was added dropwise to a stirred mixture of LiAlH₄ (120 mg, 3.24 mmol) in anhydrous THF (10.0 mL) at 0° C. The reaction was stirred for 40 min and then treated successively with water (2.0 mL) and 10% NaOH (0.9 mL). After stirring for 2 h the resulting mixture was extracted with EtOAc (50 mL). The solvent was removed under reduced pressure to afford tert-butyl 3-(2-hydroxyethoxy)pyrrolidine-1-carboxylate (644 mg, 86%) as colorless oil. LCMS (ESI): m/z=176.2 (M−55).

step 3: To an ice cold solution of tert-butyl 3-(2-hydroxyethoxy)pyrrolidine-1-carboxylate (644 mg, 2.78 mmol) in DCM (10 mL) and THF (4.0 mL) was added TEA (0.77 mL) and TsCl (0.63 g, 0.69 mmol). The mixture was stirred at RT overnight. DMAP (0.18 g) was then added, and the reaction mixture was stirred for 2 h. A saturated aqueous NaHCO₃ solution (2.0 ml) was added and the layers separated. The organic layer was further washed with saturated CuSO₄ (2×1.0 mL), water (10 mL) and brine and then concentrated to dryness. The resulting solid was filtered off, washed with water and dried in vacuo to afford tert-butyl 3-(2-(tosyloxy)ethoxy)pyrrolidine-1-carboxylate as yellow oil (530 mg, 48%). LCMS (ESI): m/z=330.2 [M−55]⁺.

### Referential Example 7

### [2-chloro-4-(3-methylpyrazin-2-yl)phenyl]boronic acid

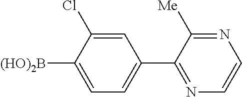 [SMILES: Cc1nccnc1-c1ccc(B(O)O)c(Cl)c1]

step 1: A 10 L 4-necked round-bottom flask was purged and maintained under a nitrogen atmosphere and then charged with 4-bromo-2-chloroaniline (300 g, 1.45 mol, 1.00 equiv) and oxolane (3000 mL). A solution of NaHMDS (1601 mL, 3.2 mol, 2.20 equiv) in THF was added dropwise with stirring at 0° C. over 3 hr. To this was added di-tert-butyl dicarbonate (381 g, 1.75 mol, 1.20 equiv) dropwise with stirring at 0° C. over 1 hr. The resulting solution was stirred overnight at RT. This reaction was repeated 1 more time. The reaction was then quenched by the addition 5 L of water/ice and the resulting solution was extracted with 5 L of EtOAc. The combined organic layers were dried over anhydrous Na₂SO₄ and concentrated under vacuum to afford 955 g (crude) of tert-butyl N-(4-bromo-2-chlorophenyl)carbamate as a brown oil.

step 2: A 10 L 4-necked round-bottom flask was purged and maintained under a nitrogen atmosphere then charged with tert-butyl N-(4-bromo-2-chlorophenyl)carbamate (477 g, 1.00 equiv, crude), dioxane (3500 mL), 4,4,5,5-tetramethyl-2-(tetramethyl-1,3,2-dioxaborolan-2-yl)-1,3,2-dioxaborolane (371 g, 1.46 mol, 1.00 equiv), KOAc (428 g, 4.36 mol, 3.00 equiv), and Pd(dppf)Cl₂ (18 g, 24.60 mmol, 0.02 equiv). The resulting solution was stirred at 90° C. for 4 h. This reaction was repeated for 1 more time. The solids were filtered out and the product was used in the next step without further purification.

step 3: A 10 L 4-necked round-bottom flask was purged and maintained under a nitrogen atmosphere and then charged with tert-butyl N-[2-chloro-4-(tetramethyl-1,3,2-dioxaborolan-2-yl)phenyl]carbamate from step 2 (1.00 equiv), 2-chloro-3-methylpyrazine (187 g, 1.45 mol, 1.00 equiv), a solution of Na₂CO₃ (312 g, 2.92 mol, 2.00 equiv) in water (1460 mL) and (PPh₃)₄Pd(II) (10 g). The resulting solution was stirred overnight at 85° C. This reaction was repeated 1 more time. The reaction mixture was cooled to RT, diluted with 10 L of water and extracted with 10 L of EtOAc. The organic layer was washed with 3×10 L of water, dried (Na₂SO₄), filtered and concentrated in vacuo. The residue was purified on a SiO₂ column eluting with EtOAc/PE gradient (1:10 to 1:5) to afford 350 g (75%) of tert-butyl N-[2-chloro-4-(3-methylpyrazin-2-yl)phenyl]carbamate as a white solid.

step 4: A 10 L 4-necked round-bottom flask was charged with tert-butyl N-[2-chloro-4-(3-methylpyrazin-2-yl)phenyl]carbamate (350 g, 1.09 mol, 1.00 equiv) and EtOAc (5 L) and then hydrogen chloride (gas) was introduced. The resulting solution was stirred at RT for 4 h and concentrated under vacuum. The solid was diluted with 3 L of water. The pH value of the solution was adjusted to 8 with K₂CO₃ and the resulting solution was extracted with 2×3 L of EtOAc. The combined organic layers were dried over anhydrous Na₂SO₄ and concentrated under vacuum to afford 240 g of 2-chloro-4-(3-methylpyrazin-2-yl)aniline as a white solid.

step 5: A 10 L 4-necked round-bottom flask was placed 2-chloro-4-(3-methylpyrazin-2-yl)aniline (120 g, 546.27 mmol, 1.00 equiv), water (2400 mL), CH₃CN (800 mL), con HCl (280 mL), followed by the dropwise addition of a solution of sodium nitrite (45.4 g, 658.02 mmol, 1.20 equiv) in water (500 mL) with stirring at 0° C. over 1 hr. The resulting solution was stirred at 0° C. for 1 h. To this was added dropwise a solution of NaI (164.25 g, 1.10 mol, 2.00 equiv) in water (500 mL) with stirring at 0° C. over 1 h. The resulting solution was stirred at 0° C. for 30 min. This reaction was repeated 1 more time. The resulting solution was diluted with 5 L of water and extracted with 10 L of EtOAc. The combined organic layers were washed with 1×10 L of Na₂SO₃, 1×10 L of water and 1×10 L of brine, dried over anhydrous Na₂SO₄ and concentrated under vacuum. The residue was purified on a SiO₂ column eluting with EtOAc/PE (1:5) to afford 230 g (64%) of 2-(3-chloro-4-iodophenyl)-3-methylpyrazine as a white solid.

step 6: A 3 L 4-necked round-bottom flask purged and maintained with an inert atmosphere of nitrogen then charged with 2-(3-chloro-4-iodophenyl)-3-methylpyrazine (110 g, 332.78 mmol, 1.00 equiv), B(i-PrO)₃ (125.3 g, 666.49 mmol, 2.00 equiv), oxolane (1500 mL). The solution was cooled to −78° C. and solution of butyllithium (180 mL, 433 mmol, 1.30 equiv) was added dropwise with stirring at −78° C. over 1 h. The resulting solution was stirred for 30 min. This reaction was repeated 1 more time. The reaction was then quenched by the addition of 3 L of NaOH (3 N) and extracted with 3×1.5 L of EtOAc. The aqueous layers were combined and the pH value of the solution was adjusted to 6 with aq HCl (6 mol/L). The solids were collected by filtration and dried in an oven under reduced pressure to afford 103 g (62%) of [2-chloro-4-(3-methylpyrazin-2-yl)phenyl]boronic acid as a white solid. ¹H-NMR (300 MHz, MeOH-d₄): δ 8.54-8.51 (dd, J=2.4, 2H), 7.63-7.49 (m, 3H), 2.62 (s, 3H); MS (ES, m/z): 249 [M+H]⁺.

### Referential Example 8

### 2-(2-(tert-Butoxycarbonylamino)ethoxy)ethyl 4-methylbenzenesulfonate

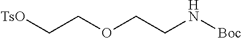 [SMILES: Cc1ccc(S(=O)(=O)OCCOCCNC(=O)OC(C)(C)C)cc1]

step 1: di-tert-Butyl dicarbonate (12.6 g, 58 mmol) was added to a mixture of 2-(2-aminoethoxy)ethanol (5.0 g, 48 mmol) in EtOH (100 mL). The reaction mixture was stirred overnight at RT and concentrated in vacuo to afford tert-butyl 2-(2-hydroxyethoxy)ethylcarbamate (10 g, crude) as a yellow oil, which was used in the next step without further purification. LCMS (ESI): m/z=206.2 [M+1]⁺.

step 2: DMAP (0.3 g, 2 mmol), TEA (7.4 g, 73 mmol) and tosyl chloride (6.8 g, 36 mmol) were added to a mixture of tert-butyl-2-(2-hydroxyethoxy)ethylcarbamate (5.0 g, 24 mmol) in DCM (60 mL). The mixture was stirred at RT overnight, concentrated in vacuo and purified by SiO₂ chromatography using PE/EtOAc (3:1) as eluent to afford 2-(2-(tert-butoxycarbonylamino)ethoxy)ethyl 4-methylbenzenesulfonate (8.0 g, 92%) as a yellow oil. LCMS (ESI): m/z=360.1 [M+1]⁺.

### Referential Example 9

### tert-Butyl 3-(tosyloxy)-1-oxa-7-azaspiro[4.4]nonane-7-carboxylate

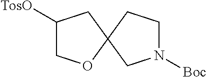

Tosyl chloride (4.7 g, 24.64 mmol), DMAP (150 mg, 1.2 mmol) and TEA (3.74 g, 36.96 mmol) were added to a solution of tert-butyl 3-hydroxy-1-oxa-7-azaspiro[4.4]nonane-7-carboxylate (3 g, 12.32 mmol; diastereomer mixture) in DCM (20 mL). The reaction mixture was stirred at RT overnight, diluted with DCM (30 mL) and washed with brine (3×50 mL). The organic layer was dried (Na₂SO₄), concentrated in vacuo, and purified by SiO₂ chromatography eluting first with DCM/PE (1:1) and then DCM/MeOH (100:1) as eluents tert-butyl 3-(tosyloxy)-1-oxa-7-azaspiro[4.4]nonane-7-carboxylate as a clear oil (3.62 g, 74%). LCMS (ESI): m/z=298 [M+H-Boc]⁺.

### Referential Example 10

### tert-Butyl 3-(2-hydroxyethoxy)azetidine-1-carboxylate

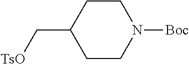 [SMILES: Cc1ccc(S(=O)(=O)OCC2CCN(C(=O)OC(C)(C)C)CC2)cc1]

A mixture of tert-butyl 4-(hydroxymethyl)piperidine-1-carboxylate (4.0 g, 18.6 mmol), tosyl chloride (4.25 g, 22.3 mmol), TEA (5.4 mL, 37.2 mmol), DMAP (568 mg, 4.65 mmol) in DCM (64 mL) and THF (16 mL) was stirred at 40° C. overnight. The reaction mixture was then concentrated, diluted with EtOAc (100 mL), and washed with an aqueous HCl solution (0.5 M, 40 mL) and then brine (2×50 mL). The organic layer was dried (MgSO₄), filtered, and concentrated. The residue was purified by SiO₂ chromatography, eluting with a PE/EtOAc gradient (100% to 50% in 50 min) to afford the target compound (5.2 g, 76%) as a white solid. LCMS (ESI): m/z=314.2 [M−55]⁺.

### Referential Example 11

### ((1s,4s)-4-(tert-Butoxycarbonylamino)cyclohexyl)methyl 4-methylbenzenesulfonate

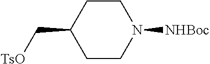

step 1: A mixture of ((1S,4S)aminocyclohexyl)methanol (400 mg, 3.10 mmol), di-tert-butyl dicarbonate (743 mg, 3.41 mmol) and TEA (627 mg, 6.20 mmol) in DCM (10 mL) was stirred at 25° C. for 1 h. The mixture was diluted with DCM (20 mL) and washed with an aqueous HCl solution (1 N, 20 mL×2) and brine (20 mL). The organic layers were dried (Na₂SO₄) and concentrated in vacuo to give the crude product tert-butyl (1s,4s)-4-(hydroxymethyl)cyclohexylcarbamate as a white solid (365 mg), which was used in the next step without further purification.

step 2: A mixture of tert-butyl (1S,4S)-4-(hydroxymethyl)cyclohexylcarbamate (480 mg, 2.09 mmol), TEA (423 mg, 4.18 mmol), tosyl chloride (478 mg, 2.51 mmol), and DMAP (26 mg, 0.21 mmol) in DCM (10 mL) was stirred at RT overnight. The reaction mixture was then concentrated in vacuo and purified by SiO₂ chromatography, eluting with PE/EtOAc (1:3) to afford ((1s,4s)-4-(tert-butoxycarbonylamino)cyclohexyl)methyl 4-methylbenzenesulfonate as a white solid (600 mg, 75%). MS (ESI): m/z=328.1 [M−55]⁺.

### Referential Example 12

### tert-Butyl 2-oxo-4-(2-(tosyloxy)ethyl)piperidine-1-carboxylate

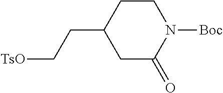 [SMILES: Cc1ccc(S(=O)(=O)OCCC2CCN(C(=O)OC(C)(C)C)C(=O)C2)cc1]

step 1: A mixture of 2-(piperidin-4-yl)ethanol (1.00 g, 7.74 mmol), di-tert-butyl dicarbonate (1.86 g, 8.51 mmol) and TEA (1.57 g, 15.48 mmol) in DCM (20 mL) was stirred at 25° C. for 1 h. The mixture was then diluted with DCM (20 mL) and washed with HCl (1 N, 2×20 mL) and brine (20 mL). The organic phase was dried (Na₂SO₄) and concentrated in vacuo to afford the crude product tert-butyl 4-(2-hydroxyethyl)piperidine-1-carboxylate as a white solid (1.60 g, 90%), which was used in the next step without further purification. MS (ESI): 230.2 [M−55]⁺.

step 2: A mixture of tert-butyl 4-(2-hydroxyethyl)piperidine-1-carboxylate (800 mg, 3.49 mmol), TEA (706 mg, 6.98 mmol), tosyl chloride (698 mg, 3.66 mmol), and DMAP (43 mg, 0.35 mmol) in DCM (10 mL) was stirred at RT overnight. The reaction mixture was then concentrated in vacuo and purified by SiO₂ chromatography eluting with PE/EtOAc (1:3) to afford tert-butyl 4-(2-(tosyloxy)ethyl)piperidine-1-carboxylate as a white solid (400 mg, 30%). MS (ESI): m/z=328.1 [M−55]⁺.

step 3: A mixture of tert-butyl 4-(2-(tosyloxy)ethyl)piperidine-1-carboxylate (400 mg, 1.04 mmol), sodium periodate (667 mg, 3.12 mmol), and ruthenium(IV) oxide (13 mg, 0.10 mmol) in EtOAc (10 mL) and water (10 mL) was stirred at RT overnight. The mixture was filtered, and the filtrate concentrated in vacuo to afford tert-butyl 2-oxo-4-(2-(tosyloxy)ethyl)piperidine-1-carboxylate as a white solid (370 mg, 87%), which was used in subsequent transformations without further purification. MS (ESI): m/z=342.0 [M−55]⁺.

### Referential Example 13

### (S)-tert-Butyl 3-(2-(tosyloxy)ethoxy)pyrrolidine-1-carboxylate

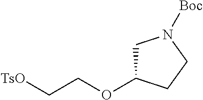 [SMILES: Cc1ccc(S(=O)(=O)OCCO[C@H]2CCN(C(=O)OC(C)(C)C)C2)cc1]

step 1: tert-Butyl 3-hydroxypyrrolidine-1-carboxylate (5.61 g, 30 mmol) was added to a suspension of sodium hydride (0.94 g, 39 mmol) in anhydrous THF (200 mL) at 0° C. The mixture was stirred for 40 min and then ethyl chloroacetate (4.14 mL, 30 mmol) was added dropwise. The resulting dark purple solution was then stirred overnight. The reaction mixture was quenched with 5% HCl solution and extracted with EtOAc (150 mL). The organic layer was washed with brine, dried (Na₂SO₄), filtered and concentrated in vacuo. The residue was purified by SiO₂ chromatography eluting with a EtOAc/PE gradient (1:5 to 1:3) to afford tert-butyl 3-(2-ethoxy-2-oxoethoxy)pyrrolidine-1-carboxylate as a yellow oil (3.4 g, 42%). LCMS (ESI): m/z=218 [M−55]⁺.

step 2: LiAlH₄ (900 mg, 24.3 mmol) was added slowly to o a 0° C. solution of tert-butyl 3-(2-ethoxy-2-oxoethoxy)pyrrolidine-1-carboxylate (6.6 g, 24.1 mmol) under N₂ in anhydrous THF (150 mL). The reaction mixture was stirred for 40 min and then quenched with water (15 mL) and 10% sodium hydroxide (6.8 mL). The mixture was extracted with EtOAc (250 mL) and concentrated in vacuo to afford tert-butyl 3-(2-hydroxyethoxy)pyrrolidine-1-carboxylate (3.92 g, 70%) as a colorless oil, which was used in the next step without further purification. LCMS (ESI): m/z=176.2 (M−55)⁺.

step 3: TEA (4.62 mL) and TsCl (3.78 g, 19.7 mmol) were added to solution of ter-butyl 3-(2-hydroxyethoxy)pyrrolidine-1-carboxylate (3.92 g, 16.9 mmol) in DCM (6 mL) and THF (24 mL) cooled to 0° C. The reaction mixture was stirred at RT overnight, then DMAP (1.08 g) was added and the reaction stirred for 2 h followed by quenching with saturated aqueous NaHCO₃ solution (12 ml). The organic layers were separated, washed with a saturated CuSO₄ solution (2×6.0 mL), water (60 mL), brine, dried (MgSO₄), filtered and concentrated in vacuo to give tert-butyl 3-(2-(tosyloxy)ethoxy)pyrrolidine-1-carboxylate (4.6 g, 70%) as a yellow oil. Chiral HPLC separation afforded (S)-tert-butyl 3-(2-(tosyloxy)ethoxy)pyrrolidine-1-carboxylate (1.84 g, 42%) as yellow oil. LCMS (ESI): m/z=330.2 [M−55]⁺.

(R)-tert-Butyl 3-(2-(tosyloxy)ethoxy)pyrrolidine-1-carboxylate was obtained in the chiral HPLC separation described in Step C of the procedure from the last paragraph (1.69 g, 37%). LCMS (ESI): m/z=330.2 [M−55]⁺.

### Referential Example 14

### tert-Butyl 3-(2-(tosyloxy)ethoxy)azetidine-1-carboxylate

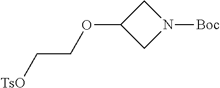 [SMILES: Cc1ccc(S(=O)(=O)OCCOC2CN(C(=O)OC(C)(C)C)C2)cc1]

step 1: Tetrabutylammonium bromide (148 mg, 0.46 mmol) was added to a solution of tert-butyl 3-hydroxyazetidine-1-carboxylate (4 g, 23 mmol) in TEA (2.467 g, 24 mmol). The reaction mixture was stirred for 5 h, and then 1,3-dioxolan-2-one (2.228 g, 25 mmol) was added, and the mixture was heated at 100° C. for 2.5 days. The mixture was then concentrated in vacuo and purified by SiO₂ chromatography eluting with a MeOH/DCM gradient (0% to 3% MeOH in 40 min), to give tert-butyl 3-(2-hydroxyethoxy)azetidine-1-carboxylate (2.63 g, 53%) as an orange colored oil. LCMS (ESI): m/z=162.1 [M−55]⁺.

step 2: A mixture of tert-butyl 3-(2-hydroxyethoxy)azetidine-1-carboxylate (2.630 g, 12.1 mmol), tosyl chloride (2.768 g, 14.5 mmol), TEA (3.5 mL, 24.2 mmol), DMAP (370 mg, 3.025 mmol) in DCM (36 mL) and THF (9 mL) was stirred at 40° C. overnight. The reaction mixture was then concentrated, diluted with DCM (50 mL) and washed with an aqueous HCl (0.5 M, 40 mL). The aqueous layer was then extracted with DCM (2×30 mL) and the combined organic layers washed with a NaHCO₃ solution (50 mL), brine (50 mL), dried (MgSO₄), filtered, and concentrated. The residue was purified by SiO₂ chromatography, eluting with a gradient of EA/mixture of PE/DCM (1/1) (3% to 6% gradient) to give tert-butyl 3-(2-(tosyloxy)ethoxy)azetidine-1-carboxylate (2.36 g, 52%) as a yellow oil. LCMS (ESI): m/z=316.0 [M−55]⁺.

### Referential Example 15

### tert-Butyl 3-(2-methylsulfonyloxyethyl)pyrrolidine-1-carboxylate

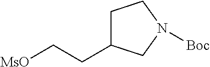 [SMILES: CC(C)(C)OC(=O)N1CCC(CCOS(C)(=O)=O)C1]

To a flame dried flask were added tert-butyl 3-(2-hydroxyethyl)pyrrolidine-1-carboxylate (200 mg, 0.883 mmol), TEA (0.25 mL, 1.77 mmol), and DCM (4.4 mL). The resulting solution was cooled to 0° C. under a nitrogen atmosphere. Methanesulfonyl chloride (0.075 mL, 0.971 mmol) was then added dropwise and the solution was stirred at 0° C. for 90 min. The reaction mixture was poured into EtOAc and washed with brine. The organic layer was dried (MgSO₄), filtered and concentrated to afford the crude product as yellow-colored oil (259 mg, 100%) that was used for subsequent transformations without further purification.

### Referential Example 16

### tert-Butyl 2-oxo-4-(2-(tosyloxy)ethyl)pyrrolidine-1-carboxylate and tert-butyl 2-oxo-3-(2-(tosyloxy)ethyl)pyrrolidine-1-carboxylate

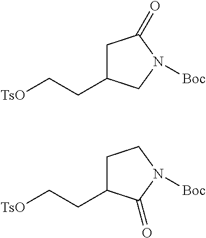 [SMILES: Cc1ccc(S(=O)(=O)OCCC2CC(=O)N(C(=O)OC(C)(C)C)C2)cc1 ; Cc1ccc(S(=O)(=O)OCCC2CCN(C(=O)OC(C)(C)C)C2=O)cc1]

To a solution of ruthenium(III) chloride (0.2 equiv., 0.88 mmol, 182 mg) and sodium periodate (3 equiv., 13.2 mmol, 238 mg) in water (2.9 mL) was added a solution of tert-butyl 3-[2-(p-tolylsulfonyloxy)ethyl]pyrrolidine-1-carboxylate (1.62 g, 4.38 mmol) in MeCN (3.7 mL). The resulting suspension was stirred vigorously at RT overnight. The reaction mixture was filtered through CELITE and rinsed with EtOAc. The reaction was quenched with sat. aq. Na₂SO₃ solution and extracted into EtOAc (3×). The combined organic layers were washed with brine, dried (MgSO₄), and concentrated to afford the crude product as a dark oil. The crude residue was adsorbed onto CELITE and purified by SiO₂ chromatography (24 g Silicycle HP cartridge) eluting with an EtOAc/heptane gradient (0 to 100% EtOAc) to separate the regioisomers.

tert-Butyl 2-oxo-4-(2-(tosyloxy)ethyl)pyrrolidine-1-carboxylate: ¹H NMR (400 MHz, CDCl₃) δ 7.79 (d, J=8.1 Hz, 2H), 7.36 (d, J=8.0 Hz, 2H), 4.07 (m, 2H), 3.90-3.78 (m, 1H), 3.29 (dd, J=10.8, 7.6 Hz, 1H), 2.55 (dd, J=16.9, 8.2 Hz, 1H), 2.46 (s, 3H), 2.40 (m, 1H), 2.14 (dd, J=16.9, 8.6 Hz, 1H), 1.81 (m, 2H), 1.52 (s, 9H).

tert-Butyl 2-oxo-3-(2-(tosyloxy)ethyl)pyrrolidine-1-carboxylate: ¹H NMR (400 MHz, CDCl₃) δ 7.81-7.75 (m, 2H), 7.35 (m, 2H), 4.25-4.15 (m, 2H), 3.76 (ddd, J=10.8, 8.8, 1.9 Hz, 1H), 3.54 (ddd, J=10.9, 10.2, 6.9 Hz, 1H), 2.59 (m, 1H), 2.46 (s, 3H), 2.29-2.15 (m, 2H), 1.79-1.60 (m, 2H), 1.55-1.49 (s, 9H).

### Referential Example 17

### tert-Butyl 3-(tosyloxy)-1-oxa-8-azaspiro[4.5]decane-8-carboxylate

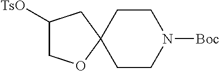 [SMILES: Cc1ccc(S(=O)(=O)OC2COC3(CCN(C(=O)OC(C)(C)C)CC3)C2)cc1]

Tosyl chloride (1.5 g, 7.8 mmol), DMAP (47.6 mg, 0.39 mmol) and TEA (1.18 g, 11.7 mmol) were added to a solution of tert-butyl 3-hydroxy-1-oxa-8-azaspiro[4.5]decane-8-carboxylate (1 g, 3.9 mmol) in DCM (20 mL). The mixture was stirred at RT overnight and then diluted with DCM (30 mL) and washed with brine (3×50 mL). The organic layers were dried over Na₂SO₄ and concentrated in vacuo. The crude product was purified by silica gel chromatography eluting with DCM/PE (1:1) to DCM:MeOH (100:1) to afford tert-butyl 3-(tosyloxy)-1-oxa-7-azaspiro[4.4]nonane-7-carboxylate as a colorless oil (1.13 g, 70%) LCMS (ESI): m/z=312 [M+H-Boc]⁺.

### Referential Example 18

### (R)-tert-Butyl 2-(((methylsulfonyl)oxy)methyl)morpholine-4-carboxylate

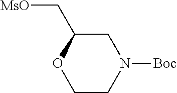 [SMILES: CC(C)(C)OC(=O)N1CCO[C@@H](COS(C)(=O)=O)C1]

To a solution of tert-butyl (2R)-2-(hydroxymethyl)morpholine-4-carboxylate (550 mg) in anhydrous DCM (7.2 mL) at 0° C. was added TEA (388 mg, 109 mmol). After 5 min. methanesulfonyl chloride (350 mg, 3.80 mmol) was added, and the reaction mixture was stirred at RT under N₂ for 3 h. The reaction mixture was diluted with EtOAc. The organic layer was washed with brine, dried (Na₂SO₄), filtered, and concentrated to give 740 mg (99%) of (R)-tert-butyl 2-(((methylsulfonyl)oxy)methyl)morpholine-4-carboxylate as an oil. ¹H NMR (400 MHz, CDCl₃) δ 4.23 (d, J=4.9 Hz, 2H), 4.04-3.78 (m, 3H), 3.73-3.65 (m, 1H), 3.54 (td, J=11.6, 2.8 Hz, 1H), 3.06 (s, 3H), 3.01-2.88 (m, 1H), 2.84-2.68 (m, 1H), 1.47 (s, 9H).

### Referential Example 18

### Benzyl 2-(2-((methylsulfonyl)oxy)ethyl)-morpholine-4-carboxylate

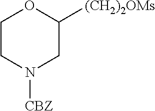

step 1: To a solution of 2-morpholin-2-ylethanol (1.0 g, 7.62 mmol) and TEA (1.0 g, 9.9108 mmol) in anhydrous DCM (15 mL) at 0° C. was added dropwise benzyl chloroformate (1.3 g, 7.62 mmol). The reaction mixture was slowly warmed to RT, stirred for 20 h, and diluted with EtOAc. The organic layer was washed with brine, dried (Na₂SO₄), filtered, and concentrated under reduced pressure. The crude residue was purified by SiO₂ chromatography eluting with heptane/EtOAc to afford 1.71 g (84.5%) of benzyl 2-(2-hydroxyethyl)morpholine-4-carboxylate as clear oil. ¹H NMR (400 MHz, CDCl₃) δ 7.42-7.28 (m, 5H), 5.15 (d, J=1.7 Hz, 2H), 4.09-3.83 (m, 3H), 3.79 (dd, J=6.2, 5.1 Hz, 2H), 3.68-3.48 (m, 2H), 3.01 (br s, 1H), 2.75 (br s, 1H), 2.19-2.01 (m, 1H), 1.82-1.62 (m, 2H).

step 2: To a solution of benzyl 2-(2-hydroxyethyl)morpholine-4-carboxylate (1.71 g, 6.45 mmol) in anhydrous DCM (17 mL) at 0° C. was added TEA (988 mg, 9.67 mmol). After 5 min. methanesulfonyl chloride (886 mg, 7.73 mmol) was added, and the resultant heterogeneous reaction mixture was stirred at RT under N₂ for 3 h. The reaction mixture was diluted with EtOAc. The organic layer was washed with brine, dried (Na₂SO₄), filtered, and concentrated to give 2.18 g (98.5% yield) of benzyl 2-(2-((methylsulfonyl)oxy)ethyl)-morpholine-4-carboxylate as an oil. ¹H NMR (400 MHz, CDCl₃) δ 7.40-7.29 (m, 5H), 5.15 (d, J=1.5 Hz, 2H), 4.45-4.28 (m, 2H), 4.11-3.79 (m, 3H), 3.62-3.45 (m, 2H), 2.99 (s, 4H), 2.71 (br s, 1H), 1.97-1.75 (m, 2H).

### Referential Example 19

### 1-Oxa-6-aza-spiro[2.5]octane-6-carboxylic acid benzyl ester

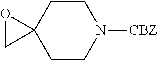

At 0° C., sodium hydride (600 mg, 60% in oil mineral, 15 mmol) was added to a solution of trimethylsulfoxonium iodide (3.3 g, 15 mmol) in DMSO (50 mL). The mixture was stirred for 40 min at RT, and then 4-oxo-piperidine-1-carboxylic acid benzyl ester (2.33 g, 10 mmol) was added. The mixture was stirred at 55° C. for 2 h, cooled to RT and poured into ice water. The crude mixture was extracted with EtOAc (2×50 mL) and the combined organic layers dried over anhydrous Na₂SO₄ and concentrated in vacuo. The residue was purified by silica gel chromatography eluting with EtOAc/PE (1:1) to afford 1-oxa-6-aza-spiro[2.5]octane-6-carboxylic acid benzyl ester as a colorless oil (1.7 g, 69%). LCMS (ESI): m/z=248.0 [M+1]⁺.

### Referential Example 20

### tert-Butyl 4-(2-aminoethyl)-3-oxopiperazine-1-carboxylate

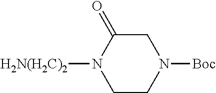

step 1: To a mixture of NaH (430 mg, 1.8 mmol) in DMF (5 mL) was added tert-butyl 3-oxopiperazine-1-carboxylate (3.0 g, 1.5 mmol) and 2-bromoacetonitrile (1.8 g, 1.5 mmol) at 0° C. The mixture was stirred at RT overnight. The reaction mixture was then diluted with EtOAc (10 mL) and H₂O (10 mL). The separated organic layer was washed with brine, dried (MgSO₄), filtered and concentrated in vacuo. The residue was purified by SiO₂ chromatography eluting with DCM/MeOH (40:1) to afford tert-butyl 4-(cyanomethyl)-3-oxopiperazine-1-carboxylate as yellow oil (3.0 g, 76%). LCMS (ESI): m/z=240.1 [M+1]⁺

step 2: A mixture of tert-butyl 4-(cyanomethyl)-3-oxopiperazine-1-carboxylate (3.0 g, 12.5 mmol), PtO₂ (300 mg, 1.32 mmol) in EtOH (20 mL) was stirred at 20° C. under H₂ (50 psi) overnight. The Pd/C catalyst was removed by filtration, and the filtrate was concentrated under reduced pressure to afford tert-butyl 4-(2-aminoethyl)-3-oxopiperazine-1-carboxylate (2.8 g, 93%) as yellow solid. LCMS (ESI): m/z=244.2 [M+1]⁺.

### Referential Example 21

### tert-Butyl 3-(1-(tosyloxy)propan-2-yloxy)azetidine-1-carboxylate

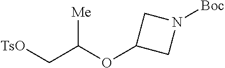 [SMILES: Cc1ccc(S(=O)(=O)OCC(C)OC2CN(C(=O)OC(C)(C)C)C2)cc1]

step 1. To a stirring solution of tert-butyl 3-hydroxyazetidine-1-carboxylate (4.00 g, 23.09 mmol) in dry DMF (20 mL) was added NaH (1.11 g, 46.18 mmol, 60% in mineral) in portions at 0° C. After the suspension was stirred at 0° C. for 30 min, ethyl 2-bromopropanoate (8.36 g, 46.18 mmol) was added. The resulting mixture was stirred at RT under N₂ overnight. The reaction was quenched by addition of saturated aqueous NH₄Cl solution (40 mL) and extracted with EtOAc (50 mL×3). The combined organic layers were dried (Na₂SO₄), filtered, and evaporated under reduced pressure. The residue was purified by SiO₂ chromatography eluting with PE/EtOAc (10:1) to afford tert-butyl 3-(1-ethoxy-1-oxopropan-2-yloxy)azetidine-1-carboxylate 1 as a colorless oil (1.00 g, 16%). LCMS (ESI): m/z=218.1 [M−55]⁺.

step 2. A suspension of LiALH₄ (625 mg, 16.47 mmol) in dry THF (20 mL) was cooled to 0° C. tert-Butyl 3-(1-ethoxy-1-oxopropan-2-yloxy)azetidine-1-carboxylate (3.00 g, 10.98 mmol) was added in several portions. The mixture was stirred at RT overnight. The reaction was quenched by addition of saturated aqueous NH₄Cl solution (40 mL), and extracted with EtOAc (50 mL×3). The combined organic layers were dried over anhydrous Na₂SO₄, filtered, and evaporated under reduced pressure to afford crude product tert-butyl 3-(1-hydroxypropan-2-yloxy)azetidine-1-carboxylate as a colorless oil (1.50 g, crude). LCMS (ESI): m/z=176.1 [M−55]⁺.

step 3. A solution of tert-butyl 3-(1-hydroxypropan-2-yloxy)azetidine-1-carboxylate (1.30 g, 5.62 mmol), 4-methylbenzene-1-sulfonyl chloride (1.61 g, 8.43 mmol), TEA (1.14 g, 11.24 mmol) and N,N-dimethyl-4-aminopyridine (68 mg, 0.56 mmol) in DCM (30 mL) was stirred at RT overnight. The reaction mixture was then concentrated under reduced pressure. The residue was purified by silica gel chromatography using PE/EtOAc (5:1) as eluting solvent to obtain the target compound tert-butyl 3-(1-(tosyloxy)propan-2-yloxy)azetidine-1-carboxylate (1.20 g, 55%, two steps) as a colorless oil. LCMS (ESI): m/z=330.0 [M−55]⁺.

### Referential Example 22

### tert-Butyl 3-(2-(tosyloxy)propoxy)azetidine-1-carboxylate

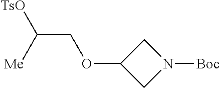 [SMILES: Cc1ccc(S(=O)(=O)OC(C)COC2CN(C(=O)OC(C)(C)C)C2)cc1]

step 1: To a solution of tert-butyl 3-hydroxyazetidine (2.5 g, 14.5 mmol) in DMF (100 mL) was added NaH (1.45 g, 36.3 mmol) at 0° C. and stirred for 30 min. After addition of 4-methyl-1,3-dioxolane-2-one (3 g, 29 mmol) the reaction was stirred at 120° C. overnight. The reaction mixture was cooled to RT and quenched by adding a saturated aqueous NH₄HCO₃ solution (5 mL). The mixture was concentrated under reduced pressure to afford tert-butyl 3-(2-hydroxypropoxy)azetidine-1-carboxylate (4 g) as yellow solid, which was used in the next step without further purification. LCMS (ESI): m/z=232.1 [M+1]⁺

step 2: A mixture of tert-butyl 3-(2-hydroxypropoxy)azetidine-1-carboxylate (4.0 g, 17 mmol), DMAP (0.15 g, 1 mmol), TEA (3.7 g, 36.5 mmol) and tosyl chloride (4.9 g, 26 mmol) was stirred in DCM (60 mL) at RT overnight. After concentration under reduced pressure, the crude product was purified by SiO₂ chromatography eluting with PE/EtOAc (3:1) to afford tert-butyl 3-(2-(tosyloxy)propoxy)azetidine-1-carboxylate (4.0 g, 60%, two steps) as yellow oil. LCMS (ESI): m/z=386.2 [M+1]⁺.

### Referential Example 23

### 2-(3-(2-Hydroxyethoxy)cyclobutyl)isoindoline-1,3-dione

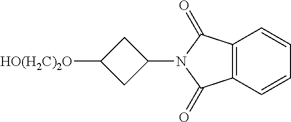

step 1: A mixture of 3-aminocyclobutanol hydrochloride (1.91 g, 15.47 mmol), isobenzofuran-1,3-dione (2.52 g, 17.01 mmol) and DIPEA (6.0 g, 46.40 mmol) in PhMe (100 mL) was stirred at 110° C. for 18 h. The reaction mixture was concentrated and purified by SiO₂ chromatography eluting with PE/EA (3:1) to afford 2-(3-hydroxycyclobutyl)isoindoline-1,3-dione as white solid (2.1 g, 62.6%). LCMS (ESI): m/z=218.1 [M+1]⁺

step 2: A mixture of 2-(3-hydroxycyclobutyl)isoindoline-1,3-dione (1.70 g, 7.83 mmol), 1,3-dioxolan-2-one (759 mg, 8.62 mmol) and tetrabutylammonium fluoride (51 mg, 0.157 mmol) in TEA (870 mg, 8.62 mmol) was stirred at 120° C. for 18 h. The reaction mixture was concentrated and purified by SiO₂ chromatography eluting with PE/EA (3:1) to afford 2-(3-(2-hydroxyethoxy)cyclobutyl)isoindoline-1,3-dione as yellow solid (243 mg, 11.8%). LCMS (ESI): m/z=262.0 [M+1]⁺.

### Referential Example 24

### 4-Chloro-2-(methylthio)pyrimidine-5-carbaldehyde

step 1: To ethyl 4-chloro-2-(methylthio)pyrimidine-5-carboxylate (370.0 g, 1.59 mol) in DCM (7.4 L) at −78° C. was added diisobutylaluminum hydride (1.5 M in toluene, 2.1 L, 3.18 mol). The reaction mixture was allowed to warm to 0° C. over 2 h. To the reaction was added a 20% Rochelle's salt solution (5.0 L). The emulsion was stirred for 30 min. The mixture was filtered through a bed of CELITE. The organic layer was separated and the aqueous layer extracted with EtOAc (1.0 L×3). The combined organic layers were dried (_{(Na2SO4)}), filtered, and concentrated in vacuo to give the crude product (4-chloro-2-(methylthio)pyrimidin-5-yl)methanol as a solid. The crude product was purified by SiO₂ chromatography eluting with DCM/EtOAc (1:1) to afford the pure product (4-chloro-2-(methylthio)pyrimidin-5-yl)methanol (133.0 g, 44.0%) as a solid. ¹H NMR (400 MHz, CDCl₃) δ 8.55 (s, 1H), 4.74 (s, 2H), 2.58 (s, 3H); MS: 191 [M+H]⁺.

step 2: Manganese (IV) oxide (137.4 g, 1.58 mol) was added to a solution of (4-chloro-2-(methylthio)pyrimidin-5-yl)methanol (30.0 g, 0.158 mol) in DCM (1.5 L). The reaction was stirred overnight, filtered through CELITE, and the filtrate was concentrated in vacuo to give the crude product 4-chloro-2-(methylthio)pyrimidine-5-carbaldehyde as a white solid. The crude product was purified by SiO₂ chromatography eluting with PE/EA (10:1) to afford pure 4-chloro-2-(methylthio)pyrimidine-5-carbaldehyde (21.0 g, 74.1%) as a white solid. ¹H NMR (400 MHz, CDCl₃) δ 10.31 (s, 1H), 8.87 (s, 1H), 2.64 (s, 3H); MS: 189 [M+H]⁺.

### Referential Example 25

### 4-Amino-2-(methylthio)pyridine-5-carbaldehyde

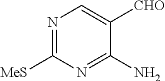 [SMILES: CSc1ncc(C=O)c(N)n1]

step 1. To a 2 L three-necked flask was added ethyl 4-chloro-2-(methylthio)pyrimidine-5-carboxylate (100 g, 0.43 mol), THF (500 mL), and TEA (186 mL, 1.29 mol). Ammonium hydroxide (28% in water, 400 mL) was added by portions keeping the internal temperature below 30° C. After 2 h, water (1000 mL) was added and THF was distilled off under vacuum. The resulting solid was filtrated and dried in vacuo at 50° C. to afford ethyl 4-amino-2-(methylthio)pyrimidine-5-carboxylate (90 g, 99%). LC/MS: m/z=213.9 [M+H]+; ¹H-NMR (DMSO-d6, 400 MHz) δ 8.57 (s, 1H), 8.03 (br, 1H), 7.65 (br, 1H), 4.27 (q, J=7.5 Hz, 2H), 2.46 (s, 3H), 1.29 (t, J=7.2 Hz, 3H).

step 2: To a 3 L three-necked flask was added LiAlH₄ (25.7 g, 0.68 mol) and THF (1 L), and the suspension was cooled to −10 to 0° C. A solution of ethyl 4-amino-2-(methylthio)pyrimidine-5-carboxylate (120 g, 0.56 mol) in THF (1 L) was slowly added through an addition funnel to the stirred mixture. The reaction mixture was then allowed to warm to 20-25° C. After 2 h, the mixture was cooled to 0° C. and slowly quenched with water (26 mL). Then 10% sodium hydroxide (26 mL) was added, followed by agitation for 2 h and addition of water (78 mL). After filtration, the mixture was concentrated to dryness under reduced pressure. Crude (4-amino-2-(methylthio)pyrimidin-5-yl)methanol (80 g, 83%) was obtained, which was used in the next step without further purification. LC/MS: m/z=171.8 [M+H]⁺.

step 3: To a 3 L three-necked flask was added (4-amino-2-(methylthio)pyrimidin-5-yl)methanol (100 g, 0.58 mol), activated MnO₂ (152 g, 1.75 mol) and THF (2000 mL). The mixture was heated to 40-45° C. for 16 h. The reaction mixture was cooled to 20-25° C., filtered through a CELITE pad, and rinsed with THF (400 mL×3). The combined filtrates were concentrated to dryness. Pure 4-amino-2-(methylthio)pyrimidine-5-carbaldehyde (51 g, 51.6%) was obtained by trituration in 200 mL EtOAc/n-heptane (1:5) at 20-25° C., followed by removal of the solvent in vacuo; ¹H-NMR (CDCl₃, 400 MHz) δ 9.80 (s, 1H), 8.44 (s, 1H), 8.22 (br, 1H), 5.77 (br, 1H), 2.57 (s, 3H); LC/MS: m/z=169.8 [M+H]⁺.

### Referential Example 26

### [2-Chloro-4-(6-methylpyrazin-2-yl)phenyl]boronic acid

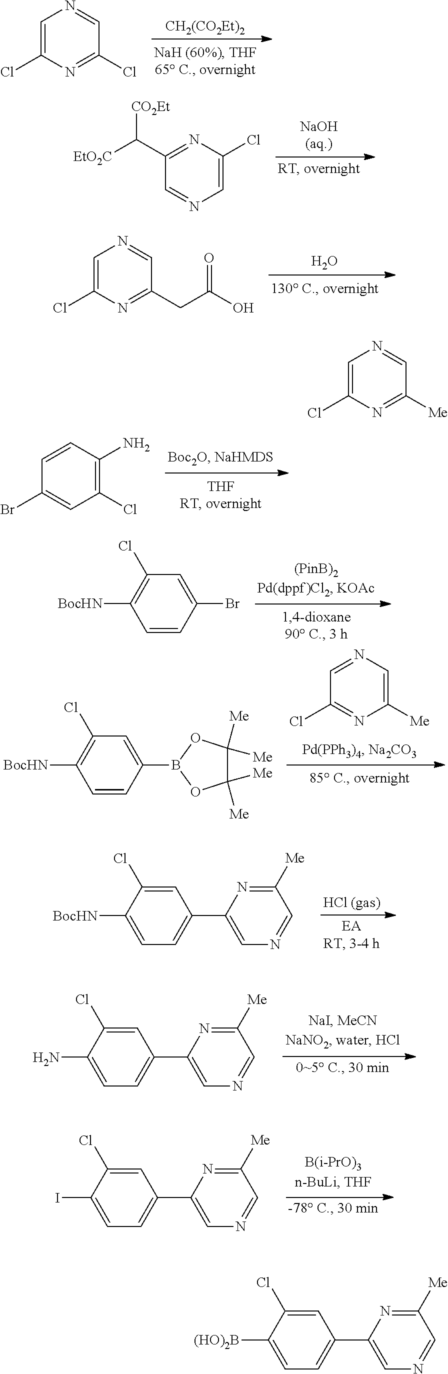

step 1: Into a 20-L 3-necked round-bottom flask was placed THF (6 L) and NaH (325 g, 8.12 mol, 2.00 equiv, 60%). To this was added 2,6-dichloropyrazine (600 g, 4.03 mol, 1.00 equiv) in several batches while maintaining the temperature below 10° C., followed by the addition of 1,3-diethyl propanedioate (1386 g, 8.65 mol, 2.13 equiv) dropwise with stirring while maintaining the temperature below 10° C. over 3 h. The resulting solution was heated to reflux overnight, cooled, quenched by the addition of water/ice and extracted with 2×3 L of EtOAc. The organic layers were combined, dried (Na₂SO₄), filtered and concentrated in vacuo to afford 2000 g (crude) of 1,3-diethyl 2-(6-chloropyrazin-2-yl)propanedioate as a brown oil.

step 2: Into a 20-L 3-necked round-bottom flask was placed water (9 L), NaOH (900 g, 22.50 mol, 3.07 equiv) in portions, followed by the addition of 1,3-diethyl 2-(6-chloropyrazin-2-yl)propanedioate (2 kg, 1.00 equiv, crude) dropwise with stirring while maintaining the temperature below 10° C. The resulting solution was stirred overnight at RT and cooled to 0° C. The pH value of the solution was adjusted to ca. 9-10 with HCl (37%) and the resulting solution was extracted with 3×2 L of EtOAc. The organic layers were combined, dried (Na₂SO₄), filtered and concentrated in vacuo. The residue was washed with PE (1×1.5 L) and MTBE (1×1.5 L), dried (Na₂SO₄), and concentrated in vacuo to afford 463.6 g (37%) of 2-(6-chloropyrazin-2-yl)acetic acid as a yellow solid.

step 3: Into a 5-L sealed tube was placed 2-(6-chloropyrazin-2-yl)acetic acid (220 g, 1.27 mol, 1.00 equiv) and water (3 L). The resulting solution was stirred overnight at 130° C., cooled and extracted with Et₂O 2×3 L of ether. The organic layers were combined, dried (Na₂SO₄), filtered and concentrated in vacuo to afford 238 g (73%) of 2-chloro-6-methylpyrazine as a yellow solid.

step 4: Into a 20-L 3-necked round-bottom flask was placed 4-bromo-2-chloroaniline (800 g, 3.87 mol, 1.00 equiv) and THF (10 L), followed by the dropwise addition of NaHMDS (3.9 L, 7.8 mol, 2.00 equiv, 2 mol/L) with stirring while maintaining the temperature below 10° C. The resulting solution was stirred at RT for 1 h. To this was added Boc₂O (1.012 kg, 4.64 mol, 1.20 equiv) dropwise with stirring while maintaining the temperature below 10° C. The resulting solution was stirred overnight at RT, quenched by the addition of 10 L of water/ice and extracted with EtOAc (3×5 L). The organic layers were combined, washed with brine (1×5) of, dried (Na₂SO₄), filtered and concentrated under vacuum to afford 2.3 kg (crude) of tert-butyl N-(4-bromo-2-chlorophenyl)carbamate as a brown oil.

step 5: A 10-L 4-necked round-bottom flask purged and maintained with an inert atmosphere of nitrogen was charged with tert-butyl N-(4-bromo-2-chlorophenyl)carbamate (1.15 kg, 3.75 mol, 1.00 equiv), dioxane (3.5 L), KOAc (429 g, 4.37 mol, 1.17 equiv), 4,4,5,5-tetramethyl-2-(tetramethyl-1,3,2-dioxaborolan-2-yl)-1,3,2-dioxaborolane (371 g, 1.46 mol, 0.39 equiv), and Pd(dppf)Cl₂ (18 g). The resulting solution was stirred at 90° C. for 3 h. This reaction was repeated for 1 more time. The reaction was cooled and the solids were filtered. The product, tert-butyl N-[2-chloro-4-(tetramethyl-1,3,2-dioxaborolan-2-yl)phenyl]carbamate was used in the next step directly without further purification.

step 6: Into a 20-L 4-necked round-bottom flask purged and maintained with an inert atmosphere of nitrogen was placed a solution of tert-butyl N-[2-chloro-4-(tetramethyl-1,3,2-dioxaborolan-2-yl)phenyl]carbamate, Na₂CO₃ (2 N, 2.90 L), 2-chloro-6-methylpyrazine (236 g, 1.84 mol, 0.65 equiv; obtained in step 3), and Pd(PPh₃)₄ (20 g). The resulting solution was stirred overnight at 85° C. and cooled. The solids were filtered out and the filtrate was diluted with 3 L of water. The resulting solution was extracted with EtOAc (3×3 L). The organic layers were combined, washed with water (3×3 L) and brine (1×3 L), dried (Na₂SO₄), filtered and concentrated in vacuo. The residue was purified by SiO₂ chromatography eluting with EtOAc:PE (1:1) to afford 340 g (38%) of tert-butyl N-[2-chloro-4-(6-methylpyrazin-2-yl)phenyl]carbamate as a yellow solid.

step 7: A 10-L 4-necked round-bottom flask was charged with tert-butyl N-[2-chloro-4-(6-methylpyrazin-2-yl)phenyl]carbamate (340 g, 1.06 mol, 1.00 equiv) and EtOAc (5 L). Hydrogen chloride (gas) was introduced in at 0° C. and the resulting solution was stirred at RT for 3-4 h. The resulting mixture was concentrated in vacuo, diluted with water (1 L) and extracted with EtOAc (3×1 L). The organic layers were combined, dried over anhydrous (Na₂SO₄), filtered and concentrated in vacuo to afford 215 g (92%) of 2-chloro-4-(6-methylpyrazin-2-yl)aniline as a yellow solid.

step 8: A 10-L 4-necked round-bottom flask was charged with 2-chloro-4-(6-methylpyrazin-2-yl)aniline (215 g, 978.74 mmol, 1.00 equiv), MeCN (1.43 L), HCl (con., 501 mL), water (4.3 L), followed by the addition of a solution of NaNO₂ (80.9 g, 1.17 mol, 1.20 equiv) in water (500 mL) dropwise with stirring while maintaining the temperature at 0-5° C. The resulting solution was stirred at 0-5° C. ° C. for 40 min. To this was added a solution of NaI (293 g, 1.95 mol, 2.00 equiv) in water (500 mL) dropwise with stirring at 0-5° C. The resulting solution was stirred at 0-5° C. for 30 min, diluted with of water and extracted with EtOAc (3×2 L). The combined organic layers were washed with Na₂SO₃ (2×2 L), water (2×2 L) and brine (2×2 L), dried (Na₂SO₄), filtered and concentrated under vacuum. The residue was applied by SiO₂ chromatography eluting with EtOAc/petroleum ether (1:2) to afford 196 g (61%) of 2-(3-chloro-4-iodophenyl)-6-methylpyrazine as a yellow solid.

step 9: A 3-L 4-necked round-bottom flask purged and maintained with an inert atmosphere of nitrogen was charged with 2-(3-chloro-4-iodophenyl)-6-methylpyrazine (110 g, 332.78 mmol, 1.00 equiv), tris(propan-2-yl)borate (125.3 g, 666.23 mmol, 2.00 equiv), THF (1.5 L). n-BuLi (180 mL, 433 mmol, 1.30 equiv, 2.4 M) was added dropwise with stirring at −78° C. The resulting solution was stirred at −78° C. for 30 min. This reaction was repeated for 2 more times. The reaction was quenched by the addition of 3% NaOH until the pH=12 and the resulting solution was extracted with EtOAc (3×2 L). The aqueous layers were combined and the pH value of the solution was adjusted to 4-5 with HCl (6 N). The precipitates were collected by filtration, washed with 2×1 L of water and 2×1 L of MTBE, dried in an oven under reduced pressure to afford 152 g (61%) of [2-chloro-4-(6-methylpyrazin-2-yl)phenyl]boronic acid as a light yellow solid.: 249 [M+H]⁺. ¹H-NMR (MeOH-d₄, 300 MHz): δ 2.64 (3H, s), 7.49˜7.52 (1H, d, J=7.8 Hz), 7.98˜8.01 (1H, d, J=1.2 Hz), 8.11 (1H, s), 8.48 (1H, s), 8.91 (1H, s); LC-MS (ES, m/z).

### Referential Example 27

### Methyl 2-(2-methyl-4-(6-methylpyridin-2-yl)phenyl)acetate

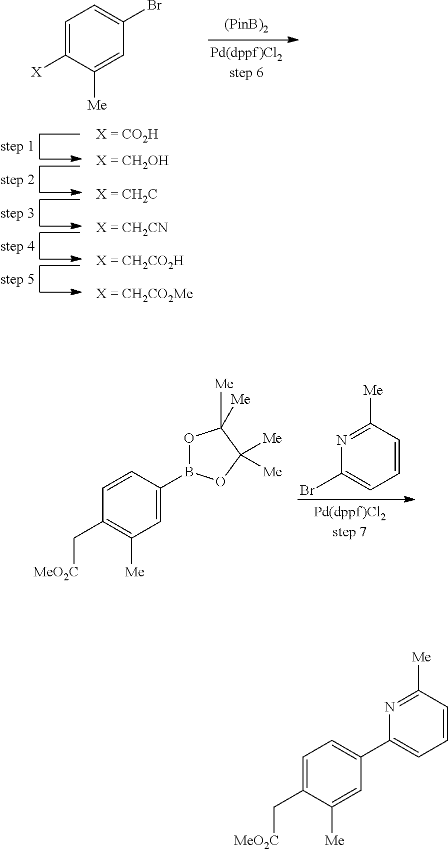

step 1: To a solution of 4-bromo-2-methylbenzoic acid 1 (215.0 g, 1.0 mol) in THF (1 L) was added LiAlH₄ (76.0 g, 1.0 mol) portions at 0° C. The reaction was stirred at this temperature for 1 h and then the reaction was quenched with 1 N HCl (2.0 L). The mixture was extracted with EtOAc (2×1000 mL) and the organic layers were washed with brine (2×1000 mL) and dried over anhydrous (Na₂SO₄), filtered and concentrated in vacuo. The residue was purified by SiO₂ chromatography eluting with EtOAc/PE (1/5) to afford (4-bromo-2-methylphenyl)methanol as yellow solid (125 g, 62% yield). ¹H NMR (400 MHz, CDCl₃): δ 7.31 (m, 2H), 7.19 (d, J=8.8 Hz, 1H), 4.59 (s, 2H), 2.28 (s, 3H), 1.95 (s, 1H).

step 2: To a solution of (4-bromo-2-methylphenyl)methanol (200.0 g, 1.0 mol) in DCM (500 mL) was added SOCl₂ (100 mL, 1.2 mol) dropwise at 0° C. The reaction mixture was heated to reflux and stirred at this temperature for 4 h. The reaction mixture was cooled to RT and concentrated in vacuo. The residue was diluted with EtOAc (1 L) and washed with water (2×500 mL), sat. NaHCO₃ (1000 mL) and brine (1 L). The organic layer was dried (Na₂SO₄), filtered and concentrated in vacuo to afford 4-bromo-1-(chloromethyl)-2-methylbenzene as a yellow oil, which was used in the next step without further purification (200 g, 100% yield).

step 3: To a solution of 4-bromo-1-(chloromethyl)-2-methylbenzene (200.0 g, 1.0 mol) in EtOH (500 mL) was added NaCN (72 g, 2.0 mol). The reaction mixture was heated to reflux and stirred at this temperature for 12 h. The reaction mixture was cooled to RT and concentrated in vacuo. The residue was diluted with EtOAc (1 L) and washed with water (2×500 mL), sat. NaHCO₃ (1 L) and brine (1 L). The organic layer was dried (Na₂SO₄), filtered and concentrated in vacuo to afford 2-(4-bromo-2-methylphenyl)acetonitrile as yellow oil, which was used in the next step without further purification (200 g, 100% yield). ¹H NMR (400 MHz, CDCl₃): δ 7.32 (s, 1H), 7.27 (d, J=8.0 Hz, 1H), 7.06 (d, J=8.0 Hz, 1H), 3.66 (s, 2H), 2.31 (s, 3H)

step 4: To a solution of 2-(4-bromo-2-methylphenyl)acetonitrile (200.0 g, 0.66 mol) in EtOH (500 mL) was added the aqueous KOH (10 M, 400 mL, 4 mol). The reaction was heated to reflux and stirred at this temperature for 2 h. The reaction mixture was cooled to RT and concentrated in vacuo. The residue was dissolved in water and then washed with Et₂O (2×500 mL). The aqueous layer cooled to 0° C. and the pH adjusted to 5 with conc. HCl. The precipitate was filtered and washed with water and then dried in vacuum to afford 2-(4-bromo-2-methylphenyl)acetic acid as yellow solid (150 g, 65.5% yield from (4-bromo-2-methylphenyl)methanol).

step 5: To a solution of 2-(4-bromo-2-methylphenyl)acetic acid (150.0 g, 1 mol) in MeOH (500 mL) at 0° C. was added dropwise SOCl₂ (50 mL, 1.2 mol). The reaction was heated to reflux and stirred at this temperature for 4 h. The reaction mixture was cooled to RT and concentrated in vacuo. The residue was diluted with EtOAc (1 L) and washed with water (2×500 mL), sat. NaHCO₃ (1 L) and brine (1 L). The organic layer was dried (Na₂SO₄), filtered and evaporated to afford methyl 2-(4-bromo-2-methylphenyl)acetate as yellow oil, which was used in the next step without further purification (130 g, 81% yield). ¹H NMR (400 MHz, CDCl₃): δ 7.36 (s, 1H), 7.34 (d, J=8.0 Hz, 1H), 7.23 (d, J=8.0 Hz, 1H), 3.68 (s, 3H), 3.58 (s, 2H), 2.27 (s, 3H).

step 6: A mixture of methyl 2-(4-bromo-2-methylphenyl)acetate (63.0 g, 259 mmol), bis(pinacolato)diboron (79.0 g, 311 mmol), KOAc (50.0 g, 518 mmol) and Pd(dppf)Cl₂ (6.1 g, 7.5 mmol) were heated to reflux in dry DMSO (1 L) under N₂ for 18 h. This mixture was filtered, diluted with water (1 L) and extracted with EtOAc (3×800 mL). The organic layer was dried (Na₂SO₄), filtered and concentrated in vacuo to afford crude methyl 2-(2-methyl-4-(4,4,5,5-tetramethyl-1,3,2-dioxaborolan-2-yl)phenyl)acetate, which was used in the next step without further purification (76 g, 100%).

step 7: A solution of methyl 2-(2-methyl-4-(4,4,5,5-tetramethyl-1,3,2-dioxaborolan-2-yl)phenyl)acetate (75.4 g, 259 mmol), Na₂CO₃ (83.0 g, 780 mmol), Pd(dppf)Cl₂ (6.3 g, 7.7 mmol) and 2-bromo-6-methylpyridine (54.0 g, 312 mmol) in dioxane/H₂O (4:1, 800 mL) under N₂ was heated and stirred at 100° C. for 18 h. The resulting mixture was diluted with H₂O (200 ml) and extracted with EtOAc (2×750 mL). The organic layers were combined, washed with brine (2×500 mL), dried (Na₂SO₄), filtered and concentrated in vacuo. The crude material was then purified by SiO₂ chromatography eluting with a PE/EtOAc gradient (10:1 to 5:1) to afford methyl 2-(2-methyl-4-(6-methylpyridin-2-yl)phenyl)acetate as yellow oil (25 g, 39%). ¹H NMR (400 MHz, CDCl₃): δ 7.81 (s, 1H), 7.73 (d, J=8.0 Hz, 1H), 7.59 (t, 1H), 7.46 d, J=8.0 Hz, 1H), 7.28 (d, J=7.6 Hz, 1H), 7.06 (d, J=7.6 Hz, 1H), 3.69 (s, 3H), 3.67 (s, 2H), 2.60 (s, 3H), 2.38 (s, 3H); MS: m/z 255.9 [M+1]⁺.

### Referential Example 28

### Methyl 2-[2-chloro-4-(6-methylpyridin-2-yl)phenyl]acetate

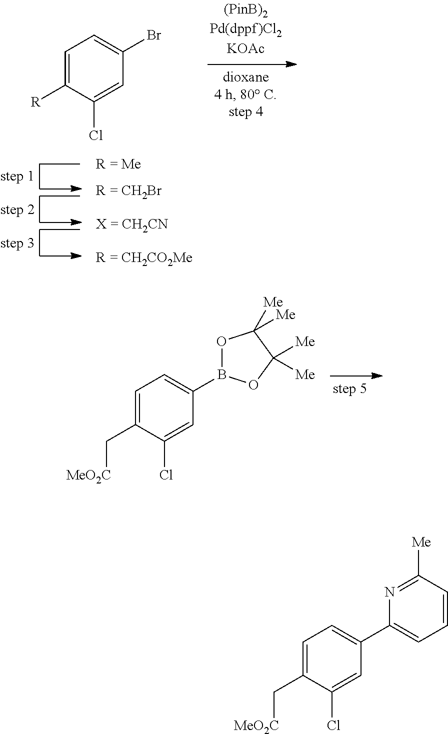

step 1: A 10-L 4-necked round-bottom flask purged and maintained with an inert atmosphere of nitrogen was charged with CCl₄ (5760 mL), 4-bromo-2-chloro-1-methylbenzene (800 g, 3.89 mol, 1.00 equiv), NBS (761.6 g, 4.28 mol, 1.10 equiv), and benzoyl peroxide (47.8 g, 186.54 mmol, 0.05 equiv). The resulting solution was stirred at 78° C. for 5 h and filtered. The filtrate was washed with H₂O (1×3 L) and brine (1×3 L) of and dried (Na₂SO₄), filtered and concentrated in vacuo to afford 1.2 kg (crude) of 4-bromo-1-(bromomethyl)-2-chlorobenzene as brown oil.

step 2: Into a 20-L 4-necked round-bottom flask was placed water (4.5 L), DCM (4.5 L), 4-bromo-1-(bromomethyl)-2-chlorobenzene (1200 g, 4.22 mol, 1.00 equiv) and tetrabutyl ammonium bromide (126 g, 390.86 mmol, 0.10 equiv) followed by the addition of a solution of NaCN (292 g) in water (9 L) dropwise with stirring at RT. The resulting solution was stirred at RT for 15 h. The organic layers were washed with brine (1×500 mL), dried (Na₂SO₄), filtered and concentrated in vacuo. The residue was purified by SiO₂ chromatography eluting with a EtOAc/petroleum ether gradient (1:10-1:5) to afford 550 g (57%) of 2-(4-bromo-2-chlorophenyl)acetonitrile as a white solid.

step 3: A 10-L 4-necked round-bottom flask was charged with MeOH (2950 mL) and 2-(4-bromo-2-chlorophenyl)acetonitrile (295 g, 1.28 mol, 1.00 equiv). Thionyl chloride (1500 mL) was added dropwise with stirring at 0° C. over 5 h. The resulting solution was stirred at RT overnight. This reaction was repeated. The combined reaction mixtures were concentrated and diluted with EtOAc (5 L). The resulting mixture was washed with H₂O (1×3 L), NaHCO₃ (1×3 L), and brine (1×3 L), dried over (Na₂SO₄), filtered and concentrated in vacuo. The residue was purified by SiO₂ chromatography eluting with EtOAc/PE gradient (1:0 to 20:1) to afford 600 g (89%) of methyl 2-(4-bromo-2-chlorophenyl)acetate as light yellow oil.

step 4: A 10-L 4-necked round-bottom flask purged and maintained with an inert atmosphere of nitrogen was charged with dioxane (3.5 L), methyl 2-(4-bromo-2-chlorophenyl)acetate (310 g, 1.18 mol, 1.00 equiv), KOAC (347 g, 3.54 mol, 3.00 equiv), 4,4,5,5-tetramethyl-2-(tetramethyl-1,3,2-dioxaborolan-2-yl)-1,3,2-dioxaborolane (299.4 g, 1.18 mol, 1.00 equiv), and Pd(dppf)Cl₂ (20 g). The resulting solution was stirred at 80° C. for 4 h. This reaction was repeated. The combined reaction solution was filtered. The resulting solution, containing crude methyl 2-(2-chloro-4-(4,4,5,5-tetramethyl-1,3,2-dioxaborolan-2-yl)phenyl)acetate, was concentrated and used in the next step without further purification.

step 5: A 10-L 4-necked round-bottom flask purged and maintained with an inert atmosphere of nitrogen was charged with the solution from Step 4, sodium carbonate (2N, 1178 mL), 2-bromo-6-methylpyridine (202 g, 1.17 mol, 1.00 equiv), and Pd(PPh₃)₄ (15 g, 12.98 mmol, 0.01 equiv). The resulting solution was stirred at 85° C. overnight. The reaction was repeated. The combined reaction mixtures were cooled to RT, filtered, and diluted with water (5 L). The resulting solution was extracted with EtOAc (3×5 L). The combined organic layers were washed with brine (1×3 L), dried (Na₂SO₄), filtered and concentrated in vacuo. The residue was purified using by SiO₂ chromatography eluting with EtOAc/PE (1:50) to afford 252 g (39%) of methyl 2-[2-chloro-4-(6-methylpyridin-2-yl)phenyl]acetate as yellow oil. (ES, m/z): [M+H⁺]=276. ¹H-NMR (CDCl₃): δ 8.08 (s, 1H), 7.87-7.84 (dd, J=6.0 Hz, 1H), 7.68-7.63 (t, J=7.8 Hz, 1H), 7.52-7.49 (d, J=1.8 Hz, 1H), 7.40-7.38 (d, J=7.8 Hz, 1H), 7.15-7.12 (d, J=7.5 Hz, 1H), 3.85 (s, 2H), 3.74 (s, 3H), 2.64 (s, 3H).

### Referential Example 29

### Methyl 2-(2-chloro-4-(5-methylpyridazin-3-yl)phenyl)acetate

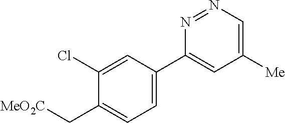 [SMILES: COC(=O)Cc1ccc(-c2cc(C)cnn2)cc1Cl]

A mixture of methyl 2-(2-chloro-4-(4,4,5,5-tetramethyl-1,3,2-dioxaborolan-2-yl)phenyl)acetate (682 mg, 2.2 mmol), 3-chloro-5-methylpyridazine (297 mg in 1.188 g dioxane, 2.3 mmol), KOAc (431 mg, 4.4 mmol), Pd(dppf)Cl₂(II) (160 mg, 0.22 mmol) in dioxane (22 mL) and water (16 mL) was heated at 90° C. under N₂ overnight. The reaction mixture was then diluted with EtOAc (100 mL) and washed with water (50 mL). The aqueous layer was extracted with EtOAc (3×30 mL). The combined organic layers were washed with brine (2×50 mL), dried (MgSO₄), filtered and concentrated in vacuo. The residue was then purified by SiO₂ chromatography eluting PE/EtOAc (55-60%) to give the target compound (186 mg, 30%) as a yellow solid. LCMS (ESI): m/z=277.0 [M+1]⁺.

### Referential Example 30

### Ethyl 2-(2-chloro-4-(6-methylpyrazin-2-yl)phenyl)acetate

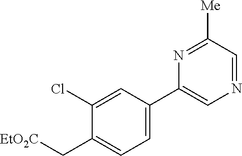 [SMILES: CCOC(=O)Cc1ccc(-c2cncc(C)n2)cc1Cl]

step 1: To a 3 L three-necked flask under nitrogen was added zinc dust (117 g, 1.79 mol) and THF (0.3 L). Chlorotrimethylsilane (10.4 mL) was added dropwise at a rate which maintained the internal temperature below 27° C. The mixture was stirred at 25-27° C. for 30 min and then heated to 30° C. A solution of ethyl bromoacetate (150 g, 0.90 mol) in THF (1.2 L) was slowly added dropwise to the reaction mixture (the internal temperature should maintained below 50° C.). After the addition was complete, the reaction mixture was allowed to cool back to 25-30° C. The mixture was filtered through CELITE pad under nitrogen to afford a yellow solution containing bromo-(2-ethoxy-2-oxoethyl)zinc. The concentration was titrated to be 0.50 M.

step 2: A 3 L three-necked flask under nitrogen was charged with 4-bromo-2-chloro-1-iodo-benzene (80 g, 0.25 mol), Pd(0)(dba)₂ (7.2 g, 0.0125 mol), Xantphos (7.2 g, 0.0125 mol) and THF (800 mL). The mixture was degassed and backfilled with nitrogen three times. Bromo-(2-ethoxy-2-oxoethyl)zinc (960 mL, 0.48 mol) was added to the reaction mixture and heated to 65° C. After 1 h, the reaction mixture was cooled to 30° C. and quenched with aq. 1N HCl (400 mL) and 25% brine (400 mL). The organic layer was separated. It was then filtered through CELITE pad and concentrated in vacuo. The crude product was purified by SiO₂ chromatography eluting with an EtOAc/heptane gradient (0 to 30% EtOAc). Ethyl 2-(4-bromo-2-chlorophenyl)acetate was obtained as light yellow oil (60 g, 86%). ¹H-NMR (CDCl₃, 400 MHz) δ 7.48 (d, J=2.0 Hz, 1H), 7.29 (dd, J₁=8.0 Hz, J₂=2.0 Hz, 1H), 7.08 (d, J=8 Hz, 1H), 4.10 (q, J=6.8 Hz, 2H), 3.64 (s, 2H), 1.18 (t, J=6.8 Hz, 3H)

step 3: A 500 mL three-necked flask was charged with ethyl 2-(4-bromo-2-chlorophenyl)acetate (50 g, 0.18 mol), 4,4,4′,4′,5,5,5′,5′-octamethyl-2,2′-bi(1,3,2-dioxaborolane) (62.5 g, 0.24 mol), potassium acetate (33 g, 0.34 mol), Pd(dppf)Cl₂(II)*DCM (10.4 g, 0.012 mol) and 1,4-dioxane (250 mL). The mixture was degassed and backfilled with nitrogen three times then heated at reflux for 20 h. This mixture was filtered, 25% brine (200 mL) was added and the resulting mixture was extracted with EtOAc (3×300 mL). The organic layer was dried over anhydrous (Na₂SO₄), filtered and concentrated in vacuo. The crude material was then purified by SiO₂ chromatography eluting with an EtOAc/heptane gradient (5 to 25% EtOAc) to afford ethyl 2-(2-chloro-4-(4,4,5,5-tetramethyl-1,3,2-dioxaborolan-2-yl)phenyl)acetate. (54 g, 90% yield).

step 4: A 2 L three-necked flask was charged with ethyl 2-(2-chloro-4-(4,4,5,5-tetramethyl-1,3,2-dioxaborolan-2-yl)phenyl)acetate (54 g, 0.17 mol), KOAc (33 g, 0.34 mol), Pd(dppf)Cl₂(II).DCM (10.4 g, 0.012 mol), 2-chloro-6 methylpyrazine (21.3 g, 0.17 mol) and 1,4-dioxane/water (2:1, 750 mL). The mixture was degassed and backfilled with nitrogen three times then heated to 100° C. for 15 h. After cooling to 20-25° C., it was diluted with water (200 mL) and extracted with EtOAc (300 mL×3). The combined organic layer was washed with 25% brine (100 mL×2), and dried (Na₂SO₄). The crude material was purified by SiO₂ chromatography eluting with an EtOAc/heptane gradient (5 to 25% EtOAc), followed by slurry in n-heptane. The product ethyl 2-(2-chloro-4-(6-methylpyrazin-2-yl)phenyl)acetate was obtained as white solid with >98% purity (18 g, 37.5%). ¹H-NMR (CDCl₃, 400 MHz) δ 8.81 (s, 1H), 8.42 (s, 1H), 8.10 (d, J=1.6 Hz, 1H), 7.85 (d, J=8 Hz, 1H), 7.43 (d, J=8 Hz, 1H), 4.20 (q, J=7.2 Hz, 2H), 3.84 (s, 2H), 2.64 (s, 3H), 1.28 (t, J=7.2 Hz, 3H).

### Referential Example 31

### 6-(2-Chloro-4-(6-methylpyrazin-2-yl)phenyl)-2-(methylthio)pyrido[2,3-d]pyrimidin-7(8H)-one

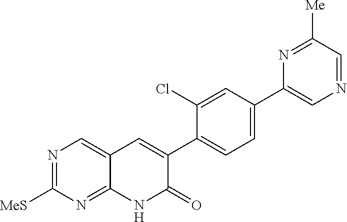 [SMILES: CSc1ncc2cc(-c3ccc(-c4cncc(C)n4)cc3Cl)c(=O)[nH]c2n1]

A 500 mL 3-neck round-bottom flask was charged with 4-amino-2-methylsulfanyl-pyrimidine-5-carbaldehyde (11.0 g, 65.0 mmol), 325-mesh K₂CO₃ (27.0 g, 3.0 equiv), ethyl 2-[2-chloro-4-(6-methylpyrazin-2-yl)phenyl]acetate (18.0 g, 61.9 mmol, 0.95 equiv), and DMF (110.0 mL) and the mixture was heated for 5 h at 120° C. then cooled to RT. The mixture was added water (220 mL) and solids precipitated. The slurry was stirred for 1 h and filtered and the wet cake was rinsed with water (55 mL×3) and dried at 50° C. under vacuum to afford an orange-colored solid 21.4 g (87%). ¹H NMR (300 MHz, DMSO-d₆) δ 12.69 (s, 1H), 9.17 (s, 1H), 8.90 (s, 1H), 8.58 (s, 1H), 8.29 (d, J=1.8 Hz, 1H), 8.18 (dd, J=8.0, 1.8 Hz, 1H), 8.01 (s, 1H), 7.58 (d, J=8.0 Hz, 1H), 2.60 (s, 3H), 2.59 (s, 3H).

Structural analogs of 6-(2-chloro-4-(6-methylpyrazin-2-yl)phenyl)-2-(methylthio)pyrido[2,3-d]pyrimidin-7(8H)-one, namely 6-(2-chloro-4-(6-methylpyridin-2-yl)phenyl)-2-(methylthio)pyrido[2,3-d]pyrimidin-7(8H)-one, 6-(2-methyl-4-(6-methylpyrazin-2-yl)phenyl)-2-(methylthio)pyrido[2,3-d]pyrimidin-7(8H)-one, and 6-(2-methyl-4-(6-methylpyridin-2-yl)phenyl)-2-(methylthio)pyrido[2,3-d]pyrimidin-7(8H)-one, we prepared in a similar manner using appropriate starting materials.

### Referential Example 32

### Methyl 2-(2-chloro-4-(2-methylthiazol-4-yl)phenyl)acetate

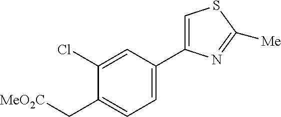 [SMILES: COC(=O)Cc1ccc(-c2csc(C)n2)cc1Cl]

step 1: A mixture of methyl 2-(4-bromo-2-chlorophenyl)acetate (13 g, 49.3 mmol), (PinB)₂ (16.3 g, 64.1 mmol), KOAc (9.7 g, 98.7 mmol) and Pd(dppf)Cl₂ (1.8 g, 2.47 mmol) was refluxed in dioxane (250 mL) under N₂ for 18 h. After cooling to RT, the mixture was partitioned between EtOAc (600 mL) and H₂O. The organic layer was separated and dried (Na₂SO₄), filtered and concentrated in vacuo. The residue was purified by SiO₂ chromatography eluting with a PE/EtOAc gradient (15:1 to 5:1) as eluting solvents to give methyl 2-(2-chloro-4-(4,4,5,5-tetramethyl-1,3,2-dioxaborolan-2-yl)phenyl)acetate (15 g, 97% yield). LCMS (ESI): m/z=311.0 [M+H]⁺.

step 2: To a solution of methyl 2-(2-chloro-4-(4,4,5,5-tetramethyl-1,3,2-dioxaborolan-2-yl)phenyl)acetate (149 mg, 0.48 mmol), 2-bromo-1,1-diethoxyethane (60 mg, 0.33 mmol), K₂CO₃ (90 mg, 0.65 mmol) and Pd(dppf)Cl₂ (8 mg, 0.01 mmol) in dioxane (5 mL) was added water (1 mL). The mixture was stirred at 100° C. for 16 h under N₂. After cooling to RT, EtOAc (150 mL) and H₂O were added. The organic layer was separated and dried (Na₂SO₄), filtered and concentrated in vacuo. The reside was purified by SiO₂ chromatography eluting with a petroleum ether/EtOAc gradient (1:0 to 8:1) to afford methyl 2-(2-chloro-4-(2-methylthiazol-4-yl)phenyl)acetate (100 mg, 74%) as colorless oil. LCMS (ESI): m/z=282.1 [M+H]⁺

### Referential example 33

### 2-(2-(Bromomethyl)-1,3-dioxan-5-yl)isoindoline-1,3-dione

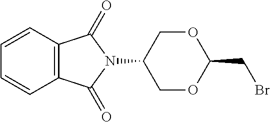 [SMILES: O=C1c2ccccc2C(=O)N1[C@H]1CO[C@H](CBr)OC1]

step 1. A 100 mL three-necked flask was charged with 2-aminopropane-1,3-diol (5.0 g, 54.9 mmol, 1.0 eq.), phthalic anhydride (8.1 g, 54.9 mmol, 1.0 eq) and DMF (50 mL). The mixture was heated to 90° C. for 4 h, cooled to 20-25° C. then concentrated to dryness. The residue was taken up in EtOAc (50 mL), and, after filtration, the product was obtained as orange solid (11.2 g, 92%). ¹H NMR (DMSO-d₆, 400 MHz) δ 7.85 (s, 1H), 4.89 (m, 2H), 4.24 (m, 1H), 3.82-3.77 (m, 2H), 3.68-3.65 (m, 2H); LCMS (ESI): m/z=222 [M+H]⁺.

step 2. A 500 mL three-necked flask was charged with 2-(1,3-dihydroxypropan-2-yl)isoindoline-1,3-dione (4.0 g, 18.1 mmol, 1.0 eq.), 2-bromo-1,1-diethoxyethane (4.3 g, 21.7 mmol, 1.2 eq.), tosic acid hydrate (0.69 g, 3.6 mmol, 0.2 eq.) and toluene (120 mL). The mixture was heated to 110° C. for 20 h and then cooled to RT. The mixture was washed with saturated aq. NaHCO₃ (50 mL) and brine (50 mL). The organic layer was dried (Na₂SO₄), filtered and evaporated to dryness with anhydrous sodium sulfate. Following concentration to dryness the residue was reslurried in EtOAc to give a white solid (3.1 g, 53%). ¹H NMR (DMSO-d₆, 400 MHz) δ 7.88-7.85 (m, 4H), 4.84 (t, J=4 Hz, 1H), 4.31 (m, 3H), 4.12 (m, 2H), 3.51 (m, 2H); LCMS (ESI): m/z=327 [M+H]⁺.

### Referential Example 34

### tert-Butyl 3-[(2-hydroxyethane)sulfonyl]azetidine-1-carboxylate

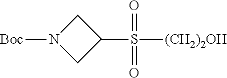

step 1: A 1 L 3-necked round-bottom flask, purged and maintained with an inert atmosphere of nitrogen was charged with a solution of tert-butyl 3-iodoazetidine-1-carboxylate (29 g, 102.43 mmol, 1.00 equiv) in DMF (300 mL), ethyl 2-sulfanylacetate (17.2 g, 143.13 mmol, 1.40 equiv) and K₂CO₃ (28.3 g, 204.76 mmol, 2.00 equiv). The resulting solution was stirred overnight at 50° C., cooled to RT, diluted with 500 mL of ice/water and extracted with EtOAc (3×200 mL). The combined organic layers were washed with H₂O (2×200 mL) and brine (1×200 mL), dried (Na₂SO₄), filtered and concentrated in vacuo. The residue was purified by SiO₂ chromatography eluting with A EtOAc/PE gradient (o to 10% EtOAc) to afford 20 g (71%) of tert-butyl 3-[(2-ethoxy-2-oxoethyl)sulfanyl]azetidine-1-carboxylate as a light yellow oil.

step 2: A 250-mL 3-necked round-bottom flask purged and maintained with an inert atmosphere of nitrogen was charged with a solution of tert-butyl 3-[(2-ethoxy-2 oxoethyl)sulfanyl]azetidine-1-carboxylate (5.5 g, 19.97 mmol, 1.00 equiv) in DCM (100 mL) followed by the addition of MCPBA (8.63 g, 50.01 mmol, 2.50 equiv) in several batches at 0° C. The resulting solution was stirred overnight at RT. The resulting mixture was then washed with sat. aq. Na₂SO₃ (2×60 mL) and sat. NaHCO₃ (1×60 mL). The aqueous layers were combined and extracted with DCM (100 mL). The combined organic layers were washed with brine (100 mL), dried (Na₂SO₄), filtered and concentrated in vacuo. The residue was purified by SiO₂ chromatography eluting with a EtOAc/PE gradient (5 to 20% EtOAc) to afford of tert-butyl 3-[(2-ethoxy-2-oxoethane)sulfonyl]azetidine-1-carboxylate (5 g, 81%) as a light yellow solid.

step 3: A 1 L 3-necked round-bottom flask, purged and maintained with an inert atmosphere of nitrogen, was charged with a solution of tert-butyl 3-[(2-ethoxy-2-oxoethane)sulfonyl]azetidine-1-carboxylate (20 g, 65.07 mmol, 1.00 equiv) in THF (400 mL), followed by the addition of LiBH₄ (7.2 g, 5.00 equiv) in several batches at 0° C. The resulting solution was stirred overnight at RT, quenched by the addition of 200 mL of water/ice, diluted with EtOAc (300 mL) and extracted with EtOAc (2×200 mL). The combined organic layers were washed with brine (200 mL), dried (Na₂SO₄), filtered and concentrated in vacuo. The residue was purified on a SiO₂ column eluting with an EtOAc/PE gradient (10 to 20% EtOAc) to afford of tert-butyl 3-[(2-hydroxyethane)sulfonyl]azetidine-1-carboxylate (7 g, 41%) as a white solid. ¹H-NMR (300 MHz, CDCl₃): δ 4.35-4.21 (m, 2H), 4.19-4.06 (m, 5H), 3.22-3.18 (m, 2H), 2.76-2.71 (m, 1H), 1.46 (s, 9H); LC-MS (ESI): m/z=288 [M+Na]⁺.

### Referential Example 35

### tert-Butyl 4-cyano-4-(tosyloxymethyl)piperidine-1-carboxylate

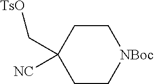 [SMILES: Cc1ccc(S(=O)(=O)OCC2(C#N)CCN(C(=O)OC(C)(C)C)CC2)cc1]

step 1: A mixture of piperidine-4-carbonitrile (1.1 g 10.0 mmol), di-tert-butyl dicarbonate (2.62 g, 12 mmol), and TEA (4.2 mL, 30 mmol) in DCM (15 mL) was stirred at RT overnight. HCl (5% wt in water) was added until pH <5 was reached. The aqueous phase was extracted with DCM. The organic layer was washed with brine, dried (Na₂SO₄), filtered and concentrated in vacuo to afford tert-butyl 4-cyanopiperidine-1-carboxylate (2.0 g, 95.2%) as yellow oil, which was used in next step without further purification. LCMS (ESI): m/z=155.1 [M−55].

step 2. To a stirring mixture of tert-butyl 4-cyanopiperidine-1-carboxylate (1.0 g crude, 4.76 mmol) in THF (10 mL) under a nitrogen atmosphere and cooled to −78° C. was added dropwise lithium bis(trimethylsilyl)amide (1M in THF, 7.14 mL, 7.14 mmol). The mixture was stirred at −78° C. for 1 h. Then ethyl carbonochloridate (771 mg, 7.14 mmol) was added, and the mixture was stirred at −78° C. for another 1 h. The mixture was quenched with saturated aqueous NH₄Cl and the aqueous phase extracted with EtOAc. The organic layer was dried (Na₂SO₄), filtered and concentrated in vacuo. The residue was purified by SiO₂ chromatography eluting with PE/EtOAc (4:1) to afford 1-tert-butyl 4-ethyl 4-cyanopiperidine-1,4-dicarboxylate (1.3 g crude, 83.1%) as a yellow oil. LCMS (ESI): m/z=183.1 [M−99].

step 3. To a stirring mixture of 1-tert-butyl 4-ethyl 4-cyanopiperidine-1,4-dicarboxylate (1.3 g crude, 4.61 mmol) in MeOH (15 mL) under a nitrogen atmosphere at 0° C. was added NaBH₄ (350 mg, 9.22 mmol). The mixture was stirred at 0° C. for 1.5 h then quenched with HCl (5% wt in water, 5 mL). The aqueous phase was extracted with EtOAc. The organic layer was dried (Na₂SO₄), filtered and concentrated in vacuo. The residue was purified by SiO₂ chromatography eluting with PE/EtOAc (4:1) to afford tert-butyl 4-cyano-4-(hydroxymethyl)piperidine-1-carboxylate (900 mg, 81.3%) as a colorless oil. LCMS (ESI): m/z=141.1 [M−99].

step 4: A mixture of tert-butyl 4-cyano-4-(hydroxymethyl)piperidine-1-carboxylate (300 mg 1.25 mmol), tosyl chloride (285 mg, 1.5 mmol), DMAP (16 mg, 0.13 mmol) and TEA (0.52 mL, 3.75 mmol) in DCM (5 mL) was stirred at RT overnight. The mixture was concentrated in vacuo. The residue was purified by SiO₂ chromatography eluting with PE/EtOAc (6:1) to afford tert-butyl 4-cyano-4-(tosyloxymethyl)piperidine-1-carboxylate (120 mg, 24.4%) as white solid. LCMS (ESI): m/z=295.0 [M−99].

### Referential Example 36

### tert-Butyl 3-cyano-3-(2-hydroxyethyl)pyrrolidine-1-carboxylate

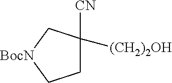

step 1: To a stirred solution of tert-butyl 3-cyanopyrrolidine-1-carboxylate (600 mg, 3.06 mmol) in THF (10 mL) was added lithium bis(trimethylsilyl)amide (1M in THF, 4.6 mL, 4.6 mmol) dropwise under N₂ at −78° C. 3-Bromoprop-1-ene (556 mg, 4.59 mmol) was added, and the mixture was stirred at −78° C. for 2 h. The mixture was warmed to RT and quenched with saturated aqueous NH₄Cl. The aqueous phase was extracted with EtOAc. The organic layer was dried (Na₂SO₄), filtered and concentrated in vacuo. The residue was purified by SiO₂ chromatography eluting with PE/EtOAc (6:1) to afford tert-butyl 3-allyl-3-cyanopyrrolidine-1-carboxylate (600 mg, 83.1%) as colorless oil. LCMS (ESI): m/z=181.1 [M−55].

step 2. NaIO₄ (2.6 g, 12.0 mmol) was added to a stirred solution of tert-butyl 3-allyl-3-cyanopyrrolidine-1-carboxylate (700 mg, 3.0 mmol) in a mixture of MeCN, CCl₄ and water (2:2:3, 40 mL respectively). The mixture was vigorously stirred for 5 min and RuCl₃ (25 mg, 0.12 mmol) was added. The mixture was stirred for a further 3 h and saturated aqueous NaHCO₃ was added until the pH was ca., 8. The aqueous solution was washed with DCM (3×20 ml). The resulting aqueous layer was then carefully acidified with 5% aqueous HCl and extracted with DCM (3×20 ml). The combined organic layers were dried (Na₂SO₄), filtered and concentrated in vacuo to afford 2-(1-(tert-butoxycarbonyl)-3-cyanopyrrolidin-3-yl)acetic acid (400 mg, crude) as white solid. LCMS (ESI): m/z=199.1 [M−55].

step 3. To a stirring mixture of 2-(1-(tert-butoxycarbonyl)-3-cyanopyrrolidin-3-yl)acetic acid (250 mg, 1.00 mmol) in THF (5 mL) at 0° C. was added dropwise BH₃ (1M in THF, 2.0 mL, 2.0 mmol) and the mixture was stirred at RT for 3 h. MeOH (5 mL) was added, and the mixture was then concentrated under reduced pressure. The residue was purified by SiO₂ chromatography using PE/EtOAc (1:1) as eluting solvents to afford tert-butyl 3-cyano-3-(2-hydroxyethyl)pyrrolidine-1-carboxylate (100 mg, 42.3%) as yellow oil. LCMS (ESI): m/z=185.1 [M−55].

### Referential Example 37

### tert-Butyl 2-(2-hydroxyethylsulfonyl)ethylcarbamate

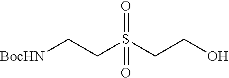 [SMILES: CC(C)(C)OC(=O)NCCS(=O)(=O)CCO]

step 1. N-tert-Boc-ethanolamine (14 g, 87 mmol), TEA (27.3 mL, 195 mmol) and DCM (180 mL) were combined in a 500 mL round bottom flask and cooled in an ice bath. p-Toluenesulfonyl chloride (23.1 g, 122 mmol), dissolved in DCM (180 mL), was added and the ice bath removed. The mixture was stirred at RT overnight. The mixture was washed with water, brine and dried (Na₂SO₄), filtered and concentrated in vacuo to afford 2-(tert-butoxycarbonylamino)ethyl 4-methylbenzenesulfonate (26 g, crude) as a light yellow oil. LCMS (ESI): m/z=338 (M+Na)⁺.

step 2. 2-Mercaptoethanol (7.25 mL, 104 mmol), DBU (15.4 mL, 103 mmol), 2-(tert-butoxy-carbonylamino)ethyl 4-methylbenzenesulfonate (26 g, crude) and PhMe (200 mL) were combined in a 500 mL round bottom flask and stirred at RT overnight. The reaction mixture was washed with water and brine and dried (Na₂SO₄), filtered and concentrated in vacuo. The crude product was purified by SiO₂ chromatography eluting with a PE/EtOAc gradient (325 to 33% EtOAc) to afford tert-butyl 2-(2-hydroxyethylthio)ethylcarbamate (10.7 g, 56% for 2 steps) as a colorless oil. TLC: R_{f}=0.35 (PE/EtOAc-2:1).

step 3. To a solution of tert-butyl 2-(2-hydroxyethylthio)ethylcarbamate (10.7 g, 48 mmol) in EtOH (150 mL) in a 500 mL RB flask was added 45 g of oxone in H₂O (150 mL). The ice bath was removed and stirring at RT continued for 2 h. The reaction mixture was diluted with H₂O (600 mL) and extracted with EtOAc (3×). The combined organic layers were washed with brine and dried (Na₂SO₄), filtered and concentrated in vacuo to afford tert-butyl 2-(2-hydroxyethylsulfonyl)ethylcarbamate (9.23 g, 75%) as a light yellow oil. ¹H NMR (400 MHz, CDCl₃) δ 5.34 (s, 1H), 4.16-4.06 (m, 2H), 3.65 (m, 2H), 3.35 (t, 2H, J=4 Hz), 3.30-3.21 (t, 2H, J=4 Hz), 1.44 (s, 9H).

### Referential Example 38

### tert-Butyl 2-(hydroxymethyl)thiomorpholine-4-carboxylate

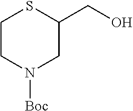 [SMILES: CC(C)(C)OC(=O)N1CCSC(CO)C1]

To a solution of 4-(tert-butoxycarbonyl)thiomorpholine-2-carboxylic acid (0.8 g, 3.24 mmol) in anhydrous THF (20 mL) was added slowly at 0° C. BH₃ solution (1 M in THF, 13.2 mL). The reaction mixture was stirred at RT overnight then quenched with saturated NaHCO₃ (50 mL). The aqueous layer was extracted with EtOAc (2×50 mL). The combined organic layers were washed with brine and dried (MgSO₄), filtered and concentrated in vacuo to afford tert-butyl 2-(hydroxymethyl)thiomorpholine-4-carboxylate (0.75 g, crude) as colorless oil, which was used without further purification.

### Referential Example 39

### tert-Butyl 2-{(2-([(4-methylbenzene)sulfonyl]oxy}ethyl)-1,1-dioxo-thiomorpholine-4-carboxylate

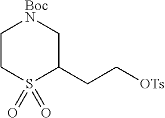 [SMILES: Cc1ccc(S(=O)(=O)OCCC2CN(C(=O)OC(C)(C)C)CCS2(=O)=O)cc1]

step 1: A mixture of 4-thiomorpholine-1,1-dione hydrochloride (4.0 g, 23.3 mmol), Et₃N (7.08 g, 69.9 mmol) and (Boc)₂O (6.62 g, 30.2 mmol) in DCM (20 mL) was stirred at RT overnight. After concentration, the residue was purified by SiO₂ chromatography eluting with EtOAc/PE (1:3) to afford tert-butyl 1,1-dioxo-4-thiomorpholine-carboxylate as colorless oil (4.5 g, 82%). LCMS (ESI): m/z=181 [M−55]⁺.

step 2: To a solution of tert-butyl 1,1-dioxo-thiomorpholine-4-carboxylate (500 mg, 2.12 mmol) in anhydrous THF (10 mL) at −78° C. was added LDA (1M in THF, 2.55 mL, 2.55 mmol) dropwise slowly. The mixture was stirred at −78° C. for 30 min. To this mixture was added ethyl 2-bromoacetate (390 mg, 2.33 mmol) dropwise at −78° C. then the reaction was stirred RT overnight. Saturated aq. NH₄Cl solution and EtOAc (20 mL) were added. The organic layer was separated, washed with brine, dried (Na₂SO₄), filtered and concentrated in vacuo. The residue was purified by SiO₂ chromatography eluting with EtOAc/PE (1:3) to afford tert-butyl 2-(2-ethoxy-2-oxoethyl)-1,1-dioxo-thiomorpholine-4-carboxylate as colorless oil (300 mg, 44%). LCMS (ESI): m/z=266 [M−55]⁺.

step 3. To a solution of tert-butyl 2-(2-ethoxy-2-oxoethyl)-1,1-dioxo-thiomorpholine-4-carboxylate (1 g crude) in EtOH (10 mL) was added NaBH₄ (710 mg, 18.7 mmol) at 0° C. The mixture was stirred at RT overnight and then concentrated under reduced pressure. The residue was partitioned between EtOAc (50 mL) and H₂O (50 mL). The organic layer was dried (Na₂SO₄), filtered and concentrated in vacuo. The residue was purified by SiO₂ chromatography eluting with EtOAc/PE (1:1) to afford tert-butyl 2-(2-hydroxyethyl)-1,1-dioxo-thiomorpholine-4-carboxylate (100 mg, 10%). LCMS (ESI): m/z=225 [M−55]⁺.

step 4. A mixture of tert-butyl 2-(2-hydroxyethyl)-1,1-dioxo-thiomorpholine-4-carboxylate (200 mg, 0.72 mmol), tosyl chloride (1.1 g, 5.73 mmol), TEA (1.45 g, 14.34 mmol) and DMAP (58 mg, 0.478 mmol) in DCM (10 mL) was stirred at RT overnight. The solution was concentrated under reduced pressure. The residue was purified by SiO₂ chromatography eluting with EtOAc/PE (1:1) as eluting solvents to afford tert-butyl 2-(2-{[(4-methylbenzene)sulfonyl]oxy}ethyl)-1,1-dioxo-thiomorpholine-4-carboxylate (150 mg, 65%). LCMS (ESI): m/z=378 [M−55]⁺.

### Referential Example 40

### Benzyl 7-(methylsulfonyloxymethyl)-5,9-dioxa-2-azaspiro[3.5]nonane-2-carboxylate

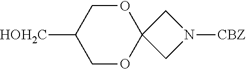

step 1. Benzyl 3-oxoazetidine-1-carboxylate (1.0 g, 4.7 mmol) and 2-(hydroxymethyl)propane-1,3-diol (650 mg, 1.05 equiv.) were dissolved in PhMe (32 mL) at RT. p-Toluenesulfonic acid (183 mg, 0.2 equiv.) was added and the reaction vessel was fitted with a Dean-Stark trap and condenser and stirred at 130° C. for 16 h. The reaction mixture was cooled to RT and treated with sat. aqueous NaHCO₃. The solution was diluted with DCM, and the layers were separated. The aqueous phase was extracted with DCM (2×), dried (Na₂SO₄), filtered and concentrated in vacuo. The crude product was absorbed onto CELITE and purified by SiO₂ chromatography eluting with a MeOH/DCM gradient (0 to 20% MeOH). Benzyl 7-(hydroxymethyl)-5,9-dioxa-2-azaspiro[3.5]nonane-2-carboxylate was obtained after drying as a light yellow oil (937.2 mg, 68%).

step 2: To a solution of benzyl 7-(hydroxymethyl)-5,9-dioxa-2-azaspiro[3.5]nonane-2-carboxylate (172 mg, 0.58 mmol) and DCM (1.6 mL) at 0° C. was added TEA (0.124 mL, 1.5 equiv.). Methanesulfonyl chloride (0.06 mL, 1.2 equiv.) was added and the reaction mixture stirred at RT under N₂ for 3 h and then poured into EtOAc. The mixture was washed with brine and the organic layer dried (Na₂SO₄), filtered and concentrated in vacuo. Further drying under high vacuum afforded benzyl 7-(methylsulfonyloxymethyl)-5,9-dioxa-2-azaspiro[3.5]nonane-2-carboxylate as a thick yellow oil (290 mg, quantitative yield, containing some residual solvent). ¹H NMR (400 MHz, DMSO-d₆) δ 747-7.23 (m, 5H), 5.05 (s, 2H), 4.62 (t, J=5.2 Hz, 1H), 4.10-3.82 (m, 6H), 3.62 (dd, J=7.2, 11.6 Hz, 2H), 3.37 (t, J=5.2 Hz, 2H), 1.87-1.74 (m, 1H). LCMS (ESI): m/z=294.1[M+H]⁺.

### Referential Example 41

### 6-(2-chloro-4-(4-oxo-5-azaspiro[2.4]heptan-5-yl)phenyl)-8-((5,5-difluoropiperidin-3-yl)methyl)-2-(methylamino)pyrido[2,3-d]pyrimidin-7(8H)-one

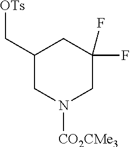

step 1: 1-tert-butyl 3-methyl 5-oxopiperidine-1,3-dicarboxylate—To a solution of 1-tert-butyl 3-methyl 5-hydroxypiperidine-1,3-dicarboxylate (2.0 g, 7.7 mmol) in DCM (20 mL) was added slowly added Dess-Martin periodinane (DMP, 6.5 mg, 15.4 mmol). The mixture was stirred at RT overnight and then filtered. The filtrate was washed with H₂O and a saturated aqueous solution of Na₂CO₃. The organic layer was dried (Na₂SO₄), filtered and concentrated in vacuo and the residue was purified by SiO₂ chromatography eluting with EtOAc/PE (5:1) to afford 1-tert-butyl 3-methyl 5-oxopiperidine-1,3-dicarboxylate as colorless oil (1.2 g, 61%). LCMS (ESI): m/z=202.0[ [M+1]−56]⁺

step 2: 1-tert-butyl 3-methyl 5,5-difluoropiperidine-1,3-dicarboxylate To a solution of 1-tert-butyl 3-methyl 5-oxopiperidine-1,3-dicarboxylate (1.1 g, 4.06 mmol) in DCM (20 mL) cooled to −78° C. was added dropwise DAST (2.0 g, 12.18 mmol). The mixture was stirred at RT for overnight and then H₂O and DCM (30 mL) were added. The organic layer was dried (Na₂SO₄), filtered and concentrated in vacuo. The residue was purified by SiO₂ chromatography eluting with EtOAc/PE (8:1) to afford 1-tert-butyl 3-methyl 5,5-difluoropiperidine-1,3-dicarboxylate as colorless oil (800 mg, 67%). LCMS (ESI): m/z=224.1 [ [M+1]−56]⁺.

step 3: tert-butyl 3,3-difluoro-5-(hydroxymethyl)piperidine-1-carboxylate—To a solution of 1-tert-butyl 3-methyl 5,5-difluoropiperidine-1,3-dicarboxylate (800 mg, 650 mmol) in DCM (10 mL) at 0° C. was added slowly NaBH₄ (650 mg, 17.2 mmol). The mixture was stirred at RT overnight and then concentrated in vacuo. The residue was diluted with EtOAc (50 mL) and H₂O (50 mL) the organic layer was dried (Na₂SO₄), filtered and concentrated in vacuo to afford tert-butyl 3,3-difluoro-5-(hydroxymethyl)piperidine-1-carboxylate as colorless oil (1.2 g, crude). LCMS (ESI): m/z=196.1 [[M+1]−56]⁺.

step 4: tert-butyl 3,3-difluoro-5-(tosyloxymethyl)piperidine-1-carboxylate To a solution of tert-butyl 3,3-difluoro-5-(hydroxymethyl)piperidine-1-carboxylate (1.2 g, 4.78 mmol) in DCM (10 mL) was added tosyl chloride (1.1 g, 5.73 mmol), TEA (1.45 g, 14.3 mmol) and DMAP (58 mg, 0.478 mmol). The mixture was stirred at RT overnight and the solution was then concentrated in vacuo. The residue was purified by SiO₂ chromatography eluting with EtOAc/PE (3:1) to afford tert-butyl 3,3-difluoro-5-(tosyloxymethyl)piperidine-1-carboxylate as white solid (1.2 g, 62%). LCMS (ESI): m/z=350.1 [ [M+1]−56]⁺.

### Referential Example 42

### tert-Butyl ((2R,5R)-2-(2-aminoethyl)-1,3-dioxan-5-yl)carbamate (anti isomer) and tert-butyl ((2S,5S)-2-(2-aminoethyl)-1,3-dioxan-5-yl)carbamate (syn isomer)

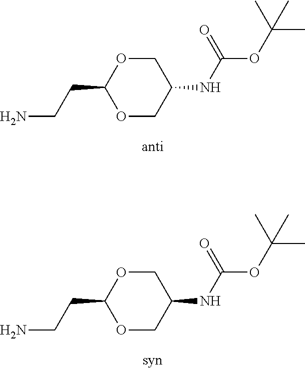 [SMILES: CC(C)(C)OC(=O)N[C@H]1CO[C@H](CCN)OC1 ; CC(C)(C)OC(=O)N[C@H]1CO[C@@H](CCN)OC1]

step 1: To a solution of 10% NaHCO₃ (aq, 3 L) was added to a solution of 3-aminopropan-1-ol (75 g, 998 mmol) in 1,4-dioxane at 0° C. Fmoc-Cl (309.6 g, 1.189 mol) was added dropwise to the reaction mixture. The solution was warmed to RT and stirred overnight. The reaction mixture was diluted with EtOAc (1 L) and H₂O (600 mL). The organic phase was separated and washed with H₂O (500 mL) and brine (500 mL), followed by drying over Na₂SO₄. The solution was concentrated in vacuo to give the crude product as a white solid. The crude product was washed with hexane (5×1 L) and dried in vacuo to afford (9H-fluoren-9-yl)methyl (3-hydroxypropyl)carbamate (313 g, 105.5%) as a white solid, which was used in the next step without further purification. ¹H-NMR (400 MHz, CDCl₃) δ (ppm): 7.76 (d, J=7.5 Hz, 2H), 7.59 (d, J=7.4 Hz, 2H), 7.40 (t, J=7.2 Hz, 2H), 7.32 (td, J=8.6, 1.1 Hz, 2H), 5.03 (brs, 1H), 4.44 (d, J=6.7 Hz, 2H), 4.21 (t, J=6.6 Hz, 1H), 3.64 (t, J=5.7 Hz, 2H), 3.30-3.37 (m, 2H), 2.00-2.20 (m, 2H), 1.64-1.73 (m, 2H).

Step B: To a solution of DMSO (177.2 g, 2.268 mol) in anhydrous DCM (800 ml), oxalyl chloride (196.9 g, 1.549 mol) in anhydrous DCM (800 mL) was slowly added at −45° C. The reaction mixture was stirred for 20 min at −45° C. and then a solution of (9H-fluoren-9-yl)methyl (3-hydroxypropyl)carbamate (307 g, 1.032 mol) in anhydrous DCM (3.4 L) was added dropwise. After stirring for 30 min at −45° C., DIPEA (399.8 g, 3.093 mol) was added dropwise. The mixture was allowed to warm to −30° C. and stirred for another 30 min. The solvent was removed and the residue taken up in EtOAc (3 L), washed with H₂O (1 L), 5% NaHCO₃ (2×800 mL), H₂O (1 L) and brine (800 ml). The organic phase was dried (Na₂SO₄), filtered and evaporated to afford crude (9H-fluoren-9-yl)methyl (3-oxopropyl)carbamate (310 g, 104.5%) as a yellow solid. LCMS (ESI): m/z=318 [M+23]⁺.

### Example 1

### 8-(2-(Azetidin-3-yloxy)ethyl)-6-(2-chloro-4-(6-methylpyrazin-2-yl)phenyl)-2-(ethylamino)pyrido[2,3-d]pyrimidin-7(8H)-one (I-11)

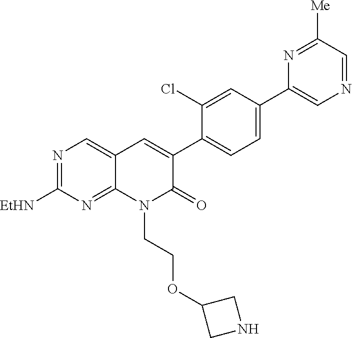

step 1: To a solution of 6-bromo-2-(methylthio)pyrido[2,3-d]pyrimidin-7(8H)-one (432 mg, 1.6 mmol) in DMF (7 mL) were added tert-butyl 3-(2-hydroxyethoxy)azetidine-1-carboxylate (0.750 g, 3.47 mmol), and PPh₃ (0.91 g, 3.47 mmol). To the above mixture was added DIAD (0.71 g, 3.47 mmol) at 0° C. The reaction mixture was stirred at RT overnight, then poured into water, extracted with EtOAc. The extracts were washed with water, dried (Na₂SO₄), filtered and concentrated in vacuo. The residue was purified by SiO₂ chromatography eluting with EtOAc/hexane (2:3) to afford tert-butyl 3-(2-(6-bromo-2-(methylthio)-7-oxopyrido[2,3-d]pyrimidin-8(7H)-yl)ethoxy)azetidine-1-carboxylate (392 mg, 52%) as yellow oil. LCMS (ESI): m/z=472.2 (M+1).

step 2. To a solution of tert-butyl 3-(2-(6-bromo-2-(methylthio)-7-oxopyrido[2,3-d]pyrimidin-8(7H)-yl)ethoxy)azetidine-1-carboxylate (392 mg, 0.83 mmol) in a mixture of DME (7 mL), EtOH (5 mL), PhMe (0.7 mL), water (1.4 mL) was added 2-chloro-4-(6-methylpyrazin-2-yl)phenylboronic acid (0.57 g, 0.82 mmol), Cs₂CO₃ (410 mg, 1.25 mol) and Pd(PPh₃)₄ (50 mg, 0.04 mmol). The mixture was heated at reflux overnight, cooled and concentrated. The residue was purified by SiO₂ chromatography eluting with EtOAc/hexane (2:3) to afford tert-butyl 3-(2-(6-(2-chloro-4-(6-methylpyrazin-2-yl)phenyl)-2-(methylthio)-7-oxopyrido[2,3-d]pyrimidin-8(7H)-yl)ethoxy)azetidine-1-carboxylate (313 mg, 63%) as yellow oil. LCMS (ESI): m/z=596.1 (M+1).

step 3. To a mixture of tert-butyl 3-(2-(6-(2-chloro-4-(6-methylpyrazin-2-yl)phenyl)-2-(methylthio)-7-oxopyrido[2,3-d]pyrimidin-8(7H)-yl)ethoxy)azetidine-1-carboxylate (313 mg, 0.53 mmol) in DCM (5.0 ml) was added dropwise to a solution of MCPBA (80%, 130 mg, 0.58 mmol) in DCM (1.0 ml) at −10-0° C. under N₂. The mixture was stirred at RT overnight. After the reaction was complete, the mixture was quenched with saturated NaHCO₃ solution. The mixture was extracted with DCM (20 mL×3). The combined organic layers were dried (Na₂SO₄), filtered, and evaporated to afford 350 mg of crude tert-butyl 3-(2-(6-(2-chloro-4-(6-methylpyrazin-2-yl)phenyl)-2-(methylsulfinyl)-7-oxopyrido[2,3-d]pyrimidin-8(7H)-yl)ethoxy)azetidine-1-carboxylate. The crude product was used in the next step without further purification. LCMS (ESI): m/z=612.2 (M+1).

step 4: A mixture of crude tert-butyl 3-(2-(6-(2-chloro-4-(6-methylpyrazin-2-yl)phenyl)-2-(methylsulfinyl)-7-oxopyrido[2,3-d]pyrimidin-8(7H)-yl)ethoxy)azetidine-1-carboxylate (350 mg, 0.53 mmol), DIPEA (0.2 mL) and ethylamine hydrochloride (215 mg, 2.6 mmol) in THF (10.0 mL) was stirred at RT under N₂ overnight. The reaction was quenched with H₂O followed by extraction with DCM (20 mL×3). The combined organic layers were (Na₂SO₄), filtered, and evaporated to afford tert-butyl 3-(2-(6-(2-chloro-4-(6-methylpyrazin-2-yl)phenyl)-2-(ethylamino)-7-oxopyrido[2,3-d]pyrimidin-8(7H)-yl)ethoxy)azetidine-1-carboxylate (400 mg, 100%) as a yellow oil, which was used in the next step without further purification. LCMS (ESI): m/z=592.1 (M+1).

step 5. To a solution of crude tert-butyl 3-(2-(6-(2-chloro-4-(6-methylpyrazin-2-yl)phenyl)-2-(ethylamino)-7-oxopyrido[2,3-d]pyrimidin-8(7H)-yl)ethoxy)azetidine-1-carboxylate (400 mg, 0.53 mmol) in dry DCM (5.0 ml) was added TFA (5.0 ml) at 0° C. The mixture was stirred for 1 h at RT. After removal of the solvent under reduced pressure, saturated NaHCO₃ and DCM were added to the residue. The separated organic layer was washed brine, dried (Na₂SO₄), filtered and concentrated in vacuo. The residue was purified by preparative HPLC to afford 8-(2-(azetidin-3-yloxy)ethyl)-6-(2-chloro-4-(6-methylpyrazin-2-yl)phenyl)-2-(ethylamino)pyrido[2,3-d]pyrimidin-7(8H)-one as white solid (110 mg, 42%). ¹H NMR (500 MHz, DMSO-d6): δ 9.16 (s, 1H), 8.65 (s, 1H), 8.57 (s, 1H), 8.28-8.27 (m, 1H), 8.16-8.14 (m, 1H), 8.03 (br s, 1H), 7.85 (s, 1H), 7.56-7.54 (m, 1H), 4.50-4.47 (m, 2H), 4.33-4.30 (m, 1H), 3.64-3.58 (m, 2H), 3.43-3.33 (m, 9H), 2.60 (s, 3H), 1.22 (t, 3H); LCMS (ESI): m/z=492.1 [M+1]⁺.

### Example 2

### 6-(2-Chloro-4-(6-methylpyrazin-2-yl)phenyl)-2-(ethylamino)-8-(2-(piperidin-3-yloxy)ethyl)pyrido[2,3-d]pyrimidin-7(8H)-one (I-14)

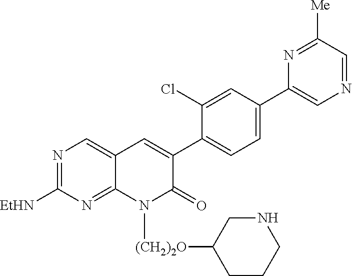

6-(2-Chloro-4-(6-methylpyrazin-2-yl)phenyl)-2-(ethylamino)-8-(2-(piperidin-3-yloxy)ethyl)pyrido[2,3-d]pyrimidin-7(8H)-one was analogously except tert-butyl 3-(2-hydroxyethoxy)piperidine-1-carboxylate replaced tert-butyl 3-(2-hydroxyethoxy)azetidine-1-carboxylate in step 1.

¹H NMR (500 MHz, CDCl₃-d3) δ 8.83 (s, 1H), 8.47 (brs, 1H), 8.43 (s, 1H), 8.17 (d, 1H), 7.94 (dd, 1H), 7.58 (s, 1H), 7.50 (d, 1H), 5.57 (brs, 1H), 4.67 (s, 2H), 3.88-3.81 (m, 2H), 3.59-3.54 (m, 2H), 3.43 (s, 1H), 3.02 (d, 1H), 2.80-2.78 (m, 1H), 2.66-2.65 (m, 2H), 2.65 (s, 3H), 1.86-1.83 (m, 1H), 1.72-1.68 (m, 1H), 1.54-1.50 (m, 1H), 1.40 (brs, 1H), 1.31 (t, 3H). MS (ESI): m/z=520.3 [M+1].

### Example 3

### 8-(((1r,4r)-4-aminocyclohexyl)methyl)-6-(2-chloro-4-(6-methylpyrazin-2-yl)phenyl)-2-(ethylamino)pyrido[2,3-d]pyrimidin-7(8H)-one (I-5)

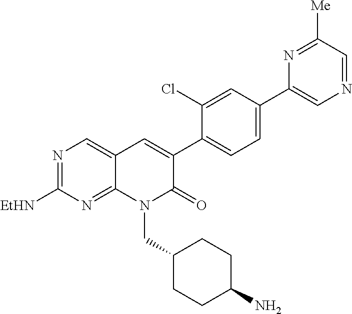

step 1: To a solution of ethyl 4-chloro-2-(methylthio)pyrimidine-5-carboxylate (3.00 g, 13.0 mmol) in dry THF (60 ml) under N₂ was added TEA (1.32 g, 13.04 mmol) and tert-butyl (1r,4r)-4-(aminomethyl)cyclohexylcarbamate (3.56 g, 15.65 mmol) at RT. The solution was stirred at RT overnight. Water (40 mL) was added and the solution was extracted with DCM (3×50 mL). The combined organic layers were dried over anhydrous Na₂SO₄, filtered, and evaporated to afford ethyl 4-(((1r,4r)-4-(tert-butoxycarbonylamino)cyclohexyl)methylamino)-2-(methylthio)pyrimidine-5-carboxylate (2.1 g, 38%) as a white solid. LCMS (ESI): m/z=424.1 [M+1]⁺.

step 2: To a solution of 4-(((1r,4r)-4-(tert-butoxycarbonylamino)cyclohexyl)methylamino)-2-(methylthio)pyrimidine-5-carboxylate (2.1 g, 4.9 mmol) in THF (35 ml) was added portionwise LiAlH₄ (240 mg, 6.21 mmol) at −20° C. under N₂. The mixture was then stirred at −20° C. for 2 h. The mixture was cooled to 0° C. and H₂O (0.5 ml) and 10% aqueous NaOH (0.5 ml) were added. The mixture was extracted with DCM (3×50 ml) and the combined organic layers were dried over anhydrous Na₂SO₄, filtered and evaporated. The crude product was purified by SiO₂ column chromatography eluting with EtOAc/PE gradient (1:1 to 2:1) to afford tert-butyl (1r,4r)-4-((5-(hydroxymethyl)-2-(methylthio)pyrimidin-4-ylamino)methyl)cyclohexylcarbamate (1.2 g, 63%) as a white solid. LCMS (ESI): m/z=382.2 [M+1]⁺.

step 3: A mixture of tert-butyl (1r,4r)-4-((5-(hydroxymethyl)-2-(methylthio)pyrimidin-4-ylamino)methyl)cyclohexylcarbamate (1.2 g, 3.13 mmol), MnO₂ (3.72 g, 42.5 mmol) in DCM (50 ml) was heated to reflux under N₂ overnight. The mixture was cooled to RT, filtered and evaporated to afford tert-butyl (1r,4r)-4-((5-formyl-2-(methylthio)pyrimidin-4-ylamino)methyl)cyclohexylcarbamate (1.18 g, 99%) as a white solid. The compound was used directly in the next step without further purification. LCMS (ESI): m/z=379.2 (M+1).

step 4: A mixture of tert-butyl (1r,4r)-4-((5-formyl-2-(methylthio)pyrimidin-4-ylamino)methyl)cyclohexylcarbamate (60 mg, 0.16 mmol), methyl 2-(2-chloro-4-(6-methylpyrazin-2-yl)phenyl)acetate (43.6 mg, 0.16 mmol), K₂CO₃ (65 mg, 0.47 mmol) in DMF (1.0 ml) was heated at 70° C. overnight under an atmosphere of N₂. After cooling to RT, ice was added. The mixture was filtered and the filter cake was triturated with DCM and the organics were dried over anhydrous Na₂SO₄, filtered and concentrated to afford tert-butyl (1r,4r)-4-((6-(2-chloro-4-(6-methylpyrazin-2-yl)phenyl)-2-(methylthio)-7-oxopyrido[2,3-d]pyrimidin-8(7H)-yl)methyl)cyclohexylcarbamate (57 mg, 60%) as a yellow solid. The compound was used in the next step without further purification. LCMS (ESI): m/z=607.2 (M+1).

step 5: To a mixture of tert-butyl (1r,4r)-4-((6-(2-chloro-4-(6-methylpyrazin-2-yl)phenyl)-2-(methylthio)-7-oxopyrido[2,3-d]pyrimidin-8(7H)-yl)methyl)cyclohexylcarbamate (57 mg, 0.09 mmol) in DCM (5.0 ml) under N₂ was added dropwise to a solution of MCPBA (80%, 22 mg, 0.10 mmol) in DCM (1.0 ml) at −10° C. The mixture was stirred at RT overnight and the mixture quenched with saturated aqueous NaHCO₃. The mixture was extracted with DCM (3×10 mL) and the combined organic layers dried over anhydrous Na₂SO₄, filtered and evaporated to afford 70 mg of crude tert-butyl (1r,4r)-4-((6-(2-chloro-4-(6-methylpyrazin-2-yl)phenyl)-2-(methylsulfinyl)-7-oxopyrido[2,3-d]pyrimidin-8(7H)-yl)methyl)cyclohexylcarbamate. The crude product was used in the next step without further purification. LCMS (ESI): m/z=623.2 (M+1).

step 6: A mixture of crude tert-butyl (1r,4r)-4-((6-(2-chloro-4-(6-methylpyrazin-2-yl)phenyl)-2-(methylsulfinyl)-7-oxopyrido[2,3-d]pyrimidin-8(7H)-yl)methyl)cyclohexylcarbamate (70 mg, 0.09 mmol), DIPEA (163 mg, 1.26 mmol) and ethanamine hydrochloride (40 mg, 0.5 mmol) in THF (5.0 mL) was stirred at RT under N₂ overnight. The mixture was quenched with H₂O and extracted with DCM (3×50 mL). The combined organic layers were dried over anhydrous Na₂SO₄, filtered and evaporated to afford tert-butyl (1r,4r)-4-((6-(2-chloro-4-(6-methylpyrazin-2-yl)phenyl)-2-(ethylamino)-7-oxopyrido[2,3-d]pyrimidin-8(7H)-yl)methyl)cyclohexylcarbamate (90 mg, 100%) as a yellow solid, which was used in the next step without further purification. LCMS (ESI): m/z=604.4 (M+1).

step 7: To a solution of tert-butyl (1r,4r)-4-((6-(2-chloro-4-(6-methylpyrazin-2-yl)phenyl)-2-(ethylamino)-7-oxopyrido[2,3-d]pyrimidin-8(7H)-yl)methyl)cyclohexylcarbamate (90 mg, 0.09 mmol) in MeOH (1.0 mL) was added dropwise methanolic HCl (2.0 mL, 4N) at a rate to maintain the temperature below 0° C. The reaction mixture was then stirred for 2 h at RT. The mixture was concentrated and purified by prep-HPLC to afford 8-(((1r,4r)-4-aminocyclohexyl)methyl)-6-(2-chloro-4-(6-methylpyrazin-2-yl)phenyl)-2-(ethylamino)pyrido[2,3-d]pyrimidin-7(8H)-one (23.1 mg, 51%). ¹H NMR (500 MHz, MeOD-d₄) δ 9.16 (s, 1H), 8.64 (s, 1H), 8.57 (s, 1H), 8.27 (s, 1H), 8.16-8.15 (m, 1H), 8.02-8.00 (m, 1H), 7.83 (s, 1H), 7.57-7.55 (m, 1H), 4.19-4.17 (m, 2H), 3.40-3.34 (m, 2H), 2.60 (s, 3H), 1.85-1.84 (m, 1H), 1.76-1.74 (m, 2H), 1.63-1.60 (m, 2H), 2.21-1.01 (m, 5H), 0.96-0.91 (m, 2H). LCMS (ESI): m/z=504.4 (M+1).

### Example 4

### 6-(2-Chloro-4-(6-methylpyrazin-2-yl)phenyl)-2-(ethylamino)-8-(((1r,4r)-4-(methylamino)cyclohexyl)methyl)pyrido[2,3-d]pyrimidin-7(8H)-one (I-17)

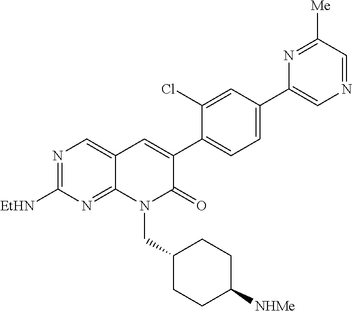

The title compound was prepared in accord with Example 3 using appropriate starting materials. ¹H NMR (500 MHz, CDCl3-d3) δ 8.83 (s, 1H), 8.47 (brs, 1H), 8.43 (s, 1H), 8.16 (s, 1H), 7.94 (dd, 1H), 7.58 (s, 1H), 7.51 (d, 1H), 5.52 (brs, 1H), 4.32 (s, 2H), 3.55 (t, 2H), 2.65 (s, 3H), 2.43 (s, 3H), 2.37-2.33 (m, 1H), 1.98 (d, 3H), 1.78 (d, 2H), 1.31 (t, 3H), 1.25-1.19 (m, 2H), 1.07-1.01 (m, 2H). MS (ESI): m/z=518.1 [M+1].

### Example 5

### 8-(((1r,4r)-4-Aminocyclohexyl)methyl)-6-(2-chloro-4-(6-methylpyridin-2-yl)phenyl)-2-(ethylamino)pyrido[2,3-d]pyrimidin-7(8H)-one (I-15)

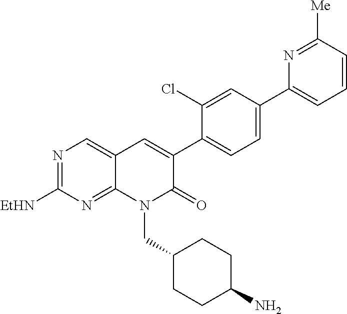

The title compound can be prepared in accord with Example 3 by replacing methyl 2-(2-chloro-4-(6-methylpyrazin-2-yl)phenyl)acetate with methyl 2-(2-chloro-4-(6-methylpyridin-2-yl)phenyl)acetate in step 4. [M+1]. ¹H NMR (500 MHz, DMSO-d₆) δ 8.64 (s, 1H), 8.21 (s, 1H), 8.10-8.06 (d, 1H), 8.00-7.96 (brs, 1H), 7.89-7.85 (d, 1H), 7.84-7.79 (m, 2H), 7.52-7.48 (d, 1H), 7.30-7.26 (d, 1H), 4.20-4.13 (d, 2H), 3.45-3.37 (t, 3H), 2.57 (s, 3H), 1.90-1.82 (brs, 1H), 1.82-1.75 (d, 2H), 1.68-1.59 (d, 2H), 1.22-1.09 (m, 5H), 1.03-0.95 (m, 2H); MS (ESI): m/z=503.1.

### Example 6

### 8-(((1r,4r)-4-Aminocyclohexyl)methyl)-6-(2-chloro-4-(3-methylpyridin-2-yl)phenyl)-2-(ethylamino)pyrido[2,3-d]pyrimidin-7(8H)-one (I-16)

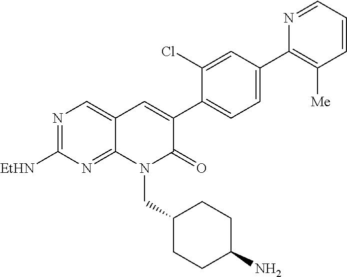

The title compound can be prepared in accord with Example 3 by replacing methyl 2-(2-chloro-4-(6-methylpyrazin-2-yl)phenyl)acetate with methyl 2-(2-chloro-4-(3-methylpyridin-2-yl)phenyl)acetate in step 4. ¹H NMR (500 MHz, DMSO-d₆) δ 8.62 (s, 1H), 8.54-8.51 (d, 1H), 8.01-7.95 (t, 1H), 7.84 (s, 1H), 7.79-7.75 (d, 1H), 7.69 (s, 1H), 7.60-7.56 (d, 1H), 7.50-7.46 (d, 1H), 7.37-7.32 (m, 7H), 4.20-4.13 (d, 2H), 3.45-3.37 (t, 3H), 2.40 (s, 3H), 1.92-1.79 (d, 3H), 1.70-1.60 (d, 2H), 1.23-1.01 (m, 7H); MS (ESI): [M+1] m/z=503.1.

### Example 7

### 8-(((1r,4r)-4-Aminocyclohexyl)methyl)-6-(2-chloro-4-(6-methylpyrazin-2-yl)phenyl)-2-(methylamino)pyrido[2,3-d]pyrimidin-7(8H)-one (I-7)

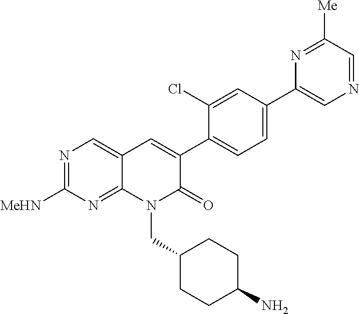 [SMILES: CNc1ncc2cc(-c3ccc(-c4cncc(C)n4)cc3Cl)c(=O)n(C[C@H]3CC[C@H](N)CC3)c2n1]

The title compound can be prepared in accord with Example 3 by replacing ethanamine hydrochloride with methanamine hydrochloride in step 6. ¹H NMR (500 MHz, CDCl₃) 8.827 (s, 1H), 8.471 (s, 1H), 8.426 (s, 1H), 8.166 (s, 1H), 7.949-7.930 (d, 1H), 7.588 (s, 1H), 7.525-7.509 (d, 1H), 5.459 (m, 1H), 4.341 (m, 2H), 3.108-3.099 (m, 2H), 2.649-2.609 (m, 4H), 1.986 (m, 1H), 1.873-1.852 (m, 1H), 1.742-1.738 (m, 2H), 1.252-1.201 (t, 3H), 1.072-1.025 (m, 2H); LCMS (ESI): m/z=490.3[M+1]⁺.

### Example 8

### 2-Amino-8-(((1r,4r)-4-aminocyclohexyl)methyl)-6-(2-chloro-4-(6-methylpyrazin-2-yl)phenyl)pyrido[2,3-d]pyrimidin-7(8H)-one (I-9)

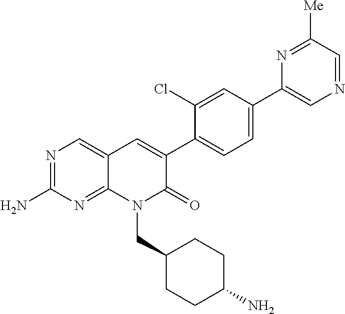 [SMILES: Cc1cncc(-c2ccc(-c3cc4cnc(N)nc4n(C[C@H]4CC[C@H](N)CC4)c3=O)c(Cl)c2)n1]

The title compound can be prepared in accord with Example 3 by replacing ethanamine hydrochloride with ammonium hydrochloride in step 6.

¹H NMR (500 MHz, CDCl₃ CDCl3-d₃) δ 8.828 (s, 1H), 8.516 (s, 1H), 8.432 (s, 1H), 8.168 (s, 1H), 7.945 (d, J=7.5 Hz, 1H), 7.602 (s, 1H), 7.508 (d, J=9 Hz, 1H), 5.331 (s, 1H), 4.292 (d, J=8 Hz, 2H), 2.650 (s, 4H), 1.892 (s, 1H), 1.860 (d, J=8.5 Hz, 2H), 1.734 (d, J=15.5 Hz, 3H), 1.308-1.199 (m, 4H), 1.045-1024 (m, 2H); LCMS [M+1]⁺ (ESI): m/z=476.

### Example 9

### 6-(2-Chloro-4-(6-methylpyrazin-2-yl)phenyl)-8-(((1r,4r)-4-(dimethylamino)cyclohexyl)methyl)-2-(ethylamino)pyrido[2,3-d]pyrimidin-7(8H)-one (I-10)

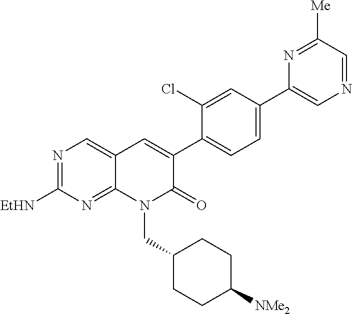

To the product of step 4 in Example 3, tert-butyl (1r,4r)-4-((6-(2-chloro-4-(6-methylpyrazin-2-yl)phenyl)-2-(methylthio)-7-oxopyrido[2,3-d]pyrimidin-8(7H)-yl)methyl)cyclohexylcarbamate (250 mg, 0.493 mmol) in MeOH (20 mL) was added HCHO/H₂O (1 mL), NaBH(OAc)₃ (404 mg, 2.47 mmol) and HOAc (0.5 mL) at RT. After stirring overnight at RT the mixture was quenched with water (3.0 mL) and extracted with DCM (3×50 mL). The organic layer was washed with brine and dried over MgSO₄. After removal of solvent under reduced pressure, 6-(2-chloro-4-(6-methylpyrazin-2-yl)phenyl)-8-(((1r,4r)-4-(dimethylamino)cyclohexyl)methyl)-2-(methylthio)pyrido[2,3-d]pyrimidin-7(8H)-one (200 mg, 84%) was obtained as a yellow solid. LCMS (ESI): m/z=535.2 [M+1]⁺

The product from the above reaction was converted to the final product using analogous procedures described in Example 3, steps 5 and 6. ¹H NMR (500 MHz, CDCl3 CDCl3-d₃) δ 8.826 (s, 1H), 8.467 (s, 1H), 8.426 (s, 1H), 8.165 (s, 1H), 7.948-7.928 (d, 1H), 7.579 (s, 1H), 7.521-7.505 (d, 1H), 5.518 (m, 1H), 4.315 (m, 2H), 3.563-3.537 (m, 2H), 2.648 (s, 3H), 2.257 (s, 6H), 2.184 (m, 1H), 2.920-2.897 (m, 2H), 1.914-1.803 (m, 5H), 1.333-1.305 (t, 3H), 1.254-1.150 (m, 4H). LCMS (ESI): [M+1]⁺ m/z=532.2.

### Example 10

### 6-(2-Chloro-4-(6-methylpyrazin-2-yl)phenyl)-2-(ethylamino)-8-(piperidin-4-ylmethyl)pyrido[2,3-d]pyrimidin-7(8H)-one (I-6)

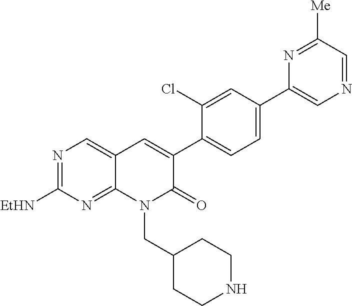

step 1: To a solution of 6-bromo-2-(methylthio)pyrido[2,3-d]pyrimidin-7(8H)-one (435 mg, 1.6 mmol) in DMF (3 mL) was added tert-butyl 4-(hydroxymethyl)piperidine-1-carboxylate (750 mg, 3.5 mmol) and Ph₃P (970 mg, 3.5 mmol) under a nitrogen atmosphere. The mixture was cooled to 0° C. and DIAD (710 mg, 3.5 mmol) was added dropwise while maintaining the temperature between 0-5° C. The mixture was stirred at RT overnight. The reaction mixture was poured into ice water and extracted with EtOAc (3×100 mL). The organic layer was washed with saturated NaCl solution (4×100 mL), dried (MgSO₄), filtered and concentrated. The residue was purified by SiO₂ chromatography eluting with a PE/EtOAc gradient (10/1 to 6/1 to 4/1) to afford tert-butyl 4-((6-bromo-2-(methylthio)-7-oxopyrido[2,3-d]pyrimidin-8(7H)-yl)methyl)piperidine-1-carboxylate (485 mg, 65%) as white solid. LCMS (ESI): m/z=414.0 [M−56]+.

step 2: A mixture of tert-butyl 4-((6-bromo-2-(methylthio)-7-oxopyrido[2,3-d]pyrimidin-8(7H)-yl)methyl)piperidine-1-carboxylate (239 mg, 0.5 mmol), MCPBA (80%, 275 mg, 1.2 mmol) in DCM (18 mL) was stirred at RT overnight. The reaction mixture was washed with saturated NaHCO₃ solution (2×50 mL) and saturated NaCl solution (2×50 mL). The organic layer was dried (MgSO₄), filtered and concentrated in vacuo to afford crude tert-butyl 4-((6-bromo-2-(methylsulfonyl)-7-oxopyrido[2,3-d]pyrimidin-8(7H)-yl)methyl)piperidine-1-carboxylate (279 mg, 100%) as yellow solid. LCMS (ESI): m/z=445.0 [M−56]⁺.

step 3: A mixture of tert-butyl 4-((6-bromo-2-(methylsulfonyl)-7-oxopyrido[2,3-d]pyrimidin-8(7H)-yl)methyl)piperidine-1-carboxylate (160 mg, 0.32 mmol), ethylamine hydrochloride (259 mg, 3.2 mmol), DIPEA (1.5 mL) in IPA (4 mL) was heated at 90° C. in a sealed tube with stirring overnight. The reaction mixture was concentrated and the residue purified by SiO₂ chromatography eluting with a DCM/MeOH gradient (80/1 to 40/1) to afford tert-butyl 4-((6-bromo-2-(ethylamino)-7-oxopyrido[2,3-d]pyrimidin-8(7H)-yl)methyl)piperidine-1-carboxylate (108 mg, 72%) as white solid. LCMS (ESI): m/z=410.0 [M−56]+.

step 4: tert-Butyl 4-((6-bromo-2-(ethylamino)-7-oxopyrido[2,3-d]pyrimidin-8(7H)-yl)methyl)piperidine-1-carboxylate (100 mg, 0.021 mmol) was dissolved in a mixture of ethylene glycol dimethyl ether (3 mL), ethanol (2 mL), toluene (0.3 mL) and water (0.6 mL). Then 2-chloro-4-(6-methylpyrazin-2-yl)phenylboronic acid (61 mg, 0.21 mmol), Cs₂CO₃ (107 mg, 0.33 mmol), and (PPh₃)₄Pd(II) (13 mg, 0.011 mmol) were added. The mixture was heated at 85° C. with stirring overnight. The reaction mixture was concentrated and dissolved in DCM (50 mL). The solution was washed with saturated NaCl solution (2×30 mL), dried over MgSO₄, filtered and concentrated to afford crude tert-butyl 4-((6-(2-chloro-4-(6-methylpyrazin-2-yl)phenyl)-2-(ethylamino)-7-oxopyrido[2,3-d]pyrimidin-8(7H)-yl)methyl)piperidine-1-carboxylate (203 mg, 100%) as pale yellow solid. LCMS (ESI: m/z=490.1 [M−100]⁺.

step 5: TFA (10 mL) was slowly added into the solution of tert-butyl 4-((6-(2-chloro-4-(6-methylpyrazin-2-yl)phenyl)-2-(ethylamino)-7-oxopyrido[2,3-d]pyrimidin-8(7H)-yl)methyl)piperidine-1-carboxylate (203 mg, 0.34 mmol) in DCM (10 mL) in an ice-water bath with stirring. The mixture was then stirred at RT for 1 h. After evaporation, ammonia (7N in MeOH, 10 mL) was added to the residue. The mixture was concentrated and the residue purified by prep-HPLC to afford 6-(2-chloro-4-(6-methylpyrazin-2-yl)phenyl)-2-(ethylamino)-8-(piperidin-4-ylmethyl)pyrido[2,3-d]pyrimidin-7(8H)-one (46 mg, 28%) as a white solid. ¹H NMR (500 MHz, DMSO-d₆): δ 9.16 (s, 1H), 8.64 (s, 1H), 8.57 (s, 1H), 8.27 (s, 1H), 8.16-8.14 (d, 1H), 7.99 (s, 1H), 7.83 (s, 1H), 7.56-7.54 (d, 1H), 4.21-4.20 (d, 2H), 3.41-3.37 (m, 2H), 2.93-2.91 (d, 2H), 2.60 (s, 3H), 2.39-2.34 (t, 2H), 2.00 (br s, 1H), 1.51-1.49 (d, 2H), 1.23-1.19 (m, 6H). MS (ESI): m/z=490.2[M]+.

### Example 11

### 8-(3-Aminopropyl)-6-[2-chloro-4-(6-methylpyrazin-2-yl)phenyl]-2-(ethylamino)pyrido[2,3-d]pyrimidin-7-one (I-1)

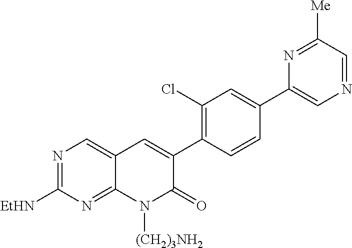

step 1: A mixture of 6-[2-chloro-4-(6-methylpyrazin-2-yl)phenyl]-2-methylsulfanyl-8H-pyrido[2,3-d]pyrimidin-7-one (450 mg, 1.14 mmol), Cs₂CO₃ (1.11 g, 3.41 mmol), and tert-butyl N-(3-bromopropyl)carbamate (541.3 mg, 2.28 mmol) in anhydrous DMF (5370 mg, 73.5 mmol, 5.69 mL) was stirred at RT overnight. The solvents were then removed and the crude product used in the next step without further purification. MS (ESI) m/z 553.4 [M+1]⁺

step 2: To tert-butyl N-[3-[6-[2-chloro-4-(6-methylpyrazin-2-yl)phenyl]-2-methylsulfanyl-7-oxo-pyrido[2,3-d]pyrimidin-8-yl]propyl]carbamate (628.6 mg, 1.14 mmol) in anhydrous DCM (18.21 mL) at 0° C. was added portionwise MCPBA (1.10 equiv, 1.25 mmol, 280.2 mg). The reaction mixture was stirred at 0° C. for 15 min and then quenched with saturated aq. NaHCO₃ solution (25 mL) and extracted with DCM (3×30 mL). The organic layers were combined and reduced in vacuo. The crude product was used in the next step without further purification. MS (ESI) m/z: 569.3 [M+1]⁺.

step 3: A mixture of tert-butyl N-[3-[6-[2-chloro-4-(6-methylpyrazin-2-yl)phenyl]-2-methylsulfinyl-7-oxo-pyrido[2,3-d]pyrimidin-8-yl]propyl]carbamate (323.4 mg, 0.569 mmol), ethanamine in THF (2.84 mmol, 1.421 mL), and DIPEA (4.00 equiv, 2.27 mmol, 293.8 mg, 0.40 mL) in anhydrous THF (64.9 equiv, 36.9 mmol, 3 mL) was stirred at RT for 2 h. The solvents were removed to afford tert-butyl N-[3-[6-[2-chloro-4-(6-methylpyrazin-2-yl)phenyl]-2-(ethylamino)-7-oxo-pyrido[2,3-d]pyrimidin-8-yl]propyl]carbamate as an crude orange material which was progressed to the next step without further purification. MS (ESI) m/z: 550.3 [M+1]⁺.

step 4: tert-Butyl N-[3-[6-[2-chloro-4-(6-methylpyrazin-2-yl)phenyl]-2-(ethylamino)-7-oxo-pyrido[2,3-d]pyrimidin-8-yl]propyl]carbamate (312 mg, 0.567 mmol) was taken up in a solution of MeOH (2 mL) and DCM (2 mL). An HCl solution (4. 0M in dioxane, 1.42 mL) was added. The reaction mixture was stirred overnight at RT. The solvents were removed in vacuo, and the product purified by reverse-phase HPLC to afford 8-(3-aminopropyl)-6-[2-chloro-4-(6-methylpyrazin-2-yl)phenyl]-2-(ethylamino)pyrido[2,3-d]pyrimidin-7-one (61 mg, 0.122 mmol, 21.4% yield) as an off-white solid.

MS (ESI) m/z: 450.2 [M+1]⁺. ¹H NMR (400 MHz, DMSO-d6) δ 9.15 (s, 1H), 8.74-8.63 (m, 1H), 8.59-8.45 (m, 1H), 8.27 (d, J=1.8 Hz, 1H), 8.15 (dd, J=8.0, 1.8 Hz, 1H), 8.02-7.95 (m, 1H), 7.84 (s, 1H), 7.56 (d, J=8.0 Hz, 1H), 4.42-4.33 (m, 2H), 3.48-3.36 (m, 2H), 2.66-2.58 (m, 5H), 1.86-1.77 (m, 3H), 1.26-1.11 (m, 4H).

### Example 12

### 8-(4-Aminobutyl)-6-[2-chloro-4-(6-methylpyrazin-2-yl)phenyl]-2-(ethylamino)pyrido[2,3-d]pyrimidin-7-one (I-2)

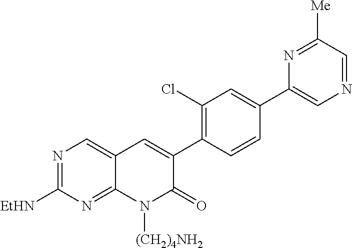

The title compound was prepared in accord with Example 11 except in step 1, tert-butyl N-(3-bromopropyl)carbamate was replaced with tert-butyl N-(4-bromobutyl)carbamate. ¹H NMR (400 MHz, DMSO-d₆) δ 9.15 (s, 1H), 8.65 (s, 1H), 8.57 (s, 1H), 8.27 (d, J=1.7 Hz, 1H), 8.15 (dd, J=8.0, 1.8 Hz, 1H), 7.97 (d, J=6.3 Hz, 1H), 7.86-7.78 (m, 1H), 7.55 (d, J=7.9 Hz, 1H), 4.36-4.27 (m, 2H), 3.47-3.35 (m, 2H), 2.70-2.62 (m, 2H), 2.60 (s, 3H), 1.77-1.67 (m, 3H), 1.54-1.42 (m, 3H), 1.25-1.17 (m, 3H); MS (ESI) m/z: 464.2 [M+1]⁺.

### Example 13

### 8-(3-Aminopropyl)-6-[2-chloro-4-(6-methylpyrazin-2-yl)phenyl]-2-(methylamino)pyrido[2,3-d]pyrimidin-7-one (I-18)

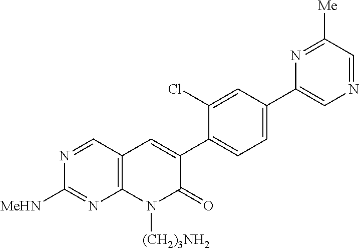

The title compound prepared in accord with Example 11 except in step 3, ethanamine was replaced with methanamine ¹H NMR (400 MHz, DMSO-d₆) δ 9.16 (s, 1H), 8.65 (s, 1H), 8.57 (s, 1H), 8.27 (d, J=1.8 Hz, 1H), 8.15 (dd, J=8.0, 1.8 Hz, 1H), 7.92-7.72 (m, 2H), 7.56 (d, J=8.0 Hz, 1H), 4.46-4.25 (m, 2H), 2.95-2.87 (m, 3H), 2.63-2.53 (m, 5H), 1.80 (q, J=7.1 Hz, 2H); MS (ESI) m/z: 436.1 [M+1]⁺.

### Example 14

### 8-(2-(2-Aminoethoxy)ethyl)-6-(2-chloro-4-(6-methylpyrazin-2-yl)phenyl)-2-(ethylamino)pyrido[2,3-d]pyrimidin-7(8H)-one (I-12)

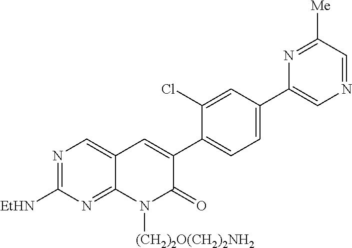

The title compound was prepared in accord with Example 11 except in step 1, tert-butyl N-(3-bromopropyl)carbamate was replaced with tert-butyl [2-(2-bromoethoxy)ethyl]carbamate (CASRN 164332-88-1). ¹H NMR (500 MHz, CDCl₃): δ 8.83 (s, 1H), 8.42 (d, 2H), 8.10 (d, 1H), 7.94 (d, 1H), 7.58 (s, 1H), 7.50 (d, 1H), 5.56 (s, 1H), 4.69 (brs, 2H), 3.85 (brs, 2H), 3.58-3.56 (m, 4H), 2.84-2.81 (m, 2H), 2.64 (s, 3H), 1.32-1.29 (m, 5H); LCMS (ESI): m/z=507.2 [M+1]⁺

### Example 15

### 6-(2-Chloro-4-(6-methylpyrazin-2-yl)phenyl)-2-(ethylamino)-8-(2-(pyrrolidin-3-yloxy)ethyl)pyrido[2,3-d]pyrimidin-7(8H)-one (I-13)

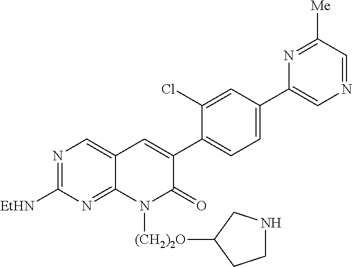

The title compound was prepared in accord with Example 11 Example 11 except in step 1, tert-butyl N-(3-bromopropyl)carbamate was replaced with tert-butyl 3-(2-(tosyloxy)ethoxy)pyrrolidine-1-carboxylate. ¹H NMR (500 MHz, CDCl3-d₃): δ 8.83 (s, 1H), 8.48 (br s, 1H), 8.43 (s, 1H), 8.17-8.16 (m, 1H), 7.96-7.93 (m, 1H), 7.58 (s, 1H), 7.50-7.47 (m, 1H), 4.65 (br s, 2H), 4.16 (br s, 1H), 3.79-3.76 (m, 2H), 3.58-3.53 (m, 2H), 3.08 (br s, 2H), 2.95-2.89 (m, 2H), 2.65 (s, 3H), 1.91-1.85 (m, 2H), 1.31 (t, 3H). LCMS (ESI): m/z=506.0 [M+1]⁺.

### Example 16

### 6-(2-Chloro-4-(6-methylpyrazin-2-yl)phenyl)-2-(ethylamino)-8-(2-(piperazin-1-yl)ethyl)pyrido[2,3-d]pyrimidin-7(8H)-one (I-4)

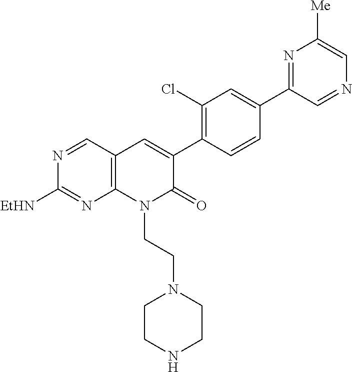

The title compound was prepared in accord with Example 11 Example 11 except in step 1, tert-butyl N-(3-bromopropyl)carbamate was replaced with tert-butyl 4-(2-methylsulfonyloxyethyl)piperazine-1-carboxylate. ¹H NMR (400 MHz, DMSO-d₆) δ 9.15 (s, 1H), 8.64 (s, 1H), 8.57 (s, 1H), 8.27 (d, J=1.8 Hz, 1H), 8.15 (dd, J=8.0, 1.8 Hz, 1H), 8.00-7.94 (m, 1H), 7.83 (s, 1H), 7.55 (d, J=8.0 Hz, 1H), 4.47-4.36 (m, 2H), 3.46-3.37 (m, 2H), 2.68-2.62 (m, 4H), 2.62-2.54 (m, 1H), 2.60 (s, 3H), 2.45-2.36 (s, 4H), 1.31-1.07 (m, 5H); MS (ESI+) m/z: 505.3 [M+1]⁺.

### Example 17

### 6-(2-Chloro-4-(6-methylpyrazin-2-yl)phenyl)-2-(ethylamino)-8-(2-oxo-2-(piperazin-1-yl)ethyl)pyrido[2,3-d]pyrimidin-7(8H)-one (I-8)

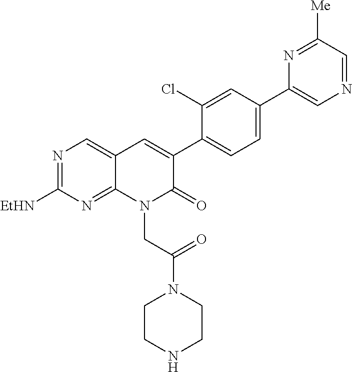

The title compound was prepared in accord with Example 11 except in step 1, tert-butyl N-(3-bromopropyl)carbamate was replaced with tert-butyl 4-(2-chloroacetyl)piperazine-1-carboxylate. ¹H NMR (500 MHz, DMSO-d₆) δ 9.15 (s, 1H), 8.66 (s, 1H), 8.56 (s, 1H), 8.27 (d, J=2.0, 1H), 8.15 (dd, J=2.0, 8.0, 1H), 8.02-8.00 (m, 1H), 7.89 (s, 1H), 7.55 (d, J=8.0, 1H), 5.12 (s, 2H), 3.56-3.54 (m, 2H), 3.36-3.34 (m, 4H), 2.77-2.75 (m, 2H), 2.74-2.72 (m, 2H), 2.47 (s, 3H), 1.14 (t, J=6.0, 3H); LCMS (ESI): m/z=519.2 [M+1]⁺.

### Example 18

### (R)-6-(2-Chloro-4-(6-methylpyrazin-2-yl)phenyl)-2-(ethylamino)-8-(2-(pyrrolidin-2-yl)ethyl)pyrido[2,3-d]pyrimidin-7(8H)-one (I-19) and (S)-6-(2-chloro-4-(6-methylpyrazin-2-yl)phenyl)-2-(ethylamino)-8-(2-(pyrrolidin-2-yl)ethyl)pyrido[2,3-d]pyrimidin-7(8H)-one (I-20)

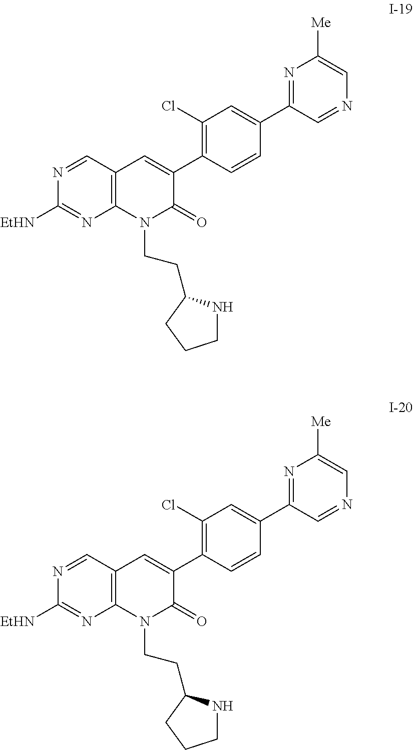

Benzyl 2-[2-[6-[2-chloro-4-(6-methylpyrazin-2-yl)phenyl]-2-(ethylamino)-7-oxo-pyrido[2,3-d]pyrimidin-8-yl]ethyl]pyrrolidine-1-carboxylate was prepared using a similar sequence as described in steps 1 to 3 of Example 11 using 6-[2-chloro-4-(6-methylpyrazin-2-yl)phenyl]-2-methylsulfanyl-8H-pyrido[2,3-d]pyrimidin-7-one and benzyl 2-(2-methylsulfonyloxyethyl)pyrrolidine-1-carboxylate as the starting materials in the step 1. Chiral SFC separation afforded the following enantiomers:

Peak 1: MS (ESI) m/z: 643.2 [M+1]⁺; SFC retention time=1.014 min.

Peak 2: MS (ESI) m/z: 643.2 [M+1]⁺; SFC retention time=1.371 min.

To benzyl 2-[2-[6-[2-chloro-4-(6-methylpyrazin-2-yl)phenyl]-2-(ethylamino)-7-oxo-pyrido[2,3-d]pyrimidin-8-yl]ethyl]pyrrolidine-1-carboxylate (Peak 1 from step 5) (125 mg, 0.20 mmol) dissolved in 1,4-dioxane (10 mL) was added 2N aq. HCl (8 mL, 16.0 mmol) and the reaction mixture was stirred at 90° C. for 16 h. The volatile solvent was concentrated, and the crude product redissolved in water (10.0 mL) and neutralized with sat. aqueous NaHCO₃ solution to pH ca. 7. The reaction mixture was extracted with EtOAc (3×50 mL). The combined organic layers were washed with water and brine, dried over Na₂SO₄, filtered and concentrated. The crude residue was purified by SiO₂ chromatography eluting with EtOAc (containing 1% TEA):MeOH (containing 1% TEA), followed by preparative HPLC to afford pure enantiomeric 6-(2-chloro-4-(6-methylpyrazin-2-yl)phenyl)-2-(ethylamino)-8-(2-(pyrrolidin-2-yl)ethyl)pyrido[2,3-d]pyrimidin-7(8H)-one as a solid (11.1 mg, 11.3%). ¹H NMR (400 MHz, DMSO-d₆) δ 9.16 (s, 1H), 8.64 (s, 1H), 8.57 (s, 1H), 8.27 (d, J=1.7 Hz, 1H), 8.15 (dd, J=8.0, 1.8 Hz, 1H), 8.02-7.95 (m, 1H), 7.83 (s, 1H), 7.55 (d, J=8.0 Hz, 1H), 4.42-4.27 (m, 2H), 3.42 (p, J=7.0 Hz, 2H), 2.99-2.79 (m, 2H), 2.67 (q, J=7.8 Hz, 1H), 2.60 (s, 3H), 1.87-1.77 (m, 1H), 1.78-1.68 (m, 2H), 1.68-1.52 (m, 2H), 1.32-1.11 (m, 5H). MS (ESI) m/z: 490.2 [M+1]⁺.

The enantiomer represented by peak 2 described above was deprotected as described for its opposite enantiomer above.

¹H NMR (400 MHz, DMSO-d₆) δ 9.16 (s, 1H), 8.65 (s, 1H), 8.57 (s, 1H), 8.27 (d, J=1.7 Hz, 1H), 8.15 (dd, J=8.0, 1.8 Hz, 1H), 8.02-7.95 (m, 1H), 7.83 (s, 1H), 7.55 (d, J=8.0 Hz, 1H), 4.41-4.28 (m, 2H), 3.42 (p, J=7.0 Hz, 2H), 2.97-2.88 (m, 1H), 2.88-2.78 (m, 1H), 2.72-2.63 (m, 1H), 2.60 (s, 3H), 1.87-1.77 (m, 1H), 1.79-1.67 (m, 2H), 1.70-1.53 (m, 2H), 1.32-1.12 (m, 5H). MS (ESI) m/z: 490.2 [M+1]⁺.

### Example 19

### (S)-6-(2-chloro-4-(6-methylpyrazin-2-yl)phenyl)-2-(ethylamino)-8-(2-(morpholin-2-yl)ethyl)pyrido[2,3-d]pyrimidin-7(8H)-one (I-21) and (R)-6-(2-chloro-4-(6-methylpyrazin-2-yl)phenyl)-2-(ethylamino)-8-(2-(morpholin-2-yl)ethyl)pyrido[2,3-d]pyrimidin-7(8H)-one (I-22)

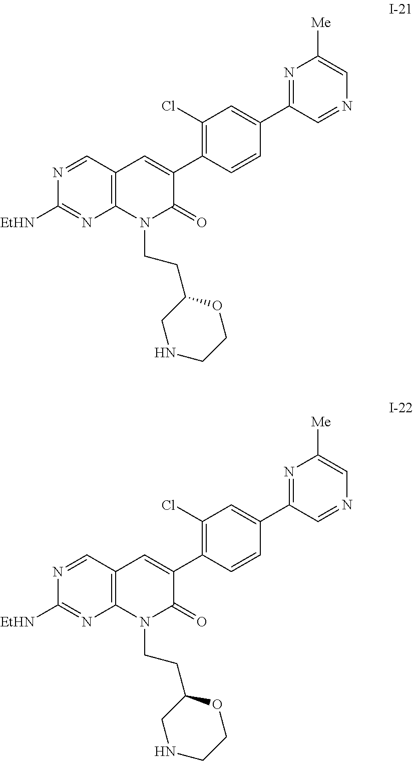

The title compounds were prepared in accord with Example 18 using benzyl 2-(2-((methylsulfonyl)oxy)ethyl)morpholine-4-carboxylate in place of benzyl 2-(2-methylsulfonyloxyethyl)pyrrolidine-1-carboxylate as the starting material.

Peak 1 enantiomer: ¹H NMR (400 MHz, DMSO-d₆) δ 9.15 (s, 1H), 8.65 (s, 1H), 8.57 (s, 1H), 8.27 (d, J=1.7 Hz, 1H), 8.15 (dd, J=8.0, 1.8 Hz, 1H), 7.97 (br s, 1H), 7.84 (s, 1H), 7.55 (d, J=7.9 Hz, 1H), 4.50-4.25 (m, 2H), 3.88-3.80 (m, 1H), 3.63 (br s, 1H), 3.59-3.49 (m, 1H), 3.43 (p, J=7.0 Hz, 2H), 3.07 (d, J=12.6 Hz, 1H), 2.96 (d, J=12.5 Hz, 1H), 2.88-2.78 (m, 1H), 2.69-2.62 (m, 1H), 2.60 (s, 3H), 1.87-1.75 (m, 2H), 1.26-1.14 (m, 4H). MS (ESI) m/z: 506.2 [M+1]⁺.

Peak 2 enantiomer: ¹H NMR (400 MHz, DMSO-d₆) δ 9.16 (s, 1H), 8.66 (s, 1H), 8.57 (s, 1H), 8.28 (d, J=1.7 Hz, 1H), 8.15 (dd, J=8.0, 1.8 Hz, 1H), 8.01 (t, J=5.8 Hz, 1H), 7.85 (s, 1H), 7.55 (d, J=8.0 Hz, 1H), 4.49-4.30 (m, 2H), 3.89 (d, J=11.5 Hz, 1H), 3.69 (br s, 1H), 3.59 (t, J=11.8 Hz, 1H), 3.47-3.37 (m, 2H), 3.15 (d, J=12.3 Hz, 1H), 3.06 (d, J=12.9 Hz, 1H), 2.95-2.84 (m, 1H), 2.71 (t, J=11.7 Hz, 1H), 2.60 (s, 3H), 1.89-1.75 (m, 2H), 1.27-1.12 (m, 4H). MS (ESI) m/z: 506.2 [M+1]⁺.

### Example 20

### 6-(2-Chloro-4-(6-methylpyrazin-2-yl)phenyl)-2-(ethylamino)-8-(2-(4-methylpiperazin-1-yl)ethyl)pyrido[2,3-d]pyrimidin-7(8H)-one (I-27)

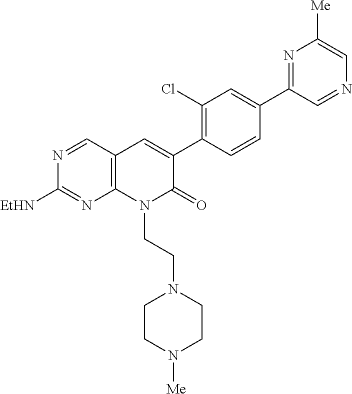

step 1: A mixture of 6-[2-chloro-4-(6-methylpyrazin-2-yl)phenyl]-2-methylsulfanyl-8H-pyrido[2,3-d]pyrimidin-7-one (300 mg), 1-(2-chloroethyl)-4-methyl-piperazine hydrochloride (271.6 mg, 1.36 mmol), cesium carbonate (2.1 g, 6.44 mmol) in anhydrous DMF (9.5 mL) in a sealed tube was stirred at 120° C. for 20 h. Water (100 mL) was added to the cooled reaction mixture and the reaction mixture was extracted with EtOAc (150 mL×3). The organic layers were washed with brine, dried over Na₂SO₄, filtered, and concentrated under reduced pressure. The crude residue was purified by SiO₂ chromatography eluting with EtOAc:MeOH to afford 6-[2-chloro-4-(6-methylpyrazin-2-yl)phenyl]-8-[2-(4-methylpiperazin-1-yl)ethyl]-2-methylsulfanyl-pyrido[2,3-d]pyrimidin-7-one as a solid (89.9 mg, 22.7%). ¹H NMR (400 MHz, CDCl₃) δ 8.82 (s, 1H), 8.65 (s, 1H), 8.42 (s, 1H), 8.17 (d, J=1.7 Hz, 1H), 7.95 (dd, J=8.0, 1.8 Hz, 1H), 7.68 (s, 1H), 7.49 (d, J=8.0 Hz, 1H), 4.70-4.58 (m, 2H), 2.79-2.74 (m, 2H), 2.64 (d, J=1.6 Hz, 10H), 2.48-2.39 (m, 4H), 2.28 (s, 3H). MS (ESI) m/z: 522.2 [M+1]⁺.

step 2: To 6-[2-chloro-4-(6-methylpyrazin-2-yl)phenyl]-8-[2-(4-methylpiperazin-1-yl)ethyl]-2-methylsulfanyl-pyrido[2,3-d]pyrimidin-7-one (89 mg) in anhydrous DCM (5.5 mL) at 0° C. was added MCPBA (42 mg, 0.19 mmol). The reaction mixture was stirred at 0° C. for 1 h, and additional MCPBA (21 mg, 0.095 mmol) was added. After stirring at RT for 0.5 h, the reaction mixture was quenched with saturated aqueous NaHCO₃ solution (10 mL). Volatile solvent was removed under reduced pressure, and the residue was extracted with EtOAc (25 mL×3). The combined organic layers were washed with water and brine, dried over Na₂SO₄, filtered, and concentrated under reduced pressure to give crude 6-[2-chloro-4-(6-methylpyrazin-2-yl)phenyl]-8-[2-(4-methylpiperazin-1-yl)ethyl]-2-methylsulfinyl-pyrido[2,3-d]pyrimidin-7-one (90 mg, 98.1%). MS (ESI) m/z: 538.2 [M+1]⁺.

step 3: A mixture of 6-[2-chloro-4-(6-methylpyrazin-2-yl)phenyl]-8-[2-(4-methylpiperazin-1-yl)ethyl]-2-methylsulfinyl-pyrido[2,3-d]pyrimidin-7-one (90 mg), ethylamine (2.0 M) in THF (8.5 mL), and DCM (2.1 mL) in a seal vial was stirred at 50° C. for 4 days. The reaction mixture was concentrated under reduced pressure. The crude mixture was purified by preparative HPLC to give the title compound as a solid (6.0 mg, 6.9%). ¹H NMR (400 MHz, DMSO-d₆) δ 9.15 (s, 1H), 8.64 (s, 1H), 8.57 (s, 1H), 8.27 (d, J=1.7 Hz, 1H), 8.15 (dd, J=8.0, 1.9 Hz, 1H), 7.96 (s, 1H), 7.83 (s, 1H), 7.55 (d, J=8.0 Hz, 1H), 4.46-4.36 (m, 2H), 3.46-3.36 (m, 2H), 3.27-3.18 (m, 4H), 2.65-2.56 (m, 5H), 2.28 (m, 4H), 2.13 (s, 3H), 1.20 (t, J=7.3 Hz, 3H). MS (ESI) m/z: 519.2 [M+1]⁺.

### Example 21

### 4-[6-[2-Chloro-4-(6-methylpyrazin-2-yl)phenyl]-2-(ethylamino)-7-oxo-pyrido[2,3-d]pyrimidin-8-yl]butanamide (I-3)

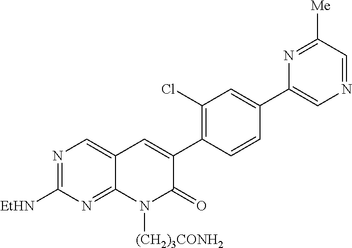

The title compound was prepared in accord with Example 20 except in step 1,1-(2-chloroethyl)-4-methyl-piperazine hydrochloride was replaced with 4-bromo-butanamide. ¹H NMR (400 MHz, DMSO-d₆) δ 9.20-9.13 (m, 1H), 8.75-8.62 (m, 1H), 8.57 (s, 1H), 8.27 (d, J=1.8 Hz, 1H), 8.15 (dd, J=8.0, 1.8 Hz, 1H), 7.99-7.91 (m, 1H), 7.83 (s, 1H), 7.57 (d, J=8.0 Hz, 1H), 7.25 (s, 1H), 6.70 (s, 1H), 4.37-4.24 (m, 2H), 3.48-3.36 (m, 2H), 3.28 (s, OH), 2.60 (s, 3H), 2.17-2.09 (m, 2H), 1.95-1.86 (m, 2H), 1.24-1.16 (m, 3H); MS (ESI) m/z: 478.3 [M+1]+.

### Example 22

### 6-(2-chloro-4-(6-methylpyrazin-2-yl)phenyl)-2-(ethylamino)-8-(2-(4-(oxetan-3-yl)piperazin-1-yl)ethyl)pyrido[2,3-d]pyrimidin-7(8H)-one (I-23)

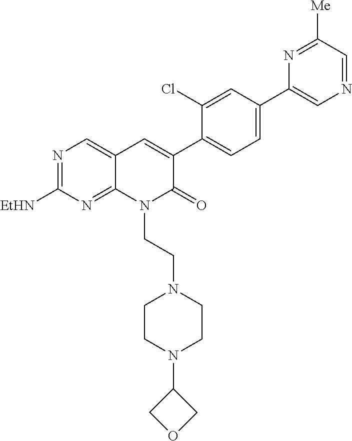

step 1: 6-[2-Chloro-4-(6-methylpyrazin-2-yl)phenyl]-2-methylsulfanyl-8-(2-piperazin-1-ylethyl)pyrido[2,3-d]pyrimidin-7-one was prepared using procedure analogous to that described in step 1-of Example 11 except in step 1, tert-butyl 4-(2-methylsulfonyloxyethyl)piperazine-1-carboxylate replaced tert-butyl N-(3-bromopropyl)carbamate. The crude product was used in the next step without further purification. MS (ESI) m/z: 508.2 [M+1]⁺.

step 2: To a stirred solution of 6-[2-chloro-4-(6-methylpyrazin-2-yl)phenyl]-2-methylsulfanyl-8-(2-piperazin-1-ylethyl)pyrido[2,3-d]pyrimidin-7-one (97 mg, 0.19 mmol) in DCE (9.6 mL) was added glacial HOAc (13.8 mg, 0.23 mmol) followed by 3-oxetanone (21.3 mg, 0.29 mmol). After 15 minutes, sodium triacetoxyborohydride (101 mg, 0.48 mmol) was added, and the reaction mixture was stirred at RT under N₂ for 4 h. The reaction was diluted with EtOAc. The organic layer was washed with sat. aq. NaHCO₃ solution, water, and brine, dried over Na₂SO₄, filtered, and concentrated under reduced pressure. The crude residue was purified by SiO₂ chromatography eluting with EtOAc/MeOH to give 6-[2-chloro-4-(6-methylpyrazin-2-yl)phenyl]-2-methylsulfanyl-8-[2-[4-(oxetan-3-yl)piperazin-1-yl]ethyl]pyrido[2,3-d]pyrimidin-7-one as an oil (78.6 mg, 73%). MS (ESI) m/z: 564.2 [M+1]⁺.

The oxidation of the sulfide to the corresponding sulfone and displacement with ethanamine were conducted in analogy to steps 2 and 3 of Example 20. ¹H NMR (400 MHz, DMSO-d₆) δ 9.15 (s, 1H), 8.65 (s, 1H), 8.57 (s, 1H), 8.27 (s, 1H), 8.17-8.13 (m, 1H), 7.96 (br s, 1H), 7.83 (d, J=3.8 Hz, 1H), 7.55 (dd, J=8.0, 2.7 Hz, 1H), 4.99 (t, J=5.8 Hz, 1H), 4.57-4.36 (m, 5H), 3.46-3.20 (m, 4H), 3.06-2.88 (m, 2H), 2.82-2.52 (m, 5H), 2.60 (s, 3H), 2.28-2.17 (m, 2H), 1.25-1.13 (m, 3H). MS (ESI) m/z: 561.2 [M+1]⁺.

### Example 23

### 8-(3-amino-2-hydroxy-propyl)-6-[2-chloro-4-(6-methylpyrazin-2-yl)phenyl]-2-(ethylamino)pyrido[2,3-d]pyrimidin-7-one (I-24)

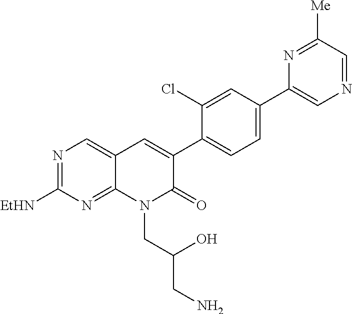

step 1: A mixture of 6-[2-chloro-4-(6-methylpyrazin-2-yl)phenyl]-2-methylsulfanyl-8H-pyrido[2,3-d]pyrimidin-7-one (491 mg, 1.24 mmol), cesium carbonate (492 mg, 1.51 mmol, 1.22 equiv.) and 2-(chloromethyl)oxirane (120 μL, 1.53 mmol, 1.23 equiv.) in DMF (5.0 ml) was stirred at ambient temperature for 48 h. The mixture was partitioned between water and EtOAc, and the separated organic phase washed with water and brine, dried over Na₂SO₄, filtered and concentrated in vacuo to afford a brown oil (568 mg). The crude residue was purified by automated flash chromatography on SiO₂ using a EtOAc/heptane gradient (10 to 100% EtOAc) to afford pure 6-[2-chloro-4-(6-methylpyrazin-2-yl)phenyl]-2-methylsulfanyl-8-(oxiran-2-ylmethyl)pyrido[2,3-d]pyrimidin-7-one (257 mg, 46%). ¹H NMR (400 MHz, CDCl₃) 8.84 (s, 1H), 8.69 (s, 1H), 8.45 (s, 1H), 8.20 (d, J=1.7 Hz, 1H), 7.98 (dd, J=8.0, 1.8 Hz, 1H), 7.74 (s, 1H), 7.52 (d, J=8.0 Hz, 1H), 4.87 (dd, J=13.3, 4.8 Hz, 1H), 4.58 (dd, J=13.3, 5.5 Hz, 1H), 3.43 (ddd, J=8.3, 5.2, 3.1 Hz, 1H), 2.82 (d, J=3.2 Hz, 2H), 2.67 (s, 3H), 2.65 (s, 3H). MS (ESI) m/z: 452/454 [M+1]⁺.

step 2: A mixture of 6-[2-chloro-4-(6-methylpyrazin-2-yl)phenyl]-2-methylsulfanyl-8-(oxiran-2-ylmethyl)pyrido[2,3-d]pyrimidin-7-one (151 mg, 0.334 mmol), sodium azide (44.2 mg, 0.680 mmol, 2.03 equiv.) and ammonium chloride (35.8 mg, 0.669 mmol, 2.00 equiv.) in DMF (5.0 ml) was heated to 50° C. for 22 h. The cooled mixture was partitioned between 2-methyltetrahydrofuran and water, and the separated organic phase washed with water and brine, and dried Na₂SO₄, filtered and concentrated in vacuo to afford 8-(3-azido-2-hydroxy-propyl)-6-[2-chloro-4-(6-methylpyrazin-2-yl)phenyl]-2-methylsulfanyl-pyrido[2,3-d]pyrimidin-7-one as a pale yellow solid (165 mg, 100%), used subsequently without further purification. ¹H NMR (400 MHz, DMSO-d₆) δ 9.18 (s, 1H), 8.95 (s, 1H), 8.58 (s, 1H), 8.31 (d, J=1.8 Hz, 1H), 8.20 (dd, J=8.0, 1.8 Hz, 1H), 8.09 (s, 1H), 7.59 (d, J=8.1 Hz, 1H), 5.49 (d, J=5.8 Hz, 1H), 4.58 (dd, J=12.8, 7.0 Hz, 1H), 4.36 (dd, J=12.9, 6.3 Hz, 1H), 4.20 (p, J=6.0 Hz, 1H), 2.65 (s, 3H), 2.61 (s, 3H); 2 protons obscured. MS (ESI) m/z: 495/497 [M+1]⁺.

step 3: A suspension of 8-(3-azido-2-hydroxy-propyl)-6-[2-chloro-4-(6-methylpyrazin-2-yl)phenyl]-2-methylsulfanyl-pyrido[2,3-d]pyrimidin-7-one (163 mg, 0.330 mmol) in THF (5.0 ml) was treated with MCPBA (105 mg, 0.467 mmol, 1.41 equiv.) at ambient temperature for 1 h. The mixture was diluted with 2-methyltetrahydrofuran and washed sequentially with water, saturated aqueous NaHCO₃ and brine, dried over Na₂SO₄, filtered and concentrated in vacuo to afford a mixture (˜1:1) of 8-(3-azido-2-hydroxy-propyl)-6-[2-chloro-4-(6-methylpyrazin-2-yl)phenyl]-2-methylsulfenyl-pyrido[2,3-d]pyrimidin-7-one and 8-(3-azido-2-hydroxy-propyl)-6-[2-chloro-4-(6-methylpyrazin-2-yl)phenyl]-2-methylsulfonyl-pyrido[2,3-d]pyrimidin-7-one as a pale yellow solid (190.4 mg), used subsequently without purification. MS (ESI) m/z: 511/513 [M+1]⁺ and 527/529 [M+1]⁺.

step 4: A mixture (1:1) of 8-(3-azido-2-hydroxy-propyl)-6-[2-chloro-4-(6-methylpyrazin-2-yl)phenyl]-2-methylsulfenyl-pyrido[2,3-d]pyrimidin-7-one and 8-(3-azido-2-hydroxy-propyl)-6-[2-chloro-4-(6-methylpyrazin-2-yl)phenyl]-2-methylsulfonyl-pyrido[2,3-d]pyrimidin-7-one (0.330 mmol) was suspended in a solution of ethylamine in THF (5.0 mL, 2.0 M, 10 mmol, 30 equiv.) at ambient temperature to give a pale yellow solution. After 16 h the mixture was diluted with 2-methyltetrahydrofuran, washed with saturated aqueous NaHCO₃ and brine, dried over Na₂SO₄, filtered and concentrated in vacuo to afford 8-(3-azido-2-hydroxy-propyl)-6-[2-chloro-4-(6-methylpyrazin-2-yl)phenyl]-2-(ethylamino)pyrido[2,3-d]pyrimidin-7-one as a pale yellow solid (161.0 mg, 99%), used subsequently without purification. MS (ESI) m/z: 492/494 [M+1]⁺.

step 5: A mixture of 8-(3-azido-2-hydroxy-propyl)-6-[2-chloro-4-(6-methylpyrazin-2-yl)phenyl]-2-(ethylamino)pyrido[2,3-d]pyrimidin-7-one (0.330 mmol), tris(2-carboxyethyl)phosphine hydrochloride (473 mg, 1.65 mmol, 5.00 equiv.), TEA (230 μL, 1.65 mmol, 5.00 equiv.) and water (500 μL) in THF (10 mL) was stirred at RT for 3 days. The mixture was treated with saturated aqueous NaHCO₃ and extracted twice into 2-methyltetrahydrofuran. The combined organic phases were washed with water and brine, dried over Na₂SO₄, filtered, and concentrated in vacuo to afford a yellow solid (119.1 mg, 77% crude yield for 3 steps). The residue was submitted to purification by reverse phase HPLC (C18, gradient of acetonitrile in water with 0.1% ammonium hydroxide) to afford 8-(3-amino-2-hydroxy-propyl)-6-[2-chloro-4-(6-methylpyrazin-2-yl)phenyl]-2-(ethylamino)pyrido[2,3-d]pyrimidin-7-one as a pale yellow solid (15.1 mg, 9.8%). ¹H NMR (400 MHz, DMSO-d₆) δ 9.15 (s, 1H), 8.72-8.62 (m, 1H), 8.57 (s, 1H), 8.27 (d, J=1.7 Hz, 1H), 8.15 (dd, J=8.0, 1.9 Hz, 1H), 8.0-7.7 (m, 1H), 7.84 (s, 1H), 7.55 (d, J=8.0 Hz, 1H), 4.9-4.6 (m, 1H), 4.5-4.2 (m, 2H), 4.3-3.8 (m, 1H), 3.42 (p, J=6.8 Hz, 2H), 2.60 (s, 3H), 1.3-1.16 (m, 3H); 4 protons obscured. ¹H NMR (300 MHz, DMSO-d6, 359K) δ 9.06 (s, 1H), 8.62 (s, 1H), 8.52 (s, 1H), 8.22 (d, J=1.4 Hz, 1H), 8.09 (dd, J=8.0, 1.7 Hz, 1H), 7.78 (s, 1H), 7.59-7.50 (m, 1H), 7.53 (d, J=7.8 Hz, 1H), 4.45-4.30 (m, 2H), 3.97-3.86 (m, 1H), 3.45 (p, J=6.5 Hz, 2H), 2.59 (s, 3H), 2.65-2.45 (m, 2H), 1.21 (t, J=7.1 Hz, 3H); 3 protons obscured. MS (ESI) m/z: 466.2/468.2 [M+1]⁺.

### Example 24

### 6-[2-chloro-4-(6-methylpyrazin-2-yl)phenyl]-2-(ethylamino)-8-[3-(ethylamino)-2-hydroxy-propyl]pyrido[2,3-d]pyrimidin-7-one (I-25)

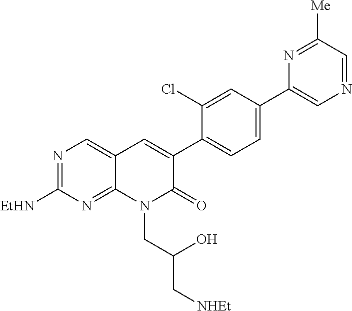

step 1: A solution of 6-[2-chloro-4-(6-methylpyrazin-2-yl)phenyl]-2-methylsulfanyl-8-(oxiran-2-ylmethyl)pyrido[2,3-d]pyrimidin-7-one (86.7 mg, 0.192 mmol) in DCM (2.0 ml) was treated MCPBA (51.0 mg, 0.228 mmol, 1.19 equiv.) at ambient temperature for 1 h. The mixture was concentrated in vacuo, the residue was redissolved in THF and concentrated to afford a mixture (˜2:1) of 6-[2-chloro-4-(6-methylpyrazin-2-yl)phenyl]-2-methylsulfonyl-8-(oxiran-2-ylmethyl)pyrido[2,3-d]pyrimidin-7-one and 6-[2-chloro-4-(6-methylpyrazin-2-yl)phenyl]-2-methylsulfenyl-8-(oxiran-2-ylmethyl)pyrido[2,3-d]pyrimidin-7-one as a yellow solid, used subsequently without purification. MS (ESI) m/z: 468/470 [M+1]⁺ and 484/486 [M+1]⁺.

step 2: The crude mixture of 6-[2-chloro-4-(6-methylpyrazin-2-yl)phenyl]-2-methylsulfonyl-8-(oxiran-2-ylmethyl)pyrido[2,3-d]pyrimidin-7-one and 6-[2-chloro-4-(6-methylpyrazin-2-yl)phenyl]-2-methylsulfenyl-8-(oxiran-2-ylmethyl)pyrido[2,3-d]pyrimidin-7-one (0.192 mmol) was suspended in a solution of ethylamine in THF (3.0 mL, 2.0 M, 6.0 mmol, 31 equiv.) at ambient temperature for 3 d, and then heated to 60° C. for 22 h. The cooled mixture was diluted with EtOAc and washed with saturated aqueous NaHCO₃ and brine, dried over Na₂SO₄, filtered and concentrated in vacuo to afford a yellow solid (82.9 mg). The crude residue was submitted to purification by reverse phase HPLC (C18, gradient of acetonitrile in water with 0.1% ammonium hydroxide) to afford 6-[2-chloro-4-(6-methylpyrazin-2-yl)phenyl]-2-(ethylamino)-8-[3-(ethylamino)-2-hydroxy-propyl]pyrido[2,3-d]pyrimidin-7-one as a cream-colored solid (26.6 mg, 28% for 2 steps). ¹H NMR (400 MHz, DMSO-d6) δ 9.15 (s, 1H), 8.71-8.61 (m, 1H), 8.57 (s, 1H), 8.27 (d, J=1.7 Hz, 1H), 8.15 (dd, J=8.1, 1.8 Hz, 1H), 7.97-7.88 (m, 1H), 7.83 (s, 1H), 7.55 (d, J=8.0 Hz, 1H), 4.77-4.70 (m, 1H), 4.48-4.38 (m, 1H), 4.36-4.26 (m, 1H), 4.11-4.01 (m, 1H), 3.42 (p, J=6.9 Hz, 2H), 2.60 (s, 3H), 1.20 (t, J=7.6 Hz, 3H), 0.97 (1, J=7.1 Hz, 3H); 5 protons obscured. MS (ESI) m/z: 494.2/496.2 [M+1]⁺.

### Example 25

### 6-[2-chloro-4-(6-methylpyrazin-2-yl)phenyl]-2-(ethylamino)-8-(2-morpholino-2-oxo-ethyl)pyrido[2,3-d]pyrimidin-7-one (I-26)

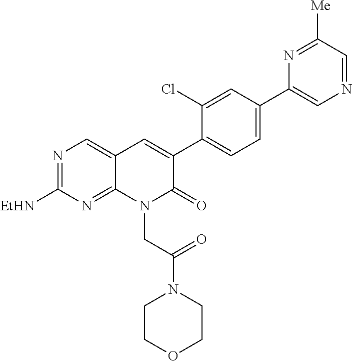

step 1: A suspension of 6-[2-chloro-4-(6-methylpyrazin-2-yl)phenyl]-2-methylsulfanyl-8H-pyrido[2,3-d]pyrimidin-7-one (173 mg, 0.438 mmol) and Cs₂CO₃ (184 mg, 0.565 mmol, 1.29 equiv.) in DMF (5.0 ml) was treated with 2-chloro-1-morpholino-ethanone (84.1 mg, 0.514 mmol, 1.17 equiv.) at RT for 16 h. A further portion of 2-chloro-1-morpholino-ethanone (80.3 mg, 0.491 mmol, 1.12 equiv.) was added and the mixture stirred RT for 3 d. The mixture was diluted with water and extracted twice with 2-methyltetrahydrofuran, and undissolved suspended solid recovered by filtration, washed with 2-methyltetrahydrofuran and dried in vacuo (86.6 mg). The combined organic phases were washed with brine, dried over Na₂SO₄, filtered and concentrated in vacuo to afford an orange-brown solid (164.8 mg). The solids were combined to afford 6-[2-chloro-4-(6-methylpyrazin-2-yl)phenyl]-2-methylsulfanyl-8-(2-morpholino-2-oxo-ethyl)pyrido[2,3-d]pyrimidin-7-one as an orange-brown solid (251 mg), used subsequently without purification. MS (ESI) m/z: 523/525 [M+1]⁺.

step 2: A suspension of crude 6-[2-chloro-4-(6-methylpyrazin-2-yl)phenyl]-2-methylsulfanyl-8-(2-morpholino-2-oxo-ethyl)pyrido[2,3-d]pyrimidin-7-one (0.438 mmol) and MCPBA (157.1 mg, 0.7010 mmol, 1.600 equiv.) in THF (10 ml) was stirred at RT for 18 h. The mixture was concentrated in vacuo to afford 6-[2-chloro-4-(6-methylpyrazin-2-yl)phenyl]-2-methylsulfonyl-8-(2-morpholino-2-oxo-ethyl)pyrido[2,3-d]pyrimidin-7-one as a yellow oil, used subsequently without purification. MS (ESI) m/z: 555/557 [M+1]⁺.

step 3: The residue from step 2 was treated with a solution of ethylamine in THF (5.0 ml, 2.0 M, 10 mmol, 23 equiv.) at RT for 2 h. The mixture was diluted with 2-methyltetrahydrofuran and washed with saturated aqueous NaHCO₃ and brine, dried over Na₂SO₄, filtered and concentrated in vacuo to afford a pale yellow solid (138 mg, 61% crude yield for 3 steps). The crude residue was submitted to purification by reverse phase HPLC (C18, gradient of acetonitrile in water with 0.1% ammonium hydroxide) to afford 6-[2-chloro-4-(6-methylpyrazin-2-yl)phenyl]-2-(ethylamino)-8-(2-morpholino-2-oxo-ethyl)pyrido[2,3-d]pyrimidin-7-one as a cream-colored solid (13.1 mg, 5.8%). ¹H NMR (400 MHz, DMSO-d6) δ 9.15 (s, 1H), 8.75-8.64 (m, 1H), 8.57 (s, 1H), 8.27 (d, J=1.8 Hz, 1H), 8.16 (dd, J=8.0, 1.9 Hz, 1H), 8.01-7.96 (m, 1H), 7.89 (s, 1H), 7.55 (d, J=8.0 Hz, 1H), 5.16 (s, 2H), 3.73-3.63 (m, 4H), 3.62-3.52 (m, 2H), 3.50-3.41 (m, 2H), 3.40-3.29 (m, 2H), 2.60 (s, 3H), 1.15 (t, J=7.1 Hz, 3H). MS (ESI) m/z: 520.2/522.2 [M+1]⁺.

### Example 26

### 8-(1-(2-Aminoacetyl)pyrrolidin-3-yl)-6-(2-chloro-4-(6-methylpyrazin-2-yl)phenyl)-2-(ethylamino)pyrido[2,3-d]pyrimidin-7(8H)-one (I-28)

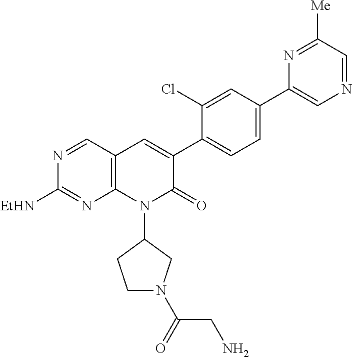

step 1: 6-[2-Chloro-4-(6-methylpyrazin-2-yl)phenyl]-2-methylsulfanyl-8H-pyrido[2,3-d]pyrimidin-7-one (1.19 g, 3.01 mmol), tert-butyl 3-methylsulfonyloxypyrrolidine-1-carboxylate (1.7 equiv., 5.11 mmol, 1360 mg) and cesium carbonate (2.5 equiv., 7.51 mmol, 2450 mg) were mixed in DMF (17.4 mL), and the reaction was stirred at 90° C. for 18 h. The mixture was diluted with EtOAc and 10% citric acid was added to adjust the pH to 9, and the layers were separated. The aqueous layer was extracted with EtOAc, and the organic layers were combined, washed with water and brine, dried (MgSO₄), filtered, and finally concentrated in vacuo. The crude residue was purified by SiO₂ chromatography eluting with DCM/EtOAc to afford 1307 mg (77% yield) off-white foam. LCMS and NMR analysis indicated a mixture of two regioisomers (i.e., tert-butyl 3-(6-(2-chloro-4-(6-methylpyrazin-2-yl)phenyl)-2-(methylthio)-7-oxopyrido[2,3-d]pyrimidin-8(7H)-yl)pyrrolidine-1-carboxylate and tert-butyl 3-((6-(2-chloro-4-(6-methylpyrazin-2-yl)phenyl)-2-(methylthio)pyrido[2,3-d]pyrimidin-7-yl)oxy)pyrrolidine-1-carboxylate). The mixture was used in the next step without further purification. MS (ESI) m/z: 565 [M+1]⁺.

step 2: The mixture from step 1 was dissolved in DCM (15 mL) and HOAc (1.05 equiv., 2.429 mmol, 0.1392 mL). MCPBA (1.05 equiv., 2.429 mmol, 544.3 mg) was added in one portion at 0° C., and the reaction was stirred at 0° C. for 1 h. The reaction mixture was evaporated, and the yellow gel was dissolved in a solution of ethylamine (2 mol/L) in THF (17.0 equiv., 39.32 mmol, 19.66 mL). The yellow solution was kept at 25° C. for 18 h. Na₂SO₃ (1 teaspoon) was added, and the suspension was stirred at 25° C. for 45 min. The mixture was diluted with EtOAc. After filtration and evaporation, the crude residue was purified by SiO₂ chromatography eluting with DCM/EtOAc to afford a mixture of tert-butyl 3-(6-(2-chloro-4-(6-methylpyrazin-2-yl)phenyl)-2-(ethylamino)-7-oxopyrido[2,3-d]pyrimidin-8(7H)-yl)pyrrolidine-1-carboxylate and tert-butyl 3-((6-(2-chloro-4-(6-methylpyrazin-2-yl)phenyl)-2-(ethylamino)pyrido[2,3-d]pyrimidin-7-yl)oxy)pyrrolidine-1-carboxylate (1.00 g, 77% yield overall) as off-white solid. The mixture was used without in the next step without further purification. MS (ESI) m/z: 562 [M+1]⁺.

step 3: The mixture obtained in step 2 (450 mg, 0.8006 mmol) was mixed with HCl (4 mol/L) in 1,4-dioxane (44.0 equiv., 35.23 mmol, 8.806 mL) and maintained at 25° C. for 4 h. The reaction mixture was diluted with EtOAc and NaHCO₃ (s, aq) to pH 9 and the layers separated. The aqueous layer was extracted with EtOAc. The organic layers were combined, washed with water and brine, dried (MgSO₄), filtered, and finally concentrated in vacuo. The product was obtained as a mixture of 6-(2-chloro-4-(6-methylpyrazin-2-yl)phenyl)-2-(ethylamino)-8-(pyrrolidin-3-yl)pyrido[2,3-d]pyrimidin-7(8H)-one and 6-(2-chloro-4-(6-methylpyrazin-2-yl)phenyl)-N-ethyl-7-(pyrrolidin-3-yloxy)pyrido[2,3-d]pyrimidin-2-amine (335 mg, 84% yield), which was used in the next step without further purification. MS (ESI) m/z: 462 [M+1]⁺.

step 4: Boc-Gly-OH (4.0 equiv., 0.8745 mmol, 153.2 mg) and HATU (4.0 equiv., 0.8745 mmol, 339.3 mg) were mixed in 1,4-dioxane (1.0 mL) at 25° C. for 20 min. The white suspension was transferred into a solution of 6-[2-chloro-4-(6-methylpyrazin-2-yl)phenyl]-N-ethyl-7-pyrrolidin-3-yloxy-pyrido[2,3-d]pyrimidin-2-amine and 6-[2-chloro-4-(6-methylpyrazin-2-yl)phenyl]-2-(ethylamino)-8-pyrrolidin-3-yl-pyrido[2,3-d]pyrimidin-7-one (101 mg, 0.2186 mmol) in 1,4-dioxane (1.0 mL) and DIPEA (5.0 equiv., 1.093 mmol, 0.191 mL). The reaction was kept at 25° C. for 4 h. The crude was diluted with EtOAc and the pH was adjusted to ˜9 by addition of 10% citric acid. The layers were separated, and the aqueous layer was extracted by EtOAc. The organic layers were combined, washed with water and brine, dried (MgSO₄), filtered, and finally concentrated in vacuo. The crude residue was purified by SiO₂ chromatography eluting with DCM/EtOAc to give the two isomers:

tert-butyl (2-(3-(6-(2-chloro-4-(6-methylpyrazin-2-yl)phenyl)-2-(ethylamino)-7-oxopyrido[2,3-d]pyrimidin-8(7H)-yl)pyrrolidin-1-yl)-2-oxoethyl)carbamate: 63 mg, 46% yield, MS (ESI) m/z: 619 [M+1]⁺.

tert-butyl (2-(3-((6-(2-chloro-4-(6-methylpyrazin-2-yl)phenyl)-2-(ethylamino)pyrido[2,3-d]pyrimidin-7-yl)oxy)pyrrolidin-1-yl)-2-oxoethyl)carbamate: 44 mg, 33% yield, MS (ESI) m/z: 619 [M+1]⁺.

step 5: tert-Butyl N-[2-[3-[6-[2-chloro-4-(6-methylpyrazin-2-yl)phenyl]-2-(ethylamino)-7-oxo-pyrido[2,3-d]pyrimidin-8-yl]pyrrolidin-1-yl]-2-oxo-ethyl]carbamate (63 mg, 0.1018 mmol) was mixed with HCl (4 mol/L) in 1,4-dioxane (41 equiv., 4.172 mmol, 1.043 mL), and the reaction was maintained at 40° C. for 4 h. The mixture was diluted with EtOAc and NaHCO₃ (s, aq) to pH 9, and the layers were separated. The aqueous layer was extracted with 10% MeOH/DCM. The organic layers were combined, washed with water and brine, dried (MgSO₄), filtered, and finally concentrated in vacuo. The crude product (53 mg) was purified by RP-HPLC to give the title compound 8-(1-(2-aminoacetyl)pyrrolidin-3-yl)-6-(2-chloro-4-(6-methylpyrazin-2-yl)phenyl)-2-(ethylamino)pyrido[2,3-d]pyrimidin-7(8H)-one (12 mg, 23% yield) as white powder. ¹H NMR (400 MHz, DMSO-d6) δ 9.16 (s, 1H), 8.66 (s, 1H), 8.57 (s, 1H), 8.27 (s, 1H), 8.15 (dd, J=8.0, 1.5 Hz, 1H), 8.08-7.94 (m, 1H), 7.85 (d, J=4.7 Hz, 1H), 7.57 (d, J=8.0 Hz, 1H), 6.43-6.08 (m, 2H), 4.07-3.88 (m, 1H), 3.88-3.61 (m, 2H), 3.57-3.38 (m, 2H), 2.88-2.69 (m, 1H), 2.60 (s, 3H), 2.30-2.01 (m, 2H), 1.46 (d, J=16.3 Hz, 1H), 1.15 (t, J=7.1 Hz, 3H). MS (ESI) m/z: 519 [M+1]⁺.

### Example 27

### 8-(1-(2-Aminoacetyl)piperidin-4-yl)-6-(2-chloro-4-(6-methylpyrazin-2-yl)phenyl)-2-(ethylamino)pyrido[2,3-d]pyrimidin-7(8H)-one (I-48)

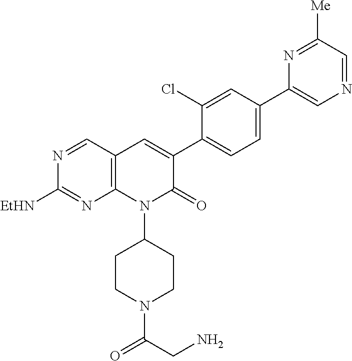

The title compound was prepared in a similar manner as described for I-28 described above. ¹H NMR (400 MHz, DMSO-d6) δ 9.14 (s, 1H), 8.63 (s, 1H), 8.57 (s, 1H), 8.26 (s, 1H), 8.14 (d, J=9.6 Hz, 1H), 7.95 (s, 1H), 7.81 (s, 1H), 7.55 (d, J=8.1 Hz, 1H), 5.62 (s, 2H), 4.58 (m, 2H), 3.91 (m, 2H), 3.60 (m, 3H), 2.60 (s, 3H), 1.66 (m, 3H), 1.17 (t, J=7.1 Hz, 3H). MS (ESI) m/z: 533 [M+1]⁺.

### Example 28

### 6-[2-chloro-4-(6-methylpyrazin-2-yl)phenyl]-2-(ethylamino)-8-[(3-methyl-3-azabicyclo[3.1.0]hexan-6-yl)methyl]pyrido[2,3-d]pyrimidin-7-one (I-128)

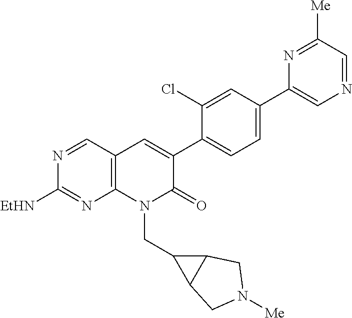

tert-Butyl 6-(((methylene-(p-tolyl)sulfinyl)oxy)methyl)-3-azabicyclo[3.1.0]hexane-3-carboxylate was prepared by reacting tert-butyl 6-(hydroxymethyl)-3-azabicyclo[3.1.0]hexane-3-carboxylate [CASRN 850808-43-4] and tosyl chloride using conventional procedures.

The title compound was prepared as described in Example 11 except in step 1 tert-butyl N-(3-bromopropyl)carbamate was replaced with tert-butyl 6-(hydroxymethyl)-3-azabicyclo[3.1.0]hexane-3-carboxylate and the reaction was run using NaH as the base in DMF at 85° C. After removal of the Boc group in step 4 (TFA) the nitrogen was methylated by reductive alkylation (i. formaldehyde, HOAc/MeOH; ii. NaBH(OAc)₃) to afford I-128. ¹H NMR (500 MHz, DMSO-d₆) δ 9.16 (s, 1H), 8.57 (s, 1H), 8.28 (d, J=1.6 Hz, 1H), 8.16 (dd, J=1.5, 8 Hz, 1H), 8.04-7.96 (m, 1H), 7.88-7.84 (m, 1H), 7.57 (d, J=8 Hz, 1H), 4.28-4.08 (m, 2H), 3.47-3.35 (m, 2H), 2.94 (br s, 2H), 2.60 (s, 3H), 2.36-2.18 (m, 3H), 1.75-1.59 (m, 3H), 1.27-1.12 (m, 4H); MS [M+H]⁺=502.1.

### Example 29

### 8-[2-(2-aminoethoxy)ethyl]-6-[2-ethyl-4-(6-methylpyrazin-2-yl)phenyl]-2-(methylamino)pyrido[2,3-d]pyrimidin-7-one (I-129)

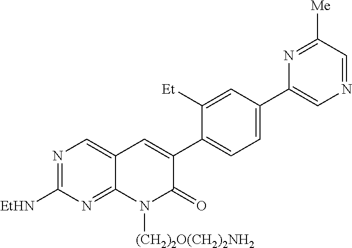

6-(2-ethyl-4-(6-methylpyrazin-2-yl)phenyl)-2-(methylthio)pyrido[2,3-d]pyrimidin-7(8H)-one was prepared by condensation of methyl 2-(2-ethyl-4-(6-methylpyrazin-2-yl)phenyl)acetate and 4-amino-2-(methylthio)pyrimidine-5-carbaldehyde (K₂CO₃, DMF, 85° C.).

The title compound was prepared by alkylation of 6-(2-ethyl-4-(6-methylpyrazin-2-yl)phenyl)-2-(methylthio)pyrido[2,3-d]pyrimidin-7(8H)-one with tert butyl [2 (2-bromoethoxy)ethyl]carbamate. Introduction of the methyl amine and deprotection were carried out using standard protocols to afford I-129. ¹H NMR (500 MHz, MeOH-d₄) δ 8.92 (s, 1H), 8.59 (s, 1H), 8.45 (s, 1H), 8.06 (d, J=1.5 Hz, 1H), 7.94 (dd, J=2.5, 8 Hz, 1H), 7.73 (s, 1H), 7.33 (d, J=8 Hz, 1H), 4.79-4.64 (m, 2H), 3.95-3.82 (m, 2H), 3.63 (t, J=5 Hz, 2H), 3.05 (s, 3H), 2.83 (t, J=5.5 Hz, 2H), 1.21 (t, J=5.5 Hz, 3H); MS [M+H]⁺=460.1.

### Example 30

### 6-[2-chloro-4-(6-methylpyrazin-2-yl)phenyl]-2-(ethylamino)-8-[2-(3-oxopiperazin-1-yl)ethyl]pyrido[2,3-d]pyrimidin-7-one (I-130)

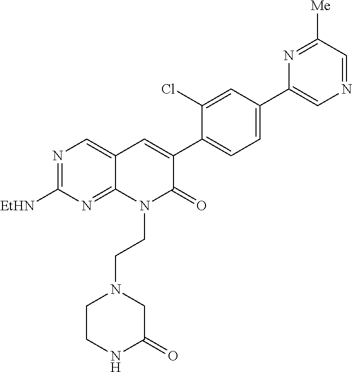

6-(2-chloro-4-(6-methylpyrazin-2-yl)phenyl)-2-(methylsulfinyl)pyrido[2,3-d]pyrimidin-7(8H)-one was condensed with N-ethyl-4-methoxy-benzenemethanamine [CASRN 22993-76-6] and the product alkylated with 1,2-dibromoethane to afford 8-(2-bromoethyl)-6-(2-chloro-4-(6-methylpyrazin-2-yl)phenyl)-2-(ethyl(4-methoxybenzyl)amino)pyrido[2,3-d]pyrimidin-7(8H)-one which was treated with piperazin-2-one to afford 6-(2-chloro-4-(6-methylpyrazin-2-yl)phenyl)-2-(ethyl(4-methoxybenzyl)amino)-8-(2-(3-oxopiperazin-1-yl)ethyl)pyrido[2,3-d]pyrimidin-7(8H)-one. Removal of the 4-methoxy-benzyl group with TFA (80° C., 18 h) afforded I-130. ¹H NMR (500 MHz, DMSO-d₆) δ 9.15 (s, 1H), 8.65 (s, 1H), 8.57 (s, 1H), 8.27 (d, J=1.5 Hz, 1H), 8.15 (dd, J=1.5, 8.0 Hz, 1H), 8.01-7.81 (m, 1H), 7.84 (s, 1H), 7.70 (s, 1H), 7.55 (d, J=8.0 Hz, 1H), 4.47-4.41 (m, 2H), 3.45-3.39 (m, 2H), 3.11-3.02 (m, 4H), 2.72-2.69 (m, 4H), 2.60 (s, 3H), 1.22-1.17 (m, 3H); MS [M+H]⁺=518.0

### Example 31

### 8-(3-aminopropyl)-6-[2-ethyl-4-(6-methylpyrazin-2-yl)phenyl]-2-(methylamino)pyrido[2,3-d]pyrimidin-7-one (I-131)

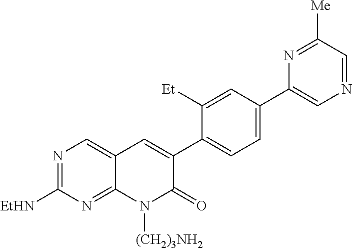

Condensation of 6-(2-ethyl-4-(6-methylpyrazin-2-yl)phenyl)-2-(methylthio)pyrido[2,3-d]pyrimidin-7(8H)-one and 3-(tert-butoxycarbonylamino)propyl 4-methylbenzenesulfonate (Cs₂CO₃, DMF, 80° C.) afforded tert-butyl 3-(6-(2-ethyl-4-(6-methylpyrazin-2-yl)phenyl)-2-(methylthio)-7-oxopyrido[2,3-d]pyrimidin-8(7H)-yl)propylcarbamate. Introduction of the methylamine and deprotection were carried out by standard protocols to afford the title compound. ¹H NMR (500 MHz, MeOH-d₄) δ 8.93 (s, 1H), 8.61 (s, 1H), 8.45 (s, 1H), 8.06 (d, J=1.5 Hz, 1H), 7.94 (dd, J=2, 7.5 Hz, 1H), 7.74 (s, 1H), 7.34 (d, J=7.5 Hz, 1H), 4.64-4.46 (m, 2H), 3.07 (s, 3H), 2.77-2.71 (m, 2H), 2.69-2.63 (m, 2H), 2.10-1.97 (m, 5H), 1.21 (t, J=7.5 Hz, 3H); MS [M+H]⁺=430.0.

8-(5-aminopentyl)-6-[2-chloro-4-(6-methylpyrazin-2-yl)phenyl]-2-(methylamino)pyrido[2,3-d]pyrimidin-7-one (I-138) was prepared analogously except 3-(tert-butoxycarbonylamino)propyl 4-methylbenze was replaced with 5-(tert-butoxycarbonylamino)pentyl 4-methylbenzenesulfonate and 6-(2-chloro-4-(6-methylpyrazin-2-yl)phenyl)-2-(methylthio)pyrido[2,3-d]pyrimidin-7(8H)-one was the starting material. ¹H NMR (400 MHz, DMSO-d₆) δ 9.15 (s, 1H), 8.67-8.62 (m, 1H), 8.57 (s, 1H), 8.27 (d, J=1.8 Hz, 1H), 8.15 (dd, J=8.0, 1.8 Hz, 1H), 7.90-7.80 (m, 2H), 7.56 (d, J=8.0 Hz, 1H), 6.68-6.52 (m, 1H), 4.43-4.17 (m, 3H), 2.92 (d, J=4.8 Hz, 3H), 2.60 (s, 3H), 2.55-2.51 (m, 2H), 1.75-1.59 (m, 2H), 1.49-1.27 (m, 4H).) δ 9.15 (s, 1H), 8.67-8.62 (m, 1H), 8.57 (s, 1H), 8.27 (d, J=1.8 Hz, 1H), 8.15 (dd, J=8.0, 1.8 Hz, 1H), 7.90-7.80 (m, 2H), 7.56 (d, J=8.0 Hz, 1H), 6.68-6.52 (m, 1H), 4.43-4.17 (m, 3H), 2.92 (d, J=4.8 Hz, 3H), 2.60 (s, 3H), 2.55-2.51 (m, 2H), 1.75-1.59 (m, 2H), 1.49-1.27 (m, 4H).

### Example 32

### 8-(3-hydroxypropyl)-2-(methylamino)-6-[2-methyl-4-(6-methyl-2-pyridyl)phenyl]pyrido[2,3-d]pyrimidin-7-one (I-132)

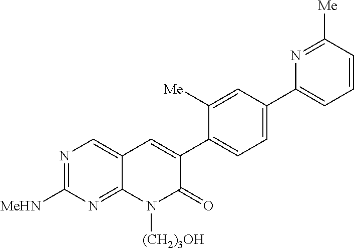

The title compound was prepared in analogy to the previous example except 6-(2-methyl-4-(6-methylpyridin-2-yl)phenyl)-2-(methylthio)pyrido[2,3-d]pyrimidin-7(8H)-one was alkylated with 3-bromo-propanol (Cs₂CO₃, DMF). Standard protocols were used to introduce the methylamine. ¹H NMR (400 MHz, DMSO-d6) δ 8.74-8.57 (m, 1H), 7.99-7.94 (m, 1H), 7.90 (dd, J=7.9, 1.9 Hz, 1H), 7.81-7.74 (m, 3H), 7.73 (s, 1H), 7.29 (d, J=7.9 Hz, 1H), 7.22 (dd, J=5.2, 3.3 Hz, 1H), 4.52-4.27 (m, 3H), 3.53-3.45 (m, 2H), 2.92 (d, J=4.7 Hz, 3H), 2.56 (s, 3H), 2.23 (s, 3H), 1.90-1.77 (m, 2H); MS [M+H]⁺=416.2.

### Example 33

### 8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one (I-133)

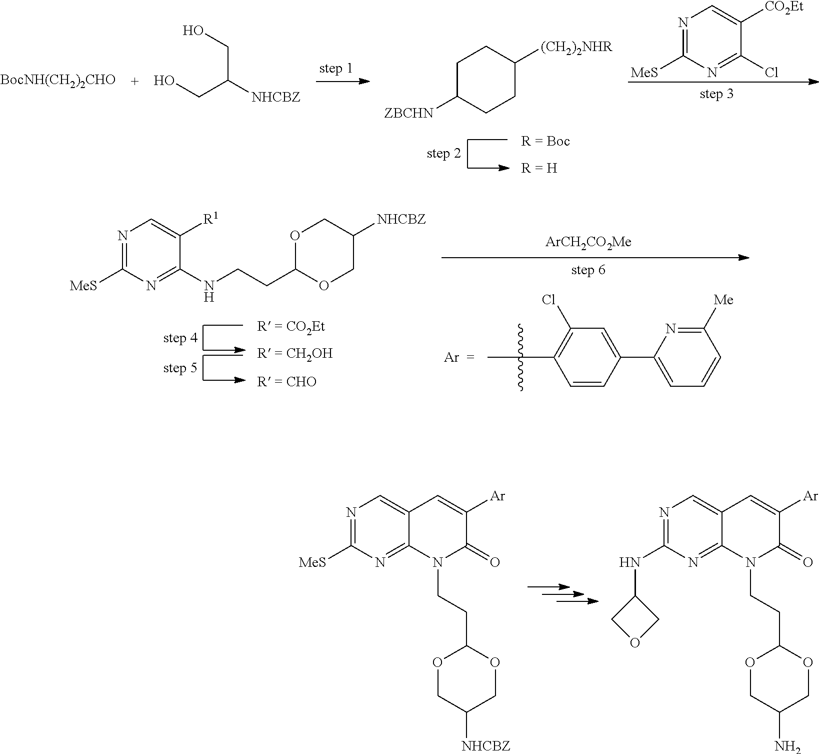

step 1: tert-Butyl N-(3-oxopropyl)carbamate (9.2 g, 53 mmol) was dissolved in toluene (250 mL) and chloroform (820 mL) then Na₂SO₄ decahydrate (12 g) and TsOH (1.4 g) were added followed by benzyl N-[2-hydroxy-1-(hydroxymethyl)ethyl]carbamate (9.2 g, 41 mmol). The mixture is stirred at RT overnight. The resulting mixture was neutralized with Na₂CO₃ and partitioned between water and DCM. The resulting aqueous layer was extracted with DCM and the combined organic phases were dried (Na₂SO₄), filtered and absorbed on CELITE®. The production was purified by SiO₂ chromatography eluting with a heptane/EtOAc gradient (0 to 100% EtOAc) to afford 11.73 g (76%) of tert-butyl N-[2-[5-(benzyloxycarbonylamino)-1,3-dioxan-2-yl]ethyl]carbamate as a white solid.

step 2: The product from step 1 (11.73 g) was dissolved in 4M HCl in dioxane and stirred at RT for 1.5 h. The solvent was removed and the resulting benzyl N-[2-(2-aminoethyl)-1,3-dioxan-5-yl]carbamate hydrochloride (8.973 g) was used without addition purification.

step 3: An oven-dried tube was charged with 4,5-bis-(diphenylphosphino)9,9-dimethylxanthene (0.149 g, 0.258 mmol), Pd(OAc)₂ (28.9 mg, 0.129 mmol), benzyl N-[2-(2-aminoethyl)-1,3-dioxan-5-yl]carbamate hydrochloride (1.6 g 5.16 mmol), ethyl 4-chloro-2-(methylthio)pyrimidine-5-carboxylate (1 g, 4.3 mmol) and Cs₂CO₃ (2.8 g), then dioxane is added and the mixture degassed with N₂ for ca. 3 min. The tube was then sealed with a Teflon screw cap and placed in a preheated oil bath at 110° C. for 3 h. After cooling of the reaction mixture the product was partitioned between H₂O and DCM. The aqueous phase was extracted with DCM and the combined extracts are washed with brine, dried (Na₂SO₄), filtered and absorbed on CELITE. The crude product was purified by SiO₂ chromatography to afford 1.76 g (87%) of ethyl 4-[2-[5-(benzyloxycarbonylamino)-1,3-dioxan-2-yl]ethylamino]-2-methylsulfanyl-pyrimidine-5-carboxylate.

step 4: A mixture of LiAlH₄ (1.05 eq LAH in 0.77M THF) in anhydrous THF (5.08 mL) cooled to 0° C. was treated dropwise with a solution of ethyl 4-[2-[5-(benzyloxycarbonylamino)-1,3-dioxan-2-yl]ethylamino]-2-methylsulfanyl-pyrimidine-5-carboxylate (1.76 g, 3.727 mmol) in anhydrous THF (8.39 mL). The reaction mixture was stirred for 15 min, and then 0.34 mL of water was added dropwise with caution. The mixture was stirred for 30 min. Then 0.16 mL of a 15% aqueous NaOH solution was added dropwise, followed by 0.48 mL of water. The resulting suspension was stirred for 17 h at RT and then filtered. The filtered residue was sequentially washed with three 25 mL portions of THF, and the combined filtrate and washings were diluted with ethyl acetate and heptane. The solvents were evaporated and the crude product redissolved in EtOAc and absorbed onto CELITE. The product was purified by SiO₂ chromatography eluting with a heptane/EtOAc gradient (20 to 100% EtOAc) to afford 735 mg of benzyl N-[2-[2-[[5-(hydroxymethyl)-2-methylsulfanyl-pyrimidin-4-yl]amino]ethyl]-1,3-dioxan-5-yl]carbamate and 226 mg of starting material

step 5: A stirred mixture of benzyl N-[2-[2-[[5-(hydroxymethyl)-2-methylsulfanyl-pyrimidin-4-yl]amino]ethyl]-1,3-dioxan-5-yl]carbamate (1.527 g, 3.514 mmol) and DCM was treated with MnO₂ (2.77 g). The resulting suspension was stirred for 24 h and then filtered through CELITE. The filter residue was washed with DCM, and the combined filtrate and washings were concentrated to give 1.493 g (98%) of benzyl N-[2-[2-[(5-formyl-2-methylsulfanyl-pyrimidin-4-yl)amino]ethyl]-1,3-dioxan-5-yl]carbamate as a white foam.

step 6: A mixture of benzyl N-[2-[2-[(5-formyl-2-methylsulfanyl-pyrimidin-4-yl)amino]ethyl]-1,3-dioxan-5-yl]carbamate (1.492 g, 3.450 mmol) and methyl 2-[2-chloro-4-(6-methylpyrazin-2-yl)phenyl]acetate (1.2 g, 3.622 mmol), K₂CO₃ (3 eq) in DMF (120 mL) was heated on the oil bath at 110 oC for 4 h. An additional 0.5 eq of methyl 2-[2-chloro-4-(6-methylpyrazin-2-yl)phenyl]acetate and K₂CO₃ (1.5 eq) were added and resulting mixture stirred at 85° C. for 3 h. At this time additional methyl 2-[2-chloro-4-(6-methylpyrazin-2-yl)phenyl]acetate and K₂CO₃ (1.5 equivalent) were added and the solution heated at 110° C. for 1 h.

After cooling to RT, water (200 mL) and heptane (ca. 50 mL) were added. The mixture was filtered and the solid washed with water and heptane to give benzyl N-[2-[2-[6-[2-chloro-4-(6-methylpyrazin-2-yl)phenyl]-2-methylsulfanyl-7-oxo-pyrido[2,3-d]pyrimidin-8-yl]ethyl]-1,3 dioxan-5-yl]carbamate as a light yellow solid (2.097 g, 92%).

Standard protocols were used to introduce the oxetan-3-ylamino and the CBZ group was removed with TFA to afford I-133. ¹H NMR (400 MHz, DMSO-d₆) δ 8.70 (s, 1H), 8.63 (s, 1H), 8.22 (d, J=1.7 Hz, 1H), 8.08 (dd, J=8.1, 1.8 Hz, 1H), 7.91-7.76 (m, 3H), 7.50 (d, J=8.0 Hz, 1H), 7.32-7.24 (m, 1H), 4.96 (s, 1H), 4.87 (m, 2H), 4.71 (t, J=4.7 Hz, 1H), 4.61 (t, J=6.2 Hz, 2H), 4.39 (dd, J=8.6, 6.2 Hz, 2H), 3.86 (d, J=11.0 Hz, 2H), 3.71 (d, J=10.9 Hz, 2H), 2.57 (s, 3H), 2.56 (m, 1H), 1.90-1.65 (m, 4H); MS [M+H]+=549.2.

The following were prepared analogously:

8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one (I-134) ¹H NMR (400 MHz, DMSO-d₆) δ 8.67 (m, 1H), 8.22 (d, J=1.7 Hz, 1H), 8.08 (dd, J=8.1, 1.8 Hz, 1H), 7.91-7.76 (m, 4H), 7.50 (d, J=8.0 Hz, 1H), 7.32-7.23 (m, 1H), 4.68 (I, J=4.8 Hz, 1H), 4.50-4.27 (m, 2H), 3.86-3.78 (m, 2H), 3.68 (dd, J=10.9, 1.7 Hz, 2H), 2.94 (d, J=4.8 Hz, 3H), 2.57 (s, 3H), 2.53 (m, 1H), 1.98-1.75 (s, 4H); [M+H]+=507.2

8-[2-(5-amino-1,3-dioxan-2-yl)ethyl]-6-[2-chloro-4-(6-methyl-2-pyridyl)phenyl]-2-(oxetan-3-ylamino)pyrido[2,3-d]pyrimidin-7-one (I-135) ¹H NMR (400 MHz, DMSO-d₆) δ 8.70 (s, 1H), 8.63 (m, 1H), 8.22 (d, J=1.7 Hz, 1H), 8.08 (dd, J=8.0, 1.8 Hz, 1H), 7.90-7.76 (m, 3H), 7.49 (d, J=8.0 Hz, 1H), 7.31-7.25 (m, 1H), 4.96 (m, 1H), 4.86 (m, 2H), 4.61 (t, J=6.2 Hz, 2H), 4.54 (t, J=4.9 Hz, 1H), 4.35 (dd, J=8.4, 6.5 Hz, 2H), 3.92 (dd, J=10.9, 4.9 Hz, 2H), 3.15 (t, J=10.6 Hz, 2H), 2.77 (dd, J=10.4, 5.2 Hz, 1H), 2.57 (s, 3H), 1.89 (m, 2H), 1.41 (m, 2H); [M+H]+=549.2.

8-[2-(5-amino-1,3-dioxan-2-yl)ethyl]-6-[2-chloro-4-(6-methyl-2-pyridyl)phenyl]-2-(methylamino)pyrido[2,3-d]pyrimidin-7-one (I-137) ¹H NMR (400 MHz, DMSO-d₆) δ 8.63 (m, 1H), 8.22 (d, J=1.7 Hz, 1H), 8.08 (dd, J=8.0, 1.8 Hz, 1H), 7.91-7.75 (m, 4H), 7.50 (d, J=8.0 Hz, 1H), 7.28 (d, J=7.4 Hz, 1H), 4.51 (t, J=5.0 Hz, 1H), 4.42 (m, 1H), 3.96-3.83 (m, 2H), 3.12 (t, J=10.5 Hz, 2H), 2.94 (d, J=4.8 Hz, 3H), 2.75 (m, 1H), 2.57 (s, 3H), 1.91 (m, 2H), 1.57 (m, 2H); [M+H]+=507.2

### Example 34

### 6-[2-chloro-4-(6-methyl-2-pyridyl)phenyl]-2-(ethylamino)-8-(2-morpholino-2-oxo-ethyl)pyrido[2,3-d]pyrimidin-7-one (I-136)

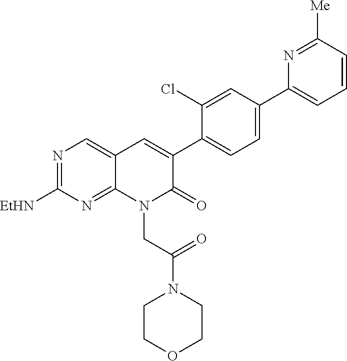

6-(2-Chloro-4-(6-methylpyridin-2-yl)phenyl)-2-(methylthio)pyrido[2,3-d]pyrimidin-7(8H)-one was alkylated with 2-chloro-1-(4-morpholinyl)-ethanone [CASRN 1440-61-5](tert-BuOK, DMF). Standard protocols were used to introduce the ethylamine. ¹H NMR (500 MHz, MeOH-d₄) δ 8.93 (s, 1H), 8.61 (s, 1H), 8.45 (s, 1H), 8.06 (d, J=1.5 Hz, 1H), 7.94 (dd, J=2, 7.5 Hz, 1H), 7.74 (s, 1H), 7.34 (d, J=7.5 Hz, 1H), 4.64-4.46 (m, 2H), 3.07 (s, 3H), 2.77-2.71 (m, 2H), 2.69-2.63 (m, 2H), 2.10-1.97 (m, 5H), 1.21 (t, J=7.5 Hz, 3H); MS [M+H]⁺=416.2.

### Example 35

### 6-[2-chloro-4-(6-methylpyrazin-2-yl)phenyl]-8-[2-(2-hydroxyethylamino)ethyl]-2-(methylamino)pyrido[2,3-d]pyrimidin-7-one (I-139)

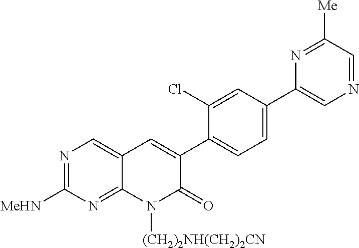

step 1: A suspension of 6-[2-chloro-4-(6-methylpyrazin-2-yl)phenyl]-2-methylsulfanyl-8H-pyrido-[2,3-d]pyrimidin-7-one (126 mg, 0.319 mmol) and Cs₂CO₃ (111 mg, 0.341 mmol, 1.07 equiv.) in DMF (5.0 ml) was treated with 3-(2-chloroethyl)-oxazolidin-2-one (62.5 mg, 0.418 mmol, 1.31 equiv.) at RT for 16 h and then heated to 60° C. for a further 14 h. The mixture was allowed to cool, diluted with water and extracted twice with EtOAc. The combined organic phases were washed with water and brine, dried (Na₂SO₄), filtered, and concentrated in vacuo to afford 3-(2-(6-(2-chloro-4-(6-methylpyrazin-2-yl)phenyl)-2-(methylthio)-7-oxopyrido[2,3-d]pyrimidin-8(7H)-yl)ethyl)oxazolidin-2-one as an orange crystalline solid (155 mg, 95%), used in the next step without purification. ¹H NMR (400 MHz, CDCl₃) δ 8.84 (s, 1H), 8.68 (s, 1H), 8.44 (s, 1H), 8.18 (d, J=1.7 Hz, 1H), 7.97 (dd, J=8.0, 1.7 Hz, 1H), 7.68 (s, 1H), 7.49 (d, J=8.0 Hz, 1H), 4.73 (t, J=5.6 Hz, 2H), 4.26 (dd, J=8.9, 7.1 Hz, 2H), 3.80-3.72 (m, 4H), 2.68 (s, 3H), 2.65 (s, 3H). MS (ESI) m/z: 509/511 [MH]⁺.

step 2: A solution of crude 3-(2-(6-(2-chloro-4-(6-methylpyrazin-2-yl)phenyl)-2-(methylthio)-7-oxopyrido[2,3-d]pyrimidin-8(7H)-yl)ethyl)oxazolidin-2-one (0.319 mmol) in DCM (10 ml) was treated with solid MCPBA (96.9 mg, 0.432 mmol, 1.35 equiv.) at RT for 1.5 h and concentrated in vacuo to afford a 1.4:1 mixture of 3-(2-(6-(2-chloro-4-(6-methylpyrazin-2-yl)phenyl)-2-(methylsulfinyl)-7-oxopyrido[2,3-d]pyrimidin-8(7H)-yl)ethyl)oxazolidin-2-one and 3-(2-(6-(2-chloro-4-(6-methylpyrazin-2-yl)phenyl)-2-(methylsulfonyl)-7-oxopyrido[2,3-d]pyrimidin-8(7H)-yl)ethyl)oxazolidin-2-one as a yellow foam. The crude residue was used without purification.

step 3: The crude mixture of sulfoxide and sulfone obtained in step 2 was treated with a solution of MeNH₂ in THF (1.6 mL, 2.0 M, 10 equiv.) at RT for 2.5 h. The mixture was treated with saturated aqueous NaHCO₃ and water, and extracted into DCM to which MeOH was added. The organic phase was washed with brine, dried (Na₂SO₄), filtered, and concentrated in vacuo to afford a yellow solid (161.8 mg, 103%). A portion of the crude residue (77.3 mg) was submitted to purification by reverse phase HPLC (C18, gradient of MeCN in water with 0.1% ammonium hydroxide) to afford 3-(2-(6-(2-chloro-4-(6-methylpyrazin-2-yl)phenyl)-2-(methylamino)-7-oxopyrido[2,3-d]pyrimidin-8(7H)-yl)ethyl)oxazolidin-2-one as a cream solid (48.8 mg, 61% for 3 steps). ¹H NMR (400 MHz, DMSO-d6) δ 9.15 (s, 1H), 8.75-8.63 (m, 1H), 8.57 (s, 1H), 8.27 (d, J=1.7 Hz, 1H), 8.15 (dd, J=8.0, 1.8 Hz, 1H), 7.93-7.67 (m, 1H), 7.84 (s, 1H), 7.52 (d, J=8.0 Hz, 1H), 4.57-4.40 (m, 2H), 4.14 (dd, J=9.0, 6.9 Hz, 2H), 3.78-3.54 (m, 4H), 2.94 (d, J=4.7 Hz, 3H), 2.60 (s, 3H). MS (ESI) m/z: 492.2/494.2 [MH]⁺.

step 4: Crude 3-(2-(6-(2-chloro-4-(6-methylpyrazin-2-yl)phenyl)-2-(methylamino)-7-oxopyrido-[2,3-d]pyrimidin-8(7H)-yl)ethyl)oxazolidin-2-one (84.5 mg) was suspended in MeOH (10 ml), treated with aqueous LiOH (1.6 mL, 1.0 M, 10 equiv.) and heated to reflux for 18 h. A second portion of aqueous LiOH was added (10 mL, 1.0 M, 63 equiv.) and heating under reflux continued for a further 22 h, followed by cooling and concentration in vacuo. The residue was treated with saturated aqueous NaHCO₃ and water and then extracted with DCM and 2-methyltetrahydrofuran. The combined organic phases were washed with brine, dried (Na₂SO₄), filtered, and concentrated in vacuo to afford a pale yellow solid (79.6 mg) which was purified by reverse phase HPLC (C18, gradient of MeCN in water with 0.1% ammonium hydroxide) to afford 6-(2-chloro-4-(6-methylpyrazin-2-yl)phenyl)-8-(2-((2-hydroxyethyl)amino)ethyl)-2-(methylamino)pyrido[2,3-d]pyrimidin-7(8H)-one as a white solid (35.0 mg, 47%). ¹H NMR (400 MHz, DMSO-d6) δ 9.16 (s, 1H), 8.75-8.61 (m, 1H), 8.57 (s, 1H), 8.28 (d, J=1.8 Hz, 1H), 8.16 (dd, J=8.0, 1.8 Hz, 1H), 7.92-7.72 (m, 1H), 7.84 (s, 1H), 7.56 (d, J=8.0 Hz, 1H), 4.43 (q, J=7.8, 6.5 Hz, 2H), 4.38-4.29 (m, 1H), 3.42 (q, J=5.6 Hz, 2H), 2.92 (d, J=4.6 Hz, 3H), 2.87 (t, J=6.9 Hz, 2H), 2.84-2.77 (m, 1H), 2.70-2.61 (m, 2H), 2.60 (s, 3H); MS (ESI) m/z: 466.2/468.2 [MH]⁺.

### Example 36

### 8-(3-aminopropyl)-6-[2-chloro-4-(2-methylthiazol-4-yl)phenyl]-2-(methylamino)pyrido[2,3-d]pyrimidin-7-one (I-140)

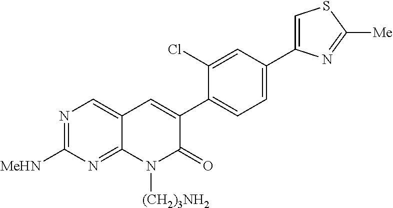

step 1: A mixture of tert-butyl 3-(6-(4-bromo-2-chlorophenyl)-2-(methylthio)-7-oxopyrido[2,3-d]pyrimidin-8(7H)-yl)propylcarbamate (216 mg, 0.4 mmol), 4,4,4′,4′,5,5,5′,5′-octamethyl-2,2′-bis(1,3,2-dioxaborolane) (305 mg, 1.2 mmol), Pd(dppf)Cl₂ (59 mg, 0.08 mmol) and KOAc (79 mg, 0.8 mmol) in dioxane (5 mL) was stirred at 80° C. under N₂ overnight. The mixture was filtered through a CELITE® pad and washed by EtOAc. The filtrate was concentrated under reduced pressure to afford 4-(8-(3-(tert-butoxycarbonylamino)propyl)-2-(methylthio)-7-oxo-7,8-dihydropyrido[2,3-d]pyrimidin-6-yl)-3-chlorophenylboronic acid as black oil (688 mg, crude) which was used in the next step without further purification.

step 2: A mixture of 4-(8-(3-(tert-butoxycarbonylamino)propyl)-2-(methylthio)-7-oxo-7,8-dihydropyrido[2,3-d]pyrimidin-6-yl)-3-chlorophenylboronic acid (688 mg, crude), 4-bromo-2-methylthiazole (254 mg, 1.4 mmol), Pd(PPh₃)₄ (71 mg, 0.061 mmol) and Na₂CO₃ (0.5 M in water, 4 mL) in MeCN (4 mL) was stirred at 90° C. under N₂ overnight. After cooling to RT, the reaction mixture was diluted with EtOAc (200 mL) and washed with aq. NaCl solution (100 mL). The organic layer was dried (MgSO₄) and concentrated under reduced pressure. The residue was purified by SiO₂ chromatography eluting with a PE/DCM/EtOAc gradient (3:3:2 to 1:1:2) to afford tert-butyl 3-(6-(2-chloro-4-(2-methylthiazol-4-yl)phenyl)-2-(methylthio)-7-oxopyrido[2,3-d]pyrimidin-8(7H)-yl)propylcarbamate as yellow oil (137 mg, 63% from two steps). LCMS (ESI): m/z=503.1 [M−55]⁺.

Standard protocols were used to introduce the methylamine and remove the Boc protecting group (TFA). ¹H NMR (500 MHz, DMSO-d₆) δ 8.72 (s, 1H), 8.13 (s, 1H), 8.08 (d, J=1.4 Hz, 1H), 7.95 (dd, J=1.5, 8.0 Hz, 1H), 7.91-7.84 (m, 1H), 7.88 (s, 1H), 7.83 (s, 1H), 7.46 (d, J=8.0 Hz, 1H), 4.35 (d, J=41.2 Hz, 2H), 2.93 (d, J=4.4 Hz, 3H), 2.74 (s, 3H), 2.61 (s, 2H), 1.87-1.72 (m, 2H); MS [M+H]⁺=441.0.

### Example 37

### 6-[2-chloro-4-(6-methyl-2-pyridyl)phenyl]-2-(methylamino)-8-(2-piperazin-1-ylsulfonylethyl)pyrido[2,3-d]pyrimidin-7-one (I-141)

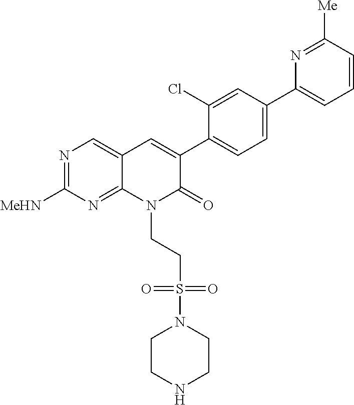 [SMILES: CNc1ncc2cc(-c3ccc(-c4cccc(C)n4)cc3Cl)c(=O)n(CCS(=O)(=O)N3CCNCC3)c2n1]

tert-Butyl 4-[(2-aminoethyl)sulfonyl]-1-piperazinecarboxylate [CASRN 917562-08-4] was condensed with 4-chloro-2-(methylthio)pyrimidine-5-carbaldehyde (TEA, THF, IPA H₂O, 0° C.) to afford tert-butyl 4-(2-(5-formyl-2-(methylthio)pyrimidin-4-ylamino)ethylsulfonyl)piperazine-1-carboxylate which was cyclized with methyl 2-[2-chloro-4-(6-methylpyridin-2-yl)phenyl]acetate (K₂CO₃, DMF, 100° C.) to afford 6-(2-chloro-4-(6-methylpyridin-2-yl)phenyl)-2-(methylthio)-8-(2-(piperazin-1-ylsulfonyl)ethyl)pyrido[2,3-d]pyrimidin-7(8H)-one. Standard protocols were used to introduce the methylamine and remove the Boc protecting group (TFA). ¹H NMR (400 MHz, DMSO-d₆) δ 8.67 (m, 1H), 8.23 (d, J=1.7 Hz, 1H), 8.09 (dd, J=8.1, 1.8 Hz, 1H), 7.94 (d, J=5.6 Hz, 1H), 7.87 (d, J=5.9 Hz, 2H), 7.81 (t, J=7.7 Hz, 1H), 7.51 (d, J=8.0 Hz, 1H), 7.33-7.24 (m, 1H), 4.74-4.54 (m, 2H), 3.43 (t, J=7.7 Hz, 2H), 3.17 (s, 1H), 3.09 (m, 3H), 2.95 (d, J=4.9 Hz, 3H), 2.70 (m, 4H), 2.57 (s, 3H), 2.35 (m, 1H); MS [M+H]⁺=554.2.

### Example 38

### 2-[6-[2-chloro-4-(6-methylpyrazin-2-yl)phenyl]-2-(methylamino)-7-oxo-pyrido[2,3-d]pyrimidin-8-yl]-N-[(3S)-pyrrolidin-3-yl]acetamide (I-142)

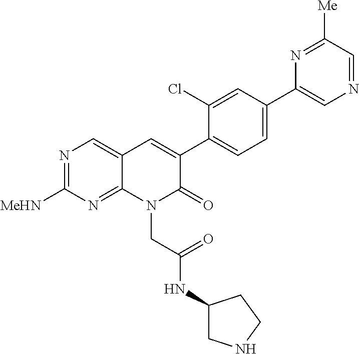 [SMILES: CNc1ncc2cc(-c3ccc(-c4cncc(C)n4)cc3Cl)c(=O)n(CC(=O)N[C@H]3CCNC3)c2n1]

6-(2-Chloro-4-(6-methylpyrazin-2-yl)phenyl)-2-(methylthio)pyrido[2,3-d]pyrimidin-7(8H)-one was alkylated with ethyl chloroacetete (Cs₂CO₃, DMF, RT 64 h). Introduction of the methyl amine was accomplished with standard protocols. After hydrolysis of the ester (LiOH, MeOH, reflux), the acid was condensed with tert-butyl (3S)-3-amino-1-pyrrolidinecarboxylate [CASRN147081-44-5](HATU, DIPEA, DMF, RT) and the Boc protecting group removed (TFA) to afford I-142. ¹H NMR (400 MHz, DMSO-d₆) 6-(2-chloro-4-(6-methylpyrazin-2-yl)phenyl)-2-(methylthio)pyrido[2,3-d]pyrimidin-7(8H)-one. ¹H NMR (400 MHz, DMSO-d₆) δ 9.15 (s, 1H), 8.78-8.63 (m, 1H), 8.57 (s, 1H), 8.27 (d, J=1.8 Hz, 1H), 8.24 (d, J=6.9 Hz, 1H), 8.15 (dd, J=8.0, 1.8 Hz, 1H), 7.89 (s, 1H), 7.85 (d, J=4.4 Hz, 1H), 7.55 (d, J=8.0 Hz, 1H), 4.93-4.81 (m, 2H), 4.12-4.02 (m, 1H), 2.87 (d, J=4.8 Hz, 3H), 2.94-2.79 (m, 2H), 2.77-2.64 (m, 1H), 2.55-2.48 (m, 3H), 1.95-1.81 (m, 1H), 1.56-1.44 (m, 1H). δ 9.15 (s, 1H), 8.78-8.63 (m, 1H), 8.57 (s, 1H), 8.27 (d, J=1.8 Hz, 1H), 8.24 (d, J=6.9 Hz, 1H), 8.15 (dd, J=8.0, 1.8 Hz, 1H), 7.89 (s, 1H), 7.85 (d, J=4.4 Hz, 1H), 7.55 (d, J=8.0 Hz, 1H), 4.93-4.81 (m, 2H), 4.12-4.02 (m, 1H), 2.87 (d, J=4.8 Hz, 3H), 2.94-2.79 (m, 2H), 2.77-2.64 (m, 1H), 2.55-2.48 (m, 3H), 1.95-1.81 (m, 1H), 1.56-1.44 (m, 1H); MS [M+H]⁺=505.2.

2-[6-[2-Chloro-4-(6-methylpyrazin-2-yl)phenyl]-2-(methylamino)-7-oxo-pyrido[2,3-d]pyrimidin-8-yl]-N-[(3R)-pyrrolidin-3-yl]acetamide (I-151) was prepared in accord with the procedure for I-142 except tert-butyl (3S)-3-amino-1-pyrrolidinecarboxylate [CASRN 147081-44-5] was replaced with tert-butyl (3R)-3-amino-1-pyrrolidinecarboxylate [CASRN 147081-49-0]. ¹H NMR (400 MHz, DMSO-d₆) δ 9.15 (s, 1H), 8.77-8.63 (m, 1H), 8.57 (s, 1H), 8.27 (d, J=1.8 Hz, 1H), 8.26-8.11 (m, 1H), 8.15 (dd, J=8.0, 1.8 Hz, 1H), 7.89 (s, 1H), 7.88-7.82 (m, 1H), 7.55 (d, J=8.1 Hz, 1H), 4.94-4.82 (m, 2H), 4.12-4.02 (m, 1H), 2.94-2.65 (m, 6H), 2.60 (s, 3H), 1.94-1.81 (m, 1H), 1.57-1.45 (m, 1H); other signals obscured; MS [M+H]⁺=505.2.

8-[2-[(3S)-3-Aminopyrrolidin-1-yl]-2-oxo-ethyl]-6-[2-chloro-4-(6-methylpyrazin-2-yl)phenyl]-2-(methylamino)pyrido[2,3-d]pyrimidin-7-one (I-143) was prepared analogously except tert-butyl (3S)-3-amino-1-pyrrolidinecarboxylate was replaced with tert-butyl N-(3S)-3-pyrrolidinylcarbamate [CASRN 122536-76-9]. ¹H NMR (400 MHz, DMSO-d₆) δ 9.15 (s, 1H), 8.77-8.63 (m, 1H), 8.57 (s, 1H), 8.28 (d, J=1.8 Hz, 1H), 8.16 (dd, J=8.1, 1.8 Hz, 1H), 7.90 (s, 1H), 7.88-7.67 (m, 1H), 7.55 (d, J=8.0 Hz, 1H), 5.12-4.92 (m, 2H), 3.79-2.99 (m, 4H), 2.94-2.80 (m, 3H), 2.60 (s, 3H), 2.08-1.51 (m, 4H); MS [M+H]⁺=505.2.

8-[2-[(3R)-3-Aminopyrrolidin-1-yl]-2-oxo-ethyl]-6-[2-chloro-4-(6-methylpyrazin-2-yl)phenyl]-2-(methylamino)pyrido[2,3-d]pyrimidin-7-one (I-152) was prepared in accord with the procedure for I-143 except tert-butyl N-(3S)-3-pyrrolidinylcarbamate was replaced with tert-butyl N-(3R)-3-pyrrolidinylcarbamate [CASRN 122536-77-09]. ¹H NMR (400 MHz, DMSO-d₆) δ 9.15 (s, 1H), 8.78-8.63 (m, 1H), 8.57 (s, 1H), 8.28 (d, J=1.7 Hz, 1H), 8.16 (dd, J=8.0, 1.8 Hz, 1H), 7.90 (s, 1H), 7.89-7.82 (m, 1H), 7.56 (d, J=7.9 Hz, 1H), 5.08-4.92 (m, 2H), 3.79-3.34 (m, 4H), 3.06-2.80 (m, 4H), 2.60 (s, 3H), 2.09-1.96 (m, 1H), 1.95-1.85 (m, 1H), 1.79-1.68 (m, 1H), 1.61-1.52 (m, 1H); MS [M+H]⁺=505.2.

8-[2-[(2R)-2-(Aminomethyl)pyrrolidin-1-yl]-2-oxo-ethyl]-6-[2-chloro-4-(6-methylpyrazin-2-yl)phenyl]-2-(methylamino)pyrido[2,3-d]pyrimidin-7-one (I-153) was prepared analogously except 2-(6-(2-chloro-4-(6-methylpyrazin-2-yl)phenyl)-2-(methylamino)-7-oxopyrido[2,3-d]pyrimidin-8(7H)-yl)acetic acid was condensed with (R)-tert-butyl pyrrolidin-2-ylmethylcarbamate [CASRN 719999-54-9]. ¹H NMR (400 MHz, DMSO-d₆) δ 9.15 (s, 1H), 8.78-8.64 (m, 1H), 8.57 (s, 1H), 8.27 (d, J=1.8 Hz, 1H), 8.16 (dd, J=8.0, 1.8 Hz, 1H), 7.90 (d, J=1.2 Hz, 1H), 7.89-7.83 (m, 1H), 7.56 (d, J=8.0 Hz, 1H), 5.26-4.99 (m, 2H), 4.10-3.59 (m, 3H), 2.96-2.80 (m, 1H), 2.84 (d, J=4.6 Hz, 3H), 2.60 (s, 3H), 2.43-2.23 (m, 4H); other signals obscured; MS [M+H]⁺=519.2.

8-[2-[(2S)-2-(Aminomethyl)pyrrolidin-1-yl]-2-oxo-ethyl]-6-[2-chloro-4-(6-methylpyrazin-2-yl)phenyl]-2-(methylamino)pyrido[2,3-d]pyrimidin-7-one (I-154) was prepared in analogy with I-153 except (S)-tert-butyl pyrrolidin-2-ylmethylcarbamate [CASRN 141774-70-1] was used ¹H NMR (400 MHz, DMSO-d₆) δ 9.15 (s, 1H), 8.78-8.63 (m, 1H), 8.57 (s, 1H), 8.27 (d, J=1.7 Hz, 1H), 8.16 (dd, J=8.1, 1.8 Hz, 1H), 7.90 (d, J=1.1 Hz, 1H), 7.89-7.84 (m, 1H), 7.55 (d, J=8.1 Hz, 1H), 5.28-4.99 (m, 2H), 4.09-3.59 (m, 3H), 2.94-2.80 (m, 4H), 2.60 (s, 3H), 2.07-1.73 (m, 4H); MS [M+H]⁺=519.2.

6-[2-Chloro-4-(6-methylpyrazin-2-yl)phenyl]-8-[2-[(1S,4S)-2 5-diazabicyclo[2.2.1]heptan-2-yl]-2-oxo-ethyl]-2-(methylamino)pyrido[2,3-d]pyrimidin-7-one (I-155) was prepared in analogy with I-153 except (1S,4S)-tert-butyl 2,5-diazabicyclo[2.2.1]heptane-2-carboxylate [CASRN 113451-59-5] was used. ¹H NMR (400 MHz, DMSO-d₆) δ 9.15 (s, 1H), 8.77-8.63 (m, 1H), 8.57 (s, 1H), 8.27 (d, J=1.7 Hz, 1H), 8.19-8.12 (m, 1H), 7.92-7.83 (m, 2H), 7.55 (dd, J=8.0, 2.8 Hz, 1H), 5.30-4.46 (m, 3H), 3.77-3.44 (m, 2H), 3.28-3.13 (m, 1H), 2.98-2.69 (m, 5H), 2.60 (s, 3H), 1.77-1.54 (m, 2H); MS [M+H]⁺=517.2.

N-(2-aminoethyl)-2-[6-[2-chloro-4-(6-methylpyrazin-2-yl)phenyl]-2-(methylamino)-7-oxo-pyrido[2,3-d]pyrimidin-8-yl]acetamide (I-156) was prepared in analogy with I-153 except tert-butyl N-(2-aminoethyl)carbamate [CASRN 57260-73-8). ¹H (400 MHz, DMSO-d₆) δ 9.15 (s, 1H), 8.77-8.63 (m, 1H), 8.57 (s, 1H), 8.27 (d, J=1.8 Hz, 1H), 8.15 (dd, J=8.1, 1.8 Hz, 1H), 8.14-8.01 (m, 1H), 7.89 (s, 1H), 7.88-7.65 (m, 1H), 7.55 (d, J=8.0 Hz, 1H), 4.94-4.83 (m, 2H), 3.11-3.03 (m, 2H), 2.93-2.83 (m, 3H), 2.60 (s, 3H), 2.58 (t, J=6.3 Hz, 2H); NH obscured; MS [M+H]⁺=479.2.

6-[2-chloro-4-(6-methylpyrazin-2-yl)phenyl]-8-[2-(3-hydroxypyrrolidin-1-yl)-2-oxo-ethyl]-2-(methylamino)pyrido[2,3-d]pyrimidin-7-one (I-157) was prepared analogously 2-(6-(2-chloro-4-(6-methylpyrazin-2-yl)phenyl)-2-(methylamino)-7-oxopyrido[2,3-d]pyrimidin-8(7H)-yl)acetic acid was condensed with 3-hydroxypyrrolidine (HATU, DIPEA, DMF, RT, 45 min). ¹H NMR (400 MHz, DMSO-d₆) δ 9.15 (s, 1H), 8.78-8.63 (m, 1H), 8.57 (s, 1H), 8.27 (d, J=1.8 Hz, 1H), 8.15 (dd, J=8.0, 1.8 Hz, 1H), 7.90 (s, 1H), 7.89-7.66 (m, 1H), 7.56 (d, J=8.0 Hz, 1H), 5.15-4.83 (m, 3H), 4.44-4.23 (m, 1H), 3.79-3.63 (m, 2H), 3.53-3.24 (m, 2H), 2.95-2.79 (m, 3H), 2.60 (s, 3H), 2.10-1.72 (m, 2H); MS [M+H]⁺=506.2.

### Example 39

### 8-[(5-amino-1,3-dioxan-2-yl)methyl]-6-[2-chloro-4-(6-methylpyrazin-2-yl)phenyl]-2-(ethylamino)pyrido[2,3-d]pyrimidin-7-one (I-144)

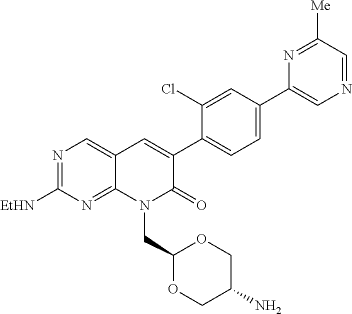

The title compound was prepared in accord with Example 38 except 6-(2-chloro-4-(6-methylpyrazin-2-yl)phenyl)-2-(methylthio)pyrido[2,3-d]pyrimidin-7(8H)-one was alkylated with anti-2-(2-(bromomethyl)-1,3-dioxan-5-yl)isoindoline-1,3-dione. Incorporation of the ethylamine moiety was carried out using standard protocols. Deprotection of the phthalimide was accomplished in the last step (MeNH₂, MeOH) to afford I-144. ¹H NMR (400 MHz, DMSO-d₆): δ 9.16 (s, 1H), 8.64 (s, 1H), 8.57 (s, 1H), 8.27 (d, J=2 Hz, 1H), 8.16 (dd, J=2.0, 8.0 Hz, 1H), 7.85 (s, 1H), 7.56 (d, J=7.5 Hz, 1H), 4.92 (t, J=5.5 Hz, 1H), 4.49-4.40 (m, 2H), 3.93-3.89 (m, 2H), 3.44-3.36 (m, 2H), 3.12 (t, J=10 Hz, 2H), 2.83-2.73 (m, 1H), 2.60 (s, 3H), 1.23 (t, J=7.5 Hz, 3H); MS [M+H]⁺=807.9.

8-[(5-Amino-1,3-dioxan-2-yl)methyl]-6-[2-chloro-4-(6-methylpyrazin-2-yl)phenyl]-2-(methylamino)pyrido[2,3-d]pyrimidin-7-one (I-145) was prepared analogously except a ethyl amine was replaced with methyl amine. ¹H NMR (500 MHz, DMSO-d₆): δ 9.17 (s, 1H), 8.65 (s, 1H), 8.58 (s, 1H), 8.28 (d, J=1.5 Hz, 1H), 8.16 (dd, J=1.5, 8.0 Hz, 1H), 7.86 (s, 1H), 7.57 (d, J=8.5 Hz, 1H), 4.99 (t, J=5.5 Hz, 1H), 4.50-4.42 (m, 2H), 3.94-3.90 (m, 2H), 3.12 (t, J=10 Hz, 2H), 2.92 (d, J=4.5 Hz, 3H), 2.82-2.73 (m, 1H), 2.60 (s, 3H); MS [M+H]⁺=494.

6-[2-Chloro-4-(6-methylpyrazin-2-yl)phenyl]-2-(methylamino)-8-[[5-(methylamino)-1,3-dioxan-2-yl]methyl]pyrido[2,3-d]pyrimidin-7-one (I-178) was prepared using similar procedures except tert-butyl (2r,5r)-2-((6-(2-chloro-4-(6-methylpyrazin-2-yl)phenyl)-2-(methylthio)-7-oxopyrido[2,3-d]pyrimidin-8(7H)-yl)methyl)-1,3-dioxan-5-ylcarbamate was N-methylated (NaH, MeI) and deprotected (TFA) prior to introduction of the methylamine on the pyrimidine ring which was accomplished using standard procedures. ¹H NMR (500 MHz, DMSO-d₆) δ 9.17 (s, 1H), 8.64 (s, 1H), 8.57 (s, 1H), 8.28 (d, J=2.5 Hz, 1H), 8.16 (dd, J=2.0, 10.0 Hz, 1H), 7.94-7.92 (m, 1H), 7.86 (s, 1H), 7.56 (d, J=10.0 Hz, 1H), 5.01-4.98 (m, 1H), 4.49 (d, J=5.5 Hz, 1H), 4.43 (d, J=5.0 Hz, 1H), 4.09 (dd, J=5.5, 13.5 Hz, 2H), 3.15 (t, J=13.0 Hz, 4H), 2.91 (d, J=6.5 Hz, 3H), 2.68-2.67 (m, 1H), 2.60 (s, 3H), 2.24 (s, 3H), 1.49 (s, 1H); MS [M+H]⁺=508.0.

2-amino-8-[(5-amino-1,3-dioxan-2-yl)methyl]-6-[2-chloro-4-(6-methylpyrazin-2-yl)phenyl]pyrido[2,3-d]pyrimidin-7-one (I-163) was prepared in accord with the procedure used for I-144 except ammonia was used in place of methyl amine. ¹H NMR (500 MHz, CDCl₃): δ 8.76 (s, 1H), 8.44 (s, 1H), 8.36 (s, 1H), 8.10 (d, J=1.5 Hz, 1H), 7.87 (dd, J=1.5, 8.0 Hz, 1H), 7.56 (s, 1H), 7.46 (d, J=8.0 Hz, 1H), 5.40 (s, 2H), 4.95 (t, J=5.0 Hz, 1H), 4.57 (d, J=5.0 Hz, 2H), 4.07-4.04 (m, 2H), 3.16-3.12 (m, 2H), 3.08-3.05 (m, 1H), 2.58 (s, 3H), 1.18 (s, 2H); MS [M+H]⁺=479.9.

8-[(5-amino-1,3-dioxan-2-yl)methyl]-6-[2-chloro-4-(6-methylpyrazin-2-yl)phenyl]-2-(oxetan-3-ylamino)pyrido[2,3-d]pyrimidin-7-one (I-164) was prepared in accord with the procedure used for I-144 except 3-amino-oxetane replaced ethylamine. ¹H NMR (500 MHz, CDCl₃): δ 8.76 (s, 1H), 8.44 (s, 1H), 8.36 (s, 1H), 8.10 (d, J=1.5 Hz, 1H), 7.87 (dd, J=1.5, 8.0 Hz, 1H), 7.56 (s, 1H), 7.46 (d, J=8.0 Hz, 1H), 6.06 (s, 1H), 5.07 (s, 1H), 4.99 (t, J=6.0 Hz, 2H), 4.88 (s, 1H), 4.61-4.55 (m, 4H), 4.06-4.02 (m, 2H), 3.12 (t, J=10 Hz, 2H), 3.05-3.02 (m, 1H), 2.57 (s, 3H), 1.18 (s, 2H).: δ 8.76 (s, 1H), 8.44 (s, 1H), 8.36 (s, 1H), 8.10 (d, J=1.5 Hz, 1H), 7.87 (dd, J=1.5, 8.0 Hz, 1H), 7.56 (s, 1H), 7.46 (d, J=8.0 Hz, 1H), 6.06 (s, 1H), 5.07 (s, 1H), 4.99 (t, J=6.0 Hz, 2H), 4.88 (s, 1H), 4.61-4.55 (m, 4H), 4.06-4.02 (m, 2H), 3.12 (t, J=10 Hz, 2H), 3.05-3.02 (m, 1H), 2.57 (s, 3H), 1.18 (s, 2H); MS [M+H]⁺=536.

8-[(5-Amino-1,3-dioxan-2-yl)methyl]-6-[2-chloro-4-(6-methyl-2-pyridyl)phenyl]-2-(methylamino)pyrido[2,3-d]pyrimidin-7-one (I-167) was prepared analogously except 6-(2-chloro-4-(6-methylpyridin-2-yl)phenyl)-2-(methylthio)pyrido[2,3-d]pyrimidin-7(8H)-one was the starting material and methyl amine replaced ethyl amine. ¹H NMR (400 MHz, DMSO-d₆) δ 8.68 (d, 1H, rotamer), 8.22 (d, J=1.7 Hz, 1H), 8.08 (dd, J=8.1, 1.8 Hz, 1H), 7.92-7.77 (m, 4H), 7.51 (d, J=8.0 Hz, 1H), 7.28 (d, J=7.4 Hz, 1H), 5.07-4.85 (m, 1H), 4.52-4.37 (m, 2H), 4.00-3.84 (m, 2H), 3.11 (t, J=10.6 Hz, 2H), 2.91 (d, J=4.7 Hz, 3H), 2.85-2.70 (m, 1H), 2.57 (s, 3H). NH2 was not observed; MS [M+H]⁺=493.2.

8-[(5-Amino-1,3-dioxan-2-yl)methyl]-2-(methylamino)-6-[2-methyl-4-(6-methyl-2-pyridyl)phenyl]pyrido[2,3-d]pyrimidin-7-one (I-170) was prepared in accord with the procedure used for I-167 except 6-(2-methyl-4-(6-methylpyridin-2-yl)phenyl)-2-(methylthio)pyrido[2,3-d]pyrimidin-7(8H)-one was the starting material. ¹H NMR (400 MHz, DMSO-d₆) δ 8.72-8.55 (m, 1H), 7.96 (s, 1H), 7.90 (dd, J=8.0, 1.8 Hz, 1H), 7.81-7.62 (m, 4H), 7.29 (d, J=7.9 Hz, 1H), 7.22 (dd, J=4.9, 3.6 Hz, 1H), 4.93 (d, J=40.2 Hz, 1H), 4.49 (s, 2H), 3.90 (dt, J=38.3, 19.1 Hz, 2H), 3.12 (t, J=10.7 Hz, 4H), 2.91 (d, J=4.7 Hz, 3H), 2.80 (d, J=5.3 Hz, 1H), 2.56 (s, 3H), 2.22 (s, 4H); MS [M+H]⁺=473.3.

6-[2-Chloro-4-(6-methyl-2-pyridyl)phenyl]-8-(5 9-dioxa-2-azaspiro[3.5]nonan-7-ylmethyl)-2-(methylamino)pyrido[2,3-d]pyrimidin-7-one (I-180) was prepared by alkylation (Cs₂CO₃, DMF, 100 oC, 48 h) with benzyl 7-(methylsulfonyloxymethyl)-5,9-dioxa-2-azaspiro[3.5]nonane-2-carboxylate to afford benzyl 7-((6-(2-chloro-4-(6-methylpyridin-2-yl)phenyl)-2-(methylthio)-7-oxopyrido[2,3-d]pyrimidin-8(7H)-yl)methyl)-5,9-dioxa-2-azaspiro[3.5]nonane-2-carboxylate. Then N-methylamine substituent was introduced using standard protocols. The final step was removal of the CBZ protecting group (TFA). ¹H NMR (400 MHz, DMSO-d₆) δ 8.65 (m, 1H), 8.22 (d, J=1.7 Hz, 1H), 8.08 (dd, J=8.0, 1.8 Hz, 1H), 7.92-7.76 (m, 4H), 7.51 (d, J=8.0 Hz, 1H), 7.32-7.23 (m, 1H), 4.33-4.10 (m, 2H), 3.84 (dd, J=11.5, 4.1 Hz, 2H), 3.66 (dd, J=11.5, 7.6 Hz, 2H), 3.50 (s, 2H), 3.42 (s, 2H), 2.93 (d, J=4.7 Hz, 3H), 2.57 (s, 3H), 2.24 (m, 1H); MS [M+H]⁺=519.2.

8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one (I-177) was prepared in accord with the procedure used to prepare I-144 except methylamine was used in place of ethylamine to displace the methylsulfinyl group. 1H NMR (500 MHz, DMSO-d6): δ 9.17 (s, 1H), 8.65 (s, 1H), 8.58 (s, 1H), 8.28 (d, J=1.5 Hz, 1H), 8.16 (dd, J=1.5, 8.0 Hz, 1H), 7.86 (s, 1H), 7.57 (d, J=8.5 Hz, 1H), 4.99 (t, J=5.5 Hz, 1H), 4.50-4.42 (m, 2H), 3.94-3.90 (m, 2H), 3.12 (t, J=10 Hz, 2H), 2.92 (d, J=4.5 Hz, 3H), 2.82-2.73 (m, 1H), 2.60 (s, 3H); MS [M+H]⁺=494.

8-[2-(5-amino-1,3-dioxan-2-yl)ethyl]-6-[2-chloro-4-(6-methylpyrazin-2-yl)phenyl]-2-(methylamino)pyrido[2,3-d]pyrimidin-7-one (I-172) was prepared by condensing tert-butyl ((2S,5S)-2-(2-aminoethyl)-1,3-dioxan-5-yl)carbamate (syn isomer) and 4-chloro-2-(methylthio)pyrimidine-5-carbaldehyde to afford tert-butyl (2s,5s)-2-(2-(5-formyl-2-(methylthio)pyrimidin-4-ylamino)ethyl)-1,3-dioxan-5-ylcarbamate and cyclizing the product with methyl 2-(2-chloro-4-(6-methylpyridin-2-yl)phenyl)acetate (K₂CO₃, DMF, 120° C.) to afford tert-butyl (2s,5s)-2-(2-(6-(2-chloro-4-(6-methylpyridin-2-yl)phenyl)-2-(methylthio)-7-oxopyrido[2,3-d]pyrimidin-8(7H)-yl)ethyl)-1,3-dioxan-5-ylcarbamate. Introduction of the methyl amine and deprotection were carried out using standard protocols. ¹H NMR (400 MHz, DMSO-d₆) δ 9.15 (s, 1H), 8.64 (m, 1H), 8.57 (s, 1H), 8.27 (d, J=1.8 Hz, 1H), 8.15 (dd, J=8.0, 1.8 Hz, 1H), 7.83 (m, 2H), 7.56 (d, J=8.0 Hz, 1H), 4.68 (t, J=4.9 Hz, 1H), 4.51-4.29 (m, 2H), 3.87-3.77 (m, 2H), 3.75-3.63 (m, 2H), 2.94 (m, 3H), 2.60 (s, 3H), 2.57-2.52 (m, 1H), 1.95 (m, 2H), 1.82 (m, 2H); MS [M+H]⁺=508.2.

6-[2-Chloro-4-(6-methylpyrazin-2-yl)phenyl]-8-[[(2R)-morpholin-2-yl]methyl]-2-(oxetan-3-ylamino)pyrido[2,3-d]pyrimidin-7-one (I-146) was prepared analogously except the lactam nitrogen was alkylated (NaH, DMF, 85° C.) with tert-butyl (2R)-2-(p-tolylsulfonyloxymethyl)morpholine-4-carboxylate (CASRN 135065-71-3) and an oxetan-3-ylamino substituent was introduced using standard protocols. The final step was removal of the Boc group (TFA). ¹H NMR (500 MHz, DMSO-d₆) δ 9.16 (s, 1H), 8.71 (s, 2H), 8.57 (s, 1H), 8.28 (s, 1H), 8.16 (d, J=7.9 Hz, 1H), 7.88 (s, 1H), 7.55 (d, J=8.0 Hz, 1H), 4.99-4.89 (m, 1H), 4.89-4.75 (m, 2H), 4.68-4.62 (m, 1H), 4.59 (t, J=6.1 Hz, 1H), 4.48-4.33 (m, 1H), 4.26-4.17 (m, 1H), 3.74 (d, J=10.7 Hz, 2H), 3.40-3.35 (m, 1H), 2.72 (d, J=11.9 Hz, 1H), 2.64 (d, J=4.8 Hz, 2H), 2.60 (s, 3H), 2.24-2.52 (m, 1H); MS [M+H]⁺=519.9.

6-[2-Chloro-4-(6-methylpyrazin-2-yl)phenyl]-8-[[(2S)-morpholin-2-yl]methyl]-2-(oxetan-3-ylamino)pyrido[2,3-d]pyrimidin-7-one (I-149) was prepared analogously except tert-butyl (2R)-2-(p-tolylsulfonyloxymethyl)morpholine-4-carboxylate was replaced with tert-butyl (2S)-2-(p-tolylsulfonyloxymethyl)morpholine-4-carboxylate [CASRN14863/8-79-0]. ¹H NMR (500 MHz, MeOH-d₄) δ 8.98 (s, 1H), 8.67 (s, 1H), 8.50 (s, 1H), 8.28 (s, 1H), 8.10 (d, J=7.8 Hz, 1H), 7.84 (s, 1H), 7.56 (d, J=7.9 Hz, 1H), 5.23-4.97 (m, 3H), 4.83-4.69 (m, 2H), 4.68-4.56 (m, 1H), 4.46-4.31 (m, 1H), 4.07-3.92 (m, 1H), 3.89 (d, J=11.1 Hz, 1H), 3.57 (t, J=11.3 Hz, 1H), 2.93 (d, J=12.0 Hz, 1H), 2.89-2.70 (m, 3H), 2.67 (s, 3H); MS [M+H]⁺=519.9.

6-[2-chloro-4-(6-methylpyrazin-2-yl)phenyl]-2-(methylamino)-8-[[(2S)-morpholin-2-yl]methyl]pyrido[2,3-d]pyrimidin-7-one (I-150) was prepared in accord with the procedure for I-149 except methyl amine replaced oxetan-3-amine. ¹H NMR (500 MHz, DMSO-d6) δ 9.16 (s, 1H), 8.65 (s, 1H), 8.57 (s, 1H), 8.27 (d, J=1.5 Hz, 1H), 8.15 (dd, J=1.6, 8.0 Hz, 1H), 7.92 (d, J=4.7 Hz, 1H), 7.86 (s, 1H), 7.56 (d, J=8.0 Hz, 1H), 4.49-4.38 (m, 1H), 4.35-4.14 (m, 1H), 3.89-3.77 (m, 1H), 3.77-3.67 (m, 1H), 3.42-3.37 (m, 1H), 2.93 (d, J=4.6 Hz, 3H), 2.69 (d, J=12.1 Hz, 1H), 2.63 (d, J=4.8 Hz, 2H), 2.60 (s, 3H), 2.54 (d, J=10.2 Hz, 1H); MS [M+H]⁺=478.0.

6-[2-Methyl-4-(6-methyl-2-pyridyl)phenyl]-8-[[(2R)-morpholin-2-yl]methyl]-2-(oxetan-3-ylamino)pyrido[2,3-d]pyrimidin-7-one (I-147) was prepared in accord with I-146 except 6-(2-methyl-4-(6-methylpyridin-2-yl)phenyl)-2-(methylthio)pyrido[2,3-d]pyrimidin-7(8H)-one replaced 6-(2-chloro-4-(6-methylpyrazin-2-yl)phenyl)-2-(methylthio)pyrido[2,3-d]pyrimidin-7(8H)-one and the alkylation with tert-butyl (2R)-2-(p-tolylsulfonyloxymethyl)morpholine-4-carboxylate was carried out with Cs₂CO₃, DMF, 85° C.). ¹H NMR (500 MHz, DMSO-d₆) δ 8.69 (s, 1H), 8.62 (br s, 1H), 7.97 (s, 1H), 7.91 (d, J=8.0 Hz, 1H), 7.80-7.74 (m, 3H), 7.28 (d, J=8.0 Hz, 1H), 7.25-7.20 (m, 1H), 5.00-4.76 (m, 3H), 4.70-4.54 (m, 2H), 4.49-4.42 (m, 1H), 4.28-4.19 (m, 1H), 3.84-3.72 (m, 2H), 3.43-3.38 (m, 1H), 2.81-2.74 (m, 1H), 2.72-2.65 (m, 2H), 2.60-2.56 (m, 1H), 2.56 (s, 3H), 2.22 (s, 3H); MS [M+H]⁺=499.0.

2-(Methylamino)-6-[2-methyl-4-(6-methyl-2-pyridyl)phenyl]-8-[[(2R)-morpholin-2-yl]methyl]pyrido[2,3-d]pyrimidin-7-one (I-148) was prepared in accord with the procedure used for I-147 except methylamine was introduced in place of oxetan-3-ylamine. ¹H NMR (500 MHz, DMSO-d₆) δ 8.63 (s, 1H), 7.96 (s, 1H), 7.91 (d, J=8.0 Hz, 1H), 7.86-7.80 (m, 1H), 7.80-7.73 (m, 3H), 7.29 (d, J=7.9 Hz, 1H), 7.24-7.20 (m, 1H), 4.53-4.40 (m, 1H), 4.41-4.20 (m, 1H), 3.93-3.80 (m, 1H), 3.79-3.66 (m, 1H), 2.97-2.87 (s, 3H), 2.72-2.60 (m, 4H), 2.56 (s, 3H), 2.23 (s, 3H); MS [M+H]⁺=457.0.

6-[2-Chloro-4-(6-methyl-2-pyridyl)phenyl]-8-[[(2R)-morpholin-2-yl]methyl]-2-(oxetan-3-ylamino)pyrido[2,3-d]pyrimidin-7-one (I-159) was prepared in accord with the procedure for I-147 except 6-(2-chloro-4-(6-methylpyridin-2-yl)phenyl)-2-(methylthio)pyrido[2,3-d]pyrimidin-7(8H)-one replaced 6-(2-methyl-4-(6-methylpyridin-2-yl)phenyl)-2-(methylthio)pyrido[2,3-d]pyrimidin-7(8H)-one. ¹H NMR (400 MHz, DMSO-d₆) δ 8.75-8.64 (m, 2H), 8.22 (d, J=1.5 Hz, 1H), 8.09 (dd, J=1.5, 8 Hz, 1H), 7.87 (d, J=8.5 Hz, 1H), 7.86 9 (s, 1H), 7.81 (q, J=8 Hz, 1H), 7.50 (d, J=8 Hz, 1H), 7.28 (d, J=8 Hz, 1H), 4.98-4.77 (m, 3H), 4.69-4.52 (m, 2H), 4.48-4.35 (m, 1H), 4.29-4.17 (m, 1H), 3.78-3.68 (m, 2H), 3.41-3.34 (m, 1H), 2.75-2.68 (m, 1H), 2.67-2.61 (m, 2H), 2.57 (s, 3H); MS [M+H]⁺=519.2.

6-(2-methyl-4-(6-methylpyridin-2-yl)phenyl)-2-(methylamino)-8-[[(2S)-morpholin-2-yl]methyl]pyrido[2,3-d]pyrimidin-7(8H)-one (I-171) was prepared analogously from 6-(2-methyl-4-(6-methylpyridin-2-yl)phenyl)-2-(methylthio)pyrido[2,3-d]pyrimidin-7(8H)-one using with tert-butyl (2S)-2-(p-tolylsulfonyloxymethyl)morpholine-4-carboxylate to alkylate the lactam (Cs₂CO₃, DMF). Introduction of the methyl amine and deprotection were carried out using standard protocols. ¹H NMR (400 MHz, CDCl₃) δ 8.45 (s, 1H), 7.87 (s, 1H), 7.80 (d, J=8 Hz, 1H), 7.63 (t, J=7.6 Hz, 1H), 7.52 (d, J=7.6 Hz, 1H), 7.46 (s, 1H), 7.29 (d, J=8 Hz, 1H), 7.09 (d, J=7.6 Hz, 1H), 5.48-5.42 (m, 1H), 4.67-4.43 (m, 2H), 4.06-3.96 (m, 1H), 3.91 (d, J=11.2 Hz, 1H), 3.58 (dt, J=2 Hz, 10.8 Hz, 1H), 3.11 (d, J=4.8 Hz, 3H), 2.95-2.86 (m, 2H), 2.85-2.76 (m, 2H), 2.64 (s, 3H), 2.31 (s, 3H); MS [M+H]⁺=457.2.

6-[2-methyl-4-(6-methyl-2-pyridyl)phenyl]-8-[[(2S)-morpholin-2-yl]methyl]-2-(oxetan-3-ylamino)pyrido[2,3-d]pyrimidin-7-one (I-187) was prepared in accord with the procedure used for I-171 except 3-amino-oxetane replaced methylamine. ¹H NMR (400 MHz, DMSO-d₆) δ 8.69 (s, 1H), 7.96 (s, 1H), 7.91 dd, J=0.8, 8 Hz, 1H), 7.80-7.73 (m, 3H), 7.28 (d, J=8 Hz, 1H), 7.26-7.18 (m, 1H), 5.07-4.79 (m, 3H), 4.68-4.53 (m, 2H), 4.55-4.39 (m, 1H), 4.28-4.16 (m, 1H), 3.81-3.70 (m, 2H), 3.45-3.26 (m, 2H), 2.77-2.60 (m, 3H), 2.56 (s, 3H), 2.22 (s, 3H); MS [M+H]⁺=499.3.

6-[2-chloro-4-(6-methyl-2-pyridyl)phenyl]-2-(methylamino)-8-[[(2S)-morpholin-2-yl]methyl]pyrido[2,3-d]pyrimidin-7-one (I-173) was prepared by alkylation (Cs₂CO₃, DMF) of 6-(2-chloro-4-(6-methylpyridin-2-yl)phenyl)-2-(methylthio)pyrido[2,3-d]pyrimidin-7(8H)-one with (S)-tert-butyl 2-(tosyloxymethyl)morpholine-4-carboxylate to afford (S)-tert-butyl 2-((6-(2-chloro-4-(6-methylpyridin-2-yl)phenyl)-2-(methylthio)-7-oxopyrido[2,3-d]pyrimidin-8(7H)-yl)methyl)morpholine-4-carboxylate. Introduction of the methyl amine and deprotection were carried out using standard protocols. ¹H NMR (400 MHz, CDCl₃) δ 8.45 (s, 1H), 7.87 (s, 1H), 7.80 (d, J=8 Hz, 1H), 7.63 (t, J=7.6 Hz, 1H), 7.52 (d, J=7.6 Hz, 1H), 7.46 (s, 1H), 7.29 (d, J=8 Hz, 1H), 7.09 (d, J=7.6 Hz, 1H), 5.48-5.42 (m, 1H), 4.67-4.43 (m, 2H), 4.06-3.96 (m, 1H), 3.91 (d, J=11.2 Hz, 1H), 3.58 (dt, J=2 Hz, 10.8 Hz, 1H), 3.11 (d, J=4.8 Hz, 3H), 2.95-2.86 (m, 2H), 2.85-2.76 (m, 2H), 2.64 (s, 3H), 2.31 (s, 3H); MS [M+H]⁺=477.2.

6-[2-chloro-4-(6-methyl-2-pyridyl)phenyl]-8-[[(2S)-morpholin-2-yl]methyl]-2-(oxetan-3-ylamino)pyrido[2,3-d]pyrimidin-7-one (I-188) was prepared in accord with the procedure used to prepare I-173 except methylamine was replaced by 3-amino-oxetane. ¹H NMR (400 MHz, DMSO-d₄) δ 8.77-8.65 (m, 2H), 8.22 (d, J=1.7 Hz, 1H), 8.09 (dd, J=0.8, 8 Hz, 1H), 7.91-7.84 (m, 2H), 7.81 (t, J=7.6 Hz, 1H), 7.50 (d, J=8 Hz, 1H), 7.28 (d=7.6 Hz, 1H), 5.10-4.76 (m, 3H), 4.71-4.54 (m, 2H), 4.53-4.36 (m, 1H), 4.31-4.16 (m, 1H), 3.90-3.71 (m, 2H), 3.45-3.36 (m, 1H), 2.82-2.62 (m, 3H), 2.57 (s, 3H), 2.56-2.53 (m, 1H); MS [M+H]⁺=519.2.

6-[2-chloro-4-(6-methyl-2-pyridyl)phenyl]-2-(methylamino)-8-[[(2S)-4-methylmorpholin-2-yl]methyl]pyrido[2,3-d]pyrimidin-7-one (I-183) was prepared by reductive methylation of I-172 (HCHO, NaBH₄). ¹H NMR (300 MHz, MeOH-d₄) δ 8.58 (s, 1H), 8.12 (d, J=1.7 Hz, 1H), 7.94 (dd, J=1.8, 8.0 Hz, 1H), 7.83-7.73 (m, 2H), 7.69 (d, J=7.7 Hz, 1H), 7.49 (d, J=8.0 Hz, 1H), 7.26 (d, J=7.5 Hz, 1H), 4.73-4.58 (m, 2H), 4.53-4.35 (m, 1H), 4.08 (s, 1H), 3.89 (d, J=10.5 Hz, 1H), 3.69-3.48 (m, 1H), 3.05 (s, 3H), 2.87-2.71 (m, 1H), 2.66 (d, J=11.0 Hz, 1H), 2.61 (s, 3H), 2.28 (s, 3H), 2.24-2.00 (m, 2H); MS [M+H]⁺=491.2.

### Example 40

### 6-[2-chloro-4-(6-methylpyrazin-2-yl)phenyl]-2-(methylamino)-8-(2-morpholinoethyl)pyrido[2,3-d]pyrimidin-7-one (I-158)

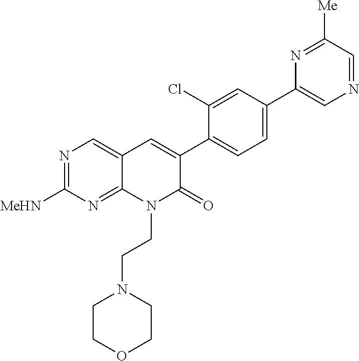 [SMILES: CNc1ncc2cc(-c3ccc(-c4cncc(C)n4)cc3Cl)c(=O)n(CCN3CCOCC3)c2n1]

6-(2-chloro-4-(6-methylpyrazin-2-yl)phenyl)-2-(methylsulfinyl)pyrido[2,3-d]pyrimidin-7(8H)-one was condensed with 1-(4-methoxyphenyl)-N-methylmethanamine (DIPEA, THF, 50° C., 18 h) to afford 6-(2-chloro-4-(6-methylpyrazin-2-yl)phenyl)-2-((4-methoxybenzyl)(methyl)amino)pyrido[2,3-d]pyrimidin-7(8H)-one. Alkylation with 1,2-dibromoethane (NaH, DMF, RT, 2 h) afforded 8-(2-bromoethyl)-6-(2-chloro-4-(6-methylpyrazin-2-yl)phenyl)-2-((4-methoxybenzyl)(methyl)amino)pyrido[2,3-d]pyrimidin-7(8H)-one which was treated with morpholine and deprotected with TFA. ¹H NMR (400 MHz, DMSO-d₆) δ 9.16 (s, 1H), 8.71 (s, 1H), 8.57 (s, 1H), 8.27 (d, J=1.0 Hz, 1H), 8.15 (dd, J=1.5, 8.0 Hz, 1H), 7.91-7.89 (m, 1H), 7.85 (s, 1H), 7.55 (d, J=8.0 Hz, 1H), 4.48-4.39 (m, 2H), 3.58-3.48 (m, 4H), 2.91 (d, J=4.5 Hz, 3H), 2.65-2.63 (m, 2H), 2.60 (s, 3H), 2.53-2.50 (m, 4H); MS [M+H]⁺=492.02.

2-(Methylamino)-6-[2-methyl-4-(6-methyl-2-pyridyl)phenyl]-8-(2-morpholinoethyl)pyrido[2,3-d]pyrimidin-7-one (I-165) was prepared in accord with the procedure for I-158 except 6-(2-methyl-4-(6-methylpyridin-2-yl)phenyl)-2-(methylthio)pyrido[2,3-d]pyrimidin-7(8H)-one replaced 6-(2-chloro-4-(6-methylpyrazin-2-yl)phenyl)-2-(methylsulfinyl)pyrido[2,3-d]pyrimidin-7(8H)-one as the starting material. ¹H NMR (500 MHz, DMSO-d₆) δ 8.63 (s, 1H), 7.96 (s, 1H), 7.91 (d, J=8.0 Hz, 1H), 7.82-7.81 (m, 1H), 7.78-7.77 (m, 2H), 7.75 (s, 1H), 7.28 (d, J=8.0 Hz, 1H), 7.23-7.21 (m, 1H), 4.48-4.38 (m, 2H), 3.53 (s, 4H), 2.91 (d, J=4.5 Hz, 3H), 2.66-2.64 (m, 2H), 2.58 (s, 3H), 2.52-2.50 (m, 4H), 2.24 (s, 3H); MS [M+H]⁺=471.0.

### Example 41

### 8-[2-(azetidin-3-ylsulfonyl)ethyl]-6-[2-chloro-4-(6-methyl-2-pyridyl)phenyl]-2-(ethylamino)pyrido[2,3-d]pyrimidin-7-one (I-160)

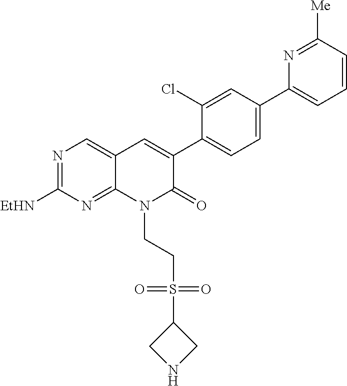

6-(2-Chloro-4-(6-methylpyridin-2-yl)phenyl)-2-(methylthio)pyrido[2,3-d]pyrimidin-7(8H)-one was alkylated with tert-butyl 3-[(2-hydroxyethane)sulfonyl]azetidine-1-carboxylate (DIAD, PPh₃, DMF, RT). Introduction of the ethylamine and removal of the Boc group were accomplished by standard protocols. ¹H NMR (400 MHz, DMSO-d₆) δ 8.67 (s, 1H), 8.23 (d, J=0.8 Hz, 1H), 8.09 (dd, J=1.6, 6.8 Hz, 1H), 7.88 (d, J=5.6 Hz, 2H), 7.81 (t, J=6 Hz, 1H), 7.50 (d, J=6.4 Hz, 1H), 7.28 (d, J=6 Hz, 1H), 4.70 (t, J=5.6 Hz, 1H), 4.62-4.55 (m, 1H), 4.53-4.45 (m, 1H), 3.78 (t, J=6 Hz, 2H), 3.62 (1, J=6.8 Hz, 2H), 3.48-3.4 (m, 4H), 2.57 (s, 3H), 1.21 (t, J=5.6 Hz, 3H); MS [M+H]⁺=538.9.

8-[2-(2-aminoethylsulfonyl)ethyl]-2-(methylamino)-6-[2-methyl-4-(6-methyl-2-pyridyl)phenyl]pyrido[2,3-d]pyrimidin-7-one (I-174) was prepared analogously except tert-butyl 3-[(2-hydroxyethane)sulfonyl]azetidine-1-carboxylate was replaced with tert-butyl 2-(2-hydroxyethylsulfonyl)ethylcarbamate (DIAD, PPh₃, DMF, RT). Introduction of the methylamine and removal of the Boc group were accomplished by standard protocols. ¹H NMR (400 MHz, MeOH-d₄) δ8.62 (brs, 1H), 7.87 (s, 1H), 7.79 (t, J=7.6 Hz, 2H), 7.75 (s, 1H), 7.66 (d, J=8 Hz, 1H), 7.33 (d, J=8 Hz, 1H), 7.25 (d, J=7.6 Hz, 1H), 5.01-4.91 (m, 2H), 3.64 (brs, 2H), 3.45 (brs, 2H), 3.19 (t, J=6.8 Hz, 2H), 3.09 (s, 3H), 2.62 (s, 3H), 2.33 (s, 3H; MS [M+H]⁺=493.0.

### Example 42

### 3-[6-[2-methyl-4-(6-methyl-2-pyridyl)phenyl]-2-(oxetan-3-ylamino)-7-oxo-pyrido[2,3-d]pyrimidin-8-yl]propane-1-sulfonamide (I-161)

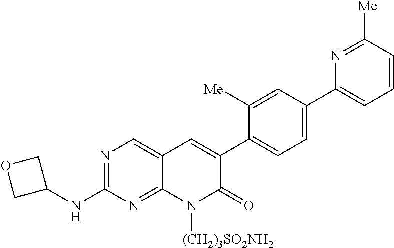

3-Amino-1-propanesulfonamide was condensed with 4-chloro-2-(methylthio)pyrimidine-5-carbaldehyde (TEA, THF, H₂O, RT) to afford 3-(5-formyl-2-(methylthio)pyrimidin-4-ylamino)propane-1-sulfonamide which was cyclized with ethyl 2-(2-methyl-4-(6-methylpyridin-2-yl)phenyl)acetate (K₂CO₃, DMF) to afford 3-(6-(2-methyl-4-(6-methylpyridin-2-yl)phenyl)-2-(methylthio)-7-oxopyrido[2,3-d]pyrimidin-8(7H)-yl)propane-1-sulfonamide. Introduction of the oxetan-3-amine was accomplished by standard protocols. ¹H NMR (500 MHz, DMSO-d₆) δ8.71 (s, 1H), 8.63 (brs, 1H), 7.97 (s, 1H), 7.91 (d, J=8 Hz, 1H), 7.80-7.76 (m, 3H), 7.29 (d, J=7.5 Hz, 1H), 7.24-7.20 (m, 1H), 6.80 (s, 2H), 5.00 (t, J=7 Hz, 1H), 4.87 (t, J=6.5 Hz, 2H), 4.61 (t, J=5.5 Hz, 2H), 4.38 (t, J=7 Hz, 2H), 3.43-3.33 (brs, 2H), 2.55 (s, 3H), 2.23 (s, 3H), 2.10 (brs, 2H); MS [M+H]⁺=521.1.

2-[6-[2-methyl-4-(6-methyl-2-pyridyl)phenyl]-2-(oxetan-3-ylamino)-7-oxo-pyrido[2,3-d]pyrimidin-8-yl]ethanesulfonate (I-166) was prepared analogously except 2-aminoethanesulfonamide replaced 3-amino-1-propanesulfonamide. ¹H NMR (500 MHz, DMSO-d₆) δ8.71 (s, 1H), 8.66 (brs, 1H), 7.97 (s, 1H), 7.91 (d, J=8 Hz, 1H), 7.80-7.76 (m, 3H), 7.28 (d, J=6 Hz, 1H), 7.24-7.20 (m, 1H), 7.11 (brs, 2H), 4.97 (t, J=5 Hz, 1H), 4.90 (t, J=6 Hz, 2H), 4.68 (t, J=7.5 Hz, 2H), 4.58 (t, J=6.5 Hz, 2H), 3.43-3.33 (m, 2H), 2.55 (s, 3H), 2.24 (s, 3H); MS [M+H]⁺=507.0.

### Example 43

### 6-[2-chloro-4-(6-methyl-2-pyridyl)phenyl]-2-(methylamino)-8-(piperazin-1-ylsulfonylmethyl)pyrido[2,3-d]pyrimidin-7-one (I-162)

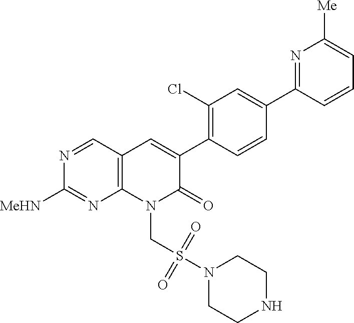 [SMILES: CNc1ncc2cc(-c3ccc(-c4cccc(C)n4)cc3Cl)c(=O)n(CS(=O)(=O)N3CCNCC3)c2n1]

6-(2-chloro-4-(6-methylpyridin-2-yl)phenyl)-2-(methylthio)pyrido[2,3-d]pyrimidin-7(8H)-one was alkylated (Cs₂CO₃, DMF, 100° C.) with tert-butyl 4-[(bromomethyl)sulfonyl]-1-piperazinecarboxylate (CASRN 1443979-93-8) to afford 6-(2-chloro-4-(6-methylpyridin-2-yl)phenyl)-2-(methylthio)pyrido[2,3-d]pyrimidin-7(8H)-one. Introduction of the methylamine and removal of the Boc were carried out with standard protocols to afford I-162. ¹H NMR (400 MHz, DMSO-d₆) δ 8.74 (m, 1H), 8.23 (d, J=1.7 Hz, 1H), 8.10 (dd, J=8.0, 1.8 Hz, 1H), 8.05 (m, 1H), 7.93 (m, 1H), 7.87 (d, J=8.1 Hz, 1H), 7.81 (t, J=7.7 Hz, 1H), 7.50 (d, J=8.0 Hz, 1H), 7.28 (dd, J=7.5, 1.0 Hz, 1H), 5.68 (m, 2H), 3.13-3.04 (m, 4H), 2.95 (d, J=4.9 Hz, 3H), 2.70-2.61 (m, 4H), 2.57 (s, 3H); MS [M+H]⁺=540.2.

### Example 44

### 8-(3-hydroxypropyl)-6-[2-methyl-4-(6-methyl-2-pyridyl)phenyl]-2-(oxetan-3-ylamino)pyrido[2,3-d]pyrimidin-7-one (I-168)

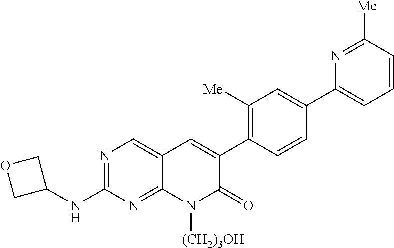

6-(2-Methyl-4-(6-methylpyridin-2-yl)phenyl)-2-(methylthio)pyrido[2,3-d]pyrimidin-7(8H)-one was alkylated with 3-bromo-propan-1-ol (Cs₂CO₃, DMF). Introduction of the 3-amino-oxetane was accomplished by standard protocols. ¹H NMR (400 MHz, DMSO-d₆) δ 8.69 (s, 1H), 8.55 (s, 1H), 7.96 (d, J=1.8 Hz, 1H), 7.90 (rd, J=8.0, 1.9 Hz, 1H), 7.81-7.72 (m, 3H), 7.28 (d, J=7.9 Hz, 1H), 7.22 (dd, J=5.0, 3.5 Hz, 1H), 5.06-4.90 (m, 1H), 4.90-4.78 (m, 2H), 4.60 (I, J=6.2 Hz, 2H), 4.56-4.47 (m, 1H), 4.38-4.30 (m, 2H), 3.56-3.46 (m, 2H), 2.55 (s, 3H), 2.23 (s, 3H), 1.87-1.72 (m, 2H); MS [M+H]⁺=458.2.

### Example 45

### 4-[[6-[2-chloro-4-(6-methyl-2-pyridyl)phenyl]-2-(methylamino)-7-oxo-pyrido[2,3-d]pyrimidin-8-yl]methyl]piperidine-4-carbonitrile (I-169)

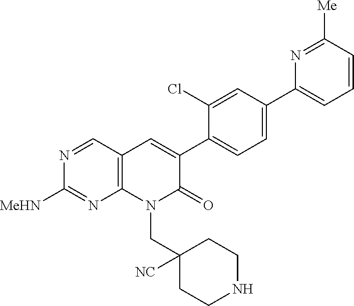 [SMILES: CNc1ncc2cc(-c3ccc(-c4cccc(C)n4)cc3Cl)c(=O)n(CC3(C#N)CCNCC3)c2n1]

### tert-Butyl 4-cyano-4-(tosyloxymethyl)piperidine-1-carboxylate

step 1: A mixture of piperidine-4-carbonitrile (1.1 g 10.0 mmol), di-tert-butyl dicarbonate (2.62 g, 12 mmol) and TEA (4.2 mL, 30 mmol) in DCM (15 mL) was stirred at RT overnight. HCl (5% wt in water) was added to adjust the pH to below 5. The aqueous phase was extracted with DCM. The organic layer was washed with brine, dried (Na₂SO₄), filtered and concentrated in vacuo to afford tert-butyl 4-cyanopiperidine-1-carboxylate (2.0 g, 95.2%) as yellow oil, which was used in next step without further purification. LCMS (ESI): m/z=155.1 [M−55].

step 2: To a stirring mixture of tert-butyl 4-cyanopiperidine-1-carboxylate (1.0 g crude, 4.76 mmol) in THF (10 mL) at −78° C. under a nitrogen atmosphere was added dropwise LHMDS (1M in THF, 7.14 mL, 7.14 mmol). The mixture was stirred at −78° C. for 1 h. Then ethyl carbonochloridate (771 mg, 7.14 mmol) was added, and the mixture was stirred at −78° C. for another 1 h. The mixture was quenched with saturated aqueous NH₄Cl and the aqueous phase extracted with EtOAc. The organic layer was dried (Na₂SO₄), filtered and concentrated in vacuo. The residue was purified by SiO₂ chromatography eluting with PE/EtOAc (4:1) to afford 1-tert-butyl 4-ethyl 4-cyanopiperidine-1,4-dicarboxylate (1.3 g crude, 83.1%) as a yellow oil. LCMS (ESI): m/z=183.1 [M−99].

step 3: To a stirring mixture of 1-tert-butyl 4-ethyl 4-cyanopiperidine-1,4-dicarboxylate (1.3 g crude, 4.61 mmol) in MeOH (15 mL) was added NaBH₄ (350 mg, 9.22 mmol) under a nitrogen atmosphere at 0° C. The mixture was stirred at 0° C. for 1.5 h then quenched with HCl (5% wt in water, 5 mL). The aqueous phase was extracted with EtOAc and the organic layer was dried (Na₂SO₄), filtered and concentrated in vacuo. The residue was purified by SiO₂ chromatography eluting with PE/EtOAc (4:1) to afford tert-butyl 4-cyano-4-(hydroxymethyl)piperidine-1-carboxylate (900 mg, 81.3%) as a colorless oil. LCMS (ESI): m/z=141.1 [M−99].

step 4: A mixture of tert-butyl 4-cyano-4-(hydroxymethyl)piperidine-1-carboxylate (300 mg 1.25 mmol), tosyl chloride (285 mg, 1.5 mmol), DMAP (16 mg, 0.13 mmol) and TEA (0.52 mL, 3.75 mmol) in DCM (5 mL) was stirred at RT overnight. The mixture was concentrated and the residue was purified by SiO₂ chromatography eluting with PE/EtOAc (6:1) to afford tert-butyl 4-cyano-4-(tosyloxymethyl)piperidine-1-carboxylate (120 mg, 24.4%) as white solid. LCMS (ESI): m/z=295.0 [M−99].

6-(2-chloro-4-(6-methylpyridin-2-yl)phenyl)-2-(methylthio)pyrido[2,3-d]pyrimidin-7(8H)-one was alkylated with tert-butyl 4-cyano-4-(tosyloxymethyl)piperidine-1-carboxylate (Cs₂CO₃, DMF, 60° C.). Oxidation of the methylthio and addition of the methylamine (MeNH₂, DIPEA) followed by deprotection (TFA) were carried out with standard protocols. ¹H NMR (500 MHz, DMSO-d₆) δ8.67 (s, 1H), 8.22 (d, J=1.5 Hz, 1H), 8.08 (dd, J=1.0 Hz, 7.5 Hz, 1H), 7.96 (t, J=4.5 Hz, 1H), 7.87 (m, 2H), 7.81 (t, J=7.5 Hz, 1H), 7.51 (d, J=7.5 Hz, 1H), 7.28 (d, J=7.5 Hz, 1H), 4.62 (s, 2H), 2.97-2.86 (m, 5H), 2.61 (t, J=12 Hz, 2H), 2.57 (s, 3H), 1.85 (d, J=13.5 Hz, 2H), 1.73-1.60 (m, 2H; MS [M+H]⁺=500.0.

### Example 46

### 6-[2-chloro-4-(6-methyl-2-pyridyl)phenyl]-2-(methylamino)-8-[(1-methyl-4-piperidyl)methyl]pyrido[2,3-d]pyrimidin-7-one (I-175)

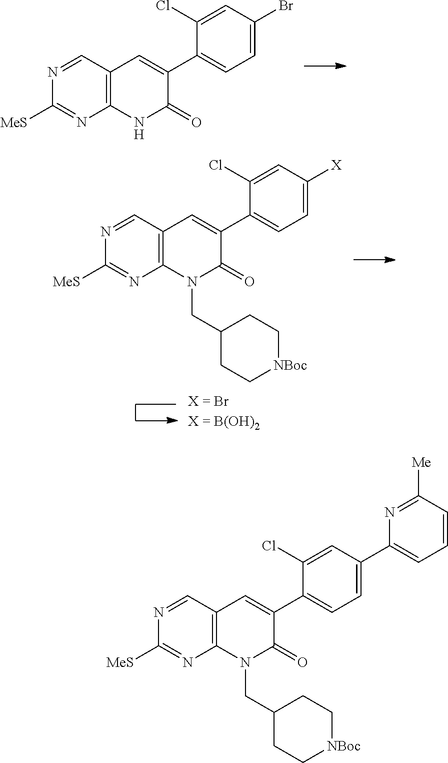

6-(4-bromo-2-chlorophenyl)-2-(methylthio)pyrido[2,3-d]pyrimidin-7(8H)-one was alkylated with tert-butyl 4-(tosyloxymethyl)piperidine-1-carboxylate to afford tert-butyl 4-((6-(4-bromo-2-chlorophenyl)-2-(methylthio)-7-oxopyrido[2,3-d]pyrimidin-8(7H)-yl)methyl)piperidine-1-carboxylate. Borylation (bis(pinacolato)diboron) and hydrolysis afforded 4-(8-((1-(tert-butoxycarbonyl)piperidin-4-yl)methyl)-2-(methylthio)-7-oxo-7,8-dihydropyrido[2,3-d]pyrimidin-6-yl)-3-chlorophenylboronic acid and Suzuki coupling (Pd(dppf)Cl₂, KOAc, dioxane, 80° C.) with 2-bromo-6-methyl-pyridine was carried out with standard protocols to afford tert-butyl 4-((6-(2-chloro-4-(6-methylpyridin-2-yl)phenyl)-2-(methylthio)-7-oxopyrido[2,3-d]pyrimidin-8(7H)-yl)methyl)piperidine-1-carboxylate. Introduction of the ethylamine and removal of the Boc group were accomplished by standard protocols. ¹H NMR (400 MHz, DMSO-d₆) δ 8.64 (s, 1H), 8.21 (d, J=1.7 Hz, 1H), 8.08 (dd, J=1.7, 8.0 Hz, 1H), 7.95-7.75 (m, 3H), 7.71 (br s, 1H), 7.51 (d, J=8.0 Hz, 1H), 7.28 (d, J=7.5 Hz, 1H), 4.43-4.14 (m, 2H), 2.91 (d, J=4.8 Hz, 3H), 2.82-2.68 (m, 2H), 2.57 (s, 3H), 2.13 (s, 3H), 1.94-1.69 (m, 3H), 1.61-1.45 (m, 2H), 1.43-1.28 (m, 2H); MS [M+H]⁺=489.2.

### Example 47

### 6-[2-chloro-4-(6-methyl-2-pyridyl)phenyl]-2-(methylamino)-8-[[(1R,5S)-3-methyl-3-azabicyclo[3.1.0]hexan-6-yl]methyl]pyrido[2,3-d]pyrimidin-7-one (I-176)

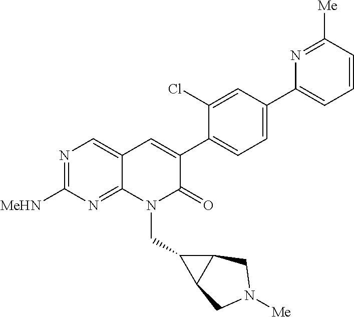 [SMILES: CNc1ncc2cc(-c3ccc(-c4cccc(C)n4)cc3Cl)c(=O)n(C[C@H]3[C@@H]4CN(C)C[C@@H]43)c2n1]

(1R,5S,6r)-3-Azabicyclo[3.1.0]hexane-6-methanol [CASRN 134575-13-6] was converted to (1R,5S,6r)-tert-butyl 6-(hydroxymethyl)-3-azabicyclo[3.1.0]hexane-3-carboxylate ((Boc)₂O, DMAP) and then brominated to afford (1R,5S,6r)-tert-butyl 6-(bromomethyl)-3-azabicyclo[3.1.0]hexane-3-carboxylate (CBr₄, PPh₃, DCM)

6-(2-chloro-4-(6-methylpyridin-2-yl)phenyl)-2-(methylthio)pyrido[2,3-d]pyrimidin-7(8H)-one was alkylated with (1R,5S,6r)-tert-butyl 6-(bromomethyl)-3-azabicyclo[3.1.0]hexane-3-carboxylate (Cs₂CO₃, DMF, 0° C. 18 h). Introduction of the methylamine and removal of the Boc group were accomplished by standard protocols to afford 8-((1R,5S,6r)-3-azabicyclo[3.1.0]hexan-6-ylmethyl)-6-(2-chloro-4-(6-methylpyridin-2-yl)phenyl)-2-(methylamino)pyrido[2,3-d]pyrimidin-7(8H)-one. Methylation of the pyrrolidine nitrogen is accomplished by reductive alkylation (HCHO, MeOH then NaBH₄). ¹H NMR (400 MHz, DMSO-d₆) δ 8.65 (s, 1H), 8.22 (d, J=1.6 Hz, 1H), 8.08 (dd, J=1.7, 8.0 Hz, 1H), 7.93-7.74 (m, 4H), 7.52 (d, J=8.0 Hz, 1H), 7.28 (d, J=7.4 Hz, 1H), 4.24-4.05 (m, 2H), 2.91 (d, J=4.7 Hz, 3H), 2.82 (d, J=8.7 Hz, 1H), 2.57 (s, 3H), 2.22-2.10 (m, 5H), 2.05-1.91 (m, 1H), 1.71-1.58 (m, 1H), 1.60-1.50 (m, 2H); MS [M+H]⁺=487.0.

2-(methylamino)-8-[(3-methyl-3-azabicyclo[3.1.0]hexan-6-yl)methyl]-6-[2-methyl-4-(6-methyl-2-pyridyl)phenyl]pyrido[2,3-d]pyrimidin-7-one (I-186) was prepared analogously starting from 6-(2-methyl-4-(6-methylpyridin-2-yl)phenyl)-2-(methylthio)pyrido[2,3-d]pyrimidin-7(8H)-one in place of 6-(2-chloro-4-(6 methylpyridin-2-yl)phenyl)-2-(methylthio)pyrido[2,3-d]pyrimidin-7(8H)-one. ¹H NMR (500 MHz, DMSO-d₆) δ 8.63 (s, 1H), 7.97 (s, 1H), 7.91 (d, J=8.0 Hz, 1H), 7.87-7.82 (m, 1H), 7.80-7.76 (m, 2H), 7.76-7.73 (m, 1H), 7.30 (d, J=7.9 Hz, 1H), 7.25-7.20 (m, 1H), 4.26-4.19 (m, 1H), 4.17-4.09 (m, 1H), 2.94-2.90 (m, 3H), 2.88-2.80 (m, 2H), 2.56 (s, 3H), 2.23 (s, 3H), 2.18 (s, 3H), 1.80-1.45 (m, 3H); MS [M+H]⁺=468.3.

### Example 48

### 6-[2-chloro-4-(2-methylthiazol-4-yl)phenyl]-2-(methylamino)-8-[[(2S)-morpholin-2-yl]methyl]pyrido[2,3-d]pyrimidin-7-one (I-179)

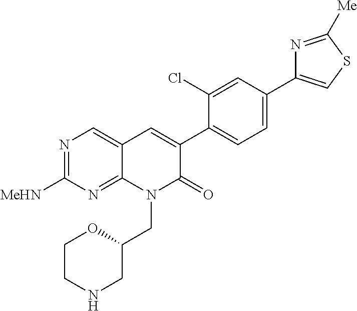 [SMILES: CNc1ncc2cc(-c3ccc(-c4csc(C)n4)cc3Cl)c(=O)n(C[C@@H]3CNCCO3)c2n1]

(S)-tert-butyl 2-((6-(4-bromo-2-chlorophenyl)-2-(methylthio)-7-oxopyrido[2,3-d]pyrimidin-8(7H)-yl)methyl)morpholine-4-carboxylate was borylated (bis(pinacolato)diboron, Pd(dppf)Cl₂, KOAc, dioxane, 80° C., 2 h) and the resulting dioxaborolane coupled with 4-bromo-2-methylthiazole (Pd(dppf)Cl₂, K₃PO₄, NaOAc, MeCN, H₂O, 100° C., 2 h) to afford (S)-tert-butyl 2-((6-(2-chloro-4-(2-methylthiazol-4-yl)phenyl)-2-(methylthio)-7-oxopyrido[2,3-d]pyrimidin-8(7H)-yl)methyl)morpholine-4-carboxylate. Introduction of the methylamine and removal of the Boc group were accomplished by standard protocols. ¹H NMR (400 MHz, CDCl₃) δ 8.45 (s, 1H), 8.00 (d, J=1.6 Hz, 1H), 7.78 (dd, J=1.6, 7.6 Hz, 1H), 7.58 (s, 1H), 7.41 (d, J=7.6 Hz, 1H), 7.36 (s, 1H), 5.52-5.50 (m, 1H), 4.61-4.52 (m, 2H), 4.00-3.91 (m, 2H), 3.60-3.55 (m, 1H), 3.11 (d, J=4.8 Hz, 3H), 2.93-2.87 (m, 2H), 2.82-2.80 (m, 2H), 2.78 (s, 3H); MS [M+H]⁺=483.0.

6-[2-chloro-4-(2-methylthiazol-4-yl)phenyl]-2-(methylamino)-8-[[(2R)-morpholin-2-yl]methyl]pyrido[2,3-d]pyrimidin-7-one (I-182) was prepared analogously from (R)-tert-butyl 2-((6-(4-bromo-2-chlorophenyl)-2-(methylthio)-7-oxopyrido[2,3-d]pyrimidin-8(7H)-yl)methyl)morpholine-4-carboxylate. ¹H NMR (400 MHz, CDCl₃) δ 8.45 (s, 1H), 8.00 (d, J=2.4 Hz, 1H), 7.78 (dd, J=2.4, 11.8 Hz, 1H), 7.58 (s, 1H), 7.41 (d, J=11.8 Hz, 1H), 7.37 (s, 1H), 5.62-5.60 (m, 1H), 4.63-4.52 (m, 2H), 4.00-3.91 (m, 2H), 3.62-3.55 (m, 1H), 3.10 (d, J=4.8 Hz, 3H), 2.95-2.91 (m, 2H), 2.87-2.78 (m, 2H), 2.78 (s, 3H); MS [M+H]⁺=483.0.

6-[2-chloro-4-(2-cyclopropylthiazol-4-yl)phenyl]-2-(methylamino)-8-[[(2S)-morpholin-2-yl]methyl]pyrido[2,3-d]pyrimidin-7-one (I-184) was prepared in accord with the procedure used to prepare I-179 except 4-bromo-2-cyclopropylthiazole replaced 4-bromo-2-methylthiazole. ¹H NMR (500 MHz, CDCl₃) δ 8.45 (brs, 1H), 7.99 (d, J=1.6 Hz, 1H), 7.78 (dd, J=1.6, 8.0 Hz, 1H), 7.57 (s, 1H), 7.40 (d, J=8.0 Hz, 1H), 7.28 (s, 1H), 5.52 (brs, 1H), 4.69-4.29 (m, 2H), 4.01 (brs, 1H), 3.91 (d, J=11.5 Hz, 1H), 3.58 (t, J=10.2 Hz, 1H), 3.11 (d, J=5.0 Hz, 3H), 2.97-2.85 (m, 2H), 2.85-2.74 (m, 2H), 2.41-2.31 (m, 1H), 1.20-1.09 (m, 4H); MS [M+H]⁺=509.2.

6-[2-chloro-4-(2-cyclopropylthiazol-4-yl)phenyl]-2-(methylamino)-8-[[(2R)-morpholin-2-yl]methyl]pyrido[2,3-d]pyrimidin-7-one (I-185) was prepared in accord with the procedure used to prepare I-182 except 4-bromo-2-cyclopropylthiazole replaced 4-bromo-2-methylthiazole. ¹H NMR (400 MHz, MeOD-d₄) δ 8.60 (s, 1H), 8.05 (d, J=1.6 Hz, 1H), 7.89 (dd, J=2.0 Hz; 8.0 Hz, 1H), 7.69 (s, 1H), 7.43 (d, J=8.0 Hz, 1H), 4.62 (brs, 1H), 4.47 (brs, 1H), 4.03 (brs, 1H), 3.89 (d, J=11.6 Hz, 2H), 3.61-3.54 (m, 1H), 3.07 (s, 3H), 2.90 (d, J=12.4 Hz, 1H), 2.85-2.75 (m, 3H), 2.46-2.42 (m, 1H), 1.25-1.20 (m, 2H), 1.14-1.10 (m, 2H); MS [M+H]⁺=509.1.

### Example 49

### 6-[2-chloro-4-(6-methylpyrazin-2-yl)phenyl]-8-(1-imino-1-oxo-thian-4-yl)-2-(methylamino)pyrido[2,3-d]pyrimidin-7-one (I-181)

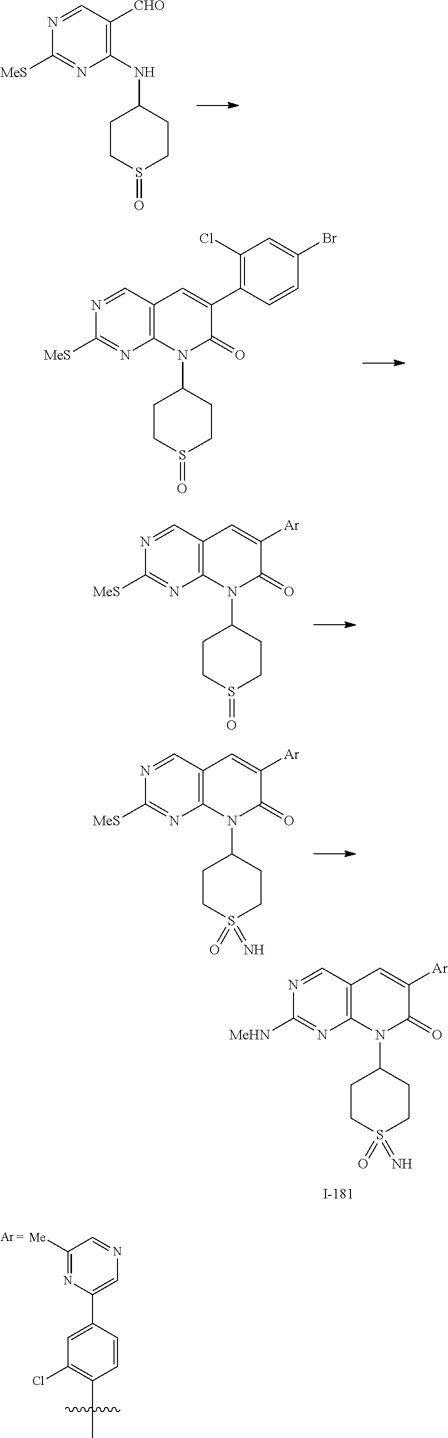

4-chloro-2-(methylthio)pyrimidine-5-carbaldehyde was condensed with 4-aminotetrahydro-2H-thiopyran-4-yl-1-oxide [CASRN 919513-33-0](TEA, THF, IPA, H₂O, 0° C.) to afford 2-(methylthio)-4-(tetrahydro-2H-thiopyran-4-ylamino)pyrimidine-5-carbaldehyde-1-oxide and subsequently cyclized with 4-bromo-2-chlorphenylacetic acid to afford 6-(4-bromo-2-chlorophenyl)-2-(methylthio)-8-(tetrahydro-2H-thiopyran-4-yl-1-oxide)pyrido[2,3-d]pyrimidin-7(8H)-one. Borylation (bis(pinacolato)diboron, Pd(dppf)Cl₂, KOAc, dioxane, 80° C., 2 h) and coupling with 2-chloropyrazine afforded 6-(2-chloro-4-(6-methylpyrazin-2-yl)phenyl)-2-(methylthio)-8-(tetrahydro-2H-thiopyran-4-yl-1-oxide)pyrido[2,3-d]pyrimidin-7(8H)-one.

Amination (CF₃CONH₂, MgO, Rh₂(OAc)₄, PhI(OAc)₂, DCM, RT, 16 h) afforded 6-[2-chloro-4-(6-methylpyrazin-2-yl)phenyl]-8-(1-imino-1-oxo-thian-4-yl)-2-(methylthio)pyrido[2,3-d]pyrimidin-7-one. Introduction of the methylamine onto the pyrimidine ring was accomplished by treatment with MeNH₂ and MeOH at 50° C. ¹H NMR (500 MHz, CDCl₃) δ 8.83 (s, 1H), 8.46 (s, 1H), 8.43 (s, 1H), 8.17 (d, J=1.6 Hz, 1H), 7.94 (dd, J=1.7, 8.0 Hz, 1H), 7.56 (s, 1H), 7.48 (d, J=8.0 Hz, 1H), 5.71-5.55 (m, 2H), 4.01-3.68 (m, 2H), 3.34-3.06 (m, 7H), 2.68 (s, 1H), 2.65 (s, 3H), 2.09-1.96 (m, 2H); MS [M+H]⁺=510.2.

### Example 50

### 6-[2-chloro-4-(4-methylthiazol-2-yl)phenyl]-2-(methylamino)-8-[[(2R)-morpholin-2-yl]methyl]pyrido[2,3-d]pyrimidin-7-one (I-189)

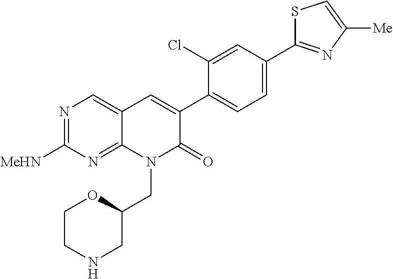 [SMILES: CNc1ncc2cc(-c3ccc(-c4nc(C)cs4)cc3Cl)c(=O)n(C[C@H]3CNCCO3)c2n1]

(S)-tert-butyl 2-((6-(4-bromo-2-chlorophenyl)-2-(methylthio)-7-oxopyrido[2,3-d]pyrimidin-8(7H)-yl)methyl)morpholine-4-carboxylate was borylated (bis(pinacolato)diboron, Pd(dppf)Cl₂, KOAc, dioxane, 80° C., 2 h) and the resulting dioxaborolane coupled with 2-bromo-4-methylthiazole (Pd(dppf)Cl₂, K₃PO₄, NaOAc, MeCN, H₂O, 100° C., 2 h) to afford (S)-tert-butyl 2-((6-(2-chloro-4-(4-methylthiazol-2-yl)phenyl)-2-(methylthio)-7-oxopyrido[2,3-d]pyrimidin-8(7H)-yl)methyl)morpholine-4-carboxylate. Introduction of the methylamine and removal of the Boc group were accomplished by standard protocols. ¹H NMR (400 MHz, CDCl₃) δ 8.45 (s, 1H), 8.06 (d, J=1.6 Hz, 1H), 7.83 (dd, J=2.0, 8.0 Hz, 1H), 7.58 (s, 1H), 7.41 (d, J=8.0 Hz, 1H), 6.92 (s, 1H), 5.70-5.58 (m, 1H), 4.61-4.50 (m, 2H), 4.00-3.89 (m, 2H), 3.59-3.55 (m, 1H), 3.11 (d, J=4.8 Hz, 3H), 2.93-2.90 (m, 2H), 2.88-2.87 (m, 2H), 2.51 (s, 3H); MS [M+H]⁺=483.0.

### Example 51

### 8-[(5-amino-1,3-dioxan-2-yl)methyl]-6-[2-chloro-4-(2-methylthiazol-4-yl)phenyl]-2-(methylamino)pyrido[2,3-d]pyrimidin-7-one (I-190)

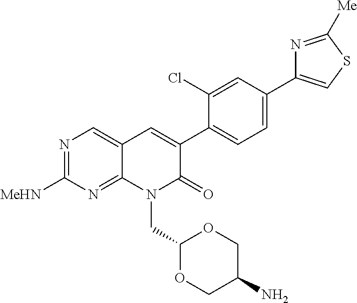 [SMILES: CNc1ncc2cc(-c3ccc(-c4csc(C)n4)cc3Cl)c(=O)n(C[C@H]3OC[C@H](N)CO3)c2n1]

Methyl 2-chloro-4-(4,4,5,5-tetramethyl-1,3,2-dioxaborolan-2-yl)-benzeneacetate [CASRN 849934-95-8] was coupled with 4-bromo-2-methyl-thiazole (Pd(dppf)Cl₂ K₂CO₃, 100° C.) to afford methyl 2-(2-chloro-4-(2-methylthiazol-4-yl)phenyl)acetate which was cyclized with 4-amino-2-(methylthio)pyrimidine-5-carbaldehyde to afford 6-(2-chloro-4-(2-methylthiazol-4-yl)phenyl)-2-(methylthio)pyrido[2,3-d]pyrimidin-7(8H)-one. Alkylation of the lactam nitrogen with 2-(2-(bromomethyl)-1,3-dioxan-5-yl)isoindoline-1,3-dione afforded 2-((2r,5r)-2-((6-(2-chloro-4-(2-methylthiazol-4-yl)phenyl)-2-(methylthio)-7-oxopyrido[2,3-d]pyrimidin-8(7H)-yl)methyl)-1,3-dioxan-5-yl)isoindoline-1,3-dione. Introduction of the methylamine and removal of the phthalimide group were accomplished by standard protocols. ¹H NMR (400 MHz, DMSO-d₆) δ 8.63 (s, 1H), 8.13 (s, 1H), 8.08 (d, J=2.0 Hz, 1H), 7.95 (dd, J=1.6, 8.0 Hz, 1H), 7.83 (s, 1H), 7.45 (d, J=8.0 Hz, 1H), 4.99-4.90 (m, 1H), 4.47-4.42 (m, 2H), 3.93-3.89 (m, 2H), 3.31 (s, 3H), 3.11 (t, J=2.8 Hz, 2H), 2.91 (d, J=4.8 Hz, 3H), 2.81-2.76 (m, 1H), 2.74 (s, 3H); MS [M+H]⁺=499.1.

Methyl 2-(2-chloro-4-(2-methylthiazol-4-yl)phenyl)acetate was cyclized with tert-butyl (2r,5r)-2-(2-(5-formyl-2-(methylthio)pyrimidin-4-ylamino)ethyl)-1,3-dioxan-5-ylcarbamate to afford tert-butyl (2r,5r)-2-(2-(6-(2-chloro-4-(2-methylthiazol-4-yl)phenyl)-2-(methylthio)-7-oxopyrido[2,3-d]pyrimidin-8(7H)-yl)ethyl)-1,3-dioxan-5-ylcarbamate. Introduction of the methyl amine and deprotection were carried out using standard protocols to afford 8-[2-(5-amino-1,3-dioxan-2-yl)ethyl]-6-[2-chloro-4-(2-methylthiazol-4-yl)phenyl]-2-(methylamino)pyrido[2,3-d]pyrimidin-7-one (I-191). ¹H NMR (400 MHz, MeOD-d₄) δ 8.45 (s, 1H), 7.94 (d, J=1.6 Hz, 1H), 7.75 (dd, J=1.6, 8 Hz, 1H), 7.65 (s, 1H), 7.62 (s, 1H), 7.31 (d, J=7.6 Hz, 1H), 4.67 (t, J=4.8 Hz, 1H), 4.48-4.47 (m, 2H), 3.95-3.68 (m, 4H), 2.95 (s, 3H), 2.66 (s, 3H), 2.58-2.52 (m, 1H), 2.04-1.89 (m, 2H); MS [M+H]⁺=513.2.

### Example 52

### 6-[2-chloro-4-(1-methylpyrazol-3-yl)phenyl]-2-(methylamino)-8-[[(2R)-morpholin-2-yl]methyl]pyrido[2,3-d]pyrimidin-7-one (I-191)

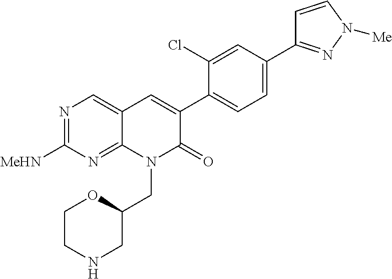 [SMILES: CNc1ncc2cc(-c3ccc(-c4ccn(C)n4)cc3Cl)c(=O)n(C[C@H]3CNCCO3)c2n1]

(R)-tert-butyl 2-((6-(4-bromo-2-chlorophenyl)-2-(methylthio)-7-oxopyrido[2,3-d]pyrimidin-8(7H)-yl)methyl)morpholine-4-carboxylate was borylated (bis(pinacolato)diboron, Pd(dppf)Cl₂, KOAc, dioxane, 80° C., 2 h) and the resulting dioxaborolane coupled with 3-bromo-1-methyl-1H-pyrazole (Pd(dppf)Cl₂, K₃PO₄, NaOAc, MeCN, H₂O, 100° C., 2 h) to afford (R)-tert-butyl 2-((6-(2-chloro-4-(2-methylthiazol-4-yl)phenyl)-2-(methylthio)-7-oxopyrido[2,3-d]pyrimidin-8(7H)-yl)methyl)morpholine-4-carboxylate. Introduction of the methylamine and removal of the Boc group were accomplished by standard protocols. ¹H NMR (400 MHz, CDCl₃) δ 8.45 (s, 1H), 7.90 (d, J=1.2 Hz, 1H), 7.78 (dd, J=1.2, 6.4 Hz, 1H), 7.58 (s, 1H), 7.38 (d, J=6.4 Hz, 1H), 7.37 (s, 1H), 6.55 (s, 1H), 5.50-5.48 (m, 1H), 4.62-4.49 (m, 2H), 3.99-3.98 (m, 1H), 3.96 (s, 3H), 3.92-3.89 (m, 1H), 3.60-3.54 (m, 1H), 3.11 (d, J=4.8 Hz, 3H), 2.92-2.87 (m, 2H), 2.82-2.77 (m, 2H); MS [M+H]⁺=466.1.

6-[2-chloro-4-(1-methyl-1 2 4-triazol-3-yl)phenyl]-2-(methylamino)-8-[[(2R)-morpholin-2-yl]methyl]pyrido[2,3-d]pyrimidin-7-one (I-192) was prepared analogously except 3-bromo-1-methyl-1H-pyrazole was replaced with 3-bromo-1-methyl-1 2 4 triazole. ¹H NMR (400 MHz, CDCl₃) δ 8.45 (s, 1H), 8.21 (s, 1H), 8.07 (s, 1H), 8.03-8.00 (m, 1H), 7.58 (s, 1H), 7.44 (d, J=8.0 Hz, 1H), 5.66-5.65 (m, 1H), 4.62-4.52 (m, 2H), 3.99-3.98 (m, 1H), 3.97 (s, 3H), 3.92-3.89 (m, 1H), 3.60-3.55 (m, 1H), 3.11 (d, J=5.2 Hz, 3H), 2.94-2.88 (m, 2H), 2.80-2.77 (m, 2H); MS [M+H]⁺=467.1.

### Example 53

### 6-[2-ethyl-4-(6-methyl-2-pyridyl)phenyl]-8-[[(2R)-morpholin-2-yl]methyl]-2-(oxetan-3-ylamino)pyrido[2,3-d]pyrimidin-7-one (I-193)

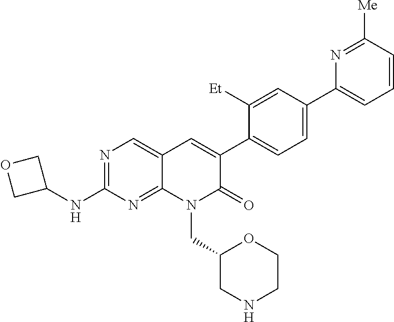 [SMILES: CCc1cc(-c2cccc(C)n2)ccc1-c1cc2cnc(NC3COC3)nc2n(C[C@H]2CNCCO2)c1=O]

(S)-tert-butyl 2-((6-(2-chloro-4-(6-methylpyridin-2-yl)phenyl)-2-(methylthio)-7-oxopyrido[2,3-d]pyrimidin-8(7H)-yl)methyl)morpholine-4-carboxylate was coupled with ethylboronic acid (Pd₂(dba)₃, Xphos, K₂CO₃, PhMe, 100° C.) to afford (S)-tert-butyl 2-((6-(2-ethyl-4-(6-methylpyridin-2-yl)phenyl)-2-(methylthio)-7-oxopyrido[2,3-d]pyrimidin-8(7H)-yl)methyl)morpholine-4-carboxylate. Introduction of the 3-amino-oxetane and deprotection were achieved by standard protocols ¹H NMR (400 MHz, DMSO-d₆) δ 8.68 (s, 1H), 8.67-8.63 (brs, 1H) 7.98 (d, J=2 Hz, 1H), 7.91-7.88 (dd, J=2, 8 Hz, 1H), 7.80-7.76 (m, 2H), 7.75 (s, 1H), 7.26-7.20 (m, 2H), 4.95-4.75 (m, 3H), 4.68-4.56 (m, 2H), 4.26-4.18 (m, 1H), 3.77-3.70 (m, 2H), 3.38-3.34 (m, 1H), 3.31 (s, 1H), 2.65-2.60 (m, 2H), 2.56 (s, 3H), 1.09 (t, J=8 Hz, 3H); MS [M+H]⁺=513.3.

6-[2-ethyl-4-(6-methyl-2-pyridyl)phenyl]-8-[[(2R)-morpholin-2-yl]methyl]-2-(oxetan-3-ylamino)pyrido[2,3-d]pyrimidin-7-one (I-194) was prepared analogously from (R)-tert-butyl 2-((6-(2-chloro-4-(6-methylpyridin-2-yl)phenyl)-2-(methylthio)-7-oxopyrido[2,3-d]pyridin-8(7H)-yl)methyl)morpholine-4-carboxylate. ¹H NMR (400 MHz, MeOD-d₄) δ8.66 (s, 1H), 7.91 (d, J=1.5 Hz, 1H), 7.83-7.77 (m, 2H), 7.75 (s, 1H), 7.67 (d, J=7.6 Hz, 1H), 7.30 (d, J=8 Hz, 1H), 7.26 (d, J=7.2 Hz, 1H), 5.10-4.98 (m, 4H), 4.86-4.72 (m, 2H), 4.71-4.64 (m, 1H), 4.44-4.36 (m, 1H), 4.00 (brs, 1H), 3.94-3.86 (m, 2H), 3.61-3.53 (m, 1H), 3.07-2.92 (m, 1H), 2.88-2.72 (m, 3H), 2.63 (s, 4H), 1.19 (t, J=7.6 Hz, 3H); MS [M+H]⁺=513.3.

### Example 54

### 6-[2-Chloro-4-[6-(hydroxymethyl)-2-pyridyl]phenyl]-2-(methylamino)-8-[[(2S)-morpholin-2-yl]methyl]pyrido[2,3-d]pyrimidin-7-one (I-195)

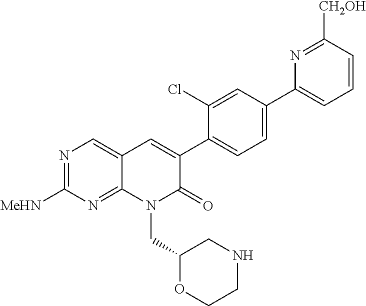 [SMILES: CNc1ncc2cc(-c3ccc(-c4cccc(CO)n4)cc3Cl)c(=O)n(C[C@@H]3CNCCO3)c2n1]

(R)-tert-Butyl 2-((6-(2-chloro-4-(4,4,5,5-tetramethyl-1,3,2-dioxaborolan-2-yl)phenyl)-2-(methylthio)-7-oxopyrido[2,3-d]pyrimidin-8(7H)-yl)methyl)morpholine-4-carboxylate was coupled with 2-bromo-6-hydroxymethylpyridine (Pd(PPh₃)₄, K₂CO₃) to afford (R)-tert-butyl 2-((6-(2-chloro-4-(6-(hydroxymethyl)pyridin-2-yl)phenyl)-2-(methylthio)-7-oxopyrido[2,3-d]pyrimidin-8(7H)-yl)methyl)morpholine-4-carboxylate. Introduction of the methylamine and deprotection was accomplished with standard protocols to afford I-195. ¹H NMR (400 MHz, DMSO-d₆) δ8.65 (s, 1H), 8.23 (d, J=1.6 Hz, 1H), 8.09 (dd, J=2, 8 Hz, 1H), 7.97-7.86 (m, 3H), 7.84 (s, 1H), 7.58-7.47 (m, 2H), 5.48 (t, J=6 Hz, 1H), 4.66 (d, J=6 Hz, 2H), 4.48-4.38 (m, 1H), 4.35-4.17 (m, 1H), 3.88-3.69 (m, 2H), 3.42-3.34 (m, 1H), 2.92 (d, J=4.4 Hz, 3H), 2.75-2.57 (m, 4H), 2.45-2.25 (m, 1H); MS [M+H]⁺=493.1.

### Example 55

### 6-[2-chloro-4-(6-methyl-2-pyridyl)phenyl]-2-(methylamino)-8-[2-(1-methylazetidin-3-yl)oxyethyl]pyrido[2,3-d]pyrimidin-7-one (I-196)

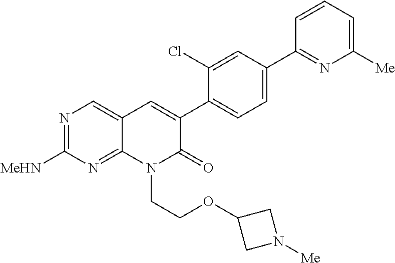 [SMILES: CNc1ncc2cc(-c3ccc(-c4cccc(C)n4)cc3Cl)c(=O)n(CCOC3CN(C)C3)c2n1]

6-(2-chloro-4-(6-methylpyridin-2-yl)phenyl)-2-(methylthio)pyrido[2,3-d]pyrimidin-7(8H)-one was alkylated with tert-butyl 3-(2-(tosyloxy)ethoxy)azetidine-1-carboxylate (Cs₂CO₃, DMF, 80° C., 2 h) to afford tert-butyl 3-(2-(6-(2-chloro-4-(6-methylpyridin-2-yl)phenyl-2-(methy thio)-7-oxopyrido[2,3-d]pyrimidin-8(7H)-yl)ethoxy)azetidine-1-carboxylate. The Boc group was removed (TFA) and the primary amine methylated (HCHO, Na BH(OAc)₃, EtOH, RT, 18 h). The methylthio was displaced with MeNH₂ (MeNH₂/EtOH, sealed tube, 80° C., 18 h) to afford I-196. ¹H NMR (400 MHz, MeOH-d₄) δ 8.60 (brs, 1H), 8.14 (s, 1H), 7.95 (d, J=8 Hz, 1H), 7.82-7.78 (m, 2H), 7.70 (d, J=7.6 Hz, 1H), 7.49 (d, J=7.6 Hz, 1H), 7.27 (d, J=7.6 Hz, 1H), 4.68 (brs, 2H), 4.26-4.21 (m, 1H), 3.77 (brs, 2H), 3.63-3.62 (m, 2H), 3.06 (s, 3H), 2.99 (brs, 2H), 2.63 (s, 3H), 2.35 (s, 3H); MS [M+H]⁺=491.0.

### Example 56

### 6-[2-chloro-4-(6-methyl-2-pyridyl)phenyl]-8-[(1,1-dioxo-1 4-thiazinan-2-yl)methyl]-2-(methylamino)pyrido[2,3-d]pyrimidin-7-one (I-197)

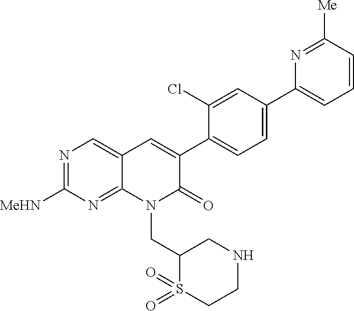 [SMILES: CNc1ncc2cc(-c3ccc(-c4cccc(C)n4)cc3Cl)c(=O)n(CC3CNCCS3(=O)=O)c2n1]

6-(4-Bromo-2-chlorophenyl)-2-(methylthio)pyrido[2,3-d]pyrimidin-7(8H)-one was alkylated (DIAD, PPh₃, DMF) with tert-butyl 2-(hydroxymethyl)thiomorpholine-4-carboxylate. Oxidation of the bis-thioether (MCPBA, DCM) afforded the bis-sulfone which was treated with methylamine to afford tert-butyl 2-((6-(4-bromo-2-chlorophenyl)-2-(methylamino)-7-oxopyrido[2,3-d]pyrimidin-8(7H)-yl)methyl)-1,1-dioxo-thiomorpholine-4-carboxylate. Borylation (KOAc, Pd(dppf)Cl₂, dioxane, 80° C.) and coupling with 2-chloro-6-methylpyridine (Pd(PPh₃)₄, Cs₂CO₃ 90° C., DME, EtOH, PhMe, H₂O) afforded tert-butyl 2-((6-(2-chloro-4-(6-methylpyridin-2-yl)phenyl)-2-(methylamino)-7-oxopyrido[2,3-d]pyrimidin-8(7H)-yl)methyl)-1,1-dioxo-thiomorpholine-4-carboxylate which was resolved by chiral chromatography and deprotected (TFA) to afford I-197. ¹H NMR (400 MHz, DMSO-d₆) δ 8.67 (s, 1H), 8.23 (d, J=1.6 Hz, 1H), 8.09 (dd, J=1.6, 8 Hz, 1H), 8.02-7.95 (m, 1H), 7.91-7.85 (m, 2H), 7.81 (t, J=8 Hz, 1H), 7.51 (d, J=7.6 Hz, 1H), 7.28 (d, J=7.6 Hz, 1H), 4.88-4.60 (m, 2H), 3.62-3.53 (m, 1H), 3.23-2.97 (m, 5H), 2.97-2.94 (m, 3H), 2.93-2.80 (m, 1H), 2.57 (s, 3H); MS [M+H]⁺=525.2.

### Example 57

### 8-[2-(5-Amino-1,3-dioxan-2-yl)ethyl]-6-[2-ethyl-4-(6-methyl-2-pyridyl)phenyl]-2-(oxetan-3-ylamino)pyrido[2,3-d]pyrimidin-7-one (I-198)

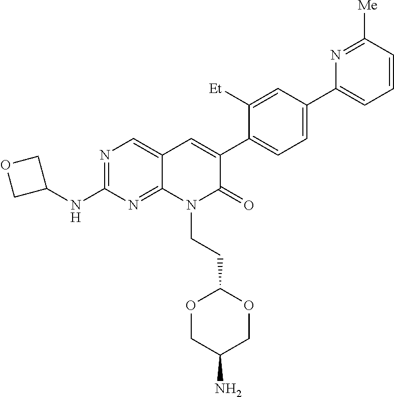 [SMILES: CCc1cc(-c2cccc(C)n2)ccc1-c1cc2cnc(NC3COC3)nc2n(CC[C@H]2OC[C@H](N)CO2)c1=O]

Methyl 2-(2-chloro-4-(6-methylpyridin-2-yl)phenyl)acetate was coupled with ethylboronic acid (Pd₂(dba)₃, Xphos, K₂CO₃, PhMe, 100° C.) to afford methyl 2-(2-ethyl-4-(6-methylpyridin-2-yl)phenyl)acetate which was condensed with tert-butyl (2r,5r)-2-(2-(5-formyl-2-(methylthio)pyrimidin-4-ylamino)ethyl)-1,3-dioxan-5-ylcarbamate and cyclized to afford tert-butyl (2r,5r)-2-(2-(6-(2-ethyl-4-(6-methylpyridin-2-yl)phenyl)-2-(methylthio)-7-oxopyrido[2,3-d]pyrimidin-8(7H)-yl)ethyl)-1,3-dioxan-5-ylcarbamate. Introduction of the oxetan-3-yl amine and deprotection were accomplished with standard protocols to afford I-198. ¹H NMR (400 MHz, DMSO-d₆) δ 8.68 (s, 1H), 8.67-8.63 (brs, 1H) 7.98 (d, J=2 Hz, 1H), 7.91-7.88 (dd, J=2, 8 Hz, 1H), 7.81-7.76 (m, 2H), 7.73 (s, 1H), 7.26-7.20 (m, 2H), 5.50-5.48 (m, 3H), 4.65-4.55 (m, 3H), 4.38-4.34 (t, J=6 Hz, 2H), 4.00-3.94 (dd, J=4, 8 Hz, 1H), 3.28-3.20 (m, 2H), 2.95-2.85 (m, 1H), 2.59 (s, 3H), 2.54-2.52 (m, 2H), 1.94-1.86 (m, 2H), 1.08 (t, J=8 Hz, 3H); MS [M+H]⁺=543.3.

### Example 58

### 3-[2-[6-[2-chloro-4-(6-methyl-2-pyridyl)phenyl]-2-(methylamino)-7-oxo-pyrido[2,3-d]pyrimidin-8-yl]ethyl]pyrrolidine-3-carbonitrile (I-199)

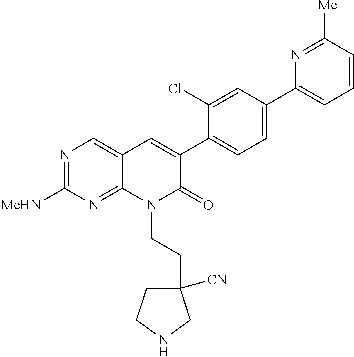 [SMILES: CNc1ncc2cc(-c3ccc(-c4cccc(C)n4)cc3Cl)c(=O)n(CCC3(C#N)CCNC3)c2n1]

6-(4-Bromo-2-chlorophenyl)-2-(methylthio)pyrido[2,3-d]pyrimidin-7(8H)-one was alkylated (DIAD, PPh₃, DMF) with tert-butyl 3-cyano-3-(2-hydroxyethyl)pyrrolidine-1-carboxylate. Borylation ((bis(pinacolato)diboron), KOAc, Pd(dppf)Cl₂, dioxane, 80° C.) and coupling with 2-chloro-6-methylpyridine (Pd(PPh₃)₄, K₂CO₃ 90° C., DME, EtOH, PhMe, H₂O) afforded tert-butyl 3-(2-(6-(2-chloro-4-(6-methylpyridin-2-yl)phenyl)-2-(methylthio)-7-oxopyrido[2,3-d]pyrimidin-8(7H)-yl)ethyl)-3-cyanopyrrolidine-1-carboxylate. Introduction of the methylamine and deprotection were accomplished with standard protocols to afford I-199. ¹H NMR (400 MHz, MeOH-d₄) δ8.55 (brs, 1H), 8.22 (q, J=3.6 Hz, 1H), 8.03 (d, J=2 Hz, 1H), 7.92 (d, J=8 Hz, 1H), 7.82 (dd, J=2, 8 Hz, 1H), 7.76 (s, 1H), 7.62 (d, J=7.6 Hz, 1H), 7.53 (d, J=8 Hz, 1H), 4.64 (brs, 2H), 3.84 (d, J=8.4 Hz, 1H), 3.56-3.33 (m, 3H), 2.99 (s, 3H), 2.71 (s, 3H), 2.66-2.57 (m, 1H), 2.30-2.17 (m, 3H); MS [M+H]⁺=500.1.

### Example 59

### 8-[(5-amino-1,3-dioxan-2-yl)methyl]-6-[2-chloro-4-(3-methyl-2-pyridyl)phenyl]-2-(methylamino)pyrido[2,3-d]pyrimidin-7-one (I-200)

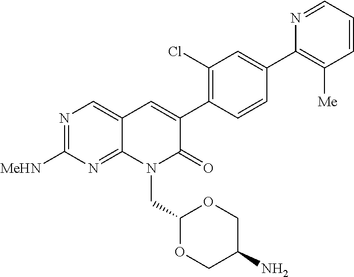 [SMILES: CNc1ncc2cc(-c3ccc(-c4ncccc4C)cc3Cl)c(=O)n(C[C@H]3OC[C@H](N)CO3)c2n1]

Methyl 2-(2-chloro-4-(4,4,5,5-tetramethyl-1,3,2-dioxaborolan-2-yl)phenyl)acetate was coupled with 2-bromo-3-methylpyridine (Pd(dppf)Cl₂, K₂CO₃, 100° C.) to afford methyl 2-(2-chloro-4-(3-methylpyridin-2-yl)phenyl)acetate which was condensed with 4-(((2r,5r)-5-(1,3-dioxoisoindolin-2-yl)-1,3-dioxan-2-yl)methylamino)-2-(methylthio)pyrimidine-5-carbaldehyde and cyclized to afford 2-((2r,5r)-2-((6-(2-chloro-4-(3-methylpyridin-2-yl)phenyl)(methylthio)-7-oxopyrido[2,3-d]pyrimidin-8(7H)-yl)methyl)-1,3-dioxan-5-yl)isoindoline-1,3-dione. Introduction of the methylamine and deprotection were accomplished with standard protocols to afford I-200. ¹H NMR (400 MHz, CDCl₃) δ 8.47 (d, J=4.0 Hz, 1H), 7.59 (s, 1H), 7.54-7.52 (d, J=8.0 Hz, 1H), 7.52 (s, 1H), 7.44-7.39 (m, 2H), 7.19 (s, 1H), 7.15-7.12 (m, 1H), 5.49 (d, J=4.4 Hz, 1H), 4.98 (s, 1H), 4.62 (d, J=2.4 Hz, 2H), 4.07-4.03 (m, 2H), 3.15-3.10 (m, 2H), 3.04 (d, J=5.2 Hz, 3H), 2.33 (s, 3H; MS [M+H]⁺=493.1.

### Example 60

### 6-[2-chloro-4-(6-methyl-2-pyridyl)phenyl]-8-[(3 3-difluoro-4-piperidyl)methyl]-2-(methylamino)pyrido[2,3-d]pyrimidin-7-one (I-201)

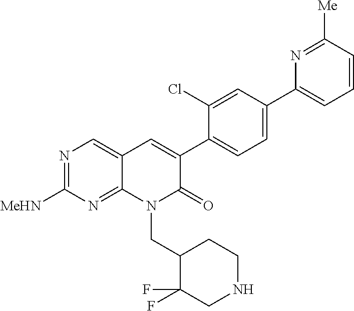 [SMILES: CNc1ncc2cc(-c3ccc(-c4cccc(C)n4)cc3Cl)c(=O)n(CC3CCNCC3(F)F)c2n1]

4-Chloro-2-(methylthio)pyrimidine-5-carbaldehyde was reacted with tert-butyl 4-(aminomethyl)-3,3-difluoropiperidine-1-carboxylate to afford tert-butyl 3,3-difluoro-4-((5-formyl-2-(methylthio)pyrimidin-4-ylamino)methyl)piperidine-1-carboxylate which was condensed with methyl 2-(2-chloro-4-(6-methylpyridin-2-yl)phenyl)acetate and cyclized to afford tert-butyl 4-((6-(2-chloro-4-(6-methylpyridin-2-yl)phenyl)-2-(methylthio)-7-oxopyrido[2,3-d]pyrimidin-8(7H)-yl)methyl)-3,3-difluoropiperidine-1-carboxylate. Introduction of the methyl amine was accomplished by treating with methylamine in a sealed tube (80° C., 16 h). Deprotection with TFA afforded I-201. ¹H NMR (400 MHz, DMSO-d₆) ¹H NMR (400 MHz, DMSO-d₆) δ 8.65 (s, 1H), 8.22 (d, J=1.6 Hz, 1H), 8.08 (dd, J=2.0, 8.4 Hz, 1H), 7.85 (m, 4H), 7.50 (d, J=8.0 Hz, 1H), 7.28 (d, J=7.6 Hz, 1H), 4.58 (m, 2H), 3.04 (m, 1H), 2.92 (d, J=4.8 Hz, 3H), 2.84 (m, 1H), 2.70 (m, 2H), 2.57 (s, 3H), 2.37 (m, 1H), 1.48 (m, 2H); MS [M+H]⁺=511.0.

### Example 61

### 6-[2-chloro-4-(6-methyl-2-pyridyl)phenyl]-8-[2-(1,1-dioxo-1 4-thiazinan-2-yl)ethyl]-2-(methylamino)pyrido[2,3-d]pyrimidin-7-one (I-202)

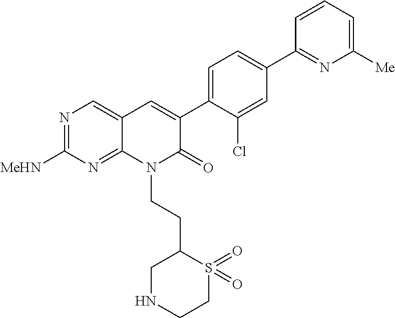 [SMILES: CNc1ncc2cc(-c3ccc(-c4cccc(C)n4)cc3Cl)c(=O)n(CCC3CNCCS3(=O)=O)c2n1]

tert-Butyl 1,1-dioxide-4-thiomorpholinecarboxylate [CASRN 215791-95-0] is alkylated with ethyl bromoacetate (LDA), reduced to the alcohol (NaBH₄) and tosylated to afford tert-butyl 2-(2-(tosyloxy)ethyl)-1,1-dioxo-thiomorpholine-4-carboxylate. Alkylation of 6-(2-chloro-4-(6-methylpyridin-2-yl)phenyl)-2-(methylthio)pyrido[2,3-d]pyrimidin-7(8H)-one with the tosylate (Cs₂CO₁, DMF) afforded tert-butyl 2-(2-(6-(2-chloro-4-(6-methylpyridin-2-yl)phenyl)-2-(methylthio)-7-oxopyrido[2,3-d]pyrimidin-8(7H)-yl)ethyl)-1,1-dioxo-thiomorpholine-4-carboxylate. Introduction of the methylamine and deprotection were accomplished with standard protocols to afford I-202. ¹H NMR (400 MHz, DMSO-d₆) δ 8.66 (s, 1H), 8.22 (d, J=1.6 Hz, 1H), 8.08 (dd, J=1.6, 8 Hz, 1H), 7.92 (d, J=4.4 Hz, 1H), 7.87 (d, J=8 Hz, 1H), 7.85 (s, 1H), 7.81 (t, J=7.6 Hz, 1H), 7.52 (d, J=8 Hz, 1H), 7.28 (d, J=7.6 Hz, 1H), 4.6-4.49 (m, 1H), 4.46-4.36 (m, 1H), 3.47-3.35 (m, 1H), 3.21-3.13 (m, 1H), 3.09-2.97 (m, 3H), 2.94 (d, J=4.4 Hz, 4H), 2.84-2.74 (m, 1H), 2.57 (s, 3H), 2.38-2.25 (m, 1H), 1.81-1.69 (m, 1H); MS [M+H]⁺=539.3.

### Example 62

### 5-[6-[2-Chloro-4-(6-methylpyrazin-2-yl)phenyl]-2-(methylamino)-7-oxo-pyrido[2,3-d]pyrimidin-8-yl]pentanamide (I-40)

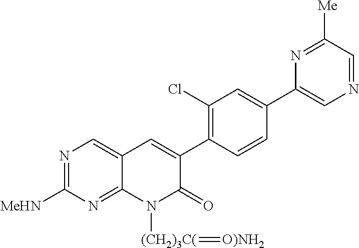

step 1: A mixture of 4-chlorobutanamide (3.5 equiv., 282.8 mg, 2.20 mmol), Cs₂CO₃ (617.3 mg, 1.894 mmol), and 6-[2-chloro-4-(6-methylpyrazin-2-yl)phenyl]-2-methylsulfanyl-8H-pyrido[2,3-d]pyrimidin-7-one (250 mg, 0.632 mmol) in anhydrous DMF (6.3 mL) was stirred at 40° C. for 16 h. The solvent was removed to afford the product 4-[6-[2-chloro-4-(6-methylpyrazin-2-yl)phenyl]-2-methylsulfanyl-7-oxo-pyrido[2,3-d]pyrimidin-8-yl]butanamide as a yellow solid. It was used in the next step without any further purification. MS (ESI) m/z 481.3 [M+1]⁺.

step 2: To 4-[6-[2-chloro-4-(6-methylpyrazin-2-yl)phenyl]-2-methylsulfanyl-7-oxo-pyrido[2,3-d]pyrimidin-8-yl]butanamide (240 mg, 0.499 mmol) in anhydrous DCM (8 mL) at 0° C. was added, portionwise, MCPBA (1.10 equiv., 0.549 mmol, 123.0 mg). The reaction mixture was stirred at 0° C. for 15 min, quenched with saturated NaHCO₃ (25 mL), and extracted with DCM (3×30 mL). The organic layers were combined and concentrated under reduced pressure to give crude 4-[6-[2-chloro-4-(6-methylpyrazin-2-yl)phenyl]-2-methylsulfinyl-7-oxo-pyrido[2,3-d]pyrimidin-8-yl]butanamide. MS (ESI) m/z 497.3 [M+1]⁺.

step 3: A mixture of 4-[6-[2-chloro-4-(6-methylpyrazin-2-yl)phenyl]-2-methylsulfinyl-7-oxo-pyrido[2,3-d]pyrimidin-8-yl]butanamide (100 mg, 0.201 mmol), MeNH₂ (2 mol/L in THF, 1.00 mmol, 0.50 mL), and DIPEA (4.0 equiv., 0.805 mmol, 0.140 mL) in anhydrous THF (0.8 mL) was stirred at RT for 30 min. The solvents were removed in vacuo, and the crude material was partitioned between DCM (40 mL) and water (20 mL). The aqueous layer was extracted with DCM (20 mL). The combined organic phases were filtered through a phase-separator cartridge. The crude product was purified by reverse-phase HPLC to afford 4-[6-[2-chloro-4-(6-methylpyrazin-2-yl)phenyl]-2-(methylamino)-7-oxo-pyrido[2,3-d]pyrimidin-8-yl]butanamide as an off-white solid (35.2 mg, 0.074 mmol, 37%). MS (ESI) m/z: 464.2 [M+1]⁺. ¹H NMR (400 MHz, DMSO-d6) δ 9.20-9.14 (m, 1H), 8.67-8.62 (m, 1H), 8.57 (s, 1H), 8.30-8.25 (m, 1H), 8.23-8.12 (m, 1H), 7.91-7.82 (m, 2H), 7.57 (d, 1H), 7.29-7.24 (m, 1H), 6.75-6.70 (m, 1H), 4.43-4.24 (m, 2H), 3.30 (s, 0H), 2.96-2.90 (m, 3H), 2.60 (s, 3H), 2.17-2.05 (m, 2H), 1.96-1.85 (m, 2H).

Compounds in Table IIa were made through similar procedures as used for the synthesis of G02850102, employing appropriate starting materials.

TABLE IIa

| | | MS m/z | |
|---|---|---|---|
| No. | Name | [M + H]+ | ¹H NMR |
| I-55 | 4-[6-[2-Chloro-4-(6- | 506.2 | (400 MHz, DMSO-d6) δ 9.15 (s, 1H), 8.71 (s, |
| | methylpyrazin-2- | | 1H), 8.63 (s, 1H), 8.57 (s, 1H), 8.27 (d, J = 1.8 Hz, |
| | yl)phenyl]-2-(oxetan-3- | | 1H), 8.15 (dd, J = 8.1, 1.8 Hz, 1H), 7.86 (s, |
| | ylamino)-7-oxo- | | 1H), 7.56 (d, J = 8.0 Hz, 1H), 7.26 (s, 1H), |
| | pyrido[2,3-d]pyrimidin-8- | | 6.72 (s, 1H), 5.02-4.97 (m, 1H), 4.96-4.75 (m, 2H), |
| | yl]butanamide | | 4.60 (t, J = 6.2 Hz, 2H), 4.29 (t, J = 7.1 Hz, 2H), |
| | | | 2.60 (s, 3H), 2.15 (t, J = 7.6 Hz, 2H), |
| | | | 1.92-1.82 (m, 2H). |
| I-41 | 4-[6-[2-Chloro-4-(6- | 520.2 | (400 MHz, DMSO-d6) δ 9.16 (s, 1H), 8.70 (s, |
| | methylpyrazin-2- | | 1H), 8.57 (s, 1H), 8.48-8.43 (m, 1H), |
| | yl)phenyl]-2-[(3- | | 8.30-8.25 (m, 1H), 8.19-8.12 (m, 1H), 7.89-7.84 (m, 1H), |
| | methyloxetan-3-yl)amino]- | | 7.59-7.52 (m, 1H), 7.33-7.28 (m, 1H), |
| | 7-oxo-pyrido[2,3- | | 6.77-6.72 (m, 1H), 4.80-4.73 (m, 214), 4.52-4.45 (m, |
| | d]pyrimidin-8- | | 2H), 4.24-4.19 (m, 2H), 2.60 (s, 3H), |
| | yl]butanamide | | 2.19-2.11 (m, 2H), 1.95-1.75 (m, 2H), 1.70 (s, 3H). |
| I-42 | 3-[6-[2-Chloro-4-(6- | 464.2 | (400 MHz, DMSO-d6) δ 9.15 (s, 1H), 8.65 (s, |
| | methylpyrazin-2- | | 1H), 8.57 (s, 1H), 8.27 (d, J = 1.8 Hz, 1H), |
| | yl)phenyl]-2-(ethylamino)- | | 8.15 (dd, J = 8.1, 1.8 Hz, 1H), 7.96 (s, 1H), 7.84 (s, |
| | 7-oxo-pyrido[2,3- | | 1H), 7.61-7.52 (m, 1H), 7.33 (s, 1H), 6.83 (s, |
| | d]pyrimidin-8- | | 1H), 4.55-4.46 (m, 2H), 3.48-3.36 (m, 2H), |
| | yl]propanamide | | 2.60 (s, 3H), 2.56-2.51 (m, 2H), 1.24-1.11 (m, |
| | | | 3H). |
| I-49 | 5-[6-[2-Chloro-4-(6- | 492.2 | (400 MHz, DMSO-d6) δ 9.16 (s, 1H), |
| | methylpyrazin-2- | | 8.67-8.62 (m, 1H), 8.57 (s, 1H), 8.30-8.24 (m, 1H), |
| | yl)phenyl]-2-(ethylamino)- | | 8.19-8.11 (m, 1H), 8.01-7.96 (m, 1H), 7.83 (s, 1H), |
| | 7-oxo-pyrido[2,3- | | 7.56 (d, J = 8.1 Hz, 1H), 7.22 (s, 1H), 6.68 (s, |
| | d]pyrimidin-8- | | 1H), 4.30 (s, 2H), 3.45-3.35 (m, 2H), 2.60 (s, |
| | yl]pentanamide | | 3H), 2.09 (t, J = 7.3 Hz, 2H), 1.70-1.65 (m, 2H), |
| | | | 1.59-1.54 (m, 2H), 1.26-1.16 (m, 3H). |
| I-71 | 4-[6-[2-Methyl-4-(6- | 485.2 | (400 MHz, DMSO-d6) δ 8.71-8.66 (m, 1H), |
| | methyl-2-pyridyl)phenyl]- | | 8.55 (s, 1H), 7.96 (s, 1H), 7.90 (dd, J = 7.8, 2.1 Hz, |
| | 2-(oxetan-3-ylamino)-7- | | 1H), 7.81-7.72 (m, 3H), 7.32-7.24 (m, |
| | oxo-pyrido[2,3- | | 2H), 7.24-7.18 (m, 1H), 6.72 (s, 1H), 4.99 (s, |
| | d]pyrimidin-8- | | 1H), 4.87 (s, 2H), 4.64-4.55 (m, 2H), 4.29 (t, J = 7.4 Hz, |
| | yl]butanamide | | 2H), 2.55 (s, 3H), 2.23 (s, 3H), 2.14 (t, J = 7.7 Hz, |
| | | | 2H), 1.91-1.83 (m, 2H). |
| I-70 | 4-[2-(Ethylamino)-6-[2- | 457.2 | (400 MHz, DMSO-d6) δ 8.62 (s, 1H), 7.96 (s, |
| | methyl-4-(6-methyl-2- | | 1H), 7.94-7.88 (m, 1H), 7.87-7.82 (m, 1H), |
| | pyridyl)phenyl]-7-oxo- | | 7.80-7.75 (m, 2H), 7.72 (s, 1H), 7.33-7.27 (m, |
| | pyrido[2,3-d]pyrimidin-8- | | 1H), 7.27-7.20 (m, 2H), 6.68 (s, 1H), 4.33 (s, |
| | yl]butanamide | | 2H), 3.48-3.36 (m, 2H), 2.55 (s, 3H), 2.24 (s, |
| | | | 3H), 2.17-2.08 (m, 2H), 1.97-1.84 (m, 2H), |
| | | | 1.19 (t, J = 7.4 Hz, 3H). |
| I-74 | 4-[2-(Ethylamino)-6-[2- | 471.2 | (400 MHz, DMSO-d6) δ 8.70-8.58 (m, 1H), |
| | methyl-4-(6-methyl-2- | | 7.98-7.95 (m, 1H), 7.93-7.82 (m, 2H), 7.77 (d, |
| | pyridyl)phenyl]-7-oxo- | | J = 5.1 Hz, 2H), 7.74-7.67 (m, 2H), 7.29 (d, J = 7.9 Hz, |
| | pyrido[2,3-d]pyrimidin-8- | | 1H), 7.24-7.18 (m, 1H), 4.42-4.23 (m, |
| | yl]-N-methyl-butanamide | | 2H), 3.47-3.34 (m, 2H), 2.56 (s, 3H), 2.54 (d, J = 4.6 Hz, |
| | | | 3H), 2.24 (s, 3H), 2.17-2.09 (m, 2H), |
| | | | 2.00-1.78 (m, 2H), 1.19 (t, J = 7.2 Hz, 3H). |
| I-87 | N-methyl-4-[6-[2-methyl- | 499.3 | (400 MHz, DMSO-d6) δ 8.69 (s, 1H), |
| | 4-(6-methyl-2- | | 8.59-8.53 (m, 1H), 7.96 (d, J = 1.7 Hz, 1H), 7.90 (dd, J = 7.9, |
| | pyridyl)phenyl]-2-(oxetan- | | 1.9 Hz, 1H), 7.81-7.68 (m, 3H), 7.28 (d, J = 7.9 Hz, |
| | 3-ylamino)-7-oxo- | | 1H), 7.25-7.17 (m, 1H), 4.98 (s, 1H), |
| | pyrido[2,3-d]pyrimidin-8- | | 4.86 (s, 2H), 4.60 (t, J = 6.2 Hz, 2H), |
| | yl]butanamide | | 4.32-4.24 (m, 2H), 2.59-2.54 (m, 6H), 2.23 (s, 3H), |
| | | | 2.20-2.11 (m, 2H), 1.90-1.85 (m, 2H). |
| I-98 | 6-[2-Chloro-4-(6- | 518.2 | (400 MHz, DMSO-d6) δ 9.15 (s, 1H), 8.66 (s, |
| | methylpyrazin-2- | | 1H), 8.57 (s, 1H), 8.27 (d, J = 1.8 Hz, 1H), |
| | yl)phenyl]-2- | | 8.15 (dd, J = 8.0, 1.8 Hz, 1H), 7.90 (m, 1H), 7.83 (s, |
| | (isopropylamino)-8-[2-(5- | | 1H), 7.55 (d, J = 8.0 Hz, 1H), 7.48 (s, 1H), |
| | oxopyrrolidin-3- | | 4.31 (m, 2H), 4.13 (m, 1H), 3.47-3.36 (m, 1H), |
| | yl)ethyl]pyrido[2,3- | | 3.03-2.91 (m, 1H), 2.60 (s, 3H), 2.41 (m, 1H), |
| | d]pyrimidin-7-one | | 2.36-2.25 (m, 1H), 1.94 (m, 1H), 1.81 (m, 2H), 1.24 (d, |
| | | | J = 6.2 Hz, 6H). |
| I-99 | 6-[2-Chloro-4-(6- | 518.2 | (400 MHz, DMSO-d6) δ 7.15 (s, 1H), 8.66 (s, |
| | methylpyrazin-2- | | 1H), 8.57 (s, 1H), 8.27 (d, J = 1.8 Hz, 1H), |
| | yl)phenyl]-2- | | 8.15 (dd, J = 8.0, 1.8 Hz, 1H), 7.89 (d, J = 7.6 Hz, 1H), |
| | (isopropylamino)-8-[2-(5- | | 7.83 (s, 1H), 7.55 (d, J = 8.0 Hz, 1H), 7.48 (s, |
| | oxopyrrolidin-3- | | 1H), 4.31 (m, 2H), 4.14 (m, 1H), 3.47-3.37 (m, |
| | yl)ethyl]pyrido[2,3- | | 1H), 3.02-2.92 (m, 1H), 2.60 (s, 3H), 2.41 (m, |
| | d]pyrimidin-7-one | | 1H), 2.36-2.25 (m, 1H), 1.94 (m, 1H), 1.80 (m, |
| | | | 2H), 1.24 (d, J = 6.2 Hz, 6H). |
| I-100 | 6-[2-Chloro-4-(6- | 518.2 | (400 MHz, DMSO-d6) δ 9.15 (s, 1H), 8.65 (s, |
| | methylpyrazin-2- | | 1H), 8.57 (s, 1H), 8.27 (d, J = 1.8 Hz, 1H), |
| | yl)phenyl]-2- | | 8.15 (dd, J = 8.0, 1.8 Hz, 1H), 7.87 (d, J = 7.6 Hz, 1H), |
| | (isopropylamino)-8-[2-(2- | | 7.83 (s, 1H), 7.60-7.53 (m, 2H), 4.39 (m, 2H), |
| | oxopyrrolidin-3- | | 4.16 (m, 1H), 3.23-3.10 (m, 2H), 2.60 (s, 3H), |
| | yl)ethyl]pyrido[2,3- | | 2.36-2.20 (m, 2H), 2.14 (m, 1H), 1.75 (m, 1H), |
| | d]pyrimidin-7-one | | 1.57 (m, 1H), 1.23 (d, J = 6.2 Hz, 6H). |
| I-101 | 6-[2-Chloro-4-(6- | 518.2 | (400 MHz, DMSO-d6) δ 9.15 (s, 1H), 8.65 (s, |
| | methylpyrazin-2- | | 1H), 8.57 (s, 1H), 8.27 (d, J = 1.7 Hz, 1H), |
| | yl)phenyl]-2- | | 8.20-8.10 (m, 1H), 7.87 (d, J = 7.6 Hz, 1H), 7.83 (s, |
| | (isopropylamino)-8-[2-(2- | | 1H), 7.61-7.51 (m, 2H), 4.39 (m, 2H), 4.17 (m, |
| | oxopyrrolidin-3- | | 1H), 3.23-3.07 (m, 2H), 2.60 (s, 3H), |
| | yl)ethyl]pyrido[2,3- | | 2.37-2.20 (m, 2H), 2.14 (m, 1H), 1.75 (m, 1H), 1.56 (m, |
| | d]pyrimidin-7-one | | 1H), 1.23 (d, J = 6.2 Hz, 6H). |
| I-102 | 6-[2-Chloro-4-(6- | 490.2 | (400 MHz, DMSO-d6) δ 9.16 (s, 1H), 8.65 (s, |
| | methylpyrazin-2- | | 1H), 8.57 (s, 1H), 8.27 (d, J = 1.7 Hz, 1H), |
| | yl)phenyl]-2- | | 8.15 (dd, J = 8.0, 1.8 Hz, 1H), 7.87 (m, 1H), 7.84 (s, |
| | (methylamino)-8-[2-(2- | | 1H), 7.57 (d, J = 8.0 Hz, 2H), 4.43 (m, 2H), |
| | oxopyrrolidin-3- | | 3.24-3.09 (m, 2H), 2.93 (d, J = 4.8 Hz, 3H), 2.60 (s, |
| | yl)ethyl]pyrido[2,3- | | 3H), 2.31 (m, 2H), 2.15 (m, 1H), 1.77 (m, 1H), |
| | d]pyrimidin-7-one | | 1.57 (m, 1H). |
| I-103 | 6-[2-Chloro-4-(6- | 490.2 | (400 MHz, DMSO-d6) δ 9.16 (s, 1H), 8.65 (s, |
| | methylpyrazin-2- | | 1H), 8.57 (s, 1H), 8.27 (d, J = 1.8 Hz, 1H), |
| | yl)phenyl]-2- | | 8.15 (dd, J = 8.0, 1.8 Hz, 1H), 7.89 (m, 1H), 7.85 (s, |
| | (methylamino)-8-[2-(2- | | 1H), 7.57 (d, J = 8.0 Hz, 2H), 4.43 (m, 2H), |
| | oxopyrrolidin-3- | | 3.25-3.08 (m, 2H), 2.93 (d, J = 4.7 Hz, 3H), 2.60 (s, |
| | yl)ethyl]pyrido[2,3- | | 3H), 2.31 (m, 2H), 2.16 (m, 1H), 1.78 (m, 1H), |
| | d]pyrimidin-7-one | | 1.57 (m, 1H). |
| I-109 | 6-[2-Chloro-4-(6- | 490.2 | (400 MHz, DMSO-d6) δ 9.15 (s, 1H), 8.69 (m, |
| | methylpyrazin-2- | | 1H), 8.57 (s, 1H), 8.27 (d, J = 1.8 Hz, 1H), |
| | yl)phenyl]-2- | | 8.15 (dd, J = 8.0, 1.8 Hz, 1H), 7.90 (m, 1H), 7.85 (s, |
| | (methylamino)-8-[2-(5- | | 1H), 7.56 (d, J = 8.0 Hz, 1H), 7.48 (s, 1H), |
| | oxopyrrolidin-3- | | 4.34 (m, 2H), 3.46-3.36 (m, 1H), 3.03-2.89 (m, 4H), |
| | yl)ethyl]pyrido[2,3- | | 2.60 (s, 3H), 2.39 (m, 1H), 2.36-2.24 (m, 1H), |
| | d]pyrimidin-7-one | | 1.93 (dd, J = 15.9, 7.5 Hz, 1H), 1.88-1.74 (m, |
| | | | 2H). |
| I-116 | 6-[2-Chloro-4-(6- | 490.2 | (400 MHz, DMSO-d6) δ 9.15 (s, 1H), 8.69 (m, |
| | methylpyrazin-2- | | 1H), 8.57 (s, 1H), 8.27 (d, J = 1.8 Hz, 1H), |
| | yl)phenyl]-2- | | 8.15 (dd, J = 8.0, 1.8 Hz, 1H), 7.90 (m, 1H), 7.85 (s, |
| | (methylamino)-8-[2-(5- | | 1H), 7.56 (d, J = 8.0 Hz, 1H), 7.48 (s, 1H), |
| | oxopyrrolidin-3- | | 4.31 (m, 2H), 3.46-3.37 (m, 1H), 3.01-2.95 (m, 1H), |
| | yl)ethyl]pyrido[2,3- | | 2.93 (d, J = 4.7 Hz, 3H), 2.60 (s, 3H), 2.39 (m, |
| | d]pyrimidin-7-one | | 1H), 2.36-2.25 (m, 1H), 1.93 (dd, J = 16.2, 7.5 Hz, |
| | | | 1H), 1.82 (m, 2H). |

### Example 63

### 8-(((1S,4S)-4-Aminocyclohexyl)methyl)-6-(2-chloro-4-(6-methylpyrazin-2-yl)phenyl)-2-(ethylamino)pyrido[2,3-d]pyrimidin-7(8H)-one (I-38)

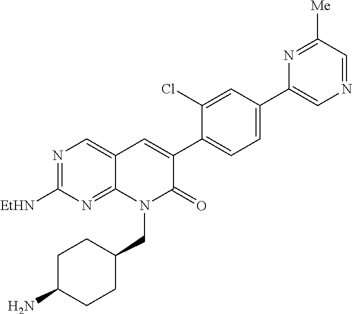

step 1: Sodium hydride (20 mg, 60% in oil mineral, 0.50 mmol) was added portionwise to a solution of 6-(2-chloro-4-(6-methylpyrazin-2-yl)phenyl)-2-(methylthio)pyrido[2,3-d]pyrimidin-7(8H)-one (100 mg, 0.25 mmol) in DMF (10 mL) that was cooled to 0° C. The suspension was stirred at RT until homogeneous, and then ((1S,4S)-4-(tert-butoxycarbonylamino)cyclohexyl)methyl 4-methylbenzenesulfonate (192 mg, 0.50 mmol) was added. The mixture was stirred at 85° C. for two h, cooled to RT, diluted with DCM (50 mL) and washed with water (3×50 mL). The organic phase was dried (Na₂SO₄), filtered, concentrated in vacuo, and purified by SiO₂ chromatography eluting with PE/EtOAc (1:1) to afford tert-butyl (1s,4s)-4-((6-(2-chloro-4-(6-methylpyrazin-2-yl)phenyl)-2-(methylthio)-7-oxopyrido[2,3-d]pyrimidin-8(7H)-yl)methyl)cyclohexylcarbamate as a yellow solid (130 mg, 86%). MS (ESI): m/z=551.0 [M−55]⁺.

step 2: MCBPA (176 mg, 0.82 mmol) was added portionwise to a solution of tert-butyl (1s,4s)-4-((6-(2-chloro-4-(6-methylpyrazin-2-yl)phenyl)-2-(methylthio)-7-oxopyrido[2,3-d]pyrimidin-8(7H)-yl)methyl)cyclohexylcarbamate (250 mg, 0.41 mmol) in DCM (5 mL) that was cooled to 0° C. The suspension was stirred at RT for two h and then quenched with saturated aqueous NH₄Cl aqueous solution. The mixture was extracted with DCM (50 mL×3), and the combined organic layers were washed water, dried (Na₂SO₄), filtered and concentrated in vacuo to give the crude product tert-butyl (1S,4S)-4-((6-(2-chloro-4-(6-methylpyrazin-2-yl)phenyl)-2-(methylsulfonyl)-7-oxopyrido[2,3-d]pyrimidin-8(7H)-yl)methyl)cyclohexylcarbamate as yellow solid (150 mg, 57%). LCMS (ESI): m/z=583.2 [M−55]⁺.

step 3: A mixture of tert-butyl (1S,4S)-4-((6-(2-chloro-4-(6-methylpyrazin-2-yl)phenyl)-2-(methylsulfonyl)-7-oxopyrido[2,3-d]pyrimidin-8(7H)-yl)methyl)cyclohexylcarbamate (150 mg, 0.23 mmol), ethylamine hydrochloride (38 mg, 0.46 mmol) and DIPEA (121 mg, 0.92 mol) in propan-2-ol (5 mL) was stirred at 80° C. for 2 h. The mixture was then concentrated in vacuo and purified by SiO₂ chromatography eluting with PE/EtOAc (1:1) to afford tert-butyl (1S,4S)-4-((6-(2-chloro-4-(6-methylpyrazin-2-yl)phenyl)-2-(ethylamino)-7-oxopyrido[2,3-d]pyrimidin-8(7H)-yl)methyl)cyclohexylcarbamate (100 mg, 71%). LCMS (ESI): m/z=604.2 [M+1]⁺.

step 4: A solution of tert-butyl (1s,4s)-4-((6-(2-chloro-4-(6-methylpyrazin-2-yl)phenyl)-2-(ethylamino)-7-oxopyrido[2,3-d]pyrimidin-8(7H)-yl)methyl)cyclohexylcarbamate (100 mg, 0.17 mmol) in TFA (2 mL) and DCM (2 mL) was stirred at 0° C. for 1 h. The reaction mixture was concentrated in vacuo and purified by preparative HPLC to afford 8-(((1S,4S)-4-aminocyclohexyl)methyl)-6-(2-chloro-4-(6-methylpyrazin-2-yl)phenyl)-2-(ethylamino)pyrido[2,3-d]pyrimidin-7(8H)-one as a white solid (60 mg, 71%). ¹H NMR (500 MHz, CD₃OD-d₄): δ 8.98 (s, 1H), 8.61 (br, 1H), 8.50 (s, 1H), 8.27 (d, J=1.6 Hz, 1H), 8.11-8.09 (dd, J=1.5, 8.5 Hz, 1H), 7.80 (s, 1H), 7.55 (d, J=8.0 Hz, 1H), 4.35 (d, J=5.7 Hz, 2H), 3.54 (dd, J=7.1, 14.3 Hz, 2H), 2.76-2.69 (m, 1H), 2.67 (s, 3H), 2.08-2.00 (m, 1H), 1.98-1.91 (m, 2H), 1.85-1.76 (m, 2H), 1.33-1.30 (m, 3H), 1.26-1.09 (m, 4H); MS (ESI): m/z=504.1 [M+1]⁺.

Compounds in Table IIb were made through similar procedures employing appropriate starting materials.

TABLE IIb

| Cpd | | MS m/z | |
|---|---|---|---|
| No. | Name | [M + H]+ | ¹H NMR |
| I-33 | 8-(((1r,4r)-4- | 531.1 | 8.666 (s, 1H), 8.150 (s, 1H), 7.983-7.963 (d, 1H), |
| | aminocyclohexyl)methyl)- | | 7.824-7.793 (m, 2H), 7.723-7.707 (d, 1H), |
| | 6-(2-chloro-4-(6- | | 7.510-7.494 (d, 1H), 7.294--7.279 (d, 1H), 5.028 (t, 3H), |
| | methylpyridin-2- | | 4.845 (m, 2H), 4.339-4.325 (d, 2H), |
| | yl)phenyl)-2-(oxetan-3- | | 2.705-2.633 (m, 4H), 1.999-1.760 (m, 5H), 1.310-1.091 (m, 4H) |
| | ylamino)pyrido[2,3- | | |
| | d]pyrimidin-7(8H)-one | | |
| I-53 | 6-[2-chloro-4-(6- | 518.0 | (500 MHz, CD₃OD-d₄): δ 8.83 (s, 1H), 8.48 (br, |
| | methylpyrazin-2- | | 1H), 8.43 (s, 1H), 8.17 (d, J = 1.6 Hz, 1H), |
| | yl)phenyl]-2-(ethylamino)- | | 7.94 (dd, J = 1.6, 8.0 Hz, 1H), 7.58 (s, 1H), 7.50 (d, J = 8.0 Hz, |
| | 8-[2-(2-oxo-4- | | 1H), 5.82 (br, 1H), 5.63 (br, 1H), |
| | piperidyl)ethyl]pyrido[2,3- | | 4.66-4.31 (m, 2H), 3.61-3.50 (m, 2H), 3.43-3.27 (m, 2H), |
| | d]pyrimidin-7-one | | 2.65 (s, 3H), 2.63-2.55 (m, 1H), 2.21-1.74 (m, |
| | | | 5H), 1.56 (s, 1H), 1.32 (t, J = 7.2 Hz, 3H) |
| I-52 | 6-[2-chloro-4-(6- | 506.1 | (500 MHz, DMSO-d₆): δ 9.15 (s, 1H), 8.68 (s, 1H), |
| | methylpyrazin-2- | | 8.57 (s, 1H), 8.27 (d, 1H), 8.16-8.14 (m, 1H), |
| | yl)phenyl]-2-(ethylamino)- | | 8.01 (s, 1H), 7.84 (s, 1H), 7.55 (d, 1H), 4.48 (d, 2H), |
| | 8-[2-[(3S)-pyrrolidin-3- | | 4.06 (s, 1H), 3.64 (s, 2H), 3.42-3.39 (m, 2H), |
| | yl]oxyethyl]pyrido[2,3- | | 2.79-2.74 (m, 2H), 2.68-2.65 (m, 2H), 2.60 (s, 3H), |
| | d]pyrimidin-7-one | | 1.74-1.67 (m, 1H), 1.60-1.58 (m, 1H), |
| | | | 1.22-1.16 (m, 3H) |
| I-51 | 6-[2-chloro-4-(6- | 506.1 | (500 MHz, DMSO-d₆): δ 9.15 (s, 1H),8.65 (s, 1H), |
| | methylpyrazin-2- | | 8.57 (s, 1H), 8.27 (d, 1H), 8.16-8.14 (m, 1H), |
| | yl)phenyl]-2-(ethylamino)- | | 8.01 (s, 1H), 7.84 (s, 1H), 7.54 (d, 1H), 4.48 (d, 2H), |
| | 8-[2-[(3R)-pyrrolidin-3- | | 4.04 (s, 1H), 3.64 (s, 2H), 3.42-3.39 (m, 2H), |
| | yl]oxyethyl]pyrido[2,3- | | 2.79-2.74 (m, 2H), 2.68-2.65 (m, 2H), 2.60 (s, 3H), |
| | d]pyrimidin-7-one | | 1.74-1.67 (m, 1H), 1.60-1.58 (m, 1H), |
| | | | 1.22-1.16 (m, 3H) |
| I-46 | 8-[2-(azetidin-3- | 478.1 | (500 MHz, DMSO-d₆): δ 9.16 (s, 1H), 8.69 (s, |
| | yloxy)ethyl]-6-[2-chloro- | | 1H), 8.57 (s, 1H), 8.27 (s, 1H), 8.16-8.11 (m, 1H), |
| | 4-(6-methylpyrazin-2- | | 7.93 (d, 1H), 7.85 (s, 1H), 7.56-7.54 (s, 1H), |
| | yl)phenyl]-2- | | 4.50-4.36 (m, 3H), 3.64-3.54 (m, 4H), 3.48-3.26 (m, |
| | (methylamino)pyrido[2,3- | | 2H), 2.92 (s, 3H), 2.58 (d, 3H) |
| | d]pyrimidin-7-one | | |
| I-47 | 8-[2-(azetidin-3- | 528.0 | (500 MHz, DMSO-d₆): δ 9.16 (s, 1H), 8.75 (s, 1H), |
| | yloxy)ethyl]-6-[2-chloro- | | 8.57-8.55 (m, 1H), 8.32-8.22 (m, 2H), |
| | 4-(6-methylpyrazin-2- | | 8.17-8.15 (m, 1H), 7.90 (s, 1H), 7.56-7.54 (m, 1H), |
| | yl)phenyl]-2-(2,2- | | 4.49-4.26 (m, 2H), 3.80 (m, 2H), 3.62-3.47 (m, 4H), |
| | difluoroethylamino)pyrido[2, | | 3.37 (brs, 6H), 2.64 (s, 3H) |
| | 3-d]pyrimidin-7-one | | |
| I-30 | 8-[2-(azetidin-3- | 491.0 | (500 MHz, DMSO-d₆): δ 8.65 (s, 1H), 8.22 (s, 1H), |
| | yloxy)ethyl]-6-[2-chloro- | | 8.09-8.07 (m, 1H), 8.02-8.01 (m, 1H), 7.87 (d, 1H), |
| | 4-(6-methyl-2- | | 7.83 (d, 1H), 7.80 (d, 1H), 7.49 (d, 1H), 7.28 (d, |
| | pyridyl)phenyl]-2- | | 1H), 4.50-4.47 (m, 2H), 4.34-4.31 (m, 1H), |
| | (ethylamino)pyrido[2,3- | | 3.63-3.54 (m, 4H), 3.44-3.37 (m, 4H), 2.56 (s, 3H), |
| | d]pyrimidin-7-one | | 1.22-1.16 (m, 3H) |
| I-31 | 8-(2-(azetidin-3- | 471.1 | (500 MHz, DMSO-d₆): δ 8.63 (s, 1H), 7.95 (s, 1H), |
| | yloxy)ethyl)-2- | | 7.91-7.89 (m, 1H), 7.78-7.77 (m, 2H), 7.74 (s, 1H), |
| | (ethylamino)-6-(2-methyl- | | 7.28 (d, 1H), 7.23-7.21 (m, 1H), 4.49 (s, 2H), |
| | 4-(6-methylpyridin-2- | | 4.32 (s, 1H), 3.62-3.54 (m, 4H), 3.43-3.33 (m, 4H), |
| | yl)phenyl)pyrido[2,3- | | 2.55 (s, 3H), 2.22 (s, 3H), 1.22-1.98 (m, 3H) |
| | d]pyrimidin-7(8H)-one | | |
| I-91 | 8-[2-(Azetidin-3- | 506.0 | (500 MHz, MeOD-d4): δ 8.96 (s, 1H), 8.61 (brs, |
| | yloxy)propyl]-6-[2-chloro- | | 1H), 8.49 (s, 1H), 8.26 (d, J = 1.5 Hz, 1H), 8.09 (dd, |
| | 4-(6-methylpyrazin-2- | | J = 2.0, 8.5 Hz, 1H), 7.81 (s, 1H), 7.54 (d, J = 8.0 Hz |
| | yl)phenyl]-2- | | 1H), 4.64 (m, 1H), 4.56 (m, 1H), 4.42 (m, 1H), |
| | (ethylamino)pyrido[2,3- | | 4.11 (m, 1H), 4.03 (m, 1H), 3.96 (m, 1H), |
| | d]pyrimidin-7-one | | 3.78 (dd, J = 6.0, 10.0 Hz, 1H), 3.78 (dd, J = 5.5, 9.5 Hz, |
| | (enantiomer 1; unknown | | 1H), 3.53 (dd, J = 7.0, 14.0 Hz, 2H), 2.65 (s, 3H), |
| | absolute configuration) | | 1.30 (m, 3H), 1.24 (m, 3H) |
| I-92 | yloxy)propyl]-6-[2-chloro- | 506.0 | (500 MHz, MeOD-d4): δ 8.96 (s, 1H), 8.60 (brs, |
| | 4-(6-methylpyrazin-2- | | 1H), 8.48 (s, 1H), 8.26 (d, J = 2.0 Hz, 1H), 8.08 (dd, |
| | yl)phenyl]-2- | | J = 1.5, 7.5 Hz, 1H), 7.79 (s, 1H), 7.54 (d, J = 8.0 Hz |
| | (ethylamino)pyrido[2,3- | | 1H), 4.66 (m, 1H), 4.45 (m, 1H), 4.35 (m, 1H), |
| | d]pyrimidin-7-one | | 4.06 (m, 1H), 3.64 (t, J = 7.0 Hz, 1H)), 3.53 (m, |
| | (enantiomer 2; unknown | | 4H), 3.36 (t, J = 7.0 Hz, 1H), 2.65 (s, 3H), 1.31 (m, |
| | absolute configuration) | | 3H), 1.20 (m, 3H) |

### Example 64

### 6-(2-Chloro-4-(6-methylpyrazin-2-yl)phenyl)-2-(ethylamino)-8-((4-hydroxypiperidin-4-yl)methyl)pyrido[2,3-d]pyrimidin-7(8H)-one (I-54)

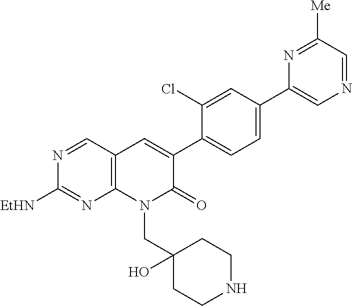

step 1: At 0° C., sodium hydride (60 mg, 60% in oil mineral, 1.5 mmol) was added to a solution of 6-[2-chloro-4-(6-methyl-pyrazin-2-yl)-phenyl]-2-methylsulfanyl-8H-pyrido[2,3-d]pyrimidin-7-one (396 mg, 1 mmol) in DMF (6 mL). The mixture was stirred for 1 h at RT, and then benzyl 1-oxa-6-aza-spiro[2.5]octane-6-carboxylate (494 g, 2 mmol) was added, followed by stirring at RT overnight. The reaction mixture was diluted with water (10 mL), acetone (5 mL) and EtOAc (50 mL). The organic layer was separated, dried over anhydrous Na₂SO₄, and concentrated in vacuo. The residue was purified by SiO₂ chromatography eluting with a MeOH/DCM gradient (1:40 to 1:20) to afford benzyl 4-((6-(2-chloro-4-(6-methylpyrazin-2-yl)phenyl)-2-(methylthio)-7-oxopyrido[2,3-d]pyrimidin-8(7H)-yl)methyl)-4-hydroxypiperidine-1-carboxylate as a yellow solid (800 mg, 60%). LCMS (ESI): m/z=644.0 [M+1]⁺.

step 2: Benzyl 4-((6-(2-chloro-4-(6-methylpyrazin-2-yl)phenyl)-2-(methylthio)-7-oxopyrido[2,3-d]pyrimidin-8(7H)-yl)methyl)-4-hydroxypiperidine-1-carboxylate was converted to benzyl 4-((6-(2-chloro-4-(6-methylpyrazin-2-yl)phenyl)-2-(methylsulfinyl)-7-oxopyrido[2,3-d]pyrimidin-8(7H)-yl)methyl)-4-hydroxypiperidine-1-carboxylate using the procedure described in Example 63, step 2. LCMS (ESI): m/z=660.0 [M+1]⁺.

step 3: Benzyl 4-((6-(2-chloro-4-(6-methylpyrazin-2-yl)phenyl)-2-(methylsulfinyl)-7-oxopyrido[2,3-d]pyrimidin-8(7H)-yl)methyl)-4-hydroxypiperidine-1-carboxylate was converted to benzyl 4-((6-(2-chloro-4-(6-methylpyrazin-2-yl)phenyl)-2-(ethylamino)-7-oxopyrido[2,3-d]pyrimidin-8(7H)-yl)methyl)-4-hydroxypiperidine-1-carboxylate using the procedure described in Example 63, step 3. LCMS (ESI): m/z=641.0 [M+1]⁺.

step 4: HBr (60% in HOAc, 1 mL) was added to a solution of benzyl 4-((6-(2-chloro-4-(6-methylpyrazin-2-yl)phenyl)-2-(ethylamino)-7-oxopyrido[2,3-d]pyrimidin-8(7H)-yl)methyl)-4-hydroxypiperidine-1-carboxylate (200 mg) in DCM (5 mL). The solution was stirred at RT overnight, and then ammonia (30% in MeOH, 10 mL) was added to adjust the pH >7 at 0° C. The mixture was concentrated in vacuo and purified by preparative HPLC to afford 6-(2-chloro-4-(6-methyl-pyrazin-2-yl)-phenyl)-2-ethylamino-8-(4-hydroxy-piperidin-4-ylmethyl)-8H-pyrido[2,3-d]pyrimidin-7-one as white solid (8.3 mg, 10%). ¹H NMR (500 MHz, CDCl₃) δ 8.83 (s, 1H), 8.61-45 (m, 1H), 8.43 (s, 1H), 8.17 (d, J=1.5 Hz, 1H), 7.95 (dd, J=1.5, 7.5 Hz, 1H), 7.62 (s, 1H), 7.50 (d. J=7.5 Hz, 1H), 5.67-5.50 (m, 1H), 4.76-4.54 (m, 2H), 3.55 (q, J=6.5 Hz, 2H), 3.08-2.85 (m, 4H), 2.65 (s, 3H), 1.81-1.70 (m, 1H), 1.40-1.18 (m, 7H); LCMS (ESI): m/z=507.0 [M+1]⁺

### Example 65

### 6-[2-Chloro-4-(6-methylpyrazin-2-yl)phenyl]-2-(ethylamino)-8-[(4-hydroxy-1-methyl-4-piperidyl)methyl]pyrido[2,3-d]pyrimidin-7-one (I-105)

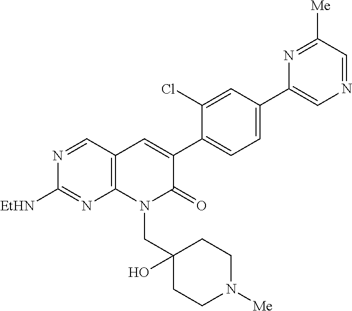

A mixture of 6-(2-chloro-4-(6-methylpyrazin-2-yl)phenyl)-2-(ethylamino)-8-((4-hydroxy-piperidin-4-yl)methyl)pyrido[2,3-d]pyrimidin-7(8H)-one (I-54, 100 mg, 0.20 mmol), formaldehyde (75 mg, 1.00 mmol, 40% in water) and HOAc (24 mg, 0.40 mmol) in MeOH (10 mL) was stirred at RT for 16 h. To this mixture was added NaBH₄ (30 mg, 0.80 mmol). The reaction mixture was stirred at RT for 1 h. The reaction was quenched by addition of saturated aqueous NH₄Cl solution (40 mL) and extracted with DCM (50 mL×3). The combined organic layers were dried (Na₂SO₄), filtered and concentrated in vacuo. The residue was purified by prep-HPLC to afford 6-(2-chloro-4-(6-methylpyrazin-2-yl)phenyl)-2-(ethylamino)-8-((4-hydroxy-1-methylpiperidin-4-yl)methyl)pyrido[2,3-d]pyrimidin-7(8H)-one as a white solid (15 mg, 14%). ¹H NMR (500 MHz, MeOH-d₄): δ 8.98 (s, 1H), 8.65 (brs, 1H), 8.50 (s, 1H), 8.28 (d, J=2.0 Hz, 1H), 8.10 (dd, J=8.0, 2.0 Hz, 1H), 7.84 (s, 1H), 7.56 (d, J=8.0 Hz 1H), 4.68 (m, 2H), 3.56 (m, 2H), 2.70 (m, 2H), 2.67 (s, 3H), 2.46 (t, J=11 Hz, 2H)), 2.31 (s, 3H), 1.87 (m, 2H), 1.70 (m, 2H), 1.32 (m, 3H); LCMS (ESI): m/z=520.1 [M+1]⁺.

### Example 66

### 2-(Isopropylamino)-6-(2-methyl-4-(6-methylpyridin-2-yl)phenyl)-8-(2-oxo-2-(piperazin-1-yl)ethyl)pyrido[2,3-d]pyrimidin-7(8H)-one (I-78)

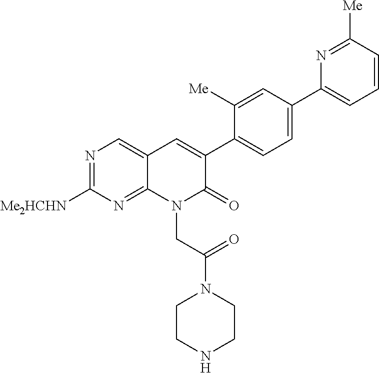

step 1: To a mixture of 6-(2-methyl-4-(6-methylpyridin-2-yl)phenyl)-2-(methylthio)pyrido-[2,3-d]pyrimidin-7(8H)-one (500 mg, 1.33 mmol) in DMF (5 mL) was added t-BuOK (280 mg, 2.5 mmol) and tert-butyl 4-(2-chloroacetyl)piperazine-1-carboxylate (395 mg, 1.5 mmol). The mixture was then stirred at RT overnight, extracted with EtOAc, washed with brine, dried (MgSO₄), filtered and concentrated in vacuo to afford tert-butyl 4-(2-(6-(2-methyl-4-(6-methylpyridin-2-yl)phenyl)-2-(methylthio)-7-oxopyrido[2,3-d]pyrimidin-8(7H)-yl)acetyl)piperazine-1-carboxylate as a yellow solid (500 mg, crude). LCMS (ESI): m/z=601.1 [M+1]⁺.

step 2: HOAc (1 mL) and MCPBA (155 mg, 0.9 mmol) was added to a mixture of tert-butyl 4-(2-(6-(2-methyl-4-(6-methylpyridin-2-yl)phenyl)-2-(methylthio)-7-oxopyrido-[2,3-d]pyrimidin-8(7H)-yl)acetyl)piperazine-1-carboxylate (500 mg, 0.83 mmol) in DCM (5 mL). The reaction was stirred overnight. The reaction was concentrated under reduced pressure then purified by SiO₂ chromatography eluting with DCM/MeOH (10:1) to afford tert-butyl-4-(2-(6-(2-methyl-4-(6-methylpyridin-2-yl)phenyl)-2-(methylsulfinyl)-7-oxopyrido-[2,3-d]pyrimidin-8(7H)-yl)acetyl)piperazine-1-carboxylate as a yellow solid (300 mg, crude). LCMS (ESI): m/z=617.3 [M+1]⁺.

step 3: DIPEA (0.5 mL) was added to a mixture of tert-butyl-4-(2-(6-(2-methyl-4-(6-methylpyridin-2-yl)phenyl)-2-(methylsulfinyl)-7-oxopyrido-[2,3-d]pyrimidin-8(7H)-yl)acetyl)piperazine-1-carboxylate (100 mg, 0.16 mmol), propan-2-amine (60 mg, 1.0 mmol) and i-PrOH (5 mL). The mixture was stirred at 80° C. for 2 h, concentrated under in vacuo and purified by SiO₂ chromatography eluting with MeOH/DCM (1:10) to afford tert-butyl 4-(2-(2-(isopropylamino)-6-(2-methyl-4-(6-methylpyridin-2-yl)phenyl)-7-oxopyrido[2,3-d]pyrimidin-8(7H)-yl)acetyl)piperazine-1-carboxylate (70 mg, 72%). LCMS (ESI): m/z=612.2 [M+1]⁺.

step 4: TFA (2 mL) was added to a mixture of tert-butyl 4-(2-(2-(isopropylamino)-6-(2-methyl-4-(6-methylpyridin-2-yl)-phenyl)-7-oxopyrido[2,3-d]pyrimidin-8(7H)-yl)acetyl)piperazine-1-carboxylate (70 mg, 0.11 mmol) and DCM (10 mL). The mixture was stirred at RT for 2 h. The mixture was then concentrated by reducing pressure whilst adding NH₃/MeOH (7 M) to adjust the pH greater than 7. The residue was then purified by prep-HPLC to afford 2-(isopropylamino)-6-(2-methyl-4-(6-methylpyridin-2-yl)phenyl)-8-(2-oxo-2-(piperazin-1-yl)ethyl)pyrido[2,3-d]pyrimidin-7(8H)-one (40 mg, 69%) as white solid. ¹H NMR (500 MHz, CDCl₃) δ 8.44 (s, 1H), 7.87 (s, 1H), 7.79 (d, J=8.0, 1H), 7.64-7.61 (m, 1H), 7.52 (d, J=8.0, 1H), 7.50 (s, 1H), 7.32 (d, J=8.0, 1H), 7.08 (d, J=8.0, 1H), 5.35-5.32 (m, 1H), 5.24 (s, 2H), 4.11-4.08 (m, 1H), 3.62-3.59 (m, 4H), 2.98-2.96 (m, 2H), 2.88-2.86 (m, 2H), 2.63 (s, 3H), 2.32 (s, 3H), 1.29-1.28 (m, 6H). LCMS (ESI): m/z=512.1 [M+1]⁺.

Compounds in Table IIc were made through similar procedures as used for the synthesis of G02851318, employing appropriate starting materials.

TABLE IIc

| | | MS m/z | |
|---|---|---|---|
| No. | Name | [M + H]+ | ¹H NMR |
| I-107 | 6-[2-Chloro-4-(6-methyl- | 504.2 | (500 MHz, CDCl₃) δ 8.48 (brs, 1H), 8.11 (s, 1H), |
| | 2-pyridyl)phenyl]-2- | | 7.88 (d, J = 7.5 Hz, 1H), 7.75-7.59 (m, 2H), |
| | (methylamino)-8-(2-oxo- | | 7.51 (brs, 1H), 7.12 (d, J = 7.5 Hz, 1H), 5.77-5.50 (m, |
| | 2-piperazin-1-yl- | | 1H), 5.26 (s, 1H), 3.78-3.51 (m, 4H), 3.15-2.79 (m, |
| | ethyl)pyrido[2,3- | | 7H), 2.62 (s, 3H). |
| | d]pyrimidin-7-one | | |
| I-34 | 6-[2-Chloro-4-(6-methyl- | 518.2 | (500 MHz, CDCl₃) δ 8.47 (s, 1H), 8.12 (s, 1H), |
| | 2-pyridyl)phenyl]-2- | | 7.88 (dd, J = 2.0, 10.0, 1H), 7.67-7.62 (m, 2H), |
| | (ethylamino)-8-(2-oxo-2- | | 7.53-7.49 (m, 2H), 7.12 (d, J = 10.0, 1H), 5.53-5.52 (m, 1H), |
| | piperazin-1-yl- | | 5.25 (s, 2H), 3.94-3.92 (m, 4H), 3.48 (s, 2H), |
| | ethyl)pyrido[2,3- | | 2.98-2.96 (m, 2H), 2.88-2.86 (m, 2H), 2.63 (s, 3H), |
| | d]pyrimidin-7-one | | 1.28-1.25 (m, 3H). |
| I-35 | 6-[2-Chloro-4-(6-methyl- | 532.2 | (500 MHz, DMSO-d₆) δ 8.46 (s, 1H), 8.12 (s, 1H), |
| | 2-pyridyl)phenyl]-2- | | 7.88 (d, J = 10.0, 1H), 7.66-7.61 (m, 2H), |
| | (isopropylamino)-8-(2- | | 7.53-7.49 (m, 2H), 7.12 (d, J = 10.0, 1H), 5.35-5.32 (m, 1H), |
| | oxo-2-piperazin-1-yl- | | 5.24 (s, 2H), 4.11-4.08 (m, 1H), 3.62-3.59 (m, 4H), |
| | ethyl)pyrido[2,3- | | 2.98-2.96 (m, 2H), 2.88-2.86 (m, 2H), 2.63 (s, 3H), |
| | d]pyrimidin-7-one | | 1.29-1.26 (m, 6H). |
| I-94 | 6-[2-Chloro-4-(6-methyl- | 547.0 | (500 MHz, CDCl3-d1) δ 8.51 (s, 1H), 8.12 (s, 1H), |
| | 2-pyridyl)phenyl]-2- | | 7.89 (dd, J = 2.0, 10.0 Hz, 1H), 7.71-7.61 (m, 2H), |
| | (oxetan-3-ylamino)-8-(2- | | 7.56-7.48 (m, 2H), 7.13 (d, J = 9.5 Hz, 1H), |
| | oxo-2-piperazin-1-yl- | | 6.18-5.92 (m, 1H), 5.23 (s, 2H), 5.16-5.02 (m, 1H), |
| | ethyl)pyrido[2,3- | | 5.00-4.89 (m, 2H), 4.78-4.54 (m, 2H), |
| | d]pyrimidin-7-one | | 3.74-3.96 (m, 4H), 3.07-2.92 (m, 2H), 2.92-2.81 (m, 2H), |
| | | | 2.63 (s, 3H) |
| I-95 | 2-(Methylamino)-6-[2- | 484.0 | (500 MHz, CDCl3-d1) δ 8.46 (brs, 1H), 7.87 (s, |
| | methyl-4-(6-methyl-2- | | 1H), 7.79 (d, J = 10.0, 1H), 7.63 (t, J = 9.5, 1H), |
| | pyridyl)phenyl]-8-(2-oxo- | | 7.56-7.48 (m, 2H), 7.33 (d, J = 9.5, 1H), 7.09 (d, J = 9.5, |
| | 2-piperazin-1-yl- | | 1H), 5.60-5.38 (m, 1H), 5.27 (brs, 2H), |
| | ethyl)pyrido[2,3- | | 3.73-3.51 (m, 4H), 3.12-3.00 (m, 3H), 3.00-2.93 (m, 2H), |
| | d]pyrimidin-7-one | | 2.93-2.82 (m, 2H), 2.63 (s, 3H), 2.33 (s, 3H) |
| I-93 | 2-(Ethylamino)-6-[2- | 498.0 | (500 MHz, CDCl3-d1) δ 8.46 (brs, 1H), 7.87 (s, |
| | methyl-4-(6-methyl-2- | | 1H), 7.79 (d, J = 10.5 Hz, 1H), 7.63 (t, J = 9.5 Hz, |
| | pyridyl)phenyl]-8-(2-oxo- | | 1H), 7.52 (d, J = 10.0 Hz, 1H), 7.50 (s, 1H), 7.33 (d, |
| | 2-piperazin-1-yl- | | J = 10.0 Hz, 1H), 7.09 (d, J = 9.5 Hz, 1H), |
| | ethyl)pyrido[2,3- | | 5.57-5.39 (m, 1H), 5.25 (brs, 2H), 3.69-3.55 (m, 4H), 3.49 (q, |
| | d]pyrimidin-7-one | | J = 7.5 Hz, 2H), 3.03-2.92 (m, 2H), 2.92-2.82 (m, |
| | | | 2H), 2.63 (s, 3H), 2.33 (s, 3H), 1.27 (t, J = 8.5 Hz, |
| | | | 3H) |
| I-106 | 6-[2-Methyl-4-(6-methyl- | 526.0 | (500 MHz, CDCl3-d1) δ 8.49 (s, 1H), 7.87 (s, 1H), |
| | 2-pyridyl)phenyl]-2- | | 7.79 (d, J = 8.0, 1H), 7.63 (t, J = 8, 1H), 7.52 (s, 1H), |
| | (oxetan-3-ylamino)-8-(2- | | 7.54-7.49 (m, 2H), 7.31 (d, J = 8.0, 1H), 7.09 (d, |
| | oxo-2-piperazin-1-yl- | | J = 8.0, 1H), 5.89 (d, J = 6.0, 1H), 5.30 (s, 2H), |
| | ethyl)pyrido[2,3- | | 5.15-5.00 (m, 1H), 5.00-4.89 (m, 2H), 4.77-4.56 (m, |
| | d]pyrimidin-7-one | | 2H), 3.71-3.56 (m, 4H), 3.11-2.98 (m, 2H), |
| | | | 2.96-2.83 (m, 2H), 2.63 (s, 3H), 2.31 (s, 3H) |

### Example 67

### 6-(2-Chloro-4-(6-methylpyrazin-2-yl)phenyl)-2-(methylamino)-8-(2-oxo-2-(piperazin-1-yl)ethyl)pyrido[2,3-d]pyrimidin-7(8H)-one (I-84)

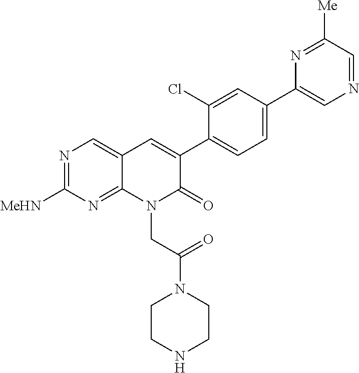 [SMILES: CNc1ncc2cc(-c3ccc(-c4cncc(C)n4)cc3Cl)c(=O)n(CC(=O)N3CCNCC3)c2n1]

step 1: A mixture of 6-[2-chloro-4-(6-methylpyrazin-2-yl)phenyl]-2-methylsulfanyl-8H-pyrido[2,3-d]pyrimidin-7-one (2.01 g, 5.08 mmol), Cs₂CO₃ (1.83 g, 5.60 mmol, 1.10 equiv.) and ethyl 2-chloroacetate (650 μL, 6.10 mmol, 1.20 equiv.) in DMF (20 mL) was stirred at RT for 64 h. The mixture was diluted with water and extracted twice into 2-methylTHF. The combined organic phases were washed with water and brine, dried (Na₂SO₄), filtered and concentrated in vacuo to afford a pale brown solid (2.46 g) which was purified by SiO₂ chromatography eluting with an EtOAc/heptane gradient (20 to 100% EtOAc) to afford ethyl 2-[6-[2-chloro-4-(6-methylpyrazin-2-yl)phenyl]-2-methylsulfanyl-7-oxo-pyrido[2,3-d]pyrimidin-8-yl]acetate as a white solid (1.77 g, 72%). ¹H NMR (400 MHz, DMSO-d6) δ 9.18 (s, 1H), 9.01 (s, 1H), 8.58 (s, 1H), 8.32 (d, J=1.7 Hz, 1H), 8.20 (dd, J=8.0, 1.8 Hz, 1H), 8.17 (s, 1H), 7.61 (d, J=8.0 Hz, 1H), 5.14 (s, 2H), 4.18 (q, J=7.1 Hz, 2H), 2.60 (s, 3H), 2.58 (s, 3H), 1.20 (t, J=7.1 Hz, 3H). MS (ESI) m/z: 482/484 [MH]⁺.

step 2: A suspension of ethyl 2-[6-[2-chloro-4-(6-methylpyrazin-2-yl)phenyl]-2-methylsulfanyl-7-oxo-pyrido[2,3-d]pyrimidin-8-yl]acetate (946 mg, 1.96 mmol) in THF (20 mL) was treated with solid MCPBA (488 mg, 2.18 mmol, 1.11 equiv.) at RT for 2 h, and then a second portion of MCPBA (89 mg, 0.40 mmol, 0.20 equiv.) was added and the reaction stirred for 1 h. The mixture was concentrated in vacuo. The crude residue was treated with a solution of methylamine in THF (2.0 M, 10 mL, 20 mmol, 10 equiv.) at RT for 30 min and again concentrated in vacuo. The residue was partitioned between saturated aqueous NaHCO₃ and DCM, and the separated organic phase washed with brine, dried (Na₂SO₄), filtered and concentrated in vacuo to afford ethyl 2-[6-[2-chloro-4-(6-methylpyrazin-2-yl)phenyl]-2-(methylamino)-7-oxo-pyrido[2,3-d]pyrimidin-8-yl]acetate as a pale yellow solid (984 mg). ¹H NMR (400 MHz, DMSO-d6) δ 9.16 (s, 1H), 8.80-8.66 (m, 1H), 8.57 (s, 1H), 8.28 (d, J=1.8 Hz, 1H), 8.16 (dd, J=8.1, 1.8 Hz, 1H), 8.00-7.94 (m, 1H), 7.94 (s, 1H), 7.56 (d, J=8.0 Hz, 1H), 5.08-4.97 (m, 2H), 4.16 (q, J=6.9 Hz, 2H), 2.93-2.83 (m, 3H), 2.60 (s, 3H), 1.20 (t, J=7.1 Hz, 3H). MS (ESI) m/z: 465/467 [MH]⁺.

step 3: Crude ethyl 2-[6-[2-chloro-4-(6-methylpyrazin-2-yl)phenyl]-2-(methylamino)-7-oxo-pyrido[2,3-d]pyrimidin-8-yl]acetate (1.96 mmol) in MeOH (20 mL) was treated with an aqueous solution of LiOH (1.0 M, 20 mL, 20 mmol, 10 equiv.) at RT for 3 h. The heterogeneous mixture was diluted with MeOH (40 mL) and heated to reflux for 2 h. The cooled mixture was concentrated in vacuo to approximately 20 mL and washed with EtOAc (discarded). The aqueous phase acidified to pH 3 with aqueous sulfuric acid (2.0 N) and extracted twice with EtOAc. The combined organic phases were washed with brine, dried (Na₂SO₄), filtered and concentrated in vacuo to afford pure 2-[6-[2-chloro-4-(6-methylpyrazin-2-yl)phenyl]-2-(methylamino)-7-oxo-pyrido[2,3-d]pyrimidin-8-yl]acetate as a pale yellow solid (834 mg, 97% for two steps). ¹H NMR (400 MHz, CDCl₃) δ 8.82 (s, 1H), 8.42 (s, 1H), 8.38 (br s, 1H), 8.15 (d, J=1.7 Hz, 1H), 7.92 (dd, J=8.0, 1.8 Hz, 1H), 7.62 (s, 1H), 7.52 (d, J=8.0 Hz, 1H), 7.33 (br s, 1H), 6.6 (br s, 1H), 5.20 (s, 2H), 2.64 (s, 3H), 2.09 (s, 3H). MS (ESI) m/z: 437/439 [MH]⁺.

step 4: 2-[6-[2-Chloro-4-(6-methylpyrazin-2-yl)phenyl]-2-(methylamino)-7-oxo-pyrido[2,3-d]pyrimidin-8-yl]acetate (47.8 mg, 0.109 mmol) and 1-[bis(dimethylamino)methylene]-1H-1,2,3-triazolo[4,5-b]pyridinium 3-oxid hexafluorophosphate (57.1 mg, 0.149 mmol, 1.36 equiv.) were suspended together in DMF (2.0 mL) and treated with DIPEA (100 μL, 0.1 mL, 0.57 mmol, 5.2 equiv.) at RT for 10 min. The resulting red-orange solution was treated with a solution of piperazine (99.0 mg, 1.14 mmol, 10.4 equiv.) in DMF (1.0 mL) at RT for 30 min. The mixture was poured into saturated aqueous NaHCO₃ and extracted with a mixture of EtOAc and 2-methylTHF. The separated organic phase was washed with water and brine, dried (Na₂SO₄), filtered and concentrated in vacuo to afford a pale solid (45.3 mg). The combined aqueous phases were extracted with DCM, and the separated organic phase was washed with brine, dried over sodium sulfate and filtered. The combined extracts were concentrated in vacuo and to an orange solid (51.4 mg). The crude residue was submitted to purification by reverse phase HPLC (C18, gradient of acetonitrile in water with 0.1% ammonium hydroxide) to afford 6-(2-chloro-4-(6-methylpyrazin-2-yl)phenyl)-2-(methylamino)-8-(2-oxo-2-(piperazin-1-yl)ethyl)pyrido[2,3-d]pyrimidin-7(8H)-one as a white solid (22.4 mg, 41%). ¹H NMR (400 MHz, DMSO-d6) δ 9.16 (s, 1H), 8.78-8.64 (m, 1H), 8.57 (s, 1H), 8.28 (d, J=1.7 Hz, 1H), 8.16 (dd, J=8.0, 1.8 Hz, 1H), 7.90 (s, 1H), 7.93-7.84 (m, 1H), 7.55 (d, J=8.0 Hz, 1H), 5.17-5.05 (m, 2H), 3.60-3.50 (m, 2H), 3.40-3.34 (m, 2H), 2.93-2.83 (m, 3H), 2.81-2.73 (m, 2H), 2.69-2.62 (m, 2H), 2.60 (s, 3H). MS (ESI) m/z: 505.2 [M+1]⁺.

### Example 68

### 2-[6-[2-Chloro-4-(6-methylpyrazin-2-yl)phenyl]-2-(methylamino)-7-oxo-pyrido[2,3-d]pyrimidin-8-yl]-N-(2-hydroxyethyl)acetamide (I-85)

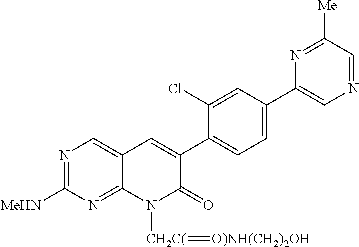

The title compound was prepared in accord with Example 67 using appropriate starting materials. ¹H NMR (400 MHz, DMSO-d₆) δ 9.15 (s, 1H), 8.77-8.63 (m, 1H), 8.57 (s, 1H), 8.28 (d, J=1.8 Hz, 1H), 8.22-8.13 (m, 1H), 8.16 (dd, J=8.1, 1.8 Hz, 1H), 7.89 (s, 1H), 7.91-7.81 (m, 1H), 7.56 (d, J=8.0 Hz, 1H), 4.96-4.84 (m, 2H), 4.66 (t, J=5.3 Hz, 1H), 3.42 (q, J=5.9 Hz, 2H), 3.19-3.11 (m, 2H), 2.93-2.83 (m, 3H), 2.60 (s, 3H). MS (ESI) m/z: 480.2 [M+1]⁺.

### Example 69

### 2-[6-[2-Chloro-4-(6-methylpyrazin-2-yl)phenyl]-2-(methylamino)-7-oxo-pyrido[2,3-d]pyrimidin-8-yl]-N,N-dimethyl-acetamide (I-86)

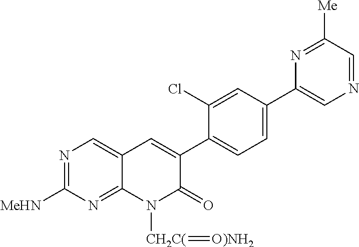

The title compound was prepared in accord with Example 67 using appropriate starting materials. ¹H NMR (400 MHz, DMSO-d₆) δ 9.16 (s, 1H), 8.77-8.64 (m, 1H), 8.57 (s, 1H), 8.28 (d, J=1.7 Hz, 1H), 8.16 (dd, J=8.0, 1.8 Hz, 1H), 7.90 (s, 1H), 7.92-7.84 (m, 1H), 7.55 (d, J=8.0 Hz, 1H), 5.16-5.06 (m, 2H), 3.19-3.12 (m, 3H), 2.92-2.80 (m, 6H), 2.60 (s, 3H). MS (ESI) m/z: 464.2 [M+1]⁺.

### Example 70

### 8-(1-(2-Aminoacetyl)piperidin-4-yl)-6-(2-chloro-4-(6-methylpyrazin-2-yl)phenyl)-2-(ethylamino)pyrido[2,3-d]pyrimidin-7(8H)-one (I-29)

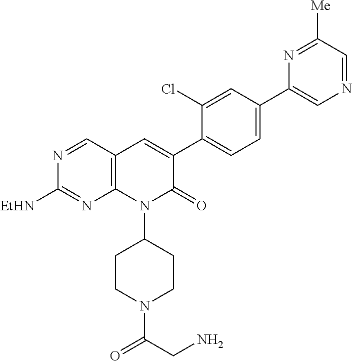

The title compound was prepared in accord with Example 67 using appropriate starting materials. ¹H NMR (400 MHz, DMSO-d₆) δ 9.14 (s, 1H), 8.63 (s, 1H), 8.57 (s, 1H), 8.26 (s, 1H), 8.14 (d, J=9.6 Hz, 1H), 7.95 (s, 1H), 7.81 (s, 1H), 7.55 (d, J=8.1 Hz, 1H), 5.62 (s, 2H), 4.58 (m, 2H), 3.91 (m, 2H), 3.60 (m, 3H), 2.60 (s, 3H), 1.66 (m, 3H), 1.17 (1, J=7.1 Hz, 3H). MS (ESI) m/z: 533 [M+1]⁺.

### Example 71

### 6-(2-Chloro-4-(6-methylpyridin-2-yl)phenyl)-2-(ethylamino)-8-(2-(piperazin-1-yl)ethyl)pyrido[2,3-d]pyrimidin-7(8H)-one (I-66)

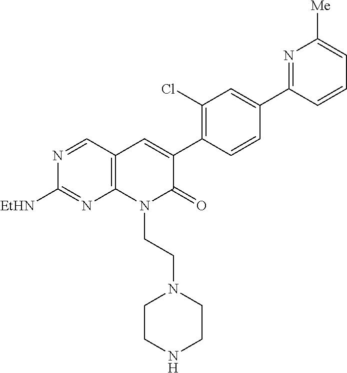

step 1: A mixture of 6-(2-chloro-4-(6-methylpyridin-2-yl)phenyl)-2-(methylthio)pyrido[2,3-d]pyrimidin-7(8H)-one (500 mg), tert-butyl 4-(2-methylsulfonyloxyethyl)piperazine-1-carboxylate (346.4 mg, 1.39 mmol) and Cs₂CO₃ (1.24 g, 3.80 mmol) and anhydrous DMF (9.7 mL) in a sealed tube was stirred at 90° C. for 1 h. The cooled reaction mixture was poured into water (100 mL) and allowed to stand overnight. The solid was filtered and concentrated under vacuum to give tert-butyl 4-(2-(6-(2-chloro-4-(6-methylpyridin-2-yl)phenyl)-2-(methylthio)-7-oxopyrido[2,3-d]pyrimidin-8(7H)-yl)ethyl)piperazine-1-carboxylate (289.1 mg, 37.6%). ¹H NMR (400 MHz, CDCl₃) δ 8.67 (s, 1H), 8.15 (d, J=1.7 Hz, 1H), 7.94 (dd, J=8.0, 1.7 Hz, 1H), 7.69-7.64 (m, 2H), 7.53 (d, J=7.9 Hz, 1H), 7.46 (d, J=8.0 Hz, 1H), 7.14 (d, J=7.6 Hz, 1H), 4.66 (br s, 2H), 3.38 (br s, 4H), 2.78 (br s, 2H), 2.66 (s, 3H), 2.64 (s, 3H), 2.54 (br s, 4H), 1.45 (s, 9H) MS (ESI) m/z: 607.2 [M+1]⁺.

step 2: To a stirred solution of glacial HOAc (52.2 mg, 0.95 mmol) in DCM (9.5 mL) at 0° C. was added tert-butyl 4-(2-(6-(2-chloro-4-(6-methylpyridin-2-yl)phenyl)-2-(methylthio)-7-oxopyrido[2,3-d]pyrimidin-8(7H)-yl)ethyl)piperazine-1-carboxylate (289 mg, 0.476 mmol) followed by MCPBA (241.6 mg, 1.19 mmol). The reaction mixture was stirred at 0° C. for 1 h. The reaction mixture was quenched with saturated aqueous NaHCO₃ solution (10 mL). Volatile solvent was removed under reduced pressure, and the residue was extracted with EtOAc (25 mL×3). The combined organic layers were washed with water and brine, dried (Na₂SO₄), filtered, and concentrated under reduced pressure to give 4-(tert-butoxycarbonyl)-1-(2-(6-(2-chloro-4-(6-methylpyridin-2-yl)phenyl)-2-(methylsulfinyl)-7-oxopyrido[2,3-d]pyrimidin-8(7H)-yl)ethyl)piperazine 1-oxide in quantitative yield. MS (ESI) m/z: 639.2 [M+1]⁺.

step 3: A mixture of 4-(tert-butoxycarbonyl)-1-(2-(6-(2-chloro-4-(6-methylpyridin-2-yl)phenyl)-2-(methylsulfinyl)-7-oxopyrido[2,3-d]pyrimidin-8(7H)-yl)ethyl)piperazine 1-oxide (75 mg) and ethylamine (2.0 M) in THF (5.9 mL) in a sealed vial was stirred at 50° C. After 20 h, bis(pinacolato)diboron (35.8 mg, 0.14 mmol) was added, and the reaction mixture was stirred at RT for 3 h. The reaction mixture was diluted with EtOAc, and the organic layer washed with water and brine, dried (Na₂SO₄), filtered, and concentrated under reduced pressure. The crude residue was purified by SiO₂ chromatography eluting with EtOAc/heptane to afford tert-butyl 4-(2-(6-(2-chloro-4-(6-methylpyridin-2-yl)phenyl)-2-(ethylamino)-7-oxopyrido[2,3-d]pyrimidin-8(7H)-yl)ethyl)piperazine-1-carboxylate (46.7 mg, 65.9%). MS (ESI) m/z: 604.2 [M+1]⁺.

step 4: To a stirred solution of tert-butyl 4-(2-(6-(2-chloro-4-(6-methylpyridin-2-yl)phenyl)-2-(ethylamino)-7-oxopyrido[2,3-d]pyrimidin-8(7H)-yl)ethyl)piperazine-1-carboxylate (46.7 mg, 0.077 mmol) in DCM (1.6 mL) and MeOH (1.6 mL) was added HCl in 1,4-dioxane (4.0 M, 1.0 mL, 4 mmol), and the reaction mixture was stirred at 50° C. for 17 h. Volatile solvents were concentrated, and the crude was redissolved in water (1.0 mL) and the pH adjusted to ca. 7 with sat. aqueous NaHCO₃. The reaction mixture was extracted with DCM (3×25 mL). The combined organic layers were washed with water and brine, dried (Na₂SO₄), filtered, and concentrated. The crude mixture was purified by preparative HPLC to give the title compound as a solid (13.3 mg, 32.6%). ¹H NMR (400 MHz, DMSO-d₆) δ 8.64 (s, 1H), 8.21 (s, 1H), 8.07 (d, J=8.1 Hz, 1H), 7.95 (br s, 1H), 7.86 (d, J=7.9 Hz, 1H), 7.83-7.78 (m, 2H), 7.49 (d, J=8.0 Hz, 1H), 7.28 (d, J=7.5 Hz, 1H), 4.50-4.34 (m, 2H), 3.41 (t, J=7.0 Hz, 2H), 2.77-2.69 (m, 4H), 2.64-2.58 (m, 4H), 2.57 (s, 3H), 2.53-2.40 (m, 3H), 1.20 (t, J=7.4 Hz, 3H). MS (ESI+) m/z: 504.2 [M+1]⁺.

Compounds in Table IId were made through similar procedures as used for the synthesis of I-66 employing appropriate starting materials.

TABLE IId

| Cpd. | | MS m/z | |
|---|---|---|---|
| No. | Name | [M + H]+ | ¹H NMR |
| I-68 | 6-(2-Chloro-4-(6- | 518.2 | (400 MHz, DMSO-d₆) δ 8.64 (s, 1H), 8.21 (s, 1H), |
| | methylpyridin-2- | | 8.07 (d, J = 8.1 Hz, 1H), 7.90-7.77 (m, 3H), |
| | yl)phenyl)-2- | | 7.49 (d, J = 8.3 Hz, 1H), 7.28 (d, J = 7.5 Hz, 1H), |
| | (isopropylamino)-8-(2- | | 4.42 (s, 2H), 4.15 (br s, 1H), 2.65 (s, 4H), |
| | (piperazin-1- | | 2.60-2.54 (m, 4H), 2.52-2.49 (m, 4H), 2.41 (s, 4H), 1.23 (d, |
| | yl)ethyl)pyrido[2,3- | | J = 6.5 Hz, 5H) |
| | d]pyrimidin-7(8H)-one | | |
| I-89 | 6-[2-Chloro-4-(6-methyl- | 540.2 | (400 MHz, DMSO-d6) d 8.72 (s, 1H), 8.22 (d, J = 1.6 Hz, |
| | 2-pyridyl)phenyl]-2-(2,2- | | 1H), 8.29-8.23 (m, 1H), 8.09 (dd, J = 8.0, |
| | difluoroethylamino)-8-(2- | | 1.8 Hz, 1H), 7.89-7.85 (m, 2H), 7.81 (t, J = 7.7 Hz, |
| | piperazin-1- | | 1H), 7.50 (d, J = 8.0 Hz, 1H), 7.28 (d, J = 7.5 Hz, |
| | ylethyl)pyrido[2,3- | | 1H), 6.41-6.07 (m, 2H), 4.43 (d, J = 8.3 Hz, |
| | d]pyrimidin-7-one | | 2H), 3.88-3.75 (m, 2H), 2.69 (t, J = 5.0 Hz, 4H), |
| | | | 2.61-2.54 (m, 5H), 2.44 (s, 4H) |
| I-72 | 2-Amino-8-(3- | 401.2 | (400 MHz, DMSO-d6) δ 8.63 (s, 1H), |
| | aminopropyl)-6-[2-methyl- | | 7.99-7.93 (m, 1H), 7.90 (dd, J = 7.8, 1.9 Hz, 1H), |
| | 4-(6-methyl-2- | | 7.82-7.75 (m, 2H), 7.73 (s, 1H), 7.29 (s, 1H), 7.27 (s, 2H), |
| | pyridyl)phenyl]pyrido[2,3- | | 7.24-7.17 (m, 1H), 4.38-4.25 (m, 2H), 2.56 (s, |
| | d]pyrimidin-7-one | | 3H), 2.55-2.53 (m, 2H), 2.23 (s, 3H), |
| | | | 1.81-1.69 (m, 2H) |
| I-88 | 2-Amino-8-(5- | 450.2 | (400 MHz, DMSO-d6) δ 9.15 (s, 1H), 8.65 (s, 1H), |
| | aminopentyl)-6-[2-chloro- | | 8.57 (s, 1H), 8.27 (d, J = 1.7 Hz, 1H), 8.15 (dd, J = 8.1, |
| | 4-(6-methylpyrazin-2- | | 1.8 Hz, 1H), 7.82 (s, 1H), 7.55 (d, J = 8.0 Hz, |
| | yl)phenyl]pyrido[2,3- | | 1H), 7.38-7.31 (m, 2H), 6.63 (s, 1H), |
| | d]pyrimidin-7-one | | 4.31-4.22 (m, 2H), 2.60 (s, 3H), 2.57-2.52 (m, 2H), |
| | | | 1.71-1.58 (m, 2H), 1.47-1.25 (m, 5H). |
| I-42 | 8-(4-Aminobutyl)-6-[2- | 450.1 | (400 MHz, DMSO-d6) δ 9.18-9.13 (m, 1H), |
| | chloro-4-(6- | | 8.67-8.62 (m, 1H), 8.61-8.55 (m, 1H), 8.27 (d, J = 1.7 Hz, |
| | methylpyrazin-2- | | 1H), 4.24-4.22 (m, 0H), 8.15 (dd, J = 8.0, |
| | yl)phenyl]-2- | | 1.8 Hz, 1H), 7.92-7.86 (m, 1H), 7.85-7.82 (m, |
| | (methylamino)pyrido[2,3- | | 1H), 7.56 (d, J = 8.0 Hz, 1H), 4.37-4.14 (m, 2H), |
| | d]pyrimidin-7-one | | 2.97-2.86 (m, 4H), 4.27-4.26 (m, 0H), |
| | | | 2.63-2.54 (m, 5H), 1.79-1.53 (m, 2H), 1.41 (dd, J = 15.4, |
| | | | 8.2 Hz, 3H) |
| I-97 | 8-[2-(2- | 445.2 | (500 MHz, DMSO-d6): δ 8.64-8.63 (m, 1H), |
| | Aminoethoxy)ethyl]-2- | | 7.96 (d, J = 8.5 Hz, 1H), 7.90 (m, 1H), 7.78-7.74 (m, |
| | (methylamino)-6-[2- | | 4H), 7.28 (d, J = 7.5 Hz, 1H), 7.22 (d, J = 8.5 Hz, |
| | methyl-4-(6-methyl-2- | | 1H), 4.54 (m, 2H), 3.72-3.68 (m, 2H), 3.44 (m, |
| | pyridyl)phenyl]pyrido[2,3- | | 2H), 2.92 (d, J = 4.0 Hz, 3H), 2.64-2.62 (m, 2H), |
| | d]pyrimidin-7-one | | 2.61 (s, 3H), 2.22 (s, 3H) |
| I-76 | 8-[2-(2- | 459.2 | (500 MHz, DMSO-d₆): δ 8.62 (m, 1H), 7.95 (s, |
| | Aminoethoxy)ethyl]-2- | | 1H), 7.89 (d, J = 9.5 Hz, 2H), 7.78-7.73 (m, |
| | (ethylamino)-6-[2-methyl- | | 3H), 7.30 (d, J = 8.0 Hz 1H), 7.21 (d, J = 3.5 Hz 1H), |
| | 4-(6-methyl-2- | | 4.52 (d, J = 5.0 Hz 2H), 3.70 (d, J = 6.0 Hz 2H), |
| | pyridyl)phenyl]pyrido[2,3- | | 3.42-3.39 (m, 4H), 2.61-2.59 (m, 2H), 2.55 (s, 3H), |
| | d]pyrimidin-7-one | | 2.22 (s, 3H), 1.45 (brs, 2H), 1.21-1.18 (m, 3H) |
| I-96 | 8-[2-(2- | 465.2 | (500 MHz, DMSO-d6): δ 8.72-8.64 (m, 1H), |
| | Aminoethoxy)ethyl]-[2- | | 8.22 (s, 1H), 8.07 (m, 1H), 7.87-7.81 (m, 4H), |
| | chloro-4-(6-methyl-2- | | 7.50 (d, J = 7.5 Hz 1H), 7.28 (d, J = 7.5 Hz 1H), 4.54 (m, |
| | pyridyl)phenyl]-2- | | 2H), 3.72-3.65 (m, 2H), 3.42 (m, 2H), 2.92 (d, J = 4.0, |
| | (methylamino)pyrido[2,3- | | 3H), 2.65-2.64 (m, 2H), 2.62 (s, 3H), |
| | d]pyrimidin-7-one | | 1.83-1.79 (brs, 2H) |
| I-64 | 8-[2-(2- | 479.1 | (500 MHz, DMSO-d₆): δ 8.64 (m, 1H), 7.95 (s, |
| | Aminoethoxy)ethyl]-6-[2- | | 1H), 7.89 (d, J = 8.0 Hz, 1H), 7.87-7.80 (m, 4H), |
| | chloro-4-(6-methyl-2- | | 7.50 (d, J = 8.0 Hz, 1H), 7.21 (d, J = 7.5 Hz, 1H), 4.52 (d, |
| | pyridyl)phenyl]-2- | | J = 4.5 Hz, 2H), 3.70 (d, J = 5.0 Hz, 2H), |
| | (ethylamino)pyrido[2,3- | | 3.42-3.39 (m, 4H), 2.61-2.56 (m, 5H), 1.50 (brs, 1H), |
| | d]pyrimidin-7-one | | 1.21-1.18 (m, 3H) |
| I-61 | 8-(2-(2- | 493.0 | (500 MHz, DMSO-d₆): δ 8.64 (s, 1H), 8.22 (d, |
| | Aminoethoxy)ethyl)-6-(2- | | J = 1.5 Hz, 1H), 8.10 (d, J = 9.5 Hz 1H), |
| | chloro-4-(6-methylpyridin- | | 7.88-7.86 (m, 2H), 7.83-7.79 (m, 2H), 7.50 (d, J = 8.0 Hz 1H), |
| | 2-yl)phenyl)-2- | | 7.28 (d, 1H), 4.50 (brs, 2H), 4.21-4.11 (m, 1H), |
| | (isopropylamino)pyrido[2, | | 3.69-3.67 (m, 2H), 3.41-3.39 (m, 2H), 2.61-2.58 (m, |
| | 3-d]pyrimidin-7(8H)-one | | 5H), 1.39 (s, 2H), 1.23-1.21 (m, 6H) |
| I-63 | 8-[2-(2- | 507.1 | (500 MHz, DMSO-d₆): δ 8.71 (m, 2H), 8.22 (d, |
| | Aminoethoxy)ethyl]-6-[2- | | J = 2.0 Hz, 1H), 8.10 (d, J = 10 Hz 1H), |
| | chloro-4-(6-methyl-2- | | 7.88-7.81 (m, 3H), 7.50 (d, J = 7.5 Hz, 1H), 7.28 (d, J = 7.0 Hz, |
| | pyridyl)phenyl]-2-(oxetan- | | 1H), 5.04 (d, J = 5.0 Hz, 1H), 4.95 (d, J = 6.1 Hz, |
| | 3-ylamino)pyrido[2,3- | | 2H), 4.83 (d, J = 4.1 Hz, 2H), 4.47 (d, J = 5.2 Hz, |
| | d]pyrimidin-7-one | | 2H), 3.67-3.65 (m, 2H), 3.42-3.39 (m, 2H), |
| | | | 2.61-2.56 (m, 5H), 1.66-1.49 (brs, 1H) |
| I-48 | 8-[2-(2- | 521.0 | (500 MHz, DMSO-d₆): δ 8.68 (s, 1H), 8.23 (d, |
| | Aminoethoxy)ethyl]-6-[2- | | J = 1.5 Hz 2H), 8.08 (d, J = 9.5 Hz 1H), |
| | chloro-4-(6-methyl-2- | | 7.88-7.85 (m, 2H), 7.82-7.81 (m, 1H), 7.50 (d, J = 8.0 Hz 1H), |
| | pyridyl)phenyl]-2-(THF-3- | | 7.28 (d, J = 7.5 Hz 1H), 4.50 (brs, 3H), 3.97 (brs, |
| | ylamino)pyrido[2,3- | | 1H), 3.87-3.85 (m, 2H), 3.69-3.67 (m, 5H), |
| | d]pyrimidin-7-one | | 2.61-2.57 (m, 6H), 2.23 (brs, 1H), 2.00 (brs, 1H), |
| | | | 1.55 (brs, 1H) |
| I-118 | 8-[2-(azetidin-3-yloxy)-1- | 506.0 | (500 MHz, CDCl₃): δ 8.82 (s, 1H), 8.46 (s, 1H), |
| | methyl-ethyl]-6-[2-chloro- | | 8.43 (s, 1H), 8.16 (m, 1H), 7.94 (dd, J = 5.0 Hz, |
| | 4-(6-methylpyrazin-2- | | 1.0 Hz, 1H), 7.54 (s, 1H), 7.50 (d, J = 5.0 Hz, 1H), |
| | yl)phenyl]-2- | | 5.85-5.83 (m, 1H), 5.51 (s, 1H), 4.40-4.35 (m, 1H), |
| | (ethylamino)pyrido[2,3- | | 3.91-3.90 (m, 1H), 3.74-3.68 (m, 2H), 3.63-3.56 (m, |
| | d]pyrimidin-7-one | | 4H), 2.65 (s, 3H), 1.65-1.58 (m, 2H), 1.33-1.26 (m, |
| | (enantiomer 1; unknown | | 6H). |
| | absolute configuration) | | |
| I-119 | 8-[2-(azetidin-3-yloxy)-1- | 506.0 | (500 MHz, CDCl3): δ 8.82 (s, 1H), 8.46 (s, 1H), |
| | methyl-ethyl]-6-[2-chloro- | | 8.43 (s, 1H), 8.16 (m, 1H), 7.94 (dd, J = 1.0 Hz, |
| | 4-(6-methylpyrazin-2- | | 5.0 Hz, 1H), 7.54 (s, 1H), 7.50 (d, J = 5.0 Hz, 1H), |
| | yl)phenyl]-2- | | 5.85-5.83 (m, 1H), 5.51 (s, 1H), 4.42-4.40 (m, 1H), |
| | (ethylamino)pyrido[2,3- | | 3.91-3.82 (m, 3H), 3.71-3.65 (m, 2H), 3.55 (s, 2H), |
| | d]pyrimidin-7-one | | 2.65 (s, 3H), 1.65-1.58 (m, 2H), 1.33-1.26 (m, 6H). |
| | (enantiomer 2; unknown | | |
| | absolute configuration) | | |
| I-111 | 6-[2-Chloro-4-(6- | 478.2 | (400 MHz, DMSO-d6) d 9.15 (s, 1H), 8.72 (s, |
| | methylpyrazin-2- | | 0.3H, rotamer), 8.65 (s, 0.7H, rotamer), 8.57 (s, |
| | yl)phenyl]-2- | | 1H), 8.27 (d, J = 1.8 Hz, 1H), 8.15 (dd, J = 8.0, 1.8 Hz, |
| | (methylamino)-8-[[(2R)- | | 1H), 7.90 (d, J = 5.2 Hz, 0.7H, rotamer), |
| | morpholin-2- | | 7.85 (s, 1H), 7.79 (br s, 0.3H, rotamer), 7.56 (d, J = 8.0 Hz, |
| | yl]methyl]pyrido[2,3- | | 1H), 4.48-4.39 (m, 1H), 4.31 (dd, J = 12.7, |
| | d]pyrimidin-7-one | | 6.8 Hz, 1H), 3.88-3.78 (m, 1H), 3.74 (d, J = 11.1 Hz, |
| | | | 1H), 3.41-3.33 (m, 1H), 2.93 (d, J = 4.7 Hz, |
| | | | 3H), 2.72-2.61 (m, 3H), 2.60 (s, 3H), |
| | | | 2.56-2.51 (m, 1H), 2.40-2.26 (m, 1H) |
| I-75 | 6-[2-Chloro-4-(6- | 518.0 | (500 MHz, CDCl₃-d₃): δ 8.83 (s, 1H), 8.46 (s, 1H), |
| | methylpyrazin-2- | | 8.43 (s, 1H), 8.16 (d, J = 1.5 Hz, 1H), 7.94 (d, J = 8.0 Hz, |
| | yl)phenyl]-2-(ethylamino)- | | 1H), 7.54 (s, 1H), 7.49 (d, J = 8.0 Hz, 1H), |
| | 8-(1-oxa-7- | | 6.33 (br, 1H), 5.53 (s, 1H), 4.52 (br, 1H), |
| | azaspiro[4.4]nonan-3- | | 4.05-3.95 (m, 1H), 3.65-3.55 (m, 2H), 3.20-2.85 (m, 4H), |
| | yl)pyrido[2,3-d]pyrimidin- | | 2.73-2.59 (m, 4H), 2.30-1.95 (m, 3H), 1.31 (t, J = 7.2 Hz, |
| | 7-one | | 3H) |
| I-59 | 6-(2-Chloro-4-(6- | 532.0 | (500 MHz, CDCl₃-d₃): δ 8.82 (s, 1H), 8.46 (s, 1H), |
| | methylpyrazin-2- | | 8.42 (s, 1H), 8.16 (d, J = 1.6 Hz, 1H), 7.94 (dd, J = 8.0, |
| | yl)phenyl)-2-(ethylamino)- | | 1.7 Hz, 1H), 7.53 (s, 1H), 7.49 (d, J = 8.0 Hz, |
| | 8-(1-oxa-8- | | 1H), 6.37 (br, 1H), 5.54 (s, 1H), 4.61 (br, 1H), |
| | azaspiro[4.5]decan-3- | | 4.03 (t, J = 8.2 Hz, 1H), 3.59 (s, 2H), 3.09-3.00 (m, 2H), |
| | yl)pyrido[2,3-d]pyrimidin- | | 2.84-2.58 (m, 6H), 2.09-1.72 (m, 5H), 1.30 (t, J = 7.2 Hz, |
| | 7(8H)-one | | 3H) |
| I-121 | 6-[2-Chloro-4-(6-methyl- | 517.0 | (500 MHz, CDCl3-d1): δ 8.46 (s, 1H), 8.12 (d, J = 1.7 Hz, |
| | 2-pyridyl)phenyl]-2- | | 1H), 7.91 (dd, J = 1.7, 8.0 Hz, 1H), 7.65 (t, |
| | (ethylamino)-8-(1-oxa-7- | | J = 7.7 Hz, 1H), 7.55-7.49 (m, 2H), 7.44 (dd, J = 8.0, |
| | azaspiro[4.4]nonan-3- | | 2.3 Hz, 1H), 7.13 (d, J = 7.6 Hz, 1H), |
| | yl)pyrido[2,3-d]pyrimidin- | | 6.33 (brs, 1H), 5.51 (s, 1H), 4.57-4.40 (m, 1H), |
| | 7-one | | 4.07-3.94 (m, 1H), 3.67-3.53 (m, 2H), 3.44 (s, 0.5 H), |
| | | | 3.24-2.86 (m, 4H), 2.72 (d, J = 11.7 Hz, 0.5 H), |
| | | | 2.63 (s, 3H), 2.34-1.96 (m, 4H), 1.31 (t, J = 7.2 Hz, |
| | | | 3H). |
| I-122 | 2-(Ethylamino)-6-[2- | 497.0 | 1 (500 MHz, CDCl3) δ 8.44 (s, 1H), 7.87 (s, 1H), |
| | methyl-4-(6-methyl-2- | | 7.80 (dd, J = 1.5, 7.9 Hz, 1H), 7.63 (t, J = 7.7 Hz, |
| | pyridyl)phenyl]-8-(1-oxa- | | 1H), 7.51 (d, J = 7.8 Hz, 1H), 7.42 (s, 1H), 7.28 (d, |
| | 7-azaspiro[4.4]nonan-3- | | J = 7.8 Hz, 1H), 7.09 (d, J = 7.6 Hz, 1H), 6.34 (brs, |
| | yl)pyrido[2,3-d]pyrimidin- | | 1H), 5.49 (s, 1H), 4.57-4.38 (m, 1H), |
| | 7-one | | 4.05-3.94 (m, 1H), 3.65-3.54 (m, 2H), 3.40 (d, J = 11.6 Hz, |
| | | | 0.5 H), 3.22-2.82 (m, 5H), 2.69 (d, J = 11.8 Hz, 0.5 |
| | | | H), 2.63 (s, 3H), 2.35-1.94 (m, 7H), 1.30 (t, J = 7.2 Hz, |
| | | | 4H). |
| I-57 | 6-(2-Chloro-4-(6- | 490.2 | (400 MHz, d6-DMSO) δ 9.14 (s, 1H), 8.64 (s, 1H), |
| | methylpyrazin-2- | | 8.56 (s, 1H), 8.26 (s, 1H), 8.14 (m, 1H), 7.95 (m, |
| | yl)phenyl)-2-(ethylamino)- | | 1H), 7.82 (s, 1H), 7.55 (m, 1H), 4.31 (m, 2H), |
| | 8-(2-(pyrrolidin-3- | | 3.42 (m, 2H), 2.94 (m, 1H), 2.73 (m, 2H), 2.60 (s, 3H), |
| | yl)ethyl)pyrido[2,3- | | 2.38 (m, 1H), 1.97 (m, 1H), 1.86 (m, 1H), 1.70 (m, |
| | d]pyrimidin-7(8H)-one | | 2H), 1.31 (m, 1H), 1.20 (m, 3H) |

### Example 72

### 6-(2-Chloro-4-(6-methylpyrazin-2-yl)phenyl)-2-(ethylamino)-8-(5-oxa-2-azaspiro[3.4]octan-7-yl)pyrido[2,3-d]pyrimidin-7(8H)-one (I-58)

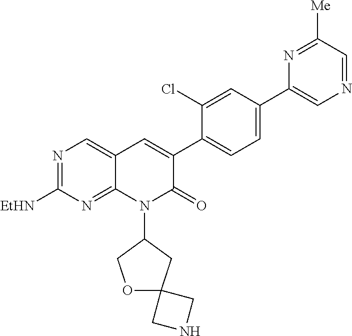

The title compound was prepared in accord with Example 71 using commercially available tert-butyl 7-bromo-5-oxa-2-azaspiro[3.4]octane-2-carboxylate as starting material and adding 1 eq. of sodium iodide in step 1. ¹H NMR (500 MHz, CD₃OD) δ 8.94 (s, 1H), 8.57 (s, 1H), 8.47 (s, 1H), 8.23 (d, J=1.6 Hz, 1H), 8.05 (dd, J=8.0, 1.7 Hz, 1H), 7.74 (s, 1H), 7.52 (d, J=8.0 Hz, 1H), 6.49-6.37 (m, 1H), 4.34 (s, 1H), 4.10-4.00 (m, 1H), 3.93 (d, J=9.6 Hz, 1H), 3.89-3.83 (m, 1H), 3.79 (d, J=9.3 Hz, 1H), 3.70 (d, J=9.3 Hz, 1H), 3.55-3.46 (m, 2H), 2.93 (br, 1H), 2.64 (s, 3H), 2.48 (br, 1H), 1.26 (t, J=7.2 Hz, 3H); LCMS (ESI): m/z=504 [M+H]⁺.

### Example 73

### 6-(2-Methyl-4-(6-methylpyridin-2-yl)phenyl)-8-(2-(morpholin-2-yl)ethyl)-2-(oxetan-3-ylamino)pyrido[2,3-d]pyrimidin-7(8H)-one (I-113) and 6-(2-methyl-4-(6-methylpyridin-2-yl)phenyl)-8-(2-(morpholin-2-yl)ethyl)-2-(oxetan-3-ylamino)pyrido[2,3-d]pyrimidin-7(8H)-one (I-114)

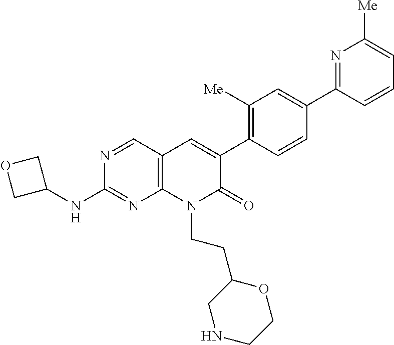 [SMILES: Cc1cccc(-c2ccc(-c3cc4cnc(NC5COC5)nc4n(CCC4CNCCO4)c3=O)c(C)c2)n1]

Racemic benzyl 2-(2-(6-(2-methyl-4-(6-methylpyridin-2-yl)phenyl)-2-(oxetan-3-ylamino)-7-oxopyrido[2,3-d]pyrimidin-8(7H)-yl)ethyl)morpholine-4-carboxylate was prepared in accord with Example 71 employing appropriate starting materials. Chiral SFC separation afforded the two enantiomers: Peak 1: RT=0.944 min. Peak 2: RT=1.297 min. MS (ESI) m/z: 647.2 [M+1]⁺.

The material represented by peak 1 above (51.0 mg, 0.079 mmol) was placed in a 10-mL microwave vial, together with ammonium formate (49.7 mg, 0.79 mmol), 10% Pd/C (10 mg), and IPA (7.9 mL). The vial was sealed and the reaction mixture was stirred in the microwave at 110° C. for 5 min. The reaction mixture was cooled and then filtered through a pad of Celite to remove Pd and formate salt. The filtrate was concentrated under reduced pressure. The crude was purified by preparative HPLC to give the title compound (enantiomer 1) as a solid (17.4 mg, 43% yield). ¹H NMR (400 MHz, DMSO-d₆) δ d 8.68 (s, 1H), 8.57 (br s, 1H), 7.96 (d, J=1.8 Hz, 1H), 7.90 (dd, J=8.0, 1.9 Hz, 1H), 7.80-7.75 (m, 2H), 7.74 (s, 1H), 7.27 (d, J=7.9 Hz, 1H), 7.22 (dd, J=5.1, 3.4 Hz, 1H), 4.98 (br s, 1H), 5.08-4.92 (m, 1H), 4.92-4.75 (m, 2H), 4.43 (dt, J=14.5, 6.7 Hz, 2H), 4.27 (ddd, J=12.4, 9.3, 5.5 Hz, 1H), 3.75-3.68 (m, 1H), 3.48-3.36 (m, 2H), 2.78 (dd, J=12.3, 2.2 Hz, 1H), 2.68-2.58 (m, 2H), 2.55 (s, 3H), 2.37 (dd, J=12.1, 9.9 Hz, 1H), 2.22 (s, 3H), 1.85-1.60 (m, 2H); 1H not seen. MS (ESI) m/z: 513.3 [M+1]⁺.

The material represented by peak 2 above (46.7 mg, 0.072 mmol) was placed in a 10-mL microwave vial, together with ammonium formate (45.5 mg, 0.72 mmol), 10% Pd/C (10 mg), and IPA (7.2 mL). The vial was sealed and the reaction mixture was stirred in the microwave at 110° C. for 10 min. The reaction mixture was cooled and then filtered through a pad of Celite to remove Pd and formate salt. The filtrate was concentrated under reduced pressure. The crude was purified by preparative HPLC to give the title compound (enantiomer 2; unknown absolute configuration) as a solid (20.5 mg, 50.8% yield). ¹H NMR (400 MHz, DMSO-d₆) δ 8.68 (s, 1H), 8.57 (br s, 1H), 7.96 (d, J=1.8 Hz, 1H), 7.90 (dd, J=8.0, 1.9 Hz, 1H), 7.80-7.75 (m, 2H), 7.74 (s, 1H), 7.27 (d, J=7.9 Hz, 1H), 7.22 (dd, J=5.1, 3.4 Hz, 1H), 4.98 (br s, 1H), 5.08-4.92 (m, 1H), 4.92-4.75 (m, 2H), 4.43 (dt, J=14.5, 6.7 Hz, 2H), 4.27 (ddd, J=12.4, 9.3, 5.5 Hz, 1H), 3.75-3.68 (m, 1H), 3.48-3.36 (m, 2H), 2.78 (dd, J=12.3, 2.2 Hz, 1H), 2.68-2.58 (m, 2H), 2.55 (s, 3H), 2.37 (dd, J=12.1, 9.9 Hz, 1H), 2.22 (s, 3H), 1.85-1.60 (m, 2H); 1H not seen. MS (ESI) m/z: 513.3 [M+1]⁺.

### Example 74

### 6-(2-Methyl-4-(6-methylpyridin-2-yl)phenyl)-2-(methylamino)-8-(2-(morpholin-2-yl)ethyl)pyrido[2,3-d]pyrimidin-7(8H)-one (I-104) and (S)-6-(2-methyl-4-(6-methylpyridin-2-yl)phenyl)-2-(methylamino)-8-(2-(morpholin-2-yl)ethyl)pyrido[2,3-d]pyrimidin-7(8H)-one (I-110)

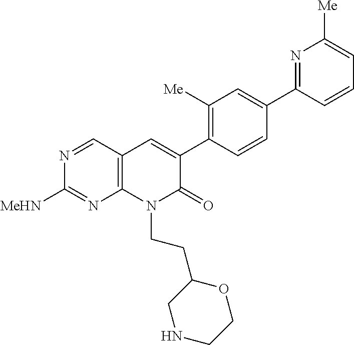 [SMILES: CNc1ncc2cc(-c3ccc(-c4cccc(C)n4)cc3C)c(=O)n(CCC3CNCCO3)c2n1]

The title compounds were prepared in accord with Example 73 using appropriate starting materials. Enantiomer 1: ¹H NMR (400 MHz, DMSO-d₆) δ 8.62 (s, 1H), 7.96 (d, J=1.7 Hz, 1H), 7.90 (dd, J=7.9, 1.9 Hz, 1H), 7.78 (s, 1H), 7.77 (d, J=1.7 Hz, 1H), 7.72 (s, 1H), 7.28 (d, J=8.0 Hz, 1H), 7.25-7.18 (m, 1H), 4.45-4.31 (m, 2H), 3.69 (dd, J=10.8, 2.6 Hz, 1H), 3.46-3.34 (m, 2H), 3.34-3.24 (m, 2H), 2.93 (d, J=4.7 Hz, 3H), 2.80 (d, J=11.9 Hz, 1H), 2.62 (dd, J=9.6, 3.1 Hz, 2H), 2.56 (s, 3H), 2.38 (t, J=11.0 Hz, 1H), 2.23 (s, 3H), 1.75 (s, 2H). MS (ESI) m/z: 471.3 [M+1]⁺.

Enantiomer 2: ¹H NMR (400 MHz, DMSO-d₆) δ 8.62 (s, 1H), 8.25 (s, 1H), 7.96 (d, J=1.8 Hz, 1H), 7.90 (dd, J=8.0, 1.9 Hz, 1H), 7.78-7.76 (m, 2H), 7.72 (s, 1H), 7.28 (d, J=7.9 Hz, 1H), 7.22 (dd, J=5.1, 3.4 Hz, 1H), 4.51-4.33 (m, 2H), 3.71 (d, J=11.1 Hz, 1H), 3.51-3.36 (m, 3H), 2.93 (d, J=4.8 Hz, 3H), 2.88-2.81 (m, 1H), 2.74-2.60 (m, 2H), 2.55 (s, 3H), 2.47-2.38 (m, 1H), 2.23 (s, 3H), 1.85-1.67 (m, 2H). MS (ESI) m/z: 471.3 [M+1]⁺.

### Example 74

### 2-(Ethylamino)-6-(2-methyl-4-(6-methylpyridin-2-yl)phenyl)-8-(2-(morpholin-2-yl)ethyl)pyrido[2,3-d]pyrimidin-7(8H)-one (I-112) and (S)-2-(ethylamino)-6-(2-methyl-4-(6-methylpyridin-2-yl)phenyl)-8-(2-(morpholin-2-yl)ethyl)pyrido[2,3-d]pyrimidin-7(8H)-one (I-115)

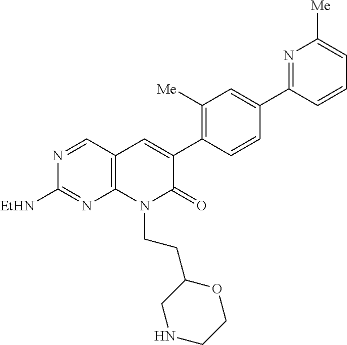

The title compounds were prepared in accord with Example 73 using appropriate starting materials.

Enantiomer 1: ¹H NMR (400 MHz, DMSO-d₆) δ 8.68 (s, 1H), 8.57 (br s, 1H), 7.96 (d, J=1.8 Hz, 1H), 7.90 (dd, J=8.0, 1.9 Hz, 1H), 7.81-7.76 (m, 2H), 7.74 (s, 1H), 7.27 (d, J=7.9 Hz, 1H), 7.22 (dd, J=5.1, 3.4 Hz, 1H), 4.98 (br s, 1H), 4.92-4.77 (m, 2H), 4.61 (td, J=6.2, 3.5 Hz, 2H), 4.47-4.38 (m, 1H), 4.32-4.23 (m, 1H), 3.71 (d, J=11.2 Hz, 1H), 3.48-3.38 (m, 2H), 2.81-2.74 (m, 1H), 2.64 (t, J=3.0 Hz, 1H), 2.55 (s, 2H), 2.37 (dd, J=12.1, 10.0 Hz, 1H), 2.22 (s, 3H), 1.80-1.60 (m, 2H), 1.23 (s, 3H). MS (ESI) m/z: 485.3 [MH]⁺.

Enantiomer 2: ¹H NMR (400 MHz, DMSO-d₆) δ 8.62 (s, 1H), 7.96 (d, J=1.9 Hz, 1H), 7.90 (dd, J=7.9, 1.9 Hz, 2H), 7.80-7.75 (m, 2H), 7.71 (s, 1H), 7.28 (d, J=7.9 Hz, 1H), 7.25-7.19 (m, 1H), 4.43 (br s, 1H), 4.33 (br s, 1H), 3.74-3.62 (m, 1H), 3.48-3.34 (m, 4H), 2.78 (d, J=11.0 Hz, 1H), 2.61 (dd, J=9.8, 3.1 Hz, 2H), 2.56 (s, 3H), 2.37 (dd, J=12.0, 9.8 Hz, 2H), 2.23 (s, 3H), 1.83-1.64 (m, 2H), 1.20 (t, J=7.1 Hz, 3H). MS (ESI) m/z: 485.3 [MH]⁺.

### Example 75

### 6-(2-Chloro-4-(6-methylpyridin-2-yl)phenyl)-2-(methylamino)-8-(2-(morpholin-2-yl)ethyl)pyrido[2,3-d]pyrimidin-7(8H)-one (I-124) and 6-(2-chloro-4-(6-methylpyridin-2-yl)phenyl)-2-(methylamino)-8-(2-(morpholin-2-yl)ethyl)pyrido[2,3-d]pyrimidin-7(8H)-one (I-125)

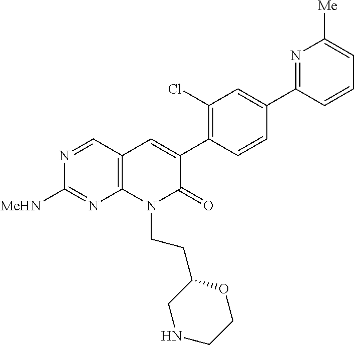 [SMILES: CNc1ncc2cc(-c3ccc(-c4cccc(C)n4)cc3Cl)c(=O)n(CC[C@H]3CNCCO3)c2n1]

Racemic benzyl 2-[2-[6-[2-chloro-4-(6-methyl-2-pyridyl)phenyl]-2-(methylamino)-7-oxo-pyrido[2,3-d]pyrimidin-8-yl]ethyl]morpholine-4-carboxylate was prepared using procedures analogous to Example 73, using appropriate starting materials. Chiral SFC separation afforded the two enantiomers: Peak 1: RT=0.978 min. Peak 2: RT=1.409 min. MS (ESI) m/z: 625.2 [M+1]⁺.

A mixture of the material represented by Peak 1 above (35.9 mg, 0.057 mmol) in dioxane (2.9 mL) and 50% aq. HCl (2.9 mL) was stirred at 100° C. for 16 h. The cooled reaction mixture was basified with 10% aq. NaOH until pH˜7, and the reaction mixture was extracted with 25% isopropanol in chloroform (3×). The combined organic layers were washed with brine, dried over Na₂SO₄, filtered, and concentrated under reduced pressure. The crude was purified by preparative HPLC to give the title compound (Enantiomer 1) as a solid (11.2 mg, 35.7% yield). ¹H NMR (400 MHz, DMSO-d₆) δ 8.64 (s, 1H), 8.22 (d, J=1.7 Hz, 1H), 8.08 (dd, J=8.1, 1.8 Hz, 1H), 7.91-7.77 (m, 4H), 7.50 (d, J=8.0 Hz, 1H), 7.28 (d, J=7.4 Hz, 1H), 4.51-4.32 (m, 2H), 4.33-4.19 (m, 1H), 3.78-3.69 (m, 1H), 3.52-3.38 (m, 2H), 2.97-2.83 (m, 4H), 2.77-2.63 (m, 2H), 2.57 (s, 3H), 2.50-2.40 (m, 1H), 1.86-1.65 (m, 2H). MS (ESI) m/z: 491.2 [M+1]⁺.

A mixture of the material represented by Peak 2 above (40 mg, 0.064 mmol) in dioxane (3.2 mL) and 50% aq. HCl (3.2 mL) was stirred at 100° C. for 16 h. The cooled reaction mixture was basified with 10% aq. NaOH until pH˜7, and the reaction mixture was extracted with 25% isopropanol in chloroform (3×). The combined organic layers were washed with brine, dried over Na₂SO₄, filtered, and concentrated under reduced pressure. The crude was purified by preparative HPLC to give the title compound (Enantiomer 2 with unknown absolute configuration) as a solid (18 mg, 63.8% yield). ¹H NMR (400 MHz, DMSO-d₆) δ 8.64 (s, 1H), 8.22 (d, J=1.8 Hz, 1H), 8.08 (dd, J=8.1, 1.8 Hz, 1H), 7.90-7.77 (m, 4H), 7.50 (d, J=8.0 Hz, 1H), 7.28 (d, J=7.0 Hz, 1H), 4.50-4.32 (m, 2H), 4.31-4.19 (m, 1H), 3.72-3.66 (m, 1H), 3.46-3.34 (m, 2H), 2.93 (d, J=4.9 Hz, 3H), 2.79 (d, J=11.3 Hz, 1H), 2.65-2.59 (m, 2H), 2.57 (s, 3H), 2.38 (t, J=11.1 Hz, 1H), 1.84-1.64 (m, 2H). MS (ESI) m/z: 491.2 [M+1]⁺.

### Example 76

### 2-(2,2-Difluoroethylamino)-6-[2-methyl-4-(6-methyl-2-pyridyl)phenyl]-8-(2-morpholin-2-ylethyl)pyrido[2,3-d]pyrimidin-7-one (I-126)

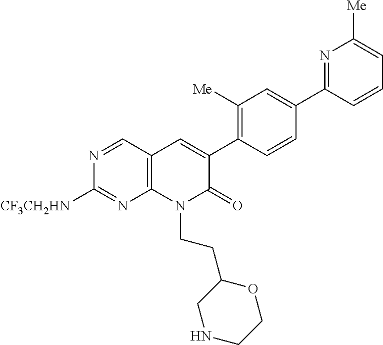

The title compound (racemic) was prepared using a similar procedure in accord with Example 75 using appropriate starting materials. ¹H NMR: (400 MHz, DMSO-d₆) d 8.62 (s, 1H), 7.96 (d, J=1.9 Hz, 1H), 7.90 (dd, J=8.0, 1.9 Hz, 1H), 7.87 (br s, 1H), 7.81-7.73 (m, 2H), 7.71 (s, 1H), 7.28 (d, J=7.9 Hz, 1H), 7.22 (dd, J=5.1, 3.4 Hz, 1H), 4.49-4.39 (m, 1H), 4.39-4.27 (m, 1H), 3.73-3.66 (m, 1H), 3.47-3.34 (m, 4H), 2.83-2.73 (m, 1H), 2.69-2.58 (m, 2H), 2.56 (s, 3H), 2.37 (dd, J=12.1, 9.8 Hz, 1H), 2.23 (s, 3H), 1.84-1.61 (m, 2H), 1.26-1.13 (m, 3H); MS (ESI) m/z: 521.3 [M+1]⁺.

### Example 77

### 2-Amino-6-[2-chloro-4-(6-methyl-2-pyridyl)phenyl]-8-(2-morpholin-2-ylethyl)pyrido[2,3-d]pyrimidin-7-one (I-127)

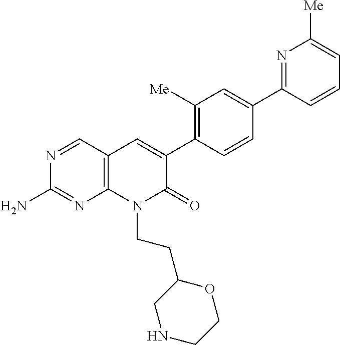 [SMILES: Cc1cccc(-c2ccc(-c3cc4cnc(N)nc4n(CCC4CNCCO4)c3=O)c(C)c2)n1]

The title compound (racemic) was prepared using a similar procedure in accord with Example 75 using appropriate starting materials. ¹H NMR: (400 MHz, DMSO-d₆) δ 8.64 (s, 1H), 8.22 (d, J=1.7 Hz, 1H), 8.08 (dd, J=8.0, 1.8 Hz, 1H), 7.89-7.78 (m, 3H), 7.50 (d, J=8.0 Hz, 1H), 7.31 (br s, 2H), 7.28 (d, J=7.4 Hz, 1H), 4.41 (ddd, J=12.3, 9.1, 6.3 Hz, 1H), 4.26 (ddd, J=12.2, 9.1, 5.1 Hz, 1H), 3.69 (dt, J=11.1, 2.6 Hz, 1H), 3.44-3.33 (m, 2H), 2.78 (dd, J=12.1, 2.3 Hz, 1H), 2.66-2.58 (m, 2H), 2.57 (s, 3H), 2.36 (dd, J=12.2, 9.9 Hz, 1H), 1.82-1.70 (m, 1H), 1.70-1.59 (m, 1H); MS (ESI) m/z: 477.2 [M+1]⁺.

### Example 77

### 6-(2-Chloro-4-(6-methylpyrazin-2-yl)phenyl)-2-(ethylamino)-8-((1-(2-hydroxyethyl)piperidin-4-yl)methyl)pyrido[2,3-d]pyrimidin-7(8H)-one (I-39)

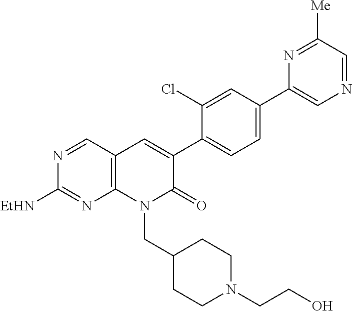

A mixture of 6-(2-chloro-4-(6-methylpyrazin-2-yl)phenyl)-2-(ethylamino)-8-(piperidin-4-ylmethyl)pyrido[2,3-d]pyrimidin-7(8H)-one (100 mg, 0.20 mmol), 2-bromoethanol (25 mg, 0.20 mmol), K₂CO₃ (55 mg, 0.40 mmol) in DMSO (3 mL) was heated at 90° C. for 1 h. The reaction mixture was then concentrated to give the crude product which was purified by preparative HPLC to afford I-39 (40.6 mg, 37%) as a white solid. ¹H NMR (500 MHz, DMSO-d₆): δ 9.16 (s, 1H), 8.64 (s, 1H), 8.57 (s, 1H), 8.27 (s, 1H), 8.15 (s, 1H), 7.99 (d, 1H), 7.83 (s, 1H), 7.55 (d, 1H), 4.32-4.22 (m, 3H), 3.46-3.40 (m, 4H), 2.83 (s, 2H), 2.60 (s, 3H), 2.33 (s, 2H), 1.88-1.86 (m, 3H), 1.54 (d, 2H), 1.34-1.29 (m, 2H), 1.23-1.15 (m, 3H). LCMS (ESI): m/z=534.3 [M+1]⁺.

### Example 78

### 2-(Ethylamino)-8-(4-hydroxybutyl)-6-[2-methyl-4-(6-methyl-2-pyridyl)phenyl]pyrido[2,3-d]pyrimidin-7-one (I-73)

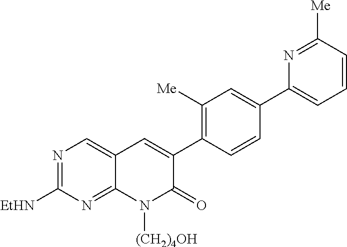

step 1: A mixture of 4-bromo-1-butanol (3.5 equiv., 1.87 mmol), Cs₂CO₃ (522.0 mg, 1.60 mmol), and 6-[2-methyl-4-(6-methyl-2-pyridyl)phenyl]-2-methylsulfanyl-8H-pyrido[2,3-d]pyrimidin-7-one (200 mg, 0.53 mmol) in anhydrous DMF (5050 mg, 69.0 mmol, 5.340 mL) was stirred at RT for 2.5 d. The reaction mixture was washed with brine and the organic layers were combined, dried (Na₂SO₄), and reduced in vacuo. The crude product, 8-(4-hydroxybutyl)-6-[2-methyl-4-(6-methyl-2-pyridyl)phenyl]-2-methylsulfanyl-pyrido[2,3-d]pyrimidin-7-one, was used in the next step without any further purification. MS (ESI) m/z: 447.4 [M+1]⁺.

step 2: To 8-(4-hydroxybutyl)-6-[2-methyl-4-(6-methyl-2-pyridyl)phenyl]-2-methylsulfanyl-pyrido[2,3-d]pyrimidin-7-one (235 mg, 0.53 mmol) in anhydrous DCM (6.7 mL) at 0° C. was added, portionwise over 4 h, MCPBA (3.0 equiv., 1.58 mmol, 353.7 mg). The reaction mixture was stirred at 0° C. for a total of 4.5 h, quenched with saturated NaHCO₃ (25 mL), and extracted with DCM (3×30 mL). The organic layers were combined and reduced in vacuo to afford crude 8-(4-hydroxybutyl)-6-[2-methyl-4-(6-methyl-2-pyridyl)phenyl]-2-methylsulfinyl-pyrido[2,3-d]pyrimidin-7-one, which was used in the next steps without further purification. MS (ESI) m/z: 463.4 [M+1]⁺.

step 3: A mixture of 8-(4-hydroxybutyl)-6-[2-methyl-4-(6-methyl-2-pyridyl)phenyl]-2-methylsulfinyl-pyrido[2,3-d]pyrimidin-7-one (115 mg, 0.249 mmol), ethylamine (2 mol/L in THF, 1.24 mmol, 0.62 mL), and DIPEA (4.0 equiv., 1.0 mmol, 0.173 mL) in anhydrous THF (2 mL) was stirred at RT overnight. The reaction mixture was poured into brine and extracted with DCM. The solvents of the combined organic layers were removed and the crude material was purified by reverse-phase HPLC. 2-(Ethylamino)-8-(4-hydroxybutyl)-6-[2-methyl-4-(6-methyl-2-pyridyl)phenyl]pyrido[2,3-d]pyrimidin-7-one was obtained as an off-white solid (19.6 mg, 0.043 mmol, 17%). ¹H NMR (400 MHz, DMSO-d₆) δ 8.62 (s, 1H), 7.96 (s, 1H), 7.90 (d, J=8.2 Hz, 1H), 7.77 (d, J=4.3 Hz, 2H), 7.71 (s, 1H), 7.28 (d, J=7.9 Hz, 1H), 7.26-7.19 (m, 1H), 4.42-4.34 (m, 1H), 4.34-4.25 (m, 2H), 3.46-3.40 (m, 3H), 2.55 (s, 3H), 2.23 (s, 3H), 1.20 (t, J=7.4 Hz, 4H), 3.40-3.37 (m, 1H), 1.88-1.62 (m, 2H), 1.58-1.44 (m, 2H); MS (ESI) m/z: 444.2 [M+1]⁺.

8-(4-Hydroxybutyl)-6-[2-methyl-4-(6-methyl-2-pyridyl)phenyl]-2-(oxetan-3-ylamino)pyrido[2,3-d]pyrimidin-7-one (I-81) was prepared analogously except oxetan-3-amine replaced ethylamine in step 3. ¹H NMR (400 MHz, DMSO-d₆) δ 8.69 (s, 1H), 8.57 (s, 1H), 7.99-7.87 (m, 2H), 7.78 (s, 1H), 7.77-7.72 (m, 2H), 7.32-7.17 (m, 2H), 4.96 (s, 1H), 4.84 (s, 2H), 4.61 (t, J=6.2 Hz, 2H), 4.45-4.37 (m, 1H), 4.33-4.24 (m, 2H), 3.49-3.39 (m, 2H), 2.56 (s, 3H), 2.22 (s, 3H), 1.73-1.64 (m, 2H), 1.56-1.44 (m, 2H); MS (ESI) m/z: 472.23 [M+1]⁺.

6-[2-Chloro-4-(6-methylpyrazin-2-yl)phenyl]-2-(ethylamino)-8-(4-hydroxybutyl)pyrido[2,3-d]pyrimidin-7-one (I-36) was prepared analogously except 6-[2-methyl-4-(6-methyl-2-pyridyl)phenyl]-2-methylsulfanyl-8H-pyrido[2,3-d]pyrimidin-7-one was replaced with 6-[2-chloro-4-(6-methylpyrazin-2-yl)phenyl]-2-methylsulfanyl-8H-pyrido[2,3-d]pyrimidin-7-one in step 1. ¹H NMR (400 MHz, DMSO-d₆) δ 9.16 (s, 1H), 8.67-8.62 (m, 1H), 8.60-8.55 (m, 1H), 8.30-8.25 (m, 1H), 8.23-8.12 (m, 1H), 8.02-7.95 (m, 1H), 7.86-7.81 (m, 1H), 7.63-7.52 (m, 1H), 4.44-4.35 (m, 1H), 4.36-4.28 (m, 2H), 3.48-3.38 (m, 4H), 2.60 (s, 3H), 1.74-1.65 (m, 2H), 1.55-1.43 (m, 2H), 1.20 (t, J=7.1 Hz, 3H); MS (ESI) m/z: 465.2 [M+1]⁺.

6-[2-Chloro-4-(6-methylpyrazin-2-yl)phenyl]-8-(4-hydroxybutyl)-2-(oxetan-3-ylamino)pyrido[2,3-d]pyrimidin-7-one (I-50) was prepared analogously except 6-[2-methyl-4-(6-methyl-2-pyridyl)phenyl]-2-methylsulfanyl-8H-pyrido[2,3-d]pyrimidin-7-one was replaced with 6-[2-chloro-4-(6-methylpyrazin-2-yl)phenyl]-2-methylsulfanyl-8H-pyrido[2,3-d]pyrimidin-7-one in step 1 and oxetan-3-amine replaced ethylamine in step 3. ¹H NMR (400 MHz, DMSO-d₆) δ 9.19-9.12 (m, 1H), 8.71 (s, 1H), 8.63 (s, 1H), 8.57 (s, 1H), 8.33-8.24 (m, 1H), 8.15 (dd, J=8.1, 1.8 Hz, 1H), 7.86 (s, 1H), 7.62-7.52 (m, 1H), 4.99-4.94 (m, 1H), 4.84 (s, 2H), 4.61 (t, J=6.3 Hz, 2H), 4.44-4.36 (m, 1H), 4.28 (t, J=7.5 Hz, 2H), 3.44 (q, J=6.0 Hz, 2H), 2.60 (s, 3H), 1.73-1.64 (m, 2H), 1.56-1.42 (m, 2H); MS (ESI) m/z: 493.17 [M+1]⁺.

6-[2-Chloro-4-(6-methylpyrazin-2-yl)phenyl]-8-[2-(2-hydroxyethoxy)ethyl]-2-(oxetan-3-ylamino)pyrido[2,3-d]pyrimidin-7-one (I-67) was prepared analogously except 6-[2-methyl-4-(6-methyl-2-pyridyl)phenyl]-2-methylsulfanyl-8H-pyrido[2,3-d]pyrimidin-7-one was replaced with 6-[2-chloro-4-(6-methylpyrazin-2-yl)phenyl]-2-methylsulfanyl-8H-pyrido[2,3-d]pyrimidin-7-one and 4-bromo-1-butanol was replaced by 2-(2-bromoethoxy)ethanol in step 1 and oxetan-3-amine replaced ethylamine in step 3. ¹H NMR (400 MHz, DMSO-d₆) δ 9.15 (s, 1H), 8.71 (s, 1H), 8.69-8.64 (m, 1H), 8.57 (s, 1H), 8.27 (d, J=1.7 Hz, 1H), 8.15 (dd, J=8.0, 1.8 Hz, 1H), 7.87 (s, 1H), 7.62 (d, J=7.9 Hz, 0H), 7.55 (d, J=8.0 Hz, 1H), 5.14-4.90 (m, 1H), 4.90-4.74 (m, 3H), 4.65-4.57 (m, 2H), 4.54 (t, J=5.1 Hz, 1H), 4.51-4.44 (m, 2H), 3.68 (t, J=6.6 Hz, 2H), 3.54-3.42 (m, 4H), 2.60 (s, 3H); MS (ESI) m/z: 509.2 [M+1]⁺.

### Example 79

### 6-(2-Chloro-4-(6-methylpyrazin-2-yl)phenyl)-8-(3,4-dihydroxybutyl)-2-(methylamino)pyrido[2,3-d]pyrimidin-7(8H)-one (I-80)

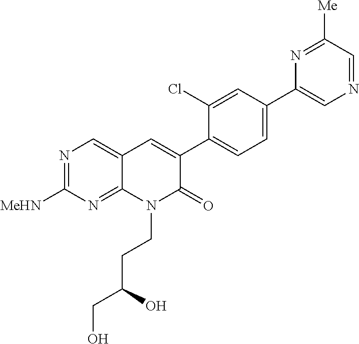 [SMILES: CNc1ncc2cc(-c3ccc(-c4cncc(C)n4)cc3Cl)c(=O)n(CC[C@@H](O)CO)c2n1]

step 1: To a suspension of NaH (60% dispersion in oil, 304 mg, 5.06 m mol) in anhydrous DMF (15 mL) was added 6-[2-chloro-4-(6-methylpyrazin-2-yl)phenyl]-2-methylsulfanyl-8H-pyrido[2,3-d]pyrimidin-7-one (2.004 g, 5.061 mmol) at RT and the reaction was stirred at 50° C. for 0.5 h. The reaction mixture was cooled down to RT and a solution of 4-bromobut-1-ene (1.59 mL, 15.18 mmol) in DMF (5 mL) was added and stirred at 50° C. for 16 h. The reaction mixture was cooled to RT and quenched with water and diluted with EtOAc. The layers were separated and the aqueous phase was extracted into EtOAc (3×). The combined organics were dried (Na₂SO₄), filtered, and absorbed onto CELITE. The crude residue was purified SiO₂ column chromatography to afford 8-(but-3-en-1-yl)-6-(2-chloro-4-(6-methylpyrazin-2-yl)phenyl)-2-(methylthio)pyrido[2,3-d]pyrimidin-7(8H)-one as a yellow solid (1.751 g, 77%). ¹H NMR (400 MHz, CDCl₃) δ 8.84 (s, 1H), 8.67 (s, 1H), 8.44 (s, 1H), 8.19 (d, J=1.6 Hz, 1H), 7.97 (dd, J=8.0, 1.7 Hz, 1H), 7.69 (s, 1H), 7.51 (d, J=8.0 Hz, 1H), 5.97-5.80 (m, 1H), 5.16-4.98 (m, 2H), 4.64-4.53 (m, 2H), 2.66 (d, J=2.8 Hz, 6H), 2.57 (q, J=7.2 Hz, 2H).

step 2: 8-(But-3-en-1-yl)-6-(2-chloro-4-(6-methylpyrazin-2-yl)phenyl)-2-(methylthio)pyrido[2,3-d]pyrimidin-7(8H)-one (1.316 g, 2.924 mmol) was dissolved in THF (2.13 L/mol, 6.23 mL) and tert-butanol (0.93 mL, 9.797 mmol). N-methyl morpholine-N-oxide (2.00 equiv., 706.4 mg, 5.849 mmol) was added followed by potassium osmate(VI) dihydrate (0.02 equiv., 19.44 mg, 0.05849 mmol). The reaction mixture was stirred at RT for 5 h. Water (6 mL) and additional potassium osmate(VI) dihydrate (20 mg, 0.058 mmol) were added. The reaction mixture was allowed to stir overnight at RT. After 16 h the reaction mixture was quenched with a saturated aqueous solution of Na₂S₂O₃. The solid was collected by filtration and was washed with water and then air dried to yield 6-(2-chloro-4-(6-methylpyrazin-2-yl)phenyl)-8-(3,4-dihydroxybutyl)-2-(methylthio)pyrido[2,3-d]pyrimidin-7(8H)-one as a tan solid (1.304 g, 92%). ¹H NMR (400 MHz, DMSO-d₆) δ 9.16 (s, 1H), 8.94 (s, 1H), 8.58 (s, 1H), 8.30 (s, 1H), 8.18 (d, J=8.2 Hz, 1H), 8.06 (s, 1H), 7.60 (d, J=8.1 Hz, 1H), 4.56 (s, 2H), 4.44 (d, J=27.3 Hz, 2H), 3.57 (s, 1H), 3.41-3.32 (m, 1H), 2.65 (s, 3H), 2.60 (s, 3H), 1.88 (s, 1H), 1.67 (s, 1H).

The product of step 2 was converted to the final product using the synthetic sequence described in steps 2 and 3 in Example 63. ¹H NMR (400 MHz, DMSO-d₆) δ 9.15 (s, 1H), 8.64 (s, 1H), 8.57 (s, 1H), 8.27 (d, J=1.8 Hz, 1H), 8.15 (dd, J=8.0, 1.8 Hz, 1H), 7.84 (s, 1H), 7.56 (d, J=8.0 Hz, 1H), 4.58-4.44 (m, 3H), 4.35 (s, 1H), 3.55 (s, 1H), 2.93 (d, J=4.6 Hz, 3H), 2.60 (s, 3H), 1.86 (s, 1H), 1.64 (s, 1H); MS (ESI) m/z: 467.16 [M+1]⁺.

### Example 80

### (R)-6-(2-Chloro-4-(6-methylpyrazin-2-yl)phenyl)-8-(3,4-dihydroxybutyl)-2-(ethylamino)pyrido[2,3-d]pyrimidin-7(8H)-one (I-79)

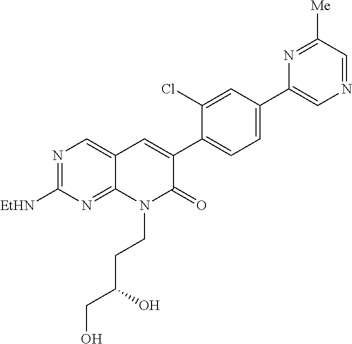

step 1: DIAD (461 mg, 2.28 mmol) was added drop wise to a mixture of 6-(2-chloro-4-(6-methylpyrazin-2-yl)phenyl)-2-(methylthio)pyrido[2,3-d]pyrimidin-7(8H)-one (300 mg, 0.76 mmol), PPh₃ (597 mg, 2.28 mmol) and (S)-2-(2,2-dimethyl-1,3-dioxolan-4-yl)ethanol (333 mg, 2.28 mmol) in dry DMF (30 mL) at 0° C. The mixture was stirred under nitrogen for 18 h, and then concentrated in vacuo. The crude product was purified by SiO₂ chromatography eluting with DCM/EtOAc (8:1) to afford (S)-6-(2-chloro-4-(6-methylpyrazin-2-yl)phenyl)-8-(2-(2,2-dimethyl-1,3-dioxolan-4-yl)ethyl)-2-(methylthio)pyrido[2,3-d]pyrimidin-7(8H)-one as a yellow solid (268 mg, 67.5%). LCMS (ESI): m/z=523.9 [M+1]⁺.

step 2: A mixture of (S)-6-(2-chloro-4-(6-methylpyrazin-2-yl)phenyl)-8-(2-(2,2-dimethyl-1,3-dioxolan-4-yl)ethyl)-2-(methylthio)pyrido[2,3-d]pyrimidin-7(8H)-one (268 mg, 0.51 mmol) and MCPBA (80%, 122 mg, 0.56 mmol) in DCM (20 mL) was stirred at RT for 4 h. The mixture was then poured into 10% NaHCO₃ (150 mL) and extracted with DCM (3×50 mL). The combined organics were concentrated in vacuo to afford crude 6-(2-chloro-4-(6-methylpyrazin-2-yl)phenyl)-8-(2-((S)-2,2-dimethyl-1,3-dioxolan-4-yl)ethyl)-2-(methylsulfinyl)pyrido[2,3-d]pyrimidin-7(8H)-one as a yellow solid (272 mg), which was used in the next step without purification. LCMS (ESI): m/z=539.8 [M+1]⁺.

step 3: A mixture of crude 6-(2-chloro-4-(6-methylpyrazin-2-yl)phenyl)-8-(2-((S)-2,2-dimethyl-1,3-dioxolan-4-yl)ethyl)-2-(methylsulfinyl)pyrido[2,3-d]pyrimidin-7(8H)-one (272 mg, 0.49 mmol), ethanamine hydrochloride (80 mg, 0.98 mmol) and DIPEA (253 mg, 1.96 mmol) in IPA (10 mL) was stirred at 50° C. for 18 h. The reaction mixture was concentrated and purified by SiO₂ chromatography using DCM/EtOAc (1:1) as eluting solvents to afford (S)-6-(2-chloro-4-(6-methylpyrazin-2-yl)phenyl)-8-(2-(2,2-dimethyl-1,3-dioxolan-4-yl)ethyl)-2-(ethylamino)pyrido[2,3-d]pyrimidin-7(8H)-one as a yellow solid (213 mg, 83.6%). LCMS (ESI): m/z=521.0 [M+1]⁺.

step 4: A mixture of (S)-6-(2-chloro-4-(6-methylpyrazin-2-yl)phenyl)-8-(2-(2,2-dimethyl-1,3-dioxolan-4-yl)ethyl)-2-(ethylamino)pyrido[2,3-d]pyrimidin-7(8H)-one (190 mg, 0.365 mmol) in HOAc (20 mL) was stirred at 60° C. for 18 h. The reaction mixture was then concentrated in vacuo and purified by preparative HPLC to give the target compound as a white solid (49 mg, 27.9%). ¹H NMR (500 MHz, MeOH-d₄) δ 8.97 (s, 1H), 8.61 (brs, 1H), 8.49 (s, 1H), 8.27 (d, 1H), 8.09 (dd, 1H), 7.80 (s, 1H), 7.55 (d, 1H), 4.69-4.64 (m, 1H), 4.56-4.54 (m, 1H), 3.75-3.73 (m, 1H), 3.59-3.52 (m, 4H), 2.67 (s, 3H), 2.02 (brs, 1H), 1.86 (brs, 1H), 1.31 (brs, 3H). LCMS (ESI) m/z: 481.0 [M+1]⁺.

(S)-6-(2-Chloro-4-(6-methylpyrazin-2-yl)phenyl)-8-(3,4-dihydroxybutyl)-2-(ethylamino)pyrido[2,3-d]pyrimidin-7(8H)-one (I-83) was prepared analogously except in step 1 using (R)-2-(2,2-dimethyl-1,3-dioxolan-4-yl)ethanol replaced (S)-2-(2,2-dimethyl-1,3-dioxolan-4-yl)ethanol in step 1. ¹H NMR (500 MHz, MeOH-d₄) δ 8.97 (s, 1H), 8.61 (brs, 1H), 8.49 (s, 1H), 8.26 (d, 1H), 8.08 (dd, 1H), 7.80 (s, 1H), 7.55 (d, 1H), 4.69-4.66 (m, 1H), 4.54 (brs, 1H), 3.74 (brs, 1H), 3.60-3.52 (m, 4H), 2.67 (s, 3H), 2.03-2.00 (m, 1H), 1.87-1.85 (m, 1H), 1.31-1.30 (m, 3H); LCMS (ESI): m/z=481.0 [M+1]⁺.

6-[2-Chloro-4-(6-methyl-2-pyridyl)phenyl]-8-[(3S)-3,4-dihydroxybutyl]-2-(ethylamino)pyrido[2,3-d]pyrimidin-7-one (I-123) was prepared analogously except 6-[2-chloro-4-(6-methylpyrazin-2-yl)phenyl]-2-methylsulfanyl-8H-pyrido[2,3-d]pyrimidin-7-one was replaced by 6-[2-chlorol-4-(6-methyl-2-pyridyl)phenyl]-2-methylsulfanyl-8H-pyrido[2,3-d]pyrimidin-7-one in step 1. ¹H NMR (500 MHz, MeOH-d₄) δ 8.61 (brs, 1H), 8.14 (d, J=2.0 Hz, 1H), 7.96 (dd, J1=2.0, 8.0 Hz, 1H), 7.81-7.79 (m, 2H), 7.71 (m, 1H), 7.51 (dd, J=3.0, 7.5 Hz, 1H), 7.28 (d, J=7.5 Hz, 1H), 4.69-4.66 (m, 1H), 4.55-4.54 (m, 1H), 3.74 (brs, 1H), 3.59-3.54 (m, 4H), 2.63 (s, 3H), 2.02 (brs, 1H), 1.86 (brs, 1H), 1.32-1.30 (m, 3H); LCMS (ESI): m/z=479.9 [M+1]⁺.

### Example 81

### 8-(2-(3-Aminocyclobutoxy)ethyl)-6-(2-chloro-4-(6-methylpyrazin-2-yl)phenyl)-2-(ethylamino)-pyrido[2,3-d]pyrimidin-7(8H)-one (I-117)

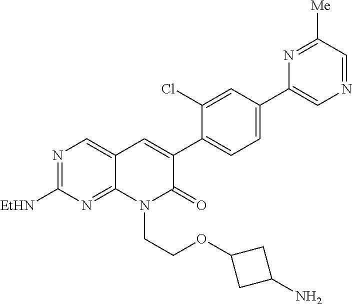

The title compound was prepared by alkylation of 6-[2-chloro-4-(6-methylpyrazin-2-yl)phenyl]-2-methylsulfanyl-8H-pyrido[2,3-d]pyrimidin-7-one with 2-(3-(2-hydroxyethoxy)cyclobutyl)isoindoline-1,3-dione (DIAD, PPh₃). The intermediate 2-(3-(2-(6-(2-chloro-4-(6-methylpyrazin-2-yl)phenyl)-2-(methylthio)-7-oxopyrido[2,3-d]pyrimidin-(7H)-yl)ethoxy)cyclobutyl)isoindoline-1,3-dione was prepared in accord with the procedure in Example 80 but (S)-2-(2,2-dimethyl-1,3-dioxolan-4-yl)ethanol was replaced with 2-(3-aminocyclobutoxy)ethanol. Introduction of the ethylamine was carried out using standard protocols. Final deprotection was accomplished by heating a mixture of 2-(3-(2-(6-(2-chloro-4-(6-methylpyrazin-2-yl)phenyl)-2-(ethylamino)-7-oxopyrido[2,3-d]pyrimidin-8(7H)-yl)ethoxy)cyclobutyl)isoindoline-1,3-dione (290 mg, 0.457 mmol) and methylamine (20 mL, 25% solution in MeOH,) at 80° C. for 3 h. The reaction mixture was concentrated and purified by prep-HPLC to afford 8-(2-(3-aminocyclobutoxy)ethyl)-6-(2-chloro-4-(6-methylpyrazin-2-yl)phenyl)-2-(ethylamino)-pyrido[2,3-d]pyrimidin-7(8H)-one as white solid (153 mg, 66.2%). ¹H NMR (500 MHz, MeOD-d4) δ 8.98 (s, 1H), 8.60 (brs, 1H), 8.50 (s, 1H), 8.27 (d, J=1.5, 1H), 8.09 (dd, J=1.5, 8.0, 1H), 7.80 (s, 1H), 7.55 (d, J=7.5, 1H), 4.66 (brs, 2H), 3.80-3.77 (m, 1H), 3.74 (brs, 2H), 3.57-3.53 (m, 2H), 2.99-2.95 (m, 1H), 2.67 (s, 3H), 2.63-2.58 (m, 2H), 1.70-1.67 (m, 2H), 1.32 (brs, 3H). ESIMS: m/z=506.0 [M+1].

### Example 82

### 8-(2-((1R,5S,6s)-3-Azabicyclo[3.1.0]hexan-6-yl)ethyl)-6-(2-chloro-4-(6-methylpyrazin-2-yl)phenyl)-2-(ethylamino)pyrido[2,3-d]pyrimidin-7(8H)-one (I-65)

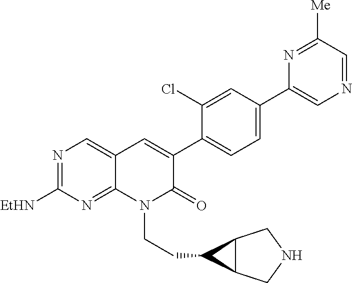

6-(2-chloro-4-(6-methylpyrazin-2-yl)phenyl)-2-(methylthio)pyrido[2,3-d]pyrimidin-7(8H)-one was alkylated with (1R,5S,6s)-tert-butyl 6-(2-hydroxyethyl)-3-azabicyclo[3.1.0]hexane-3-carboxylate (DIAD, PPh₃). Introduction of the ethylamine and deprotection of the amine were carried out by standard protocols. ¹H NMR (400 MHz, DMSO-d₆) δ 9.15 (s, 1H), 8.65 (s, 1H), 8.57 (s, 1H), 8.27 (s, 1H), 8.14 (d, J=8.1 Hz, 1H), 7.94 (s, 1H), 7.82 (s, 1H), 7.54 (d, J=8.0 Hz, 1H), 4.37 (m, 2H), 3.47-3.37 (m, 2H), 2.69 (d, J=10.4 Hz, 2H), 2.60 (s, 3H), 2.54 (m, 2H), 1.62 (m, 2H), 1.21 (m, 3H), 1.06 (m, 2H), 0.64 (m, 1H); MS (ESI) m/z: 502.2 [M+1]⁺.

### Example 83

### 8-(((1R,4R)-4-Aminocyclohexyl)methyl)-6-(2-chloro-4-(5-methylpyridazin-3-yl)phenyl)-2-(ethylamino)pyrido[2,3-d]pyrimidin-7(8H)-one (I-77)

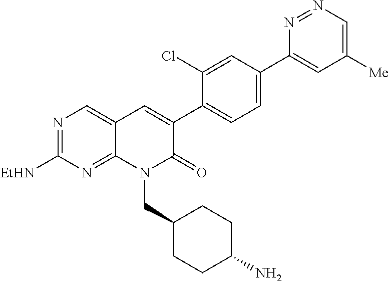

### Methyl 2-(2-chloro-4-(5-methylpyridazin-3-yl)phenyl)acetate

A mixture of methyl 2-(2-chloro-4-(4,4,5,5-tetramethyl-1,3,2-dioxaborolan-2-yl)phenyl)acetate (682 mg, 2.2 mmol), 3-chloro-5-methylpyridazine (297 mg in 1.188 g dioxane, 2.3 mmol), KOAc (431 mg, 4.4 mmol), Pd (dppf)Cl₂ (160 mg, 0.22 mmol) in dioxane (22 mL) and water (16 mL) was heated at 90° C. under N₂ overnight. The reaction mixture was then diluted with EtOAc (100 mL) and washed with water (50 mL). The aqueous layer was extracted with EtOAc (3×30 mL). The combined organic layers were washed with brine (2×50 mL), dried (MgSO₄), filtered and concentrated in vacuo. The residue was then purified by SiO₂ chromatography eluting with PE/EtOAc (55-60%) to afford the target compound (186 mg, 30%) as a yellow solid. LCMS (ESI): m/z=277.0 [M+1]⁺.

A mixture of methyl 2-(2-chloro-4-(5-methylpyridazin-3-yl)phenyl)acetate (186 mg, 0.67 mmol), tert-butyl (1R,4R)-4-((5-formyl-2-(methylthio)pyrimidin-4-ylamino)methyl)cyclohexylcarbamate (200 mg, 0.52 mmol), K₂CO₃ (216 mg, 1.56 mmol) and DMF (6 mL) was stirred overnight at 100° C. under N₂. The reaction mixture was then poured into cold ammonium chloride solution and extracted with EtOAc (3×30 mL). The combined organic layers were washed with brine (4×30 mL), dried (MgSO₄), filtered and concentrated in vacuo. The residue was purified by SiO₂ column chromatography eluting with PE/EtOAc (60%) to afford tert-butyl (1R,4R)-4-((6-(2-chloro-4-(5-methylpyridazin-3-yl)phenyl)-2-(methylthio)-7-oxopyrido[2,3-d]pyrimidin-8(7H)-yl)methyl)cyclohexylcarbamate (202 mg, 49%) as a yellow solid. LCMS (ESI): m/z=550.8 [M−56]⁺.

Introduction of the ethyl amine and removal of the Boc group were accomplished by standard protocols. ¹H NMR (500 MHz, DMSO-d₆): δ 9.14 (s, 1H), 8.66 (d, 1H), 8.29 (d, 1H), 8.22 (s, 1H), 8.18-8.16 (m, 1H), 8.00-7.98 (m, 1H), 7.84 (s, 1H), 7.59-7.57 (m, 1H), 4.18 (d, 2H), 3.40-3.38 (m, 2H), 2.47-2.45 (m, 1H), 2.42 (s, 3H), 1.85 (brs, 1H), 1.74 (d, 2H), 1.62 (d, 2H), 1.23-1.86 (m, 3H), 1.15-1.07 (m, 2H), 0.94-0.86 (m, 2H); LCMS (ESI): m/z=504.1 [M+1]⁺.

### Example 84

### 8-((1R,5S,6R)-3-Azabicyclo[3.1.0]hexan-6-ylmethyl)-6-(2-chloro-4-(6-methylpyrazin-2-yl)phenyl)-2-(ethylamino)pyrido[2,3-d]pyrimidin-7(8H)-one (I-82)

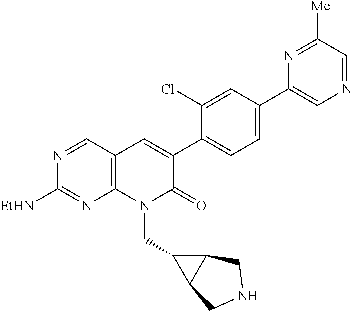

step 1: DIAD (817 mg, 4.05 mmol) was added dropwise to a mixture of 6-bromo-2-(methylthio)pyrido[2,3-d]pyrimidin-7(8H)-one (250 mg, 0.92 mmol), (1R,5S,6R)-tert-butyl 6-(hydroxymethyl)-3-azabicyclo[3.1.0]hexane-3-carboxylate (431 mg, 2.02 mmol) and PPh₃ (1058 mg, 4.04 mmol) in DMF (4 mL) while maintaining the temperature between 0-5° C. The mixture was stirred at RT overnight, then poured onto ice water and extracted with EtOAc (3×100 mL). The combined organic layers were washed with saturated brine (4×100 mL), dried (MgSO₄), filtered and concentrated. The residue was purified by SiO₂ chromatography eluting with a EtOAc/PE gradient (1:10 to 1:3) to afford (1R,5S,6S)-tert-butyl 6-((6-bromo-2-(methylthio)-7-oxopyrido[2,3-d]pyrimidin-8(7H)-yl)methyl)-3-azabicyclo[3.1.0]hexane-3-carboxylate (138 mg, 32%) as a white solid. LCMS (ESI): m/z=410.9 [M−55]⁺.

step 2: 2-Chloro-4-(6-methylpyrazin-2-yl)phenylboronic acid (77 mg, 0.27 mmol), Cs₂CO₃ (132 mg, 0.405 mmol), and Pd(PPh₃)₄ (16 mg, 0.014 mmol) was added to a solution of (1R,5S,6S)-tert-butyl 6-((6-bromo-2-(methylthio)-7-oxopyrido[2,3-d]pyrimidin-8(7H)-yl)methyl)-3-azabicyclo[3.1.0]hexane-3-carboxylate (130 mg, 0.27 mmol) in a mixture of ethylene glycol dimethyl ether (3 mL), EtOH (2 mL), PhMe (0.3 mL) and water (0.6 mL). The mixture was heated at 85° C. overnight, concentrated in vacuo and dissolved in DCM (50 mL). The mixture was then washed with brine (2×30 mL), dried (MgSO₄), filtered, concentrated in vacuo and purified by SiO₂ column chromatography eluting with MeOH:DCM (1:40) to afford (1R,5S,6S)-tert-butyl 6-((6-(2-chloro-4-(6-methylpyrazin-2-yl)phenyl)-2-(methylthio)-7-oxopyrido[2,3-d]pyrimidin-8(7H)-yl)methyl)-3-azabicyclo[3.1.0]hexane-3-carboxylate (55 mg, 32%) as a pale yellow oil. LCMS (ESI): m/z=591.1 [M+1]⁺.

Introduction of the ethyl amine and removal of the Boc group were accomplished by standard protocols. ¹H NMR (500 MHz, DMSO-d₆): δ 9.16 (s, 1H), 8.65 (s, 1H), 8.57 (s, 1H), 8.27 (s, 1H), 8.16-8.14 (m, 1H), 7.99 (s, 1H), 7.84 (s, 1H), 7.57 (d, 1H), 4.25 (d, 2H), 3.44-3.37 (m, 2H), 2.81 (d, 2H), 2.67 (d, 2H), 2.60 (s, 3H), 1.57 (s, 2H), 1.26-1.15 (m, 4H); MS (ESI): m/z=488.0 [M+1]⁺.

### Example 85

### 6-(2-Chloro-4-(6-methylpyridin-2-yl)phenyl)-2-(ethylamino)-8-(piperidin-4-ylmethyl)pyrido[2,3-d]pyrimidin-7(8H)-one (I-44)

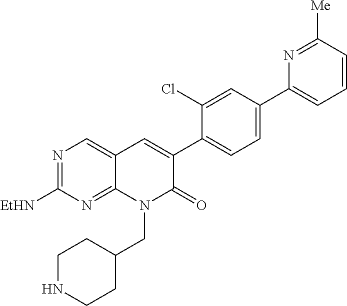

step 1: DIAD (800 mg, 3.9 mmol) was added dropwise to a 0° C. solution of 6-(4-bromo-2-chlorophenyl)-2-(methylthio)pyrido[2,3-d]pyrimidin-7(8H)-one (689 mg, 1.8 mmol), tert-butyl 4-(hydroxymethyl)piperidine-1-carboxylate (852 mg, 3.9 mmol) and PPh₃ (1038 mg, 3.9 mmol) in DMF (4 mL). The temperature was maintained between 0-5° C. during addition, and the mixture was then stirred overnight at RT. The mixture was poured into ice water and extracted with EtOAc (3×80 mL). The combined organic layers were washed with brine (4×50 mL), dried (MgSO₄), filtered and concentrated. The residue obtained was purified by SiO₂ chromatography, eluting with an EtOAc/PE gradient (1:20 to 1:10), to afford tert-butyl 4-((6-(4-bromo-2-chlorophenyl)-2-(methylthio)-7-oxopyrido[2,3-d]pyrimidin-8(7H)-yl)methyl)piperidine-1-carboxylate (622 mg, 59%) as a white solid. LCMS (ESI): m/z=579.1 [M+1]⁺.

step 2: A mixture of tert-butyl 4-((6-(4-bromo-2-chlorophenyl)-2-(methylthio)-7-oxopyrido[2,3-d]pyrimidin-8(7H)-yl)methyl)piperidine-1-carboxylate (620 mg, 1.07 mmol), 4,4,4′,4′,5,5,5′,5′-octamethyl-2,2′-bi(1,3,2-dioxaborolane) (543 mg, 2.14 mmol), potassium acetate (420 mg, 4.28 mmol) and Pd(II)(dppf)Cl₂ (87 mg, 0.12 mol) in dioxane (13 mL) was stirred at 80° C. overnight while under N₂. The mixture was then cooled to RT, filtered through CELITE, and concentrated to afford tert-butyl 4-((6-(2-chloro-4-(4,4,5,5-tetramethyl-1,3,2-dioxaborolan-2-yl)phenyl)-2-(methylthio)-7-oxopyrido[2,3-d]pyrimidin-8(7H)-yl)methyl)piperidine-1-carboxylate (1.254 g) as brown solid which was used directly in the next step without further purification. LCMS (ESI): m/z=627.3 [M+1]⁺.

step 3: tert-Butyl 4-((6-(2-chloro-4-(4,4,5,5-tetramethyl-1,3,2-dioxaborolan-2-yl)phenyl)-2-(methylthio)-7-oxopyrido[2,3-d]pyrimidin-8(7H)-yl)methyl)piperidine-1-carboxylate (1.25 g, 2 mmol), Cs₂CO₃ (953 mg, 2.92 mmol), and Pd(PPh₃)₄ (113 mg, 0.098 mmol) were added to a solution of 2-bromo-6-methylpyridine (341 mg, 1.98 mmol) in ethylene glycol dimethyl ether (11 mL), EtOH (8 mL), PhMe (1.1 mL) and water (2.1 mL). The mixture was stirred overnight at 80° C., then cooled, diluted with EtOAc (50 mL), and washed with brine (30 mL). The aqueous layer was extracted with EtOAc (2×50 mL). The organic layers were combined, washed with brine (50 mL), dried (MgSO₄), filtered and concentrated. The residue was purified by SiO₂ column chromatography eluting with EtOAc/PE gradient (1:5 to 1:3) to give tert-butyl 4-((6-(2-chloro-4-(6-methylpyridin-2-yl)phenyl)-2-(methylthio)-7-oxopyrido[2,3-d]pyrimidin-8(7H)-yl)methyl)piperidine-1-carboxylate (314 mg, 49% of two steps) as a yellow solid. LCMS (ESI): m/z=492.1 [M−100]⁺.

Introduction of the ethyl amine and removal of the Boc group were accomplished by standard protocols. ¹H NMR (500 MHz, DMSO-d₆): δ 8.66 (s, 1H), 8.22 (s, 1H), 8.21-8.07 (m, 1H), 7.97 (d, 1H), 7.86 (d, 1H), 7.82-7.76 (m, 2H), 7.50-7.49 (d, 1H), 7.28 (d, 1H), 4.20 (d, 2H), 3.41-3.37 (m, 2H), 2.90 (d, 2H), 2.52 (s, 3H), 2.36-2.32 (m, 2H), 2.00-1.98 (m, 1H), 1.49 (d, 2H), 1.23-1.17 (m, 5H). LCMS (ESI): m/z=489.1 [M+1]⁺.

### Example 86

### 6-[2-Chloro-4-(6-methylpyrazin-2-yl)phenyl]-2-(ethylamino)-8-[2-(2-oxopiperazin-1-yl)ethyl]pyrido[2,3-d]pyrimidin-7-one (I-90)

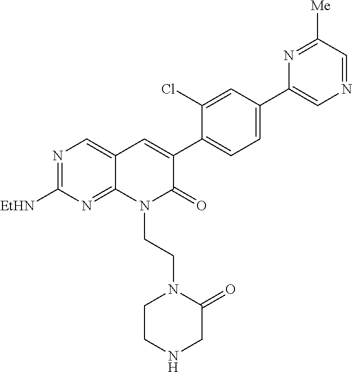

step 1: To a mixture of NaH (430 mg, 1.8 mmol) in DMF (5 mL) was added tert-butyl 3-oxopiperazine-1-carboxylate (3.0 g, 1.5 mmol) and 2-bromoacetonitrile (1.8 g, 1.5 mmol) at 0° C. The mixture was stirred at RT overnight. The reaction mixture was then diluted with EtOAc (10 mL) and H₂O (10 mL). The separated organic layer was washed with brine, dried (MgSO₄), filtered and concentrated under reduced pressure. The residue was purified by SiO₂ chromatography eluting with DCM/MeOH (40:1) to afford tert-butyl 4-(cyanomethyl)-3-oxopiperazine-1-carboxylate as yellow oil (3.0 g, 76%). LCMS (ESI): m/z=240.1 [M+1]⁺

step 2: A mixture of tert-butyl 4-(cyanomethyl)-3-oxopiperazine-1-carboxylate (3.0 g, 12.5 mmol), PtO₂ (300 mg, 1.32 mmol) in EtOH (20 mL) was stirred at 20° C. under H₂ (50 psi) overnight. The Pd/C catalyst was removed by filtration, and the filtrate was concentrated under reduced pressure to afford tert-butyl 4-(2-aminoethyl)-3-oxopiperazine-1-carboxylate (2.8 g, 93%) as yellow solid. LCMS (ESI): m/z=244.2 [M+1]⁺.

tert-Butyl 4-(2-((2-(ethylamino)-5-formylpyrimidin-4-yl)amino)ethyl)-3-oxopiperazine-1-carboxylate was prepared condensing ethyl 4-chloro-2-(methylthio)pyrimidine-5-carboxylate and tert-butyl 4-(2-aminoethyl)-3-oxopiperazine-1-carboxylate. Reduction of the ester and MnO₂ oxidation of the resulting alcohol was carried out using standard protocols to afford tert-butyl 4-(2-((2-(ethylamino)-5-formylpyrimidin-4-yl)amino)ethyl)-3-oxopiperazine-1-carboxylate. The product was converted to I-90 condensation and cyclization of methyl 4-bromo-2-chlorophenyl acetate and palladium-catalyzed coupling of [2-chloro-4-(6-methylpyrazin-2-yl)phenyl]boronic acid. ¹H NMR (500 MHz, MeOH-d₄): δ 8.96 (s, 1H), 8.59 (brs, 1H), 8.48 (s, 1H), 8.26 (s, 1H), 8.08 (d, J=8.0 Hz, 1H), 7.76 (s, 1H), 7.54 (d, J=7.5 Hz, 1H), 4.71 (m, 2H), 3.87 (m, 2H), 3.56 (m, 2H), 3.46 (t, J=5.0 Hz 2H), 3.22 (m, 2H), 2.91 (t, J=5.0 Hz, 2H), 2.65 (s, 3H), 1.29 (t, J=5.0 Hz, 3H); MS (ESI) m/z: 519.0 [M+1]⁺.

### Example 87

### 2-Amino-8-[((2s,5s)-5-amino-1,3-dioxan-2-yl)methyl]-6-[2-chloro-4-(6-methylpyrazin-2-yl)phenyl]pyrido[2,3-d]pyrimidin-7-one and 2-amino-8-[((2r,5r)-5-amino-1,3-dioxan-2-yl)methyl]-6-[2-chloro-4-(6-methylpyrazin-2-yl)phenyl]pyrido[2,3-d]pyrimidin-7-one (I-56)

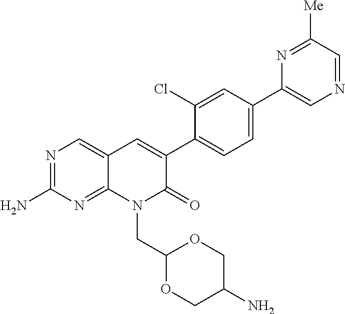 [SMILES: Cc1cncc(-c2ccc(-c3cc4cnc(N)nc4n(CC4OCC(N)CO4)c3=O)c(Cl)c2)n1]

step 1: To a suspension of NaH (60% dispersion in mineral oil, 300 mg, 7.6 mmol) in anhydrous DMF (15 mL) was added 6-[2-chloro-4-(6-methylpyrazin-2-yl)phenyl]-2-methylsulfanyl-8H-pyrido[2,3-d]pyrimidin-7-one (2.0 g, 5.1 mmol) at RT and the reaction was stirred at 50° C. for 0.5 h. The reaction mixture was cooled to RT, and a solution of 2-bromoethoxy-tert-butyl-dimethyl-silane (3.6 g, 3 equiv., 15 mmol) in DMF (1 mL) was added. The reaction was warmed once more to 50° C. and stirred for 16 h. After this period, the mixture was cooled to RT, quenched with water, and diluted with EtOAc (100 mL) and water (100 mL). The layers were separated and the aqueous phase was extracted with EtOAc (3×50 mL). The combined organic portions were dried (MgSO₄), filtered, absorbed onto CELITE, and purified by SiO₂ chromatography (40 g Gold ISCO column) eluting with an EtOAc/heptane gradient (20 to 80% EtOAc). The desired products were collected and the following products were obtained: 8-[2-[tert-butyl(dimethyl)silyl]oxyethyl]-6-[2-chloro-4-(6-methylpyrazin-2-yl)phenyl]-2-methylsulfanyl-pyrido[2,3-d]pyrimidin-7-one (323 mg, 15% yield); LCMS (ESI): m/z=554.2 (M⁺) and 6-[2-chloro-4-(6-methylpyrazin-2-yl)phenyl]-8-(2-hydroxyethyl)-2-methylsulfanyl-pyrido[2,3-d]pyrimidin-7-one (820 mg, 37% yield); LCMS (ESI): m/z=440.1 (M+H⁺).

step 2: Oxalyl chloride (0.24 mL, 1.5 equiv., 2.80 mmol) was dissolved in anhydrous DCM (0.44 M, 6.3 mL) and cooled to −78° C. DMSO (3.0 equiv., 5.59 mmol, 0.40 mL) was then added dropwise. The resulting mixture was stirred for an additional 0.5 h before 6-[2-chloro-4-(6-methylpyrazin-2-yl)phenyl]-8-(2-hydroxyethyl)-2-methylsulfanyl-pyrido[2,3-d]pyrimidin-7-one (820 mg, 1.864 mmol) in 5 mL DCM was added dropwise. The reaction mixture was stirred for an additional 1 h at −78° C. TEA (5.0 equiv., 9.32 mmol, 1.30 mL) was then added drop-wise followed by 2 mL DCM. The mixture was stirred 30 min, then warmed to 0° C. and washed with water, 0.5 M KHSO₄, and brine. Subsequent drying (MgSO₄) and concentration in vacuo afforded 2-[6-[2-chloro-4-(6-methylpyrazin-2-yl)phenyl]-2-methylsulfanyl-7-oxo-pyrido[2,3-d]pyrimidin-8-yl]acetaldehyde as a light yellow solid (648 mg, 79% yield).

step 3: 2-[6-[2-Chloro-4-(6-methylpyrazin-2-yl)phenyl]-2-methylsulfanyl-7-oxo-pyrido[2,3-d]pyrimidin-8-yl]acetaldehyde (648 mg, 1.480 mmol) was taken up in dry CHCl₃ (20 mL) and PhMe (5 mL) and tert-butyl N-[2-hydroxy-1-(hydroxymethyl)ethyl]carbamate (10.0 equiv., 14.80 mmol, 2.83 g) was added, followed by anhydrous sodium sulfate (10 equiv., 2.10 g) and pTsOH monohydrate (0.1 equiv., 0.148 mmol, 25.7 mg). The resulting reaction mixture was stirred at RT for 44 h and only 25% of product was formed by that time. Additional p-TsOH monohydrate (0.1 equiv., 0.148 mmol, 25.7 mg) was added followed by stirring for 96 h. No appreciable change was noted and the reaction was quenched with sat. NaHCO₃ and diluted further with DCM. The phases were separated and the aqueous layer was extracted with DCM (2×). The combined extracts were dried (MgSO₄), filtered and concentrated in vacuo. The crude product was purified by SiO₂ chromatography (40 g Gold column) eluting with a MeOH/DCM gradient (0-10% MeOH) to afford ca. 40 mg of tert-butyl N-[2-[[6-[2-chloro-4-(6-methylpyrazin-2-yl)phenyl]-2-methylsulfanyl-7-oxo-pyrido[2,3-d]pyrimidin-8-yl]methyl]-1,3-dioxan-5-yl]carbamate which was used in the next step. LCMS (ESI): m/z=611.2 (M⁺).

step 4: tert-Butyl N-[2-[[6-[2-chloro-4-(6-methylpyrazin-2-yl)phenyl]-2-methylsulfanyl-7-oxo-pyrido[2,3-d]pyrimidin-8-yl]methyl]-1,3-dioxan-5-yl]carbamate (ca. 400 mg, 0.655 mmol) was dissolved in DCM (5 mL), and the mixture was cooled to 0° C. MCPBA (1.1 equiv., 0.720 mmol, 161.4 mg) was then added followed by stirring for 1 h. The reaction was quenched with saturated aqueous NaHCO₃ solution and diluted with DCM. The mixture was extracted with DCM (2×) and the combined organic layers washed with brine solution (1×), dried (MgSO₄), filtered and concentrated in vacuo. to afford tert-butyl N-[2-[[6-[2-chloro-4-(6-methylpyrazin-2-yl)phenyl]-2-methylsulfinyl-7-oxo-pyrido[2,3-d]pyrimidin-8-yl]methyl]-1,3-dioxan-5-yl]carbamate (163 mg, 39% yield) as a crude yellow residue that was used in the next step without further purification.

step 5: In a 6-dram scintillation vial was placed tert-butyl N-[2-[[6-[2-chloro-4-(6-methylpyrazin-2-yl)phenyl]-2-methylsulfinyl-7-oxo-pyrido[2,3-d]pyrimidin-8-yl]methyl]-1,3-dioxan-5-yl]carbamate (163 mg, 0.26 mmol) suspended in 1.5 mL of THF. A solution of ammonia in MeOH (7 N, 5 equiv., 1.30 mmol, 0.19 mL) was then added. The reaction mixture was stirred at RT for a 16 h period. The solution was concentrated in vacuo to afford tert-butyl N-[2-[[2-amino-6-[2-chloro-4-(6-methylpyrazin-2-yl)phenyl]-7-oxo-pyrido[2,3-d]pyrimidin-8-yl]methyl]-1,3-dioxan-5-yl]carbamate which was used in the next step without further purification.

step 6. The dried product mixture containing tert-butyl N-[2-[[2-amino-6-[2-chloro-4-(6-methylpyrazin-2-yl)phenyl]-7-oxo-pyrido[2,3-d]pyrimidin-8-yl]methyl]-1,3-dioxan-5-yl]carbamate was taken up in 1 mL DCM and TFA (1 mL) was added. After stirring at RT for 1 h and concentration, the residue was taken up in minimal DMF and purified by reverse phase HPLC. The product 2-amino-8-[(5-amino-1,3-dioxan-2-yl)methyl]-6-[2-chloro-4-(6-methylpyrazin-2-yl)phenyl]pyrido[2,3-d]pyrimidin-7-one (14 mg, 12% yield over 2 steps) was isolated as a mixture of diastereomers. ¹H NMR (400 MHz, DMSO) δ 9.14 (s, 1H), 8.64 (s, 1H), 8.59 (d, J=17.3 Hz, 1H), 8.26 (d, J=1.6 Hz, 1H), 8.15 (dd, J=8.0, 1.5 Hz, 1H), 7.81 (d, J=17.0 Hz, 1H), 7.56 (d, J=8.0 Hz, 1H), 7.37 (s, 2H), 5.07 (t, J=5.3 Hz, 1H), 5.01-4.88 (m, 1H), 4.46 (dd, J=10.8, 5.5 Hz, 2H), 3.91 (dd, J=10.9, 4.9 Hz, 1H), 3.75 (dd, J=40.7, 11.1 Hz, 3H), 3.11 (t, J=10.8 Hz, 1H), 2.60 (s, 3H), 2.57 (s, 1H); LCMS (ESI): (M+H⁺) m/z=480.2.

### Example 88

Pharmaceutical compositions of the subject Compounds for administration via several routes can be prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound of formula I wherein: 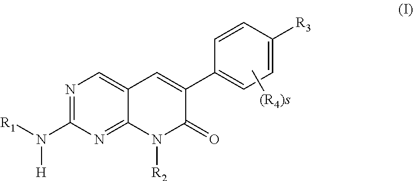
R¹ is H, C₁₋₃ alkyl, C₁₋₃ fluoroalkyl, oxetan-3-yl or 3-methyl-oxetan-3-yl;
R² is (i) C₂₋₆ hydroxyalkyl or C₂₋₆ dihydroxyalkyl,
(ii) (alkylene)ₙNR^{a}R^{b} wherein the alkylene chain is optionally substituted by a hydroxyl,
(iii) (alkylene)₁₋₃OR⁵ wherein R⁵ is (alkylene)₂₋₄NR^{a}R^{b}, (alkylene)₂₋₄OR^{f} or a heterocycle selected from azetidine, pyrrolidine, piperidine or azepine said heterocycle optionally substituted by C₁₋₆ alkyl,
(iv) (alkylene)ᵣC(O)NR^{a}R^{b},
(v) (alkylene)ₙ-CN,
(vi) (alkylene)ₜ-(C₄₋₆-cycloalkyl NR^{c}R^{d}),
(vii) heterocyclyl-(alkylene)ₙ wherein heterocyclyl refers to substituted azetidinyl, pyrrolidinyl, 4,4-difluoro-pyrrolidin-3-yl, piperidinyl, 3,3-difluoro-piperidin-3-yl, azepinyl, morpholinyl, 4-methylmorpholin-2-yl, 5-amino-1,3-dioxolan-2-yl, 5-methylamino-1,3-dioxolan-2-yl, 3-aza-bicyclo[3.1.0]hexan-6-yl, 5-oxa-2-azaspiro[3.4]octan-7-yl, 1-oxa-8-azaspiro[4.5]decan-3-yl, 1-oxa-7-azaspiro[4.4]nonan-3-yl, 5,9-dioxa-2-azaspiro[3.5]nonan-7-yl, 1,1-dioxo-4-thiazinan-2-yl or piperazinyl said heterocyclyl moiety optionally substituted by C(═O)CHR^{f}NH₂, oxo, hydroxyl, amino, C₁₋₃ alkylamino, C₁₋₃ dialkylamino, cyano, oxetan-3-yl or C₁₋₆-hydroxyalkyl, or
(viii) (alkylene)ᵣS(O)₂R⁵ wherein R⁵ is (alkylene)₀₋₃NR^{a}R^{b} azetidinyl, pyrrolidinyl or piperidinyl; or,
(ix) 1-imino-1-oxa-thianyl-4-yl or 1,1-dioxidotetrahydro-2H-thiopyran-4-yl;
R^{a} and R^{b} are (a) independently in each occurrence hydrogen, C₁₋₃ alkyl, C₂₋₄ aminoalkyl, C₂₋₄-hydroxyalkyl, pyrrolidinyl, piperidinyl or azetidinyl, or, (b) R^{a} and R^{b} together with the nitrogen to which they are attached form a four to seven-membered mono- or bicyclic-ring optionally containing another heteroatom selected from O, NR^{e} or S(O)₀₋₂ which is optionally substituted by one or two hydroxyl or (CH₂)₀₋₂N(R^{f})₂;
R^{c} and R^{d} are independently in each occurrence hydrogen, C₁₋₃ alkyl or oxetanyl;
R^{e} is hydrogen, C₁₋₃ alkyl or C₁₋₃ alkylsulfonyl;
R^{f} is hydrogen or C₁₋₃ alkyl;
R³ is heteroaryl wherein the heteroaryl ring is pyridin-2-yl, pyrazin-2-yl, thiazol-4-yl, thiazol-2-yl, pyrazol-3-yl, 1,2,4-triazol-2-yl or 1,2,4-oxadiazol-3-yl each optionally substituted by an R⁴ or a C₁₋₃ hydroxyalkyl moiety;
R⁴ is C₁₋₆ alkyl, cyclopropyl or halogen;
n is 2-6;
r is 1-6;
s is 0, 1 or 2;
t is 0-6; or,
a pharmaceutically acceptable salt thereof, with the proviso that the compound of formula I is not:
8-(trans-4-aminocyclohexyl)-6-[2-chloro-4-(6-methyl-2-pyrazinyl)phenyl]-2-(ethylamino)-pyrido[2,3-d]pyrimidin-7(8H)-one,
8-(trans-4-aminocyclohexyl)-6-[2-chloro-4-(6-methyl-2-pyrazinyl)phenyl]-2-[(1-methylethyl)amino]-pyrido[2,3-d]pyrimidin-7(8H)-one,
6-(2-chloro-4-(5-methyl-1,2,4-oxadiazol-3-yl)phenyl)-2-(ethylamino)-8-(piperidin-4-ylmethyl)pyrido[2,3-d]pyrimidin-7(8H)-one, or,
6-(2-chloro-4-(5-methyl-1,2,4-oxadiazol-3-yl)phenyl)-2-(isopropylamino)-8-(piperidin-4-ylmethyl)pyrido[2,3-d]pyrimidin-7(8H)-one.

2. A compound according to claim 1 wherein R² is (CH₂)ₙNR^{a}R^{b}.

3. A compound according to claim 1 wherein R² is (alkylene)₁₋₃OR⁵ wherein R⁵ is (alkylene)²⁻⁴NR^{a}R^{b} or optionally substituted heterocycle selected from azetidine, pyrrolidine, piperidine or azepine.

4. A compound according to claim 1 wherein R² is (CH₂)ᵣC(O)NR^{a}R^{b}.

5. A compound according to claim 1 wherein R² is:

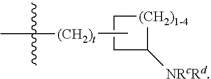

6. A compound according to claim 1 wherein R² is selected from (i) to (viii):

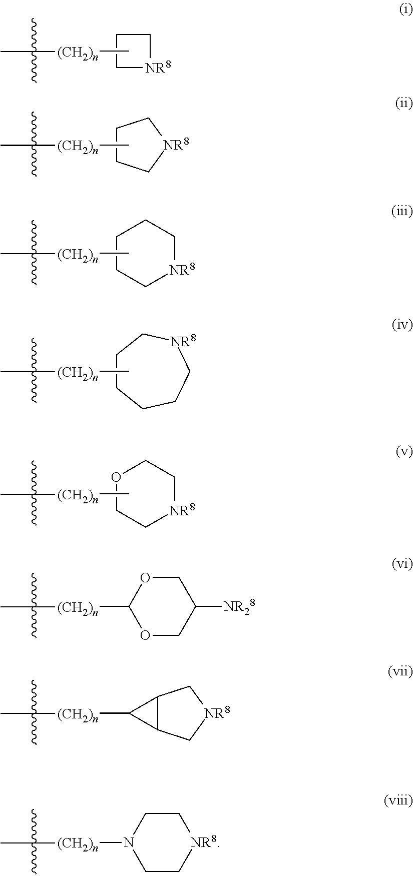

7. A compound according to claim 6 wherein R² is 2-morpholin-2-ylethyl or 2-morpholin-2-ylmethyl.

8. A compound according to claim 6 wherein R² is 2-(5-amino-1,3-dioxan-2-yl)ethyl or 2-(5-amino-1,3-dioxan-2-yl)methyl.

9. A compound according to claim 6 wherein R² is (3 3-difluoro-4-piperidyl)methyl or (3 3-difluoro-4-piperidyl)ethyl.

10. A compound according one of claims 2 to 9 wherein R³ is optionally substituted pyrazinyl and R⁴ is 2-chloro or 2-methyl.

11. A compound according one of claims 2 to 9 wherein R³ is optionally substituted pyridinyl and R⁴ is 2-chloro or 2-methyl.

12. A compound according to claim 1 selected from compounds I-1 to I-202 in Table I.

13. A method of inhibiting PAK activity in a patient in need thereof comprising the step of administering to said patient a compound according to claim 1.

14. A method of treating or ameliorating the severity of cancer or a hyperproliferative disorder in a patient in need thereof comprising administering to said patient a compound according to claim 1.

15. The method according to claim 11 wherein said cancer or hyperproliferative disorder is selected from the group consisting of adenoma, bladder cancer, brain cancer, breast cancer, colon cancer, epidermal carcinoma, follicular carcinoma, cancer of the genitourinary tract, glioblastoma, Hodgkin's disease, head and neck cancers, heptoma, keratoacanthoma, kidney cancer, large cell carcinoma, leukemias, lung adenocarcinoma, lung cancer, lymphoid disorders, melanoma and non-melanoma skin cancer, myelodysplastic syndrome, neuroblastoma, non-Hodgkins lymphoma, ovarian cancer, papillary carcinoma, pancreatic cancer, prostate cancer, rectal cancer, sarcoma, small cell carcinoma, testicular cancer, tetracarcinomas, thyroid cancer, and undifferentiated carcinoma.

16. The method according to claim 12 wherein said cancer or hyperproliferative disorder is selected from the group consisting of lung cancer, breast cancer, ovarian cancer, bladder cancer and head and neck cancer.

17. The method according to claim 12 wherein said cancer or hyperproliferative disorder is selected from the group consisting primary breast adenocarcinoma, squamous non-small cell lung cancer or a squamous head and neck cancer.

18. The method according to claim 11 wherein a compound of claim one is co-administered with at least one other chemotherapeutic agent used to treat or ameliorate cancer or a hyperproliferative disorder.

19. The method of claim 15 wherein the other chemotherapeutic agent is selected from the group consisting of inhibitor of apoptosis proteins (IAP), an EGFR inhibitor or antagonist, an inhibitor of Ras/Raf/Mek/Erk signaling cascade, an inhibitor of Akt kinase and a Src kinase inhibitor.

20. A composition comprising a compound according to claim 1 and at least one pharmaceutically acceptable carrier, excipient or diluent.

21. A p21-activated kinase (PAK) inhibitor according to claim 1 for use in treating cancer or a hyperproliferative disorder.

22. The use of a compound of claim 1 in the manufacture of a medicament for the inhibition of abnormal cell growth or for treatment of a hyperproliferative disorder in a mammal.
